(19) United States
(12) Patent Application Publication
(10) Pub. No.: US 2023/0392173 A1
(43) Pub. Date: Dec. 7, 2023

(54) ENGINEERED BIOSYNTHETIC PATHWAY FOR PRODUCTION OF 4-AMINOPHENYLETHYLAMINE BY FERMENTATION

(71) Applicant: Zymergen Inc., Emeryville, CA (US)

(72) Inventors: Zhangying Hao, Mountain View, CA (US); Shuchi Desai, Union City, CA (US); Steven M. Edgar, Albany, CA (US); Joseph Gallagher, Berkeley, CA (US); James Chandler, Albany, CA (US); Eyal Akiva, Emeryville, CA (US); Rebecca Davidson, Emeryville, CA (US); Matthew Davis, Berkeley, CA (US)

(73) Assignee: Zymergen Inc., Emeryville, CA (US)

(21) Appl. No.: 18/021,788

(22) PCT Filed: Aug. 19, 2021
(86) PCT No.: PCT/US2021/046756

§ 371 Date: Feb. 16, 2023

Related U.S. Application Data
(60) Provisional application No. 63/068,323, filed on Aug. 20, 2020.

(51) Int. Cl. C12P 13/00; C12N 15/81; C12N 9/10; C12N 9/90; C12N 9/02; C12N 9/88; C12N 1/16

(52) U.S. Cl. CPC C12P 13/001; C12N 15/815; C12N 9/1096; C12N 9/90; C12N 9/001; C12N 9/88; C12N 1/16; C12Y 206/01085; C12Y 504/99; C12Y 103/01; C12Y 401/01

(57) ABSTRACT

The present disclosure describes the engineering of microbial cells for fermentative production of 4-APEA and related products and provides novel engineered microbial cells and cultures, as well as related 4-APEA production methods.

### CROSS-REFERENCE TO RELATED APPLICATIONS

This application claims the benefit of U.S. provisional application No. 63/068,323, filed Aug. 20, 2020, which is hereby incorporated by reference in its entirety.

### STATEMENT AS TO RIGHTS TO INVENTIONS MADE UNDER FEDERALLY SPONSORED RESEARCH AND DEVELOPMENT

Not applicable.

### INCORPORATION BY REFERENCE OF THE SEQUENCE LISTING

This application includes a sequence listing which has been submitted electronically in ASCII format and is hereby incorporated by reference in its entirety. This ASCII copy, created on Aug. 19, 2021, is named ZMGNP043WO_SeqList_ST25.txt and is 448,733 bytes in size.

### FIELD OF THE DISCLOSURE

The present disclosure relates generally to the area of engineering microbes for production of 4-aminophenylethylamine by fermentation.

### BACKGROUND

4-aminophenylethylamine (4-APEA) is an aromatic amine (AA). AAs are used in the production of advanced polymer materials including functional and/or high-performance plastics. The amine group and the aromatic moiety of AAs provide nucleophilic reactivity and excellent thermomechanical performance, respectively. AAs are often polycondensed with carbonyl compounds to generate aromatic polyamides, polyimides, polyazoles, polyurea, and polyazomethines. Polycondensation of AAs with aromatic acids generates super-engineering plastics with extremely high thermomechanical properties. These include poly(p-phenylene terephthalamide (KEVLAR™) and poly(4,4′-oxydiphenylene pyromellitimide) (KAPTON™), which are used as thermostable materials in fabric for body armor and other flame-retardant materials, fiber-reinforced plastics for electronic devices, vehicle bodies, and anti-pressure cylinders.

Fermentative production of 4-APEA has been demonstrated in Escherichia coli (Masuo et al. (2016) Scientific Reports 6: 25764), but the poor tolerance of E. coli to high titers of 4-APEA makes it unsuitable for a large-scale fermentation host.

### SUMMARY

The disclosure provides engineered microbial cells, cultures of the microbial cells, and methods for producing 4-aminophenylethylamine (4-APEA), including the following:

Various embodiments contemplated herein may include, but need not be limited to, one or more of the following:

Embodiment 1: An engineered microbial cell that produces 4-aminophenylethylamine (4-APEA), wherein the engineered microbial cell has a high tolerance to toxicity associated with the production of 4-APEA, as defined by a concentration at which the growth of engineered microbial cell is slowed by half (Ki) of at least 30 grams/liter.

Embodiment 2: The engineered microbial cell of embodiment 1, wherein the engineered microbial cell comprises a fungal cell.

Embodiment 3: The engineered microbial cell of embodiment 2, wherein the engineered microbial cell comprises a yeast cell.

Embodiment 4: The engineered microbial cell of embodiment 3, wherein the yeast cell is a cell of the genus Komagataella.

Embodiment 5: The engineered microbial cell of embodiment 4, wherein the yeast cell is a cell of the species pastoris or phaffi.

Embodiment 6: The engineered microbial cell of any one of embodiments 1-5, wherein the slope at which toxicity effects are observed over increasing 4-APEA concentrations is less than 6.

Embodiment 7: The engineered microbial cell of any one of embodiments 1-6, wherein the engineered microbial cell heterologously expresses each of the following enzyme activities: 4-amino-4-deoxychorismate synthase; 4-amino-4-deoxychorismate mutase; 4-amino-4-deoxyprephenate dehydrogenase; aminotransferase (AT); and decarboxylase (DC); wherein the enzyme activities are provided by heterologously expressing genes encoding the enzymes, and at least one heterologously expressed enzyme is non-native to the engineered microbial cell.

Embodiment 8: The engineered microbial cell of embodiment 7, wherein at least two, three, four, or all of the heterologously expressed enzymes are non-native to the engineered microbial cell.

Embodiment 9: An engineered microbial cell of the genus Komagataella that produces 4-aminophenylpyruvate (4-APP).

Embodiment 10: The engineered microbial cell of embodiment 9, wherein the engineered microbial cell is a cell of the species pastoris or phaffi.

Embodiment 11: The engineered microbial cell of embodiment 9 or embodiment 10, wherein the engineered microbial cell heterologously expresses each of the following enzyme activities: 4-amino-4-deoxychorismate synthase; 4-amino-4-deoxychorismate mutase; and 4-amino-4-deoxyprephenate dehydrogenase, wherein each enzyme activity is provided by heterologously expressing genes encoding the enzymes, and at least one heterologously expressed enzyme is non-native to the engineered microbial cell.

Embodiment 12: The engineered microbial cell of any one of embodiments 9-11, wherein the engineered microbial cell additionally produces 4-aminophenylalanine (4-APhe).

Embodiment 13: The engineered microbial cell of embodiment 12, wherein the engineered microbial cell additionally heterologously expresses an aminotransferase (AT) activity.

Embodiment 14: The engineered microbial cell of any one of embodiments 9-11, wherein the engineered microbial cell additionally produces 4-aminophenylethanol.

Embodiment 15: The engineered microbial cell of embodiment 14, wherein the engineered microbial cell additionally heterologously expresses an alcohol dehydrogenase/acetaldehyde reductase enzyme.

Embodiment 16: The engineered microbial cell of any one of embodiments 9-15, wherein at least two, three, or all of the heterologously expressed enzymes are non-native to the engineered microbial cell.

Embodiment 17: The engineered microbial cell of any one of embodiments 7-16, wherein at least one of the heterologously expressed enzymes is expressed from a constitutive promoter.

Embodiment 18: The engineered microbial cell of any one of embodiments 7-16, wherein at least one of the heterologously expressed enzymes is expressed from a regulated promoter, optionally wherein the regulated promoter is a thiamine-repressed promoter.

Embodiment 19: The engineered microbial cell of any one of embodiments 1-18, wherein the engineered microbial cell comprises increased activity of one or more upstream chorismate pathway enzyme(s), said increased activity being increased relative to a control cell.

Embodiment 20: The engineered microbial cell of embodiment 19, wherein said increased activity is selected from the group consisting of glucokinase, transketolase, transaldolase, phospho-2-dehydro-3-deoxyheptonate aldolase, 3-deoxy-D-arabino-heptulosonate-7-phosphate (DAHP) synthase, 3-dehydroquinate synthase, 3-dehydroquinate dehydratase, shikimate dehydrogenase, shikimate kinase, 3-phosphoshikimate 1-carboxyvinyltransferase, chorismate synthase activity, and any combination thereof.

Embodiment 21: The engineered microbial cell of embodiment 20, wherein said increased activity comprises increased 3-dehydroquinate synthase, 3-dehydroquinate dehydratase, shikimate dehydrogenase, shikimate kinase, and 3-phosphoshikimate 1-carboxyvinyltransferase activities, which are provided by heterologously expressing a pentafunctional enzyme.

Embodiment 22: The engineered microbial cell of any one of embodiments 1-21, wherein the engineered microbial cell comprises increased activity of one or more nitrogen assimilation and utilization pathway enzyme(s).

Embodiment 23: The engineered microbial cell of 22, wherein said increased activity is selected from the group consisting of isocitrate dehydrogenase, glutamine synthetase, glutamate synthase, glutamate dehydrogenase, ammonium permease, and any combination thereof.

Embodiment 24: The engineered microbial cell of any one of embodiments 1-23, wherein the engineered microbial cell comprises reduced activity of one or more enzyme(s) that consume one or more chorismate pathway precursors, chorismate, and/or one or more intermediates in the pathway leading from chorismate to 4-APEA, and/or more enzymes that consume 4-APEA, said reduced activity being reduced relative to a control cell.

Embodiment 25: The engineered microbial cell of embodiment 24, wherein the one or more enzyme(s) that consume one or more chorismate pathway precursors are selected from the group consisting of dihydroxyacetone phosphatase, 3-dehydroshikimate dehydratase, shikimate dehydrogenase, and phosphoenolpyruvate phosphotransferase.

Embodiment 26: The engineered microbial cell of embodiment 24, wherein the one or more enzyme(s) that consume chorismate are selected from the group consisting of anthranilate synthase and chorismate mutase.

Embodiment 27: The engineered microbial cell of embodiment 24, wherein the one or more enzyme(s) that consume one or more intermediates in the pathway leading from chorismate to 4-APEA are selected from the group consisting of decarboxylase, aromatic amino acid decarboxylase, phenylpyruvate decarboxylase, pyruvate decarboxylase, aromatic amino acid ammonia lyase, and alcohol dehydrogenase/acetaldehyde reductase.

Embodiment 28: The engineered microbial cell of embodiment 24, wherein the one or more enzymes that consume 4-APEA are selected from the group consisting of phenylpyruvate dioxygenase, diamine oxidase, amine oxidase, and amino acid oxidase.

Embodiment 29: The engineered microbial cell of any one of embodiments 24-28, wherein the reduced activity is achieved by replacing a native promoter of a gene for said one or more enzymes with a less active promoter or by deleting or knocking out the gene.

Embodiment 30: The engineered microbial cell of any one of embodiments 1-29, wherein the engineered microbial cell additionally expresses a feedback-deregulated DAHP synthase.

Embodiment 31: The engineered microbial cell of any one of embodiments 1-30, wherein the engineered microbial cell comprises increased activity of one or more enzyme(s) that increase the supply of the reduced form of nicotinamide adenine dinucleotide phosphate (NADPH), said increased activity being increased relative to a control cell.

Embodiment 32: The engineered microbial cell of embodiment 31, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

Embodiment 33: The engineered microbial cell of embodiment 1, wherein the non-native enzymes comprise: a 4-amino-4-deoxychorismate synthase having at least 70% amino acid sequence identity with a 4-amino-4-deoxychorismate synthase from Pseudomonas fluorescens (strain SBW25); a 4-amino-4-deoxychorismate mutase having at least 70% amino acid sequence identity with a 4-amino-4-deoxychorismate mutase from Photorhabdus laumondii subsp. laumondii (strain DSM 15139/CIP 105565/TT01); a 4-amino-4-deoxyprephenate dehydrogenase having at least 70% amino acid sequence identity with a 4-amino-4-deoxyprephenate dehydrogenase from Pseudomonas fluorescens (strain SBW25); optionally, an aminotransferase (AT) having at least 70% amino acid sequence identity with an aminotransferase (AT) from Escherichia coli (strain K12); and optionally a decarboxylase (DC) having at least 70% amino acid sequence identity with a decarboxylase (DC) from Papaver somniferum.

Embodiment 34: The engineered microbial cell of embodiment 33, wherein the: 4-amino-4-deoxychorismate synthase from Pseudomonas fluorescens (strain SBW25) comprises SEQ ID NO:4; 4-amino-4-deoxychorismate mutase from Photorhabdus laumondii subsp. laumondii (strain DSM 15139/CIP 105565/TT01) comprises SEQ ID NO:6; 4-amino-4-deoxyprephenate dehydrogenase from Pseudomonas fluorescens (strain SBW25) comprises SEQ ID NO:8; aminotransferase (AT) from Escherichia coli (strain K12), if present, comprises SEQ ID NO:(SEQ ID NO:13); and decarboxylase (DC) from Papaver somniferum, if present, comprises SEQ ID NO:9.

Embodiment 35: The engineered microbial cell of embodiment 1, wherein the non-native enzymes comprise: a 4-amino-4-deoxychorismate synthase having at least 70% amino acid sequence identity with a 4-amino-4-deoxychorismate synthase from Streptomyces sp. CB01635; a 4-amino-4-deoxychorismate mutase having at least 70% amino acid sequence identity with a 4-amino-4-deoxychorismate mutase from Streptomyces pristinaespiralis; a 4-amino-4-deoxyprephenate dehydrogenase having at least 70% amino acid sequence identity with a 4-amino-4-deoxyprephenate dehydrogenase from Pseudomonas sp. 2822; optionally, an aminotransferase (AT) having at least 70% amino acid sequence identity with an aminotransferase (AT) from Petunia hybrida; and optionally, a decarboxylase (DC) having at least 70% amino acid sequence identity with a decarboxylase (DC) from Papaver somniferum.

Embodiment 36: The engineered microbial cell of embodiment 35, wherein the: 4-amino-4-deoxychorismate synthase from Streptomyces sp. CB01635 comprises SEQ ID NO:3; 4-amino-4-deoxychorismate mutase from Streptomyces pristinaespiralis comprises SEQ ID NO:5; 4-amino-4-deoxyprephenate dehydrogenase from Pseudomonas sp. 2822 comprises SEQ ID NO:7; aminotransferase (AT) from Petunia hybrida, if present, comprises SEQ ID NO:14; and decarboxylase (DC) from Papaver somnferum, if present, comprises SEQ ID NO:9.

Embodiment 37: The engineered microbial cell of embodiment 1, wherein the non-native enzymes comprise: a 4-amino-4-deoxychorismate synthase having at least 70% amino acid sequence identity with a 4-amino-4-deoxychorismate synthase from Streptomyces sp. CB01635; a 4-amino-4-deoxychorismate mutase having at least 70% amino acid sequence identity with a 4-amino-4-deoxychorismate mutase from Photorhabdus asymbiotica subsp. asymbiotica; a 4-amino-4-deoxyprephenate dehydrogenase having at least 70% amino acid sequence identity with a 4-amino-4-deoxyprephenate dehydrogenase from Xenorhabdus doucetiae; optionally, an aminotransferase (AT) having at least 70% amino acid sequence identity with an aminotransferase (AT) from Corynebacterium glutamicum; and optionally, a decarboxylase (DC) having at least 70% amino acid sequence identity with a decarboxylase (DC) from Papaver somnferum.

Embodiment 38: The engineered microbial cell of embodiment 35, wherein the: 4-amino-4-deoxychorismate synthase from Streptomyces sp. CB01635 comprises SEQ ID NO:3; 4-amino-4-deoxychorismate mutase from Photorhabdus asymbiotica subsp. asymbiotica comprises SEQ ID NO:25; 4-amino-4-deoxyprephenate dehydrogenase from Xenorhabdus doucetiae comprises SEQ ID NO:29; aminotransferase (AT) from Corynebacterium glutamicum, if present, comprises SEQ ID NO:16; and decarboxylase (DC) from Papaver somnferum, if present, comprises SEQ ID NO:9.

Embodiment 39: The engineered microbial cell of any one of embodiments 7-38, wherein the engineered microbial cell additionally comprises a genotype change selected from the group consisting of: p9_pENO1_KPA:GLN1, p35_pKEX2_KPA:NUFM, p9_pENO1_KPA:NUFM, p115_pTHI11_KPA:PDC2, and p5_pTDH3_KPA:PDC2.

Embodiment 40: The engineered microbial cell of any one of embodiments 1-8 and 19-39, wherein, when cultured, the engineered microbial cell produces 4-APEA at a level of at least 11 gram/liter of culture medium.

Embodiment 41: The engineered microbial cell of any one of embodiments 9-16, wherein, when cultured, the engineered microbial cell produces 4-APP at a level of at least 20 milligram/liter of culture medium, optionally wherein, when cultured, the engineered microbial cell produces 4-APhe at a level of at least 5 milligram/liter of culture medium.

Embodiment 42: A culture of engineered microbial cells according to any one of embodiments 1-41.

Embodiment 43: The culture of embodiment 42, wherein the culture comprises 4-APP, 4-A-Phe, and/or 4-APEA.

Embodiment 44: A method of culturing engineered microbial cells according to any one of embodiments 1-41, the method comprising culturing the cells under conditions suitable for producing 4-APP, 4-APhe, and/or 4-APEA.

Embodiment 45: The method of embodiment 44, wherein the method comprises fed-batch culture, with an initial glucose level in the range of 1-100 g/L, followed by controlled sugar feeding.

Embodiment 46: The method of any one of embodiment 44 or embodiment 45, wherein the culture is pH-controlled during culturing.

Embodiment 47: The method of any one of embodiments 44-46, therein the concentration of thiamine is controlled during culturing.

Embodiment 48: The method of any one of embodiments 44-47, wherein the culture is aerated during culturing.

Embodiment 49: The culture of embodiment 42 or embodiment 43 or the method of any one of embodiments 44-48, wherein the culture comprises: 4-APP at a level of at least 20 milligram/liter of culture medium; 4-APhe at a level of at least 5 milligram/liter of culture medium; and/or 4-APEA at a level of at least 15 milligram/liter of culture medium.

Embodiment 50: The engineered microbial cell of embodiment 40 or the culture or method of embodiment 49, wherein, when cultured, the engineered microbial cell, or the culture comprises, 4-APEA at a level of at least 6 gram/liter of culture medium.

Embodiment 51: The engineered microbial cell of embodiment 40 or the culture or method of embodiment 49, wherein, when cultured, the engineered microbial cell, or the culture comprises, 4-APEA at a level of at least 11 gram/liter of culture medium.

Embodiment 52: The method of any one of embodiments 44-51, wherein the method additionally comprises recovering 4-APP, 4-APhe, and/or 4-APEA from the culture.

The disclosure also provides versions of the above embodiments wherein the embodiments consists of or consists essentially of the recited elements or actions. In the case of embodiments consisting essentially of the recited elements/actions, the “basic and novel characteristics” of the embodiment are the production characteristics of an engineered microbial cell, culture, or method (e.g., the yield of a particular product, optionally expressed in terms of titer in culture medium).

Also within the scope of the present disclosure are versions of the above embodiments wherein the embodiments are carried out using any means for providing the recited elements of composition claims and any means for carrying out the recited actions of the method claims.

### BRIEF DESCRIPTION OF THE DRAWINGS

FIG. 1: Toxicity impact of 4-APEA across a range of organisms tested in an Enzyscreen system. Various organisms are grown in media that is supplemented with the molecule of interest, and the growth of the organism is monitored over time. Analysis of the growth-time-course data allows determination of toxicity properties of the molecule of interest, such as the Minimal Inhibitory Concentration (MIC), the concentration at which growth is slowed to half (Ki), and the slope at which increased toxicity effects are observed (alpha). For FIG. 1, product toxicity was measured as a function of reduced substrate uptake with increasing product concentration.

FIG. 2: Biosynthesis of 4-APEA in five enzymatic steps from chorismate.

FIG. 3: Pathway for production of chorismate.

FIG. 4: Product profile of a 4-APEA production strain cultured in microtiter plates for 48 hours. See Example 1.

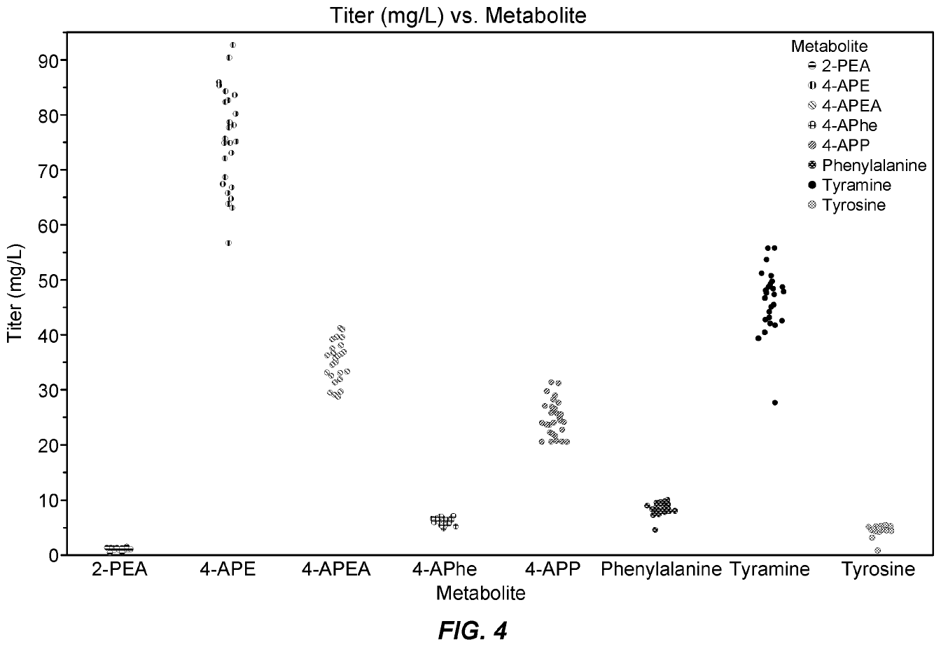

FIG. 5: A “split-marker, double-crossover” genomic integration strategy, which was developed to engineer K. pastoris strains. Two plasmids with complementary 5′ and 3′ homology arms and overlapping halves of a selectable marker, such as an antibiotic marker or an auxotrophic marker (direct repeats shown by the hashed bars) were linearized by PCR or by digestion with meganucleases and transformed as linear fragments. A triple-crossover event integrated the desired heterologous genes into the targeted locus and re-constituted the full selectable marker gene. Colonies derived from this integration event were assayed using two 3-primer reactions to confirm both the 5′ and 3′ junctions (UF/IF/wt-R and DR/IF/wt-F).

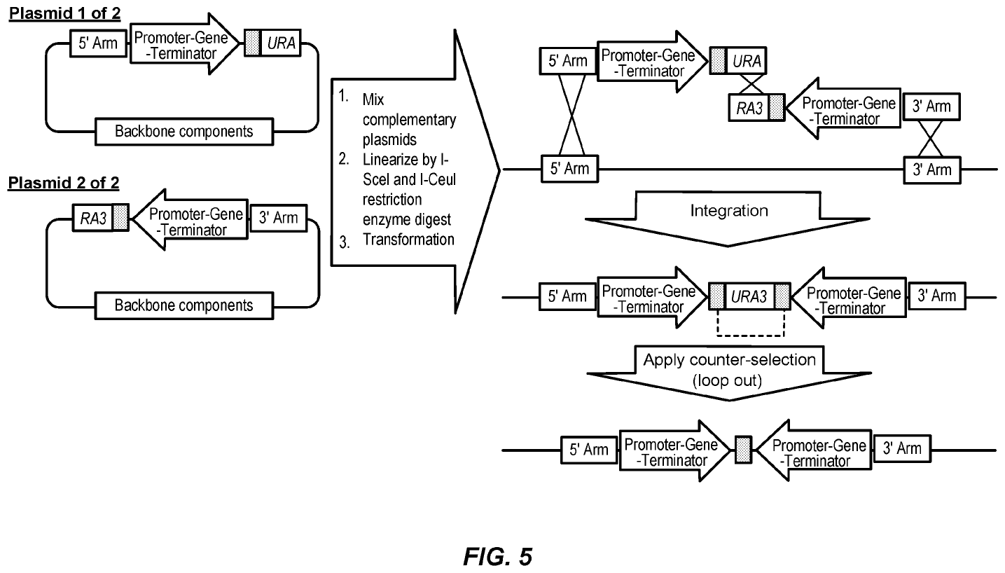

### DETAILED DESCRIPTION

The present disclosure describes the engineering of microbial cells for fermentative production of 4-aminophenylethylamine (4-APEA) and provides novel engineered microbial cells and cultures, as well as related 4-APEA production methods.

### Definitions

Terms used in the claims and specification are defined as set forth below unless otherwise specified.

The term “fermentation” is used herein to refer to a process whereby a microbial cell converts one or more substrate(s) into a desired product (such as 4-APEA) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and/or inserted into a different genomic location than that of the native sequence). “Heterologous expression” thus encompasses expression of a sequence that is non-native to the host cell, as well as expression of a sequence that is native to the host cell in a non-native context.

“Heterologous expression” encompasses expressing a polynucleotide from a constitutive promoter or from a regulated promoter.

A “regulated promoter” is a promoter that is more or less active in response to one or more parameters. For example, a thiamine-repressed promoter is one whose activity increases in response to a reduction in thiamine concentration. Illustrative thiamine-repressed promoters are typically repressed at thiamine concentrations of 50 mg/L and above, with the degree of promoter repression decreasing as the thiamine concentration approaches zero.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell. The control cell can include one or more specific modifications that are also present in the engineered cell being tested (i.e., genetic modifications that are not “being tested”).

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., 4-APEA) present in the cell culture medium in a culture of microbial cells divided by the culture volume.

The term “Ki,” as used herein, refers to the concentration of a chemical (e.g., 4-APEA) at which the maximal growth rate of cells in culture is slowed by half. Cultures of the cells are grown in media supplemented with varying concentrations of a chemical, and the cell growth is monitored over time. Exponential growth curve data at each chemical concentration are used to determine a specific growth rate p using the following equation:

X=X₀e^{ut}

where:
X=cell concentration
X₀=cell concentration at time (t)=0
μ=specific growth rate.
The maximal growth rate for each chemical concentration is determined (μ_{obs}) and the Ki is the chemical concentration at which the maximal growth rate has been slowed to half of the maximal growth rate in the absence of the chemical (μₘₐₓ). Ki (denoted K₁ below) can be determined from the equation:

$${μ}_{obs} = \frac{{μ}_{max}}{1 + {( \frac{{C}_{I}}{{K}_{I}} )}^{a}}$$

where:
C_{I}=concentration of inhibitor
K_{I}=inhibition constant—inhibitor concentration where growth rate is half of maximum
α=inhibition parameter to fit data
μₘₐₓ=the maximum growth rate of a culture in the absence of a chemical
μ_{obs}=the maximum growth rate of a culture at a given concentration of a chemical

The term “alpha” (“α”), as used herein, refers to the slope at which toxicity effects are observed. Cultures of the cells are grown in media supplemented with increasing concentrations of a chemical, and the cell growth is monitored over time. Alpha can be determined from the equation:

$${μ}_{obs} = \frac{{μ}_{max}}{1 + {( \frac{{C}_{I}}{{K}_{I}} )}^{a}}$$

where:
C_{I}=concentration of inhibitor
K_{I}=inhibition constant—inhibitor concentration where growth rate is half of maximum
α=inhibition parameter to fit data
μₘₐₓ=the maximum growth rate of a culture in the absence of a chemical
μ_{obs}=the maximum growth rate of a culture at a given concentration of a chemical

As used herein, the term “4-APEA pathway gene” refers to any gene encoding an enzyme that participates in the conversion of chorismate to 4-APEA, e.g., any one of the following enzymes: 4-amino-4-deoxychorismate synthase, 4-amino-4-deoxychorismate mutase, 4-amino-4-deoxyprephenate dehydrogenase, aminotransferase (AT), and decarboxylase (DC).

As used herein, the term “upstream chorismate pathway enzyme” refers to any enzyme that participates in the conversion of glucose to chorismate.

As used herein with respect to recovering 4-APEA from a cell culture, “recovering” refers to separating the 4-APEA from at least one other component of the cell culture medium.

### Engineering Microbes for Production of 4-Aminophenylethylamine and Precursors or Derivatives Thereof

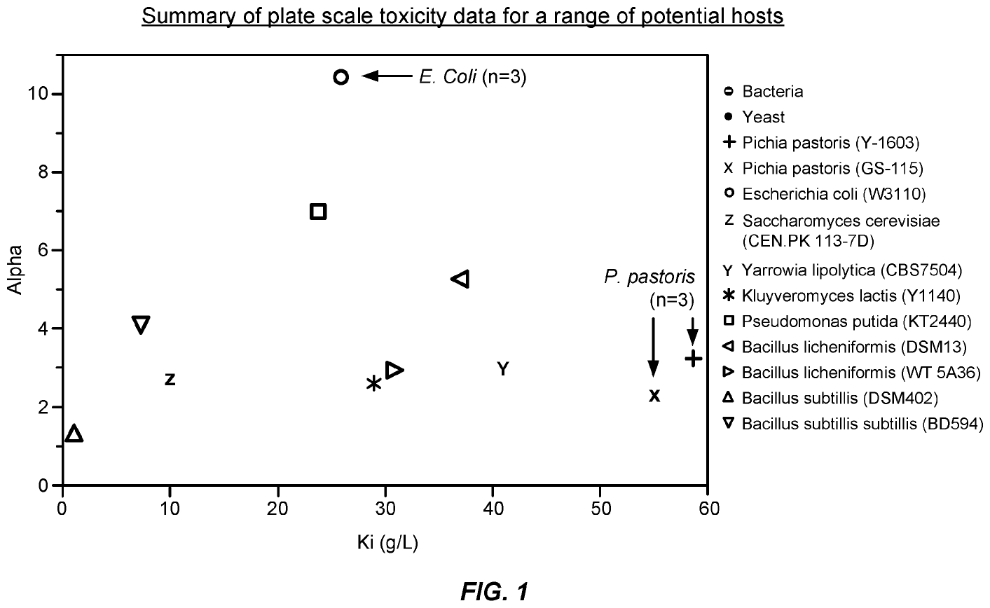

One obstacle to efficient production of 4-aminophenylethylamine (4-APEA) by fermentation is that 4-APEA is toxic to many host microbes used conventionally for fermentation. FIG. 1 depicts the concentration of 4-APEA at which an organism's growth is slowed by half (Ki) and the slope (alpha) at which toxicity effects are observed over increasing 4-APEA concentrations. A larger Ki and smaller alpha indicate higher tolerance and less susceptibility to inhibition, respectively. FIG. 1 shows that some species, such as Saccharomyces cerevisiae and Escherichia coli, suffer from significant toxicity in the presence of higher levels of 4APEA. Other fungi, including the yeasts Komagataella pastoris (also known as Pichia pastoris), Komagataella phaffi, and Yarrowia lipolytica, and other bacteria, such as Bacillus licheniformis provide better results. In particular, the substantial toxicity of 4-APEA on traditional metabolic engineering hosts such as E. coli and S. cerevisiae, shown in FIG. 1, in comparison to the more moderate effect on K. pastoris highlights the utility of K. pastoris as a production host for high-titer fermentation of 4-APEA. K. phaffi is expected to perform similarly to K. pastoris as a production host.

### 4-Aminophenylethylamine Biosynthesis Pathway

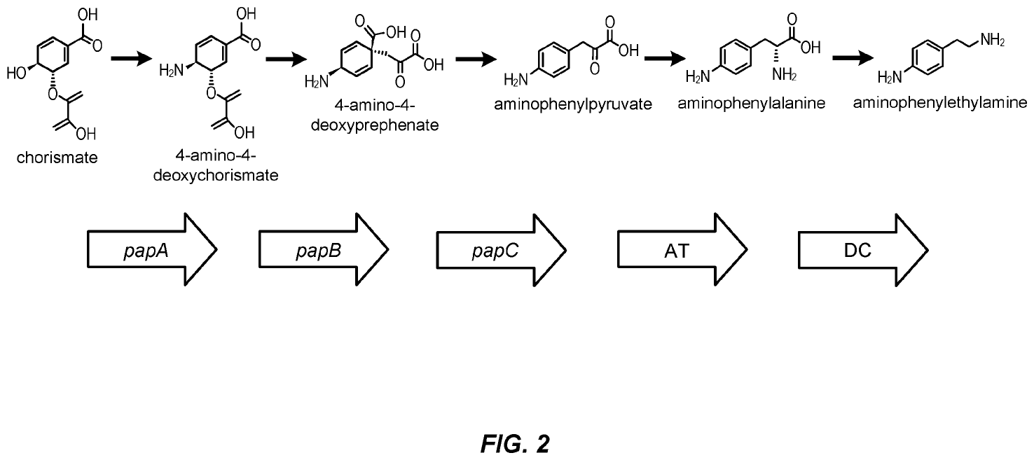

The metabolic pathway to 4-APEA is derived from the shikimate pathway metabolite, chorismate. FIG. 2 depicts an assembled pathway for biosynthesis of 4-APEA from chorismate. This pathway is sequentially made up of the following enzyme activities: 4-amino-4-deoxychorismate synthase (e.g., encoded by a papA gene in some organisms and referred to herein as “papA,” for short), 4-amino-4-deoxychorismate mutase (e.g., encoded by a papB gene in some organisms and referred to herein as “papB,” for short), 4-amino-4-deoxyprephenate dehydrogenase (e.g., encoded by a papC gene in some organisms and referred to herein as “papC,” for short), aminotransferase (referred to herein as “AT”), and decarboxylase (referred to herein as “DC”). In some cases, multiple enzyme activities may be performed by one enzyme. For example, in Komagataella pastoris, as well as other organisms, a native bifunctional enzyme that acts as a 4-amino-4-deoxychorismate synthase also has glutaminase activity.

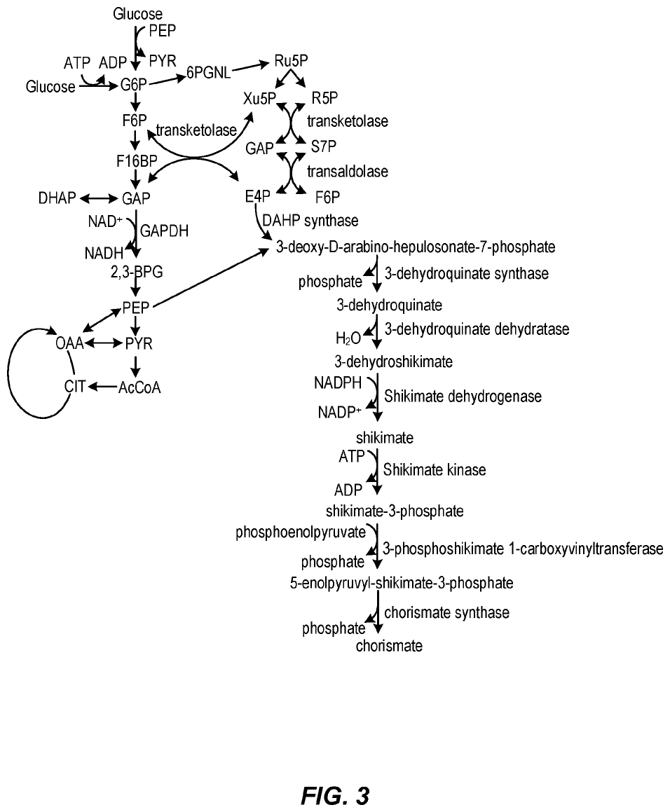

Chorismate is derived from the aromatic branch of amino acid biosynthesis, based on the precursors phosphoenolpyruvate (PEP) and erythrose-4-phosphate (E4P) (see FIG. 3). The first step of this aromatic biosynthesis pathway (carried out by 3-deoxy-D-arabinoheptulosonate 7-phosphate [DAHP] synthase) is subject to feedback inhibition by the aromatic amino acids tyrosine, tryptophan, and phenylalanine.

The production of 4-APEA by fermentation of a simple carbon source can be achieved by linking flux through the shikimate biosynthesis pathway to an introduced 4-APEA pathway including the five enzymes identified above, and optionally improving flux through this pathway, in a suitable microbial host.

Engineering for Microbial 4-Aminophenylethylamine Production

Any 4-APEA pathway enzyme that is active in the microbial cell being engineered may be introduced into the cell, typically by introducing and expressing the gene(s) encoding the enzyme(s) using standard genetic engineering techniques. Suitable 4-APEA pathway enzymes may be derived from any source, including plant, archaeal, fungal, gram-positive bacterial, and gram-negative bacterial sources (see, e.g., those described herein). In various embodiments, at least one, two, three, four, or all gene(s) introduced into the microbial cell is non-native to the cell.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from a regulable promoter (e.g., an inducible or repressible promoter). The heterologous gene(s) can optionally be codon-optimized to enhance expression in the selected microbial host cell. The codon-optimization table used in the Example is the K. pastoris Kazusa codon table at www.kazusa.or.jp/codon/cgi-bin/showcodon.cgi?species=4922.

In various embodiments, the 4-APEA titers achieved by expressing all five 4-APEA pathway enzymes are at least 5, 10, 15, 20, 25, 30, 35, 40, 45, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, or 35 gm/L. In various embodiments, the titer is in the range of 5 mg/L to 800 mg/L, 10 mg/L to 700 mg/L, 15 mg/L to 600 mg/L, 20 mg/L to 500 mg/L, 25 mg/L to 400 mg/L, 30 mg/L to 300 mg/L, 30 mg/L to 200 mg/L, 30 mg/L to 100 mg/L, 30 mg/L to 50 mg/L, or any range bounded by any of the values listed above.

Engineering for Microbial Production of 4-Aminophenylethylamine Precursors or Derivatives Thereof

In some embodiments, suitable microbial hosts, such as, e.g., Komagataella species like K. pastoris or K. phaffi, can be engineered to produce 4-aminophenylethylamine (4-APEA) precursors, such as 4-aminophenylpyruvate (4-APP) and/or 4-aminophenylalanine (4-APhe). To produce these precursors, a truncated version of the 4-APEA pathway described above can be introduced. To produce 4-APP, a microbial host cell can be engineered to heterologously express each of the following enzyme activities: 4-amino-4-deoxychorismate synthase, 4-amino-4-deoxychorismate mutase, and 4-amino-4-deoxyprephenate dehydrogenase, typically by introducing and expressing the gene(s) encoding the enzyme(s) using standard genetic engineering techniques. 4-APhe can be produced in such an engineered microbial cell by additionally engineering the cell to heterologously express an aminotransferase (AT) activity. The general considerations discussed above for introducing the full 4-APEA pathway also apply to introducing a truncated pathway. Likewise, the titers of 4-APP and/or 4-APhe achievable using the methods described herein are the same as those given above for 4-APEA.

In some embodiments, one or more enzymes other than those of the 4-APEA pathway can also be introduced to produce one or more derivatives of 4-APEA or a 4-APEA precursor. For example, an engineered microbial cell that is capable of producing 4-APP can be engineered to produce 4-aminophenylethanol by additionally engineering the cell to heterologously express the enzyme activities necessary to convert 4-APP to 4-aminophenylethanol. Conversion of aminophenylpyruvate to 4-aminophenylethanol is sequentially catalyzed by a phenylpyruvate decarboxylase (or a pyruvate decarboxylase), followed by an alcohol dehydrogenase/acetaldehyde reductase (these enzymes are facilitate the interconversion between alcohols and aldehydes or ketones with the reduction of nicotinamide adenine dinucleotide (NAD+) to NADH and are thus termed “alcohol dehydrogenases,” “acetaldehyde reductases,” or “alcohol dehydrogenase/acetaldehyde reductases”). The general considerations discussed herein for introducing the full 4-APEA pathway also apply to introducing a truncated pathway with one or more additional enzyme activities. Likewise, the titers of a resultant derivative (such as 4-aminophenylethanol) that are achievable using the methods described herein are the same as those given above for 4-APEA.

Further genetic modifications can be used to increase the yield of the desired product (e.g., 4-APEA, 4-APP, 4-APhe, and or aminophenylethanol). These are described below. For ease of discussion, the modifications are described in terms of increasing 4-APEA production, but those of skill in the art understand that these further genetic modifications apply equally to increasing the yields of 4-APEA precursors or derivatives thereof.

Increasing the Activity of Upstream Enzymes

One approach to increasing 4-APEA production in a microbial cell that is capable of such production is to increase the activity of one or more upstream enzymes in the biosynthesis pathway. Upstream pathway enzymes include all enzymes involved in the conversions from a feedstock all the way to a metabolite that can be directly converted to 4-APEA (i.e., chorismate). These enzymes are referred to herein as “upstream chorismate pathway enzymes.” Illustrative enzymes, for this purpose, include, but are not limited to, those shown in FIG. 1 in the pathway leading to this metabolite. In some embodiments, one or more upstream pathway enzymes whose activity is increased are selected from 3-deoxy-D-arabinoheptulosonate 7-phosphate (DAHP) synthase and subsequent enzymes in the pathway leading to chorismate. Examples include glucokinase, transketolase, transaldolase, phospho-2-dehydro-3-deoxyheptonate aldolase, 3-deoxy-D-arabino-heptulosonate-7-phosphate (DAHP) synthase, 3-dehydroquinate synthase, 3-dehydroquinate dehydratase, shikimate dehydrogenase, shikimate kinase, 3-phosphoshikimate 1-carboxyvinyltransferase, and chorismate synthase. In some embodiments, the activity of a pentafunctional enzyme that acts as a 3-dehydroquinate synthase, 3-dehydroquinate dehydratase, shikimate dehydrogenase, shikimate kinase, 3-phosphoshikimate 1-carboxyvinyltransferase can be increased. Suitable upstream pathway genes encoding these enzymes may be derived from any available source, including, for example, those disclosed herein. For example, the activity of the native Komagataella pastoris pentafunctional enzyme can be increased, or a non-native pentafunctional enzyme can be introduced into an engineered microbial cell.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

Alternatively, or in addition, one or more promoters can be substituted for native promoters. In certain embodiments, the replacement promoter is stronger than the native promoter and/or is a constitutive promoter. The replacement promoter can, if desired, be one that is regulable (e.g., inducible or repressible). In some embodiments a thiamine-repressed promoter can be employed, which can reduce the metabolic load on the cell.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of 4-APEA production and expressed from a strong constitutive promoter and/or can optionally be codon-optimized to enhance expression in the selected microbial host cell.

In various embodiments, the engineering of a 4-APEA-producing microbial cell to increase the activity of one or more upstream pathway enzymes increases the 4-APEA titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 4-APEA titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 4-APEA titer observed in a 4-APEA-producing microbial cell that lacks any increase in activity of upstream pathway enzymes. This reference cell may have one or more other genetic alterations aimed at increasing 4-APEA production.

In various embodiments, the 4-APEA titers achieved by increasing the activity of one or more upstream pathway enzymes are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35 or 40 gm/L. In various embodiments, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

Increasing the Activity of Nitrogen Assimilation and Utilization Enzymes

One approach to increasing 4-APEA production in a microbial cell that is capable of such production is to increase the activity of one or more nitrogen assimilation and utilization pathway enzymes. Such enzymes include any enzyme that participates in nitrogen assimilation and utilization in a manner that increases production of 4-APEA. Illustrative enzymes, for this purpose, include, but are not limited to, isocitrate dehydrogenase, glutamine synthetase, glutamate synthase, glutamate dehydrogenase, and ammonium permease. The approaches to increasing activity of upstream pathway enzymes, discussed above, apply equally to nitrogen assimilation and utilization pathway enzymes.

In various embodiments, the engineering of a 4-APEA-producing microbial cell to increase the activity of one or more nitrogen assimilation and utilization pathway enzymes increases the 4-APEA titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 4-APEA titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 4-APEA titer observed in a 4-APEA-producing microbial cell that lacks any increase in activity of upstream pathway enzymes. This reference cell may have one or more other genetic alterations aimed at increasing 4-APEA production.

In various embodiments, the 4-APEA titers achieved by increasing the activity of one or more nitrogen assimilation and utilization pathway enzymes are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35 or 40 gm/L. In various embodiments, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

Introduction of Feedback-Deregulated Enzymes

Since aromatic amino acid biosynthesis is subject to feedback inhibition, another approach to increasing 4-APEA production in a microbial cell engineered for this purpose is to introduce feedback-deregulated forms of one or more enzymes that are normally subject to feedback inhibition in the pathway leading to chorismate. DAHP synthase is an example of such an enzyme. A feedback-deregulated form can be a heterologous, wild-type enzyme that is less sensitive to feedback inhibition than the endogenous enzyme in the particular microbial host cell. Alternatively, a feedback-deregulated form can be a variant of an endogenous or heterologous enzyme that has one or more mutations rendering it less sensitive to feedback inhibition than the corresponding wild-type enzyme. Examples of the latter include variant DAHP synthases (two from S. cerevisiae, two from E. coli, and two from K. pastoris) that have known point mutations rendering them resistant to feedback inhibition, e.g., S. cerevisiae ARO4Q166K, S. cerevisiae ARO4K229L, E. coli AroGD146N, E. coli AroGP150L, K. pastoris ARO4K237L, and K. pastoris ARO4Q174K. The last 5 characters of these designations indicate amino acid substitutions, using the standard one-letter code for amino acids, with the first letter referring to the wild-type residue and the last letter referring to the replacement reside; the numbers indicate the position of the amino acid substitution in the translated protein.

In various embodiments, the engineering of a 4-APEA-producing microbial cell to express a feedback-deregulated enzymes increases the 4-APEA titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in 4-APEA titer is in the range of 10 percent to 100-fold, 2-fold to 50-fold, 5-fold to 40-fold, 10-fold to 30-fold, or any range bounded by any of the values listed above. These increases are determined relative to the 4-APEA titer observed in a 4-APEA-producing microbial cell that does not express a feedback-deregulated enzyme. This reference cell may (but need not) have other genetic alterations aimed at increasing 4-APEA production, i.e., the cell may have increased activity of an upstream pathway enzyme resulting from some means other than feedback-insensitivity.

In various embodiments, the 4-APEA titers achieved by using a feedback-deregulated enzyme to increase flux though the 4-APEA biosynthetic pathway are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35, or 40 gm/L. In various embodiments, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

The approaches of supplementing the activity of one or more endogenous enzymes and/or introducing one or more feedback-deregulated enzymes can be combined in chorismate dehydratase-expressing microbial cells to achieve even higher 4-APEA production levels.

Reduction of Consumption of Chorismate, its Precursors, and or Intermediates in the 4-APEA Pathway

Another approach to increasing 4-APEA production in a microbial cell that is capable of such production is to decrease the activity of one or more enzymes that consume one or more chorismate pathway precursors, that consume 4-chorismate itself, and/or one or more intermediates in the pathway leading from chorismate to 4-APEA. In an illustrative embodiment, the activity or expression of dihydroxyacetone phosphatase, which consumes the chorismate precursor dihydroxyacetone phosphate and converts it to dihydroxyacetone is reduced. Other illustrative enzymes that consume chorismate precursors include 3-dehydroshikimate dehydratase, shikimate dehydrogenase, and phosphoenolpyruvate phosphotransferase. Examples of enzymes that consume chorismate itself include anthranilate synthase and chorismate mutase. Illustrative enzymes that consume intermediates in the 4-APEA pathway include those that convert native aromatic amino acids to the corresponding monoamines, such as decarboxylases or aromatic amino acid decarboxylases. In embodiments not aimed at producing 4-aminophenylethanol, it can be advantageous to reduce the activity of the enzymes the covert 4-APP to this compound, namely phenylpyruvate decarboxylase (or pyruvate decarboxylase) and/or alcohol dehydrogenase/acetaldehyde reductase.

In some embodiments, the activity of one or more such enzymes is reduced by modulating the expression or activity of the native enzyme(s). The activity of such enzymes can be decreased, for example, by substituting the native promoter of the corresponding gene(s) with a less active or inactive promoter or by deleting the corresponding gene(s).

Another approach to increasing 4-APEA production in a microbial cell that is capable of such production is to increase the level of the chorismate precursor phosphoenolpyruvate (PEP) levels by uncoupling the uptake of glucose from the conversion of PEP to pyruvate which occurs by phosphoenolpyruvate phosphotransferase. Phosphoenolpyruvate phosphotransferase is also called the PTS system, and consists of three genes, ptsG, ptsH, and ptsI. Deletion or decreased expression of any one of the phosphoenolpyruvate phosphotransferase genes if present eliminates or decreases the activity of the PTS system and improves PEP availability for DAHP synthase. This approach can be used with any microbial host (typically bacterial) that has a PTS system.

In various embodiments, the engineering of a 4-APEA-producing microbial cell to reduce precursor, or chorismate, consumption by one or more side pathways increases the 4-APEA titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 4-APEA titer is in the range of 10-fold to 1000-fold, 20-fold to 500-fold, 50-fold to 400-fold, 10-fold to 300-fold, or any range bounded by any of the values listed above. These increases are determined relative to the 4-APEA titer observed in a 4-APEA-producing microbial cell that does not include genetic alterations to reduce precursor consumption. This reference cell may (but need not) have other genetic alterations aimed at increasing 4-APEA production, i.e., the cell may have increased activity of an upstream pathway enzyme.

In various embodiments, the 4-APEA titers achieved by reducing precursor, or chorismate, consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35, and 40 gm/L. In various embodiments, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

Increasing the NADPH Supply

Another approach to increasing 4-APEA production in a microbial cell that is capable of such production is to increase the supply of the reduced form of nicotinamide adenine dinucleotide phosphate (NADPH), which provides the reducing equivalents for biosynthetic reactions. For example, the activity of one or more enzymes that increase the NADPH supply can be increased by means similar to those described above for upstream pathway enzymes, e.g., by modulating the expression or activity of the native enzyme(s), replacing the native promoter(s) with a stronger and/or constitutive promoter, and/or introducing one or more gene(s) encoding enzymes that increase the NADPH supply. Illustrative enzymes, for this purpose, include, but are not limited to, pentose phosphate pathway enzymes, NADP+-dependent glyceraldehyde 3-phosphate dehydrogenase (GAPDH), and NADP+-dependent glutamate dehydrogenase. Such enzymes may be derived from any available source, including any of those described herein with respect to other enzymes. Examples include the NADPH-dependent glyceraldehyde 3-phosphate dehydrogenase (GAPDH) encoded by gapC from Clostridium acetobutylicum, the NADPH-dependent GAPDH encoded by gapB from Bacillus subtilis, and the non-phosphorylating GAPDH encoded by gapN from Streptococcus mutans.

In various embodiments, the engineering of a 4-APEA-producing microbial cell to increase the activity of one or more of such enzymes increases the 4-APEA titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 4-APEA titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 4-APEA titer observed in a 4-APEA-producing microbial cell that lacks any increase in activity of such enzymes. This reference cell may have one or more other genetic alterations aimed at increasing 4-APEA production.

In various embodiments, the 4-APEA titers achieved by reducing precursor, or 4-APEA, consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35, or 40 gm/L. In various embodiments, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

Any of the approaches for increasing 4-APEA production described above can be combined, in any combination, to achieve even higher 4-APEA production levels.

Illustrative Amino Acid and Nucleotide Sequences

The following table identifies amino acid and nucleotide sequences used in Example 1. The corresponding sequences are shown in the Sequence Listing.

TABLE A
ID NO Cross-Reference Table for Coding Sequences

| | | | | AA SEQ | NT SEQ |
|---|---|---|---|---|---|
| Zymergen Gene Name | Gene_function | UniProt | Source Organism | ID NO: | ID NO: |
| aro4_Sc_K229L* | DAHP synthase | | S. cerevisiae | 1 | 56 |
| aro1_Kpas | aro1 | | K. pastoris | 2 | 57 |
| papA_St | papA | A0A2M9JGB8 | Streptomyces sp. CB01635 | 3 | 58 |
| papA_Pf | papA | C3K4Z9 | Pseudomonas fluorescens | 4 | 59 |
| | | | (strain SBW25) | | |
| papB_Sp | papB | D9UBV6 | Streptomyces pristinaespiralis | 5 | 60 |
| papB_Pl48 | papB | Q7N1B4 | Photorhabdus laumondii | 6 | 61 |
| | | | subsp. Laumondii (strain | | |
| | | | DSM 15139/CIP 105565/TT01) | | |
| papC_Ps | papC | A0A2G5MIC2 | Pseudomonas sp. 2822 | 7 | 62 |
| papC_Pf | papC | C3K4Z8 | Pseudomonas fluorescens | 8 | 63 |
| | | | (strain SBW25) | | |
| TYDC2_Ps | DC | P54769 | Papaver somniferum | 9 | 64 |
| | | | (Opium poppy) | | |
| PheDC_Ef | DC | Q1JTV5 | Enterococcus faecium | 10 | 65 |
| | | | (Streptococcus faecium) | | |
| AADC1A_Sl | DC | Q1KSC6 | Solanum lycopersicum | 11 | 66 |
| | | | (Tomato) (Lycopersicon | | |
| | | | esculentum) | | |
| AADC1B_Sl | DC | Q1KSC5 | Solanum lycopersicum | 12 | 67 |
| | | | (Tomato) (Lycopersicon | | |
| | | | esculentum) | | |
| tyrB_Ec | AT | P04693 | Escherichia coli | 13 | 68 |
| | | | (strain K12) | | |
| AT_Ph | AT | V5M241 | Petunia hybrida | 14 | 69 |
| TAT2_At | AT | Q9LVY1 | Arabidopsis thaliana | 15 | 70 |
| aroT_Cg | AT | Q8NTT4 | C. glutamicum | 16 | 71 |
| papA_Af | papA | B8ND45 | Aspergillus flavus | 17 | 72 |
| | | | (strain ATCC 200026 ) | | |
| papA_As | papA | A0A0X4JQ12 | Arthrobacter sp. ATCC | 18 | 73 |
| | | | 21022 | | |
| papA_Fo | papA | A0A0J9VM93 | Fusarium oxysporum f. | 19 | 74 |
| | | | sp. Lycopersici | | |
| | | | (NRRL 34936) | | |
| papA_Sp | papA | A0A221P513 | Streptomyces pluripotens | 20 | 75 |
| papA_Sc | papA | P37254 | Saccharomyces cerevisiae | 21 | 76 |
| | | | (strain ATCC 204508/S288c) | | |
| | | | (Baker's yeast) | | |
| papB_Pp | papB | L1M7A5 | Pseudomonas putida CSV86 | 22 | 77 |
| papB_Pl48 | papB | A0A0S8YVA0 | Pseudomonas sp. Leaf48 | 23 | 78 |
| papB_Ph | papB | A0A0F5VI64 | Photobacterium halotolerans | 24 | 79 |
| papB_Pa | papB | B6VKG0 | Photorhabdus asymbiotica | 25 | 80 |
| | | | subsp. asymbiotica | | |
| | | | (strain ATCC 43949 ) | | |
| papB_Sp_A127G* | papB | P72541 | Streptomyces pristinaespiralis | 26 | 81 |
| papC_SCB | papC | A0A2M9JGH1 | Streptomyces sp. CB01635 | 27 | 82 |
| papC_Pwy | papC | A0A098MBF8 | Paenibacillus wynnii | 28 | 83 |
| papC_Xdo | papC | A0A068QYW6 | Xenorhabdus doucetiae | 29 | 84 |
| papC_Pfr | papC | A0A1H5DP46 | Pseudomonas frederiksbergensis | 30 | 85 |
| papC_Pte | papC | T0P9D9 | Photorhabdus temperata | 31 | 86 |
| | | | subsp. temperata M1021 | | |
| ybdL_Eco | AT | P77806 | Escherichia coli | 32 | 87 |
| | | | (strain K12) | | |
| hisC_Csu | AT | Q8R5Q4 | Caldanaerobacter | 33 | 88 |
| | | | subterraneus subsp. | | |
| | | | Tengcongensis | | |
| aro9_Sce | AT | P38840 | Saccharomyces cerevisiae | 34 | 89 |
| aatA_Ddi | AT | Q55F21 | Dictyostelium discoideum | 35 | 90 |
| tyrB_Kpe | AT | O85746 | Klebsiella pneumoniae | 36 | 91 |
| dapL_Ctr | AT | O84395 | Chlamydia trachomatis | 37 | 92 |
| tyrB_Pde | AT | P95468 | Paracoccus denitrificans | 38 | 93 |
| aro4_Sc_Q166K* | DAHP synthase | | S. cerevisiae | 39 | 94 |
| aro4_Sc_Q166K*_K2 | DAHP synthase | | S. cerevisiae | 40 | 95 |
| 29L* | | | | | |
| DC_Aor | DC | Q2U3F8 | Aspergillus oryzae | 41 | 96 |
| | | | (strain ATCC 42149 ) | | |
| mfn_Mja | DC | Q60358 | Methanocaldococcus jannaschii | 42 | 97 |
| ATZ33_Esi | DC | A0A0S3K779 | Enterococcus silesiacus | 43 | 98 |
| aro4_Kph_K219L* | DAHP synthase | F2QLN6 | Komagataella phaffii | 44 | 99 |
| | | | (strain ATCC 76273/CBS | | |
| | | | 7435/CECT 11047/NRRL | | |
| | | | Y-11430/Wegner 21-1) | | |
| | | | (Yeast) (Pichia pastoris) | | |
| aro4_Kph_Q156K | DAHP synthase | F2QLN6 | Komagataella phaffii | 45 | 100 |
| | | | (strain ATCC 76273/CBS | | |
| | | | 7435/CECT 11047/NRRL | | |
| | | | Y-11430/Wegner 21-1) | | |
| | | | (Yeast) (Pichia pastoris) | | |
| aro4_Kph_Q156K*_K | DAHP synthase | F2QLN6 | Komagataella phaffii | 46 | 101 |
| 219L* | | | (strain ATCC 76273/CBS | | |
| | | | 7435/CECT 11047/NRRL | | |
| | | | Y-11430/Wegner 21-1) | | |
| | | | (Yeast) (Pichia pastoris) | | |
| aro4_Kpa_K237L* | DAHP synthase | A0A1B2J7W6 | Komagataella pastoris | 47 | 102 |
| | | | (Yeast) (Pichia pastoris) | | |
| aro4_Kpa_Q174K* | DAHP synthase | A0A1B2J7W6 | Komagataella pastoris | 48 | 103 |
| | | | (Yeast) (Pichia pastoris) | | |
| aro4_Kpa_Q174K*_K | DAHP synthase | A0A1B2J7W6 | Komagataella pastoris | 49 | 104 |
| 237L* | | | (Yeast) (Pichia pastoris) | | |
| aro3_Kpa_K222L* | DAHP synthase | A0A1B2JG33 | Komagataella pastoris | 50 | 105 |
| | | | (Yeast) (Pichia pastoris) | | |
| Aro3_Kph_K230L* | DAHP synthase | A0A1G4KQ85 | Komagataella phaffii | 51 | 106 |
| | | | (strain ATCC 76273/CBS | | |
| | | | 7435/CECT 11047/NRRL | | |
| | | | Y-11430/Wegner 21-1) | | |
| | | | (Yeast) (Pichia pastoris) | | |
| aro3_Scer_K222L* | DAHP synthase | P14843 | Saccharomyces cerevisiae | 52 | 107 |
| | | | (strain ATCC 204508/S288c) | | |
| | | | (Baker's yeast) | | |
| aroG_Eco_D146N*_P | DAHP synthase | P0AB91 | Escherichia coli | 53 | 108 |
| 150L* | | | (strain K12) | | |
| aroF_Eco_N8K* | DAHP synthase | P00888 | Escherichia coli | 54 | 109 |
| | | | (strain K12) | | |
| aroH_Eco | DAHP synthase | P00887 | Escherichia coli | 55 | 110 |
| | | | (strain K12) | | |

The gene name follows the format XXX_YYY_ZZZ, where X is the gene name, Y is the organism identifier, and Z is mutation information if applicable.
*The encoded enzymes include the amino acid substitutions indicated using standard notation. Double mutants are indicated by the format XXX_YYY_ZZZ₁_{_{—}} ZZZ₂_{_{—}}, where ZZZ₁ indicates a first amino acid substitution and ZZZ₂_{_{—}}indicates a second amino acid substitution.

The following table identifies nucleotide sequences for regulable promoters that can be used to express any of the genes discussed herein. The corresponding sequences are shown in the Sequence Listing.

TABLE B
SEQ ID NO Cross-Reference Table for Regulable Promoters

| | | | | | | NT |
|---|---|---|---|---|---|---|
| | | | | | | SEQ |
| | Promoter | Gene | | Source | | ID |
| Class of regulation | name | name | Gene function | Organism | Inducer/repressor | NO: |
| Methanol induction | pAOX1 | AOX1 | alcohol oxidase 1 | Komagataella | Methanol induced | 111 |
| | | | | pastoris | | |
| Methanol induction | pAOX2 | AOX2 | alcohol oxidase 2 | Komagataella | Methanol induced | 112 |
| | | | | pastoris | | |
| Methanol induction | pDAS1 | DAS1 | Dihydroxyacetone synthase 1 | Komagataella | Methanol induced | 113 |
| | | | | pastoris | | |
| Methanol induction | pDAS2 | DAS2 | Dihydroxyacetone synthase 2 | Komagataella | Methanol induced | 114 |
| | | | | pastoris | | |
| Methanol induction | pFBA2 | FBA2 | fructose-1,6-bisphosphate aldolase 2 | Komagataella | Methanol induced | 115 |
| | | | | pastoris | | |
| Methanol induction | pTAL2 | TAL2 | transaldolase 2 | Komagataella | Methanol induced | 116 |
| | | | | pastoris | | |
| Methanol induction | pPMP20 | PMP20 | peroxiredoxin | Komagataella | Methanol induced | 117 |
| | | | | pastoris | | |
| Thiamine repression | pTHI11 | THI11 | 4-amino-5-hydroxymethyl-2-methylpyrimidine | Komagataella | Thiamine | 118 |
| | | | phosphate synthase | pastoris | repressed | |
| Thiamine repression | pTHI20 | THI20 | Hydroxymethylpyrimidine/phosphomethylpyrimidine | Komagataella | Thiamine | 119 |
| | | | kinase | pastoris | repressed | |
| Thiamine repression | pTHI21 | THI21 | Hydroxymethylpyrimidine (HMP) and HMP- | Komagataella | Thiamine | 120 |
| | | | phosphate kinase | pastoris | repressed | |
| Thiamine repression | pTHI5 | THI5 | 4-amino-5-hydroxymethyl-2-methylpyrimidine | Komagataella | Thiamine | 121 |
| | | | phosphate synthase | pastoris | repressed | |
| Thiamine repression | pTHI13 | THI13 | 4-amino-5-hydroxymethyl-2-methylpyrimidine | Komagataella | Thiamine | 122 |
| | | | phosphate synthase | pastoris | repressed | |
| Thiamine repression | pTHI4 | THI4 | Thiazole synthase | Komagataella | Thiamine | 123 |
| | | | | pastoris | repressed | |
| Thiamine repression | pTHI6 | THI6 | Thiamine-phosphate diphosphorylase and | Komagataella | Thiamine | 124 |
| | | | hydroxyethylthiazole kinase | pastoris | repressed | |
| Thiamine repression | pTHI80 | THI80 | Thiamine pyrophosphokinase | Komagataella | Thiamine | 125 |
| | | | | pastoris | repressed | |
| Thiamine repression | pTHI72 | THI72 | Thiamine transporter | Komagataella | Thiamine | 126 |
| | | | | pastoris | repressed | |
| Thiamine repression | pTHI73 | THI73 | Thiamine pathway transporter | Komagataella | Thiamine | 127 |
| | | | | pastoris | repressed | |
| Methionine | pMET3 | MET3 | ATP sulfurylase (sulfate adenylyltransferase) | Komagataella | Methionine | 128 |
| repression | | | | pastoris | repressed | |
| Methionine | pMET17 | MET17 | Homocysteine/cysteine synthase | Komagataella | Methionine | 129 |
| repression | | | | pastoris | repressed | |
| Methanol induction | pAOX1 | AOX1 | alcohol oxidase 1 | Komagataella | Methanol induced | 130 |
| | | | | phaffii | | |
| Methanol induction | pAOX2 | AOX2 | alcohol oxidase 2 | Komagataella | Methanol induced | 131 |
| | | | | phaffii | | |
| Methanol induction | pDAS1 | DAS1 | Dihydroxyacetone synthase 1 | Komagataella | Methanol induced | 132 |
| | | | | phaffii | | |
| Methanol induction | pDAS2 | DAS2 | Dihydroxyacetone synthase 2 | Komagataella | Methanol induced | 133 |
| | | | | phaffii | | |
| Methanol induction | pFBA2 | FBA2 | fructose-1,6-bisphosphate aldolase 2 | Komagataella | Methanol induced | 134 |
| | | | | phaffii | | |
| Methanol induction | pTAL2 | TAL2 | transaldolase 2 | Komagataella | Methanol induced | 135 |
| | | | | phaffii | | |
| Methanol induction | pPMP20 | PMP20 | peroxiredoxin | Komagataella | Methanol induced | 136 |
| | | | | phaffii | | |
| Thiamine repression | pTHI11 | THI11 | 4-amino-5-hydroxymethyl-2-methylpyrimidine | Komagataella | Thiamine | 137 |
| | | | phosphate synthase | phaffii | repressed | |
| Methionine | pMET3 | MET3 | ATP sulfurylase (sulfate adenylyltransferase) | Komagataella | Methionine | 138 |
| repression | | | | phaffii | repressed | |
| Methionine | pMET17 | MET17 | Homocysteine/cysteine synthase | Komagataella | Methionine | 139 |
| repression | | | | phaffii | repressed | |
| Low glucose | pG1 | GTH1 | high-affinity glucose transporter | Komagataella | Low glucose | 140 |
| induction | | | | pastoris | induced | |
| Low glucose | pG3 | GTH1 | high-affinity glucose transporter | Komagataella | Low glucose | 141 |
| induction | | | | pastoris | induced | |
| Low glucose | pG4 | GTH1 | high-affinity glucose transporter | Komagataella | Low glucose | 142 |
| induction | | | | pastoris | induced | |
| Low glucose | pG6 | GTH1 | high-affinity glucose transporter | Komagataella | Low glucose | 143 |
| induction | | | | pastoris | induced | |
| Low glucose | pG7 | GTH1 | high-affinity glucose transporter | Komagataella | Low glucose | 144 |
| induction | | | | pastoris | induced | |
| Low glucose | pG8 | GTH1 | high-affinity glucose transporter | Komagataella | Low glucose | 145 |
| induction | | | | pastoris | induced | |
| Lysine repression | pLYS1 | LYS1 | Saccharopine dehydrogenase (NAD+, L-lysine- | Komagataella | Lysine repressed | 146 |
| | | | forming) | pastoris | | |
| Lysine repression | pLYS9 | LYS9 | Saccharopine dehydrogenase (NADP+, L- | Komagataella | Lysine repressed | 147 |
| | | | glutamate-forming) | pastoris | | |
| Lysine repression | pLYS1 | LYS1 | Saccharopine dehydrogenase (NAD+, L-lysine- | Komagataella | Lysine repressed | 148 |
| | | | forming) | phaffii | | |
| Lysine repression | pLYS9 | LYS9 | Saccharopine dehydrogenase (NADP+, L- | Komagataella | Lysine repressed | 149 |
| | | | glutamate-forming) | phaffii | | |
| Threonine | pTHR1 | THR1 | Homoserine kinase | Komagataella | Threonine | 150 |
| repression | | | | pastoris | repressed | |
| Serine repression | pSER1 | SER1 | 3-phosphoserine aminotransferase | Komagataella | Serine repressed | 151 |
| | | | | pastoris | | |
| Zinc repression | pPIS1 | PIS1 | Phosphatidylinositol synthase | Komagataella | Zinc repressed | 152 |
| | | | | pastoris | | |
| Phosphate | pPHO5 | PHO5 | Repressible acid phosphatase | Komagataella | Phosphate | 153 |
| repression | | | | pastoris | repressed | |
| Phosphate | pPHO89 | PHO89 | Phosphate transporter | Komagataella | Phosphate | 154 |
| repression | | | | pastoris | repressed | |
| Copper induction | pCUP1 | CUP1 | Copper metallothionein 1-1 | Saccharomyces | Copper induced | 155 |
| | | | | cerevisiae | | |
| Copper induction | pLCC1 | LCC1 | laccase | Pycnoporus | Copper induced | 156 |
| | | | | coccineus | | |
| Copper repression | pCTR3 | CTR3 | Copper transport protein | Saccharomyces | Copper repressed | 157 |
| | | | | cerevisiae | | |
| Nitrogen Catabolite | pGAP1 | GAP1 | General amino acid permease | Komagataella | Low/poor nitrogen | 158 |
| Repression | | | | pastoris | conditions (proline, | |
| | | | | | urea, low ammonia | |
| | | | | | concentrations) | |
| Ethanol inducible | plCL1 | ICL1 | Isocitrate lyase | Komagataella | Ethanol induced | 159 |
| | | | | pastoris | | |

TABLE C
SEQ ID NO Cross-Reference Table for Constitutive Promoters

| | | NT SEQ |
|---|---|---|
| Promoter name | Source organism | ID NO |
| pTEF_Ag | Ashbya gossypii | 160 |
| pAOX1 | Komagataella pastoris | 161 |
| pILV5 | Komagataella pastoris | 162 |
| pGCW14 | Komagataella pastoris | 163 |
| pTEF1_Sc | Saccharomyces cerevisiae | 164 |
| pGAP | Komagataella pastoris | 165 |
| P0472 | Komagataella pastoris | 166 |
| pTDH3_KP | Komagataella pastoris | 167 |
| pENO1_KP | Komagataella pastoris | 168 |
| pKEX2_KP | Komagataella pastoris | 169 |
| p1_pTEFI_KPA | Komagataella pastoris | 170 |
| p2_pADHI_KPA | Komagataella pastoris | 171 |
| p3_pPGKI_KPA | Komagataella pastoris | 172 |
| p4_pTPI1_KPA | Komagataella pastoris | 173 |
| p5_pTDH3_KPA | Komagataella pastoris | 174 |
| p6_pRPL3_KPA | Komagataella pastoris | 175 |
| p7_pSSB2_KPA | Komagataella pastoris | 176 |
| p8_pYEF3_KPA | Komagataella pastoris | 177 |
| p9_pENO1_KPA | Komagataella pastoris | 178 |
| p10_pHHF2_KPA | Komagataella pastoris | 179 |
| p11_pHTB2_KPA | Komagataella pastoris | 180 |
| p12_pRPL18B_KPA | Komagataella pastoris | 181 |
| p13_pALD6_KPA | Komagataella pastoris | 182 |
| p14_pALD4_KPA | Komagataella pastoris | 183 |
| p15_pALD5_KPA | Komagataella pastoris | 184 |
| p16_pPAB1_KPA | Komagataella pastoris | 185 |
| p17_pRNR1_KPA | Komagataella pastoris | 186 |
| p18_pSAC6_KPA | Komagataella pastoris | 187 |
| p19_pRNR2_KPA | Komagataella pastoris | 188 |
| p20_pPOP6_KPA | Komagataella pastoris | 189 |
| p21_pRAD27_KPA | Komagataella pastoris | 190 |
| p22_pPSP2_KPA | Komagataella pastoris | 191 |
| p23_pREV1_KPA | Komagataella pastoris | 192 |
| p24_pHXT7p_KPA | Komagataella pastoris | 193 |
| p25_pGPM1_KPA | Komagataella pastoris | 194 |
| p26_pGPD1_KPA | Komagataella pastoris | 195 |
| p27_pFBA1_KPA | Komagataella pastoris | 196 |
| p28_pPDC1_KPA | Komagataella pastoris | 197 |
| p29_pPYK1_KPA | Komagataella pastoris | 198 |
| p30_pPGI1_KPA | Komagataella pastoris | 199 |
| p31_pCYC1_KPA | Komagataella pastoris | 200 |
| p32_pHSP82_KPA | Komagataella pastoris | 201 |
| p33_pILV5_KPA | Komagataella pastoris | 202 |
| p34_pKAR2_KPA | Komagataella pastoris | 203 |
| p35_pKEX2_KPA | Komagataella pastoris | 204 |
| p36_pPET9_KPA | Komagataella pastoris | 205 |
| p37_pSSA4_KPA | Komagataella pastoris | 206 |
| p38_pTEFI_SC | Saccharomyces cerevisiae | 207 |
| p39_pADHI_SC | Saccharomyces cerevisiae | 208 |
| p40_pPGKI_SC | Saccharomyces cerevisiae | 209 |
| p41_pTPI1_SC | Saccharomyces cerevisiae | 210 |
| p42_pTDH3_SC | Saccharomyces cerevisiae | 211 |
| p43_pTEF2_SC | Saccharomyces cerevisiae | 212 |
| p44_pRPL3_SC | Saccharomyces cerevisiae | 213 |
| p45_pSSB1_SC | Saccharomyces cerevisiae | 214 |
| p46_pYEF3_SC | Saccharomyces cerevisiae | 215 |
| p47_pENO2_SC | Saccharomyces cerevisiae | 216 |
| p48_pCCW12_SC | Saccharomyces cerevisiae | 217 |
| p49_pHHF2_SC | Saccharomyces cerevisiae | 218 |
| p50_pHHF1_SC | Saccharomyces cerevisiae | 219 |
| p51_pHTB2_SC | Saccharomyces cerevisiae | 220 |
| p52_pRPL18B_SC | Saccharomyces cerevisiae | 221 |
| p53_pALD6_SC | Saccharomyces cerevisiae | 222 |
| p54_pPAB1_SC | Saccharomyces cerevisiae | 223 |
| p55_pRET2_SC | Saccharomyces cerevisiae | 224 |
| p56_pRNR1_SC | Saccharomyces cerevisiae | 225 |
| p57_pSAC6_SC | Saccharomyces cerevisiae | 226 |
| p58_pRNR2_SC | Saccharomyces cerevisiae | 227 |
| p59_pPOP6_SC | Saccharomyces cerevisiae | 228 |
| p60_pRAD27_SC | Saccharomyces cerevisiae | 229 |
| p61_pPSP2_SC | Saccharomyces cerevisiae | 230 |
| p62_pREV1_SC | Saccharomyces cerevisiae | 231 |
| p63_pHXT7p_SC | Saccharomyces cerevisiae | 232 |
| p64_pGPM1_SC | Saccharomyces cerevisiae | 233 |
| p65_pGPD1_SC | Saccharomyces cerevisiae | 234 |
| p66_pGPD2_SC | Saccharomyces cerevisiae | 235 |
| p67_pFBA_SC | Saccharomyces cerevisiae | 236 |
| p68_pPDC1_SC | Saccharomyces cerevisiae | 237 |
| p69_pPYK1_SC | Saccharomyces cerevisiae | 238 |
| p70_pTDH2_SC | Saccharomyces cerevisiae | 239 |
| p71_pPGI1_SC | Saccharomyces cerevisiae | 240 |
| p72_pCYC1_SC | Saccharomyces cerevisiae | 241 |
| p73_pHSP82_SC | Saccharomyces cerevisiae | 242 |
| p74_pILV5_SC | Saccharomyces cerevisiae | 243 |
| p75_pKAR2_SC | Saccharomyces cerevisiae | 244 |
| p76_pKEX2_SC | Saccharomyces cerevisiae | 245 |
| p77_pPET9_SC | Saccharomyces cerevisiae | 246 |
| p78_pSSA4_SC | Saccharomyces cerevisiae | 247 |
| p79_pGCW14 | Komagataella pastoris | 248 |
| p80_pTEF1 | Komagataella pastoris | 249 |
| p81_pPGK1 | Komagataella pastoris | 250 |
| p83_pGAP | Komagataella pastoris | 251 |
| p91_pARG4 | Komagataella pastoris | 252 |
| p92_pILV5 | Komagataella pastoris | 253 |
| p194_pSPI1_KPA | Komagataella pastoris | 254 |
| p195_p41110_KPA | Komagataella pastoris | 255 |
| p196_pCPR1_KPA | Komagataella pastoris | 256 |
| p197_pTRX1_KPA | Komagataella pastoris | 257 |
| p198_pSDH4_KPA | Komagataella pastoris | 258 |
| p199_pBMH1_KPA | Komagataella pastoris | 259 |
| p200_pHHF2_KPA | Komagataella pastoris | 260 |
| p201_pRPS10_KPA | Komagataella pastoris | 261 |
| p202_pHSP12_KPA | Komagataella pastoris | 262 |
| p203_pGTH1_KPA | Komagataella pastoris | 263 |
| p204_p28050_KPA | Komagataella pastoris | 264 |
| p205_p06620_KPA | Komagataella pastoris | 265 |
| p206_p21610_KPA | Komagataella pastoris | 266 |
| p0472_v2 | Komagataella pastoris | 267 |
| pGCW14_v2 | Komagataella pastoris | 268 |
| pTEF1-EM7 | Komagataella pastoris | 269 |

Microbial Host Cells

Any microbe that can be used to express introduced genes and has a high tolerance to toxicity associated with the production of 4-APEA can be engineered for fermentative production of 4-APEA as described above. In certain embodiments, the microbe is one that is naturally incapable of fermentative production of 4-APEA. In some embodiments, the microbe is one that is readily cultured, such as, for example, a microbe known to be useful as a host cell in fermentative production of compounds of interest. In some embodiments, fungal cells, such as yeast cells, or bacterial cells can be engineered as described above. Examples of suitable yeast cells include yeast cells of the genus Komagataella (e.g., K. pastoris, as referred to as Pinchia pastoris) and of the genus Yarrowia (e.g., Y. lipolytica). Examples of suitable bacterial cells include bacterial cells of the genus Bacillus (e.g., B. lichenformis).

Genetic Engineering Methods

Microbial cells can be engineered for fermentative 4-APEA production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, April 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of K. pastoris cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, 4-APEA. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for 4-APEA production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous (e.g., non-native) gene, e.g., a 4-APEA pathway gene. In various embodiments, for each of the heterologous genes introduced, the microbial cell can include and express, for example: (1) a single copy of a given gene, (2) two or more copies of the gene, which can be the same or different (in other words, multiple copies of the same heterologous gene can be introduced or multiple, different genes encoding the same enzyme can be introduced), (3) a single heterologous gene that is not native to the cell and one or more additional copies of a native gene (if applicable), or (4) two or more non-native genes, which can be the same or different, and/or one or more additional copies of a native gene (if applicable).

In certain embodiments, this engineered host cell can include at least one additional genetic alteration that increases flux through any pathway leading to the production of chorismate. As discussed above, this can be accomplished by one or more of the following: increasing the activity of upstream enzymes, e.g., by introducing a feedback-deregulated version of a DAHP synthase, alone or in combination with other means for increasing the activity of upstream enzymes.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in yeast cells, namely Komagataella pastoris (See Example 1.)

Illustrative Engineered Yeast Cells

In certain embodiments, the engineered yeast (e.g., K. pastoris) cell expresses:
one or more non-native 4-amino-4-deoxychorismate synthase(s) having at least 70 percent, 75 percent, 80 percent, 85 percent, 90 percent, 95 percent or 100 percent amino acid sequence identity with a with a 4-amino-4-deoxychorismate synthase from Pseudomonas fluorescens (strain SBW25);
one or more non-native 4-amino-4-deoxychorismate mutase(s) having at least 70 percent, 75 percent, 80 percent, 85 percent, 90 percent, 95 percent or 100 percent amino acid sequence identity with a 4-amino-4-deoxychorismate mutase from Photorhabdus laumondii subsp. laumondii (strain DSM 15139/CIP 105565/TT01);
one or more non-native 4-amino-4-deoxyprephenate dehydrogenase(s) having at least 70 percent, 75 percent, 80 percent, 85 percent, 90 percent, 95 percent or 100 percent amino acid sequence identity with a 4-amino-4-deoxyprephenate dehydrogenase from Pseudomonas fluorescens (strain SBW25);
optionally, a non-native aminotransferase(s) having at least 70 percent, 75 percent, 80 percent, 85 percent, 90 percent, 95 percent or 100 percent amino acid sequence identity with an aminotransferase from Escherichia coli (strain K12);
optionally, one or more non-native decarboxylase(s) (DC) having at least 70 percent, 75 percent, 80 percent, 85 percent, 90 percent, 95 percent or 100 amino acid sequence identity with a decarboxylase (DC) from Papaver somnferum.

In particular embodiments, the:
4-amino-4-deoxychorismate synthase (papA) from Pseudomonas fluorescens (strain SBW25) includes SEQ ID NO:4;
4-amino-4-deoxychorismate mutase (papB) from Photorhabdus laumondii subsp. laumondii (strain DSM 15139/CIP 105565/TT01) includes SEQ ID NO:6;
4-amino-4-deoxyprephenate dehydrogenase (papC) from Pseudomonas fluorescens (strain SBW25) includes SEQ ID NO:8;
aminotransferase (AT) from Escherichia coli (strain K12), if present, includes SEQ ID NO:13; and
decarboxylase (DC) from Papaver somniferum, if present, includes SEQ ID NO:9. In an illustrative embodiment, a titer of about 34 mg/L 4-APEA was achieved after engineering K. pastoris to express SEQ ID NOs:4, 6, 8, 9, and 13.

In certain embodiments, the engineered yeast (e.g., K. pastoris) cell expresses:
one or more non-native 4-amino-4-deoxychorismate synthase(s) having at least 70 percent, 75 percent, 80 percent, 85 percent, 90 percent, 95 percent or 100 percent amino acid sequence identity with a with a 4-amino-4-deoxychorismate synthase from Streptomyces sp. CB01635;
one or more non-native 4-amino-4-deoxychorismate mutase(s) having at least 70 percent, 75 percent, 80 percent, 85 percent, 90 percent, 95 percent or 100 percent amino acid sequence identity with a 4-amino-4-deoxychorismate mutase from Streptomyces pristinaespiralis;
one or more non-native 4-amino-4-deoxyprephenate dehydrogenase(s) having at least 70 percent, 75 percent, 80 percent, 85 percent, 90 percent, 95 percent or 100 percent amino acid sequence identity with a 4-amino-4-deoxyprephenate dehydrogenase from Pseudomonas sp. 2822;
optionally, one or more non-native aminotransferase(s) having at least 70 percent, 75 percent, 80 percent, 85 percent, 90 percent, 95 percent or 100 percent amino acid sequence identity with an aminotransferase from Petunia hybrida;
optionally, one or more non-native decarboxylase (DC) having at least 70 percent, 75 percent, 80 percent, 85 percent, 90 percent, 95 percent or 100 amino acid sequence identity with a decarboxylase (DC) from Papaver somniferum.

In particular embodiments, the:
4-amino-4-deoxychorismate synthase (papA) from Streptomyces sp. CB01635 includes SEQ ID NO:3;
4-amino-4-deoxychorismate mutase (papB) from Streptomyces pristinaespiralis includes SEQ ID NO:5;
4-amino-4-deoxyprephenate dehydrogenase (papC) from Pseudomonas sp. 2822 includes SEQ ID NO:7;
aminotransferase (AT) from Petunia hybrida, if present, includes SEQ ID NO:14; and
decarboxylase (DC) from Papaver somniferum, if present, includes SEQ ID NO:9. In an illustrative embodiment, a titer of about 16 mg/L 4-APEA was achieved after engineering K. pastoris to express SEQ ID NOs:3, 5, 7, 9, and 14.

In embodiments aimed at producing, 4-APP, the illustrative engineered microbial cell can express just the first three of these five enzymes. To produce 4-APhe, the illustrative engineered microbial cell can express just the first four of these five enzymes. To produce 4-APEA, the illustrative engineered microbial cell can express all five of these enzymes.

### Culturing of Engineered Microbial Cells

Any of the microbial cells described herein can be cultured, e.g., for maintenance, growth, and/or for production of 4-APEA or any of the above products described herein.

In some embodiments, the cultures are grown to an optical density at 600 nm of 10-500, such as an optical density of 50-150.

In various embodiments, the cultures have 4-APEA, 4-APP, 4-APhe, or 4-aminophenylethanol titers of at least 5, 10, 15, 20, 25, 30, 35, 40, 45, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25, 30, 35, or 40 gm/L. In various embodiments, the titer is in the range of 5 mg/L to 800 mg/L, 10 mg/L to 700 mg/L, 15 mg/L to 600 mg/L, 20 mg/L to 500 mg/L, 25 mg/L to 400 mg/L, 30 mg/L to 300 mg/L, 30 mg/L to 200 mg/L, 30 mg/L to 100 mg/L, or 30 mg/L to 50 mg/L, or any range bounded by any of the values listed above. In various embodiments in which 4-APEA (or other product) yield has been increased, e.g., by any of the means described herein, the titer is in the range of 10 mg/L to 900 mg/L, 15 mg/L to 800 mg/L, 20 mg/L to 700 mg/L, 25 mg/L to 600 mg/L, 30 mg/L to 500 mg/L, 30 mg/L to 400 mg/L, 30 mg/L to 300 mg/L, 30 mg/L to 200 mg/L, 30 mg/L to 100 mg/L, or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce 4-APEA or any of the other products described herein, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture, the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Where regulated promoters are employed, the culture conditions can be adjusted to up- or down-regulate the promoter. For example, where thiamine-repressed promoters are employed, if thiamine is initially present in a sufficient amount for repression, the promoters become more active as thiamine is consumed during culturing. If it is advantageous to delay de-repression, thiamine can be added to the culture medium. In general, thiamine levels in this setting vary from 50 mg/L to 0 mg/L. Repression can occur at a thiamine concentration as low as 1 mg/L.

### 4-Aminophenylethylamine Production and Recovery

Any of the methods described herein may further include a step of recovering 4-APEA or any of the other products described herein. In some embodiments, the product contained in a so-called harvest stream is recovered/harvested from the production vessel. The harvest stream may include, for instance, cell-free or cell-containing aqueous solution coming from the production vessel, which contains the desired product as a result of the conversion of production substrate by the resting cells in the production vessel. Cells still present in the harvest stream may be separated from the desired product by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the desired product from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Komagataella pastoris Engineered to Produce 4-Aminophenylethylamine Abstract

This example describes strains of Komagataella pastoris (also called Pichia pastoris) that have been engineered to produce 4-aminophenylethylamine (4-APEA), a diamine monomer useful for the production of polymers and polyimide films. The full pathway for production of 4-APEA is not known to exist in nature but could be assembled by five enzymatic steps downstream of the common metabolite chorismate. The pathway is sequentially made up the following enzymes: 4-amino-4-deoxychorismate synthase (papA), 4-amino-4-deoxychorismate mutase (papB), 4-amino-4-deoxyprephenate dehydrogenase (papC), aminotransferase (AT), and decarboxylase (DC). Some of these enzymatic activities are known to exist in K. pastoris, but efficient and specific production of 4-APEA is challenging as 4-APEA precursors are non-native substrates for some of the pathway enzymes, and expression or flux of native pathway enzymes is likely to be suboptimal. Partial combinatorial search of diverse enzyme sequences and pathway designs, followed by testing in K. pastoris, identified multiple engineered strains that produce mg/L titers of 4-APEA. These strains and pathways may also be used to make chemicals related to 4-APEA, including 4-aminophenylalanine and 4-aminophenylpyruvate or derivatives of these. Finally, we have also demonstrated 4-APEA production in Escherichia coli, but the high toxicity of 4-APEA to E. coli makes it a poor host for large-scale fermentation host. In contrast, K. pastoris is tolerant of high concentrations of 4-APEA and is well-suited for large-scale production.

Plasmid/DNA Design

All strains tested for this work were transformed with plasmid DNA designed using proprietary software. Plasmid designs were specific to the K. pastoris host engineered in this work. The plasmid DNA was physically constructed by a standard DNA assembly method. This plasmid DNA was then used to integrate metabolic pathway inserts by a host-specific method described below.

K. pastoris Pathway Integration

A “split-marker, double-crossover” genomic integration strategy has been developed to engineer K. pastoris strains. FIG. 2 illustrates genomic integration of complementary, split-marker plasmids and verification of correct genomic integration via colony PCR in K. pastoris. Two plasmids with complementary 5′ and 3′ homology arms and overlapping halves of a selectable marker, for example an antibiotic resistance marker such as KanMX or an auxotrophic marker such as URA3, were linearized and transformed as linear fragments. Linearization was achieved either by PCR or by digestion with meganucleases. Direct repeats present in the transformed DNA fragments are shown by the hashed bars. A triple-crossover event integrated the desired heterologous genes into the targeted locus and re-constituted the full selection marker gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on counter-selection media, for example 5-FOA plates to select for the removal of URA3, leaving behind a small single copy of the original direct repeat. This genomic integration strategy can be used for gene knock-out, gene knock-in, and promoter titration in the same workflow. (Abbreviations: Primers: UF=upstream forward, DR=downstream reverse, IR=internal reverse, IF=internal forward.)

Cell Culture

The cell culture and metabolite production workflow is initiated by a hit-picking step that consolidated successfully built strains using an automated workflow that randomized strains across the plate. For each strain that was successfully built, up to eight replicates were tested from distinct colonies to test colony-to-colony variation and other process variation. If fewer than eight colonies were obtained, the existing colonies were replicated so that at least eight wells were tested from each desired genotype.

The colonies were consolidated into 96-well plates with selective medium (SD-ura or YPD with antibiotic) and cultivated for two days until saturation and then frozen with 16.6% glycerol at −80° C. for storage. The frozen glycerol stocks were then used to inoculate a primary seed stage in YPD media grown at 30° C. for 16 hours at 1000 RPM. The primary seed plates were used to inoculate a secondary seed stage in Verduyn media grown at 30° C. for 24 hours at 1000 RPM. The secondary seed plates were then used to inoculate a main cultivation plate with Verduyn media supplemented with 200 mM phthalate buffer and grown at 30° C. for 24-48 hours at 1000 RPM. Plates were removed at the desired time points and tested for cell density (OD600), glucose, and supernatant samples stored for LC-MS or HPLC analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures, but this is not generally observed

Glucose

Glucose is measured using an enzymatic assay with 16 U/mL glucose oxidase (Sigma) with 0.2 U/mL horseradish peroxidase (Sigma) and 0.2 mM Amplex red in 175 mM sodium phosphate buffer, pH 7. Oxidation of glucose generates hydrogen peroxide, which is then oxidized to reduce Amplex red, which changes absorbance at 560 nm. The change is absorbance is correlated to the glucose concentration in the sample using standards of known concentration.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

Genetic Engineering Approach and Results

Strains for 4-APEA production are described in the Tables below. Table 1 lists specific combinations of enzymes and promoters that were built. Tables 2A-C lists the titer of 4-APEA, as well as side products and intermediates, produced by strains that have been tested. A range of titers were detected, as well as the presence of side-products (including 4-aminophenylethanol and tyramine) and pathway intermediates (including 4-aminophenylpyruvate [4-APP] and 4-aminophenylalanine [4-APhe]). Tables 3A-B show further strain designs and results. Upon scaling up to production in a fermentation tank, a titer of 11.1 g/L APEA was achieved for strain 7001065091 (described below).

TABLE 1
Strain Designs for Engineering Komagataella pastoris to Produce 4-APEA

| Production | | |
|---|---|---|
| Strain ZID | Production Strain Genotype | Constuct Genotype |
| 70008 | NS3::papA_Pf/papB_Pl/papC_Pf/tyrB_EC/TYDC2_Ps | pGCW14 > papA_Pf < tTIF51_Scer/P0472 > papB_Pl < |
| 39167 | | tAOD_Scer/pGAP > papC_Pf < tCyc1_Sc |
| | | tAOX1_Rev > TYDC2_Ps_Rev < pTEF1_Sc_Rev/tARG4_Rev > |
| | | tyrB_Ec_Rev < pTDH3_Kp_Rev |
| 70008 | NS3::papA_St/papB_Sp/papC_Ps/tyrB_EC/PheDC | pGCW14 > papA_St < tTIF51_Scer/P0472 > papB_Sp < |
| 40665 | | tAOD_Scer/pGAP > papC_Ps < tCyc1_Sc |
| | | tAOX1_Rev > PheDC_Ef_Rev < pTEF1_Sc_Rev/tARG4_Rev > |
| | | tyrB_Ec_Rev < pTDH3_Kp_Rev |
| 70008 | NS3::papA_Pf/papB_Pl/papC_Pf/tyrB_EC/Phe_DC | pGCW14 > papA_Pf < tTIF51_Scer/P0472 > papB_Pl < |
| 40664 | | tAOD_Scer/pGAP > papC_Pf < tCyc1_Sc |
| | | tAOX1_Rev > PheDC_Ef_Rev < pTEF1_Sc_Rev/tARG4_Rev > |
| | | tyrB_Ec_Rev < pTDH3_Kp_Rev |
| 70008 | NS3::papA_St/papB_Sp/papC_Ps/TAT2_At/PheDC | pGCW14 > papA_St < tTIF51_Scer/P0472 > papB_Sp < |
| 39175 | | tAOD_Scer/pGAP > papC_Ps < tCyc1_Sc |
| | | tAOX1_Rev > PheDC_Ef_Rev < pTEF1_Sc_Rev/tARG4_Rev > |
| | | TAT2_At_Rev < pTDH3_Kp_Rev |
| 70008 | NS3::papA_St/papB_Sp/papC_Ps/AT_Ph/TYDC2_Ps | pGCW14 > papA_St < tTIF51_Scer/P0472 > papB_Sp < |
| 40676 | | tAOD_Scer/pGAP > papC_Ps < tCyc1_Sc |
| | | tAOX1_Rev > TYDC2_Ps_Rev < pTEF1_Sc_Rev/tARG4_Rev > |
| | | AT_Ph_Rev < pTDH3_Kp_Rev |
| 70008 | NS3::papA_Pf/papB_Pl/papC_Pf/TAT2_At/PheDC | pGCW14 > papA_Pf < tTIF51_Scer/P0472 > papB_Pl < |
| 39664 | | tAOD_Scer/pGAP > papC_Pf < tCyc1_Sc |
| | | tAOX1_Rev > PheDC_Ef_Rev < pTEF1_Sc_Rev/tARG4_Rev > |
| | | TAT2_At_Rev < pTDH3_Kp_Rev |
| 70008 | NS3::papA_Pf/papB_Pl/papC_Pf/AT_Ph/TYDC2_Ps | pGCW14 > papA_Pf < tTIF51_Scer/P0472 > papB_Pl < |
| 39164 | | tAOD_Scer/pGAP > papC_Pf < tCyc1_Sc |
| | NS3::papA_Pf/papB_Pl/papC_Pf/AT_Ph/TYDC2_Ps | tAOX1_Rev > TYDC2_Ps_Rev < pTEF1_Sc_Rev/tARG4_Rev > |
| | | AT_Ph_Rev < pTDH3_Kp_Rev |

Table 2A-2C—Titer of 4-APEA and Related Compounds in Komagataella pastoris Strains Engineered to Produce 4-APEA

2A:

| | | Metabolite Titer (mg/L) at 48 hours | | | | | | | | | |
|---|---|---|---|---|---|---|---|---|---|---|---|
| Production | | 4- | | 4- | | 4- | | 4- | | | |
| Strain ZID | Production Strain Genotype | APE | StDev | APP | StDev | Aphe | StDev | APEA | StDev | Tyramine | StDev |
| 7000839167 | NS3::papA_Pf/papB_Pl/papC_Pf/tyrB_EC/TYDC2_Ps | 73.6 | 16.3 | 23.8 | 5.5 | 5.9 | 1.2 | 33.7 | 7.1 | 44.5 | 9.7 |
| 7000840665 | NS3::papA_St/papB_Sp/papC_Ps/tyrB_EC/PheDC | 78.3 | 32.3 | 64.3 | 42.0 | 5.0 | 4.2 | 16.2 | 6.1 | 96.1 | 35.1 |

2B:

| | | Metabolite titer at 24 hours (mg/L) | | | | | | | | | |
|---|---|---|---|---|---|---|---|---|---|---|---|
| | | | | | 4-Amino- | | | | | | |
| | | | | | phenyl- | | | | | | |
| | | | Phenyl- | Amino- | ethanol + | | | | | | |
| | | | ethyl- | phenyl- | p-amino- | | | | Phenyl- | | |
| Strain ID | Strain genotype | 4-APEA | amine | acetate | benzoate | 4-APhe | 4-APP | Chorismate | alanine | Tyrosine | Tyramine |
| 7000840664 | papA_Pf/papB_Pl/ | 67.97 | 11.01 | 1.25 | 64.39 | 6.62 | 14.21 | 10.27 | 4.51 | 0.26 | 26.98 |
| | papC_Pf/PheDC_Ef_Rev/ | | | | | | | | | | |
| | tyrB_Ec_Rev | | | | | | | | | | |
| 7000840665 | papA_St/papB_Sp/ | 28.86 | 6.21 | 1.67 | 80.21 | 6.03 | 47.38 | 11.76 | 3.22 | 0.18 | 10.97 |
| | papC_Ps/PheDC_Ef_Rev/ | | | | | | | | | | |
| | tyrB_Ec_Rev | | | | | | | | | | |
| 7000839167 | papA_Pf/papB_Pl/ | 119.22 | 2.25 | 1.2 | 25.21 | 3.08 | 12.77 | 4.4 | 1.52 | 0.26 | 3.74 |
| | papC_Pf/TYDC2_Ps_Rev/ | | | | | | | | | | |
| | tyrB_Ec_Rev | | | | | | | | | | |
| 7000840676 | papA_St/papB_Sp/ | 151.75 | 2.18 | 5.03 | 156.31 | 11.35 | 87.27 | 15.53 | 5.38 | 0.27 | 0.83 |
| | papC_Ps/TYDC2_Ps_Rev/ | | | | | | | | | | |
| | AT_Ph_Rev | | | | | | | | | | |
| 7000839664 | papA_Pf/papB_Pl/papC_Pf/ | 117.2 | 2.31 | 1.92 | 44.44 | 2.01 | 18.32 | 1.59 | 1 | 0.24 | 4.24 |
| | PheDC_Ef_Rev/ | | | | | | | | | | |
| | TAT2_At_Rev | | | | | | | | | | |
| 7000839175 | papA_St/papB_Sp/ | 49.98 | 10.89 | 4.48 | 181.26 | 12.5 | 105.02 | 19.84 | 6.02 | 0.25 | 8.99 |
| | papC_Ps/PheDC_Ef_Rev/ | | | | | | | | | | |
| | TAT2_At_Rev | | | | | | | | | | |
| 7000839164 | papA_Pf/papB_Pl/ | 122.4 | 2.34 | 1.88 | 44.79 | 2.07 | 18.3 | 1.85 | 1.07 | 0.25 | 4.15 |
| | papC_Pf/TYDC2Ps_Rev/ | | | | | | | | | | |
| | AT_Ph_Rev | | | | | | | | | | |

2C: Fermentation tank production timecourse by strain 7000839167

| Time | APEA titer |
|---|---|
| (hours) | (g/L) |
| 6 | 0 |
| 25 | 0.67 |
| 30 | 1.43 |
| 49 | 4.23 |
| 54 | 4.64 |
| 74 | 6.49 |

Table 3A-3B—Titer of 4-APEA in Komagataella pastoris Strains Engineered to Produce 4-APEA

2A: Specific strain genotypes conferring high-level
constitutive and regulable production of 4-APEA

| | | APEA after | |
|---|---|---|---|
| | | 96 hour | |
| | | fermentation | |
| Strain ID | Strain type | (mg/L) | Strain genotype |
| 7001065091 | constitutive | 11172 | NS3:pGCW14:papA_St/pTDH3:papB_Pa/pGAP:papC_Xdo/ |
| | production | | p0472:aroT_Cg/pTDH3:TYDC2_Ps |
| 7001001659 | regulated | 6665 | NS3:pGCW14:papA_Pf/p0472:papB_Pl/pGAP:papC_Pf/ |
| | production | | pTDH3:tyrB_Ec/pTHI11:TYDC2_Ps:pENO1:GLN1 |
| 7000869673 | constitutive | 4257 | NS3:pGCW14:papA_Pf/p0472:papB_Pl/pGAP:papC_Pf/ |
| | production | | pTDH3:tyrB_Ec/pTHI11:TYDC2_Ps |
| 7000931166 | regulated | 4872 | NS3:pGCW14:papA_Pf/p0472:papB_Pl/pGAP:papC_Pf/ |
| | production | | pTDH3:tyrB_Ec/pTEF1:TYDC2_Ps |

3B: Increased 4-APEA production through promoter replacement or knockout of native genes

| | Parent | | Modifi- | | | | Fold |
|---|---|---|---|---|---|---|---|
| | Strain | Genotype | cation | | Target | | improvement |
| Strain ID | ID | change | type | Promoter | locus | Gene function | over parent |
| 7001023092 | 7000869673 | KO:CYB2 | gene | n/a | CYB2 | L-lactate dehydrogenase (cytochrome) | 1.07 |
| | | | deletion | | | | |
| 7001023104 | 7000869673 | KO:GAP1 | gene | n/a | GAP1 | yeast amino acid transporter | 1.05 |
| | | | deletion | | | | |
| 7001064504 | 7000931166 | KO:PDC1 | gene | n/a | PDC1 | pyruvate decarboxylase | 1.12 |
| | | | deletion | | | | |
| 7001023100 | 7000869673 | KO:PFK26 | gene | n/a | PFK26 | 6-phosphofructo-2-kinase | 1.06 |
| | | | deletion | | | | |
| 7001022086 | 7000931166 | KO:PHO13 | gene | n/a | PHO13 | phosphoglycolate phosphatase | 1.47 |
| | | | deletion | | | | |
| 7001064486 | 7000931166 | p115_pTHI11_ | promoter | p115_pTHI11_ | ABZ1 | para-aminobenzoate synthetase | 1.29 |
| | | KPA:ABZ1 | replacement | KPA | | | |
| 7001064973 | 7000869673 | p9_pENO1_ | promoter | p9_pENO1_KPA | ARO7 | chorismate mutase | 1.11 |
| | | KPA:ARO7 | replacement | | | | |
| 7001001784 | 7000931166 | p115_pTHI11_ | promoter | p115_pTHI11_ | ARP2 | hexokinase | 1.13 |
| | | KPA:ARP2 | replacement | KPA | | | |
| 7001002220 | 7000931166 | p9_pENO1_ | promoter | p9_pENO1_KPA | ATP4 | F-type H+-transporting ATPase | 1.21 |
| | | KPA:ATP4 | replacement | | | subunit b | |
| 7001001799 | 7000931166 | p5_pTDH3_ | promoter | p5_pTDH3_KPA | ATP4 | F-type H+-transporting ATPase | 1.09 |
| | | KPA:ATP4 | replacement | | | subunit b | |
| 7001001658 | 7000931166 | p9_pENO1_ | promoter | p9_pENO1_KPA | COX11 | cytochrome c oxidase assembly | 1.13 |
| | | KPA:COX11 | replacement | | | protein subunit 11 | |
| 7001001646 | 7000931166 | p5_pTDH3_ | promoter | p5_pTDH3_KPA | COX11 | cytochrome c oxidase assembly | 1.18 |
| | | KPA:COX11 | replacement | | | protein subunit 11 | |
| 7001002197 | 7000869673 | p9_pENO1_ | promoter | p9_pENO1_KPA | COX13 | cytochrome c oxidase subunit 6a | 1.13 |
| | | KPA:COX13 | replacement | | | | |
| 7000999114 | 7000869673 | p35_pKEX2_ | promoter | p35_pKEX2_KPA | COX5A | cytochrome c oxidase subunit 4 | 1.17 |
| | | KPA:COX5A | replacement | | | | |
| 7000999147 | 7000931166 | p9_pENO1_ | promoter | p9_pENO1_KPA | CYT1 | ubiquinol-cytochrome c reductase | 1.21 |
| | | KPA:CYT1 | replacement | | | cytochrome c1 subunit | |
| 7001001659 | 7000931166 | p9_pENO1_ | promoter | p9_pENO1_KPA | GLN1 | glutamine synthetase | 1.86 |
| | | KPA:GLN1 | replacement | | | | |
| 7001002203 | 7000931166 | p5_pTDH3_ | promoter | p5_pTDH3_KPA | GLN1 | glutamine synthetase | 1.23 |
| | | KPA:GLN1 | replacement | | | | |
| 7000999152 | 7000931166 | p115_pTHI11_ | promoter | p115_pTHI11_ | MEP2 | ammonium transporter, Amt family | 1.07 |
| | | KPA:MEP2 | replacement | KPA | | | |
| 7001001647 | 7000931166 | p9_pENO1_ | promoter | p9_pENO1_KPA | MEP2 | ammonium transporter, Amt family | 1.26 |
| | | KPA:MEP2 | replacement | | | | |
| 7001002668 | 7000931166 | p35_pKEX2_ | promoter | p35_pKEX2_KPA | MEP2 | ammonium transporter, Amt family | 1.25 |
| | | KPA:MEP2 | replacement | | | | |
| 7001003167 | 7000931166 | p35_pKEX2_ | promoter | p35_pKEX2_KPA | NUFM | NADH dehydrogenase (ubiquinone) | 1.76 |
| | | KPA:NUFM | replacement | | | 1 alpha subcomplex subunit 5 | |
| 7001001657 | 7000931166 | p9_pENO1_ | promoter | p9_pENO1_KPA | NUFM | NADH dehydrogenase (ubiquinone) | 1.80 |
| | | KPA:NUFM | replacement | | | 1 alpha subcomplex subunit 5 | |
| 7000999120 | 7000869673 | p5_pTDH3_ | promoter | p5_pTDH3_KPA | NUFM | NADH dehydrogenase (ubiquinone) | 1.15 |
| | | KPA:NUFM | replacement | | | su1 alpha subcomplex bunit 5 | |
| 7001001766 | 7000869673 | p35_pKEX2_ | promoter | p35_pKEX2_KPA | NUGM | NADH dehydrogenase (ubiquinone) | 1.18 |
| | | KPA:NUGM | replacement | | | 1 alpha subcomplex subunit 5 | |
| 7001064492 | 7000931166 | p115_pTHI11_ | promoter | p115_pTHI11_ | PDC2 | transcription factor | 2.03 |
| | | KPA:PDC2 | replacement | KPA | | | |
| 7001065470 | 7000931166 | p5_pTDH3_ | promoter | p5_pTDH3_KPA | PDC2 | transcription factor | 1.84 |
| | | KPA:PDC2 | replacement | | | | |
| 7000999163 | 7000931166 | p5_pTDH3_ | promoter | p5_pTDH3_KPA | PFK1:PFK2 | 6-phosphofructokinase 1 | 1.17 |
| | | KPA:PFK1:PFK2 | replacement | | | | |
| 7001001645 | 7000931166 | p9_pENO1_ | promoter | p9_pENO1_KPA | PGI1 | glucose-6-phosphate isomerase | 1.52 |
| | | KPA:PGI1 | replacement | | | | |
| 7000999115 | 7000869673 | p5_pTDH3_ | promoter | p5_pTDH3_KPA | PGI1 | glucose-6-phosphate isomerase | 1.14 |
| | | KPA:PGI1 | replacement | | | | |
| 7001002670 | 7000931166 | p5_pTDH3_ | promoter | p5_pTDH3_KPA | RPE1-1 | ribulose-phosphate 3-epimerase | 1.15 |
| | | KPA:RPE1-1 | replacement | | | | |
| 7001001764 | 7000869673 | p132_pPHO5_ | promoter | p132_pPHO5_ | SHB17 | sedoheptulose-bisphosphatase | 1.13 |
| | | KPA:SHB17 | replacement | KPA | | | |
| 7001001786 | 7000931166 | p5_pTDH3_ | promoter | p5_pTDH3_KPA | SHB17 | sedoheptulose-bisphosphatase | 1.24 |
| | | KPA:SHB17 | replacement | | | | |
| 7001064988 | 7000931166 | p120_pTHI4_ | promoter | p120_pTHI4_KPA | SHP1 | anthranilate synthase component I | 1.35 |
| | | KPA:SHP1 | replacement | | | | |
| 7001064972 | 7000869673 | p9_pENO1_ | promoter | p9_pENO1_KPA | SHP1 | anthranilate synthase component I | 1.15 |
| | | KPA:SHP1 | replacement | | | | |
| 7001064990 | 7000931166 | p35_pKEX2_ | promoter | p35_pKEX2_KPA | SHP1 | anthranilate synthase component I | 1.15 |
| | | SKPA:HP1 | replacement | | | | |
| 7001063958 | 7000869673 | p5_pTDH3_ | promoter | p5_pTDH3_KPA | SHP1 | anthranilate synthase component I | 1.17 |
| | | KPA:SHP1 | replacement | | | | |
| 7000999143 | 7000931166 | p119_pTHI13_ | promoter | p119_pTHI13_ | SOL1 | 6-phosphogluconolactonase | 1.24 |
| | | KPA:SOL1 | replacement | KPA | | | |
| 7000999160 | 7000931166 | p35_pKEX2_ | promoter | p35_pKEX2_KPA | SOL1 | 6-phosphogluconolactonase | 1.17 |
| | | KPA:SOL1 | replacement | | | | |
| 7001002206 | 7000931166 | p9_pENO1_ | promoter | p9_pENO1_KPA | SOL1 | 6-phosphogluconolactonase | 1.20 |
| | | SKPA:OL1 | replacement | | | | |
| 7001001793 | 7000931166 | p115_pTHI11_ | promoter | p115_pTHI11_ | TAL1-2 | transaldolase | 1.19 |
| | | KPA:TAL1-2 | replacement | KPA | | | |
| 7001001648 | 7000931166 | p9_pENO1_ | promoter | p9_pENO1_KPA | TAL1-2 | transaldolase | 1.29 |
| | | KPA:TAL 1-2 | replacement | | | | |
| 7001001795 | 7000931166 | p5_pTDH3_ | promoter | p5_pTDH3_KPA | TAL1-2 | transaldolase | 1.31 |
| | | KPA:TAL1-2 | replacement | | | | |
| 7001001656 | 7000931166 | p35_pKEX2_ | promoter | p35_pKEX2_KPA | TEX1 | NADH dehydrogenase (ubiquinone) | 1.22 |
| | | KPA:TEX1 | replacement | | | 1 alpha subcomplex subunit 9 | |
| 7000999156 | 7000931166 | p9_pENO1_ | promoter | p9_pENO1_KPA | TEX1 | NADH dehydrogenase (ubiquinone) | 1.14 |
| | | KPA:TEX1 | replacement | | | 1 alpha subcomplex subunit 9 | |
| 7001001790 | 7000931166 | p5_pTDH3_ | promoter | p5_pTDH3_KPA | TEX1 | NADH dehydrogenase (ubiquinone) | 1.30 |
| | | KPA:TEX1 | replacement | | | 1 alpha subcomplex subunit 9 | |
| 7001063953 | 7000869673 | p35_pKEX2_ | promoter | p35_pKEX2_KPA | TRP3 | anthranilate synthase/indole-3- | 1.15 |
| | | KPA:TRP3 | replacement | | | glycerol phosphate synthase/ | |
| | | | | | | phosphoribosylanthranilate isomerase | |
| 7001064498 | 7000931166 | p35_pKEX2_ | promoter | p35_pKEX2_KPA | TRP3 | anthranilate synthase/indole-3- | 1.19 |
| | | KPA:TRP3 | replacement | | | glycerol phosphate synthase/ | |
| | | | | | | phosphoribosylanthranilate isomerase | |
| 7001064488 | 7000931166 | p5_pTDH3_ | promoter | p5_pTDH3_KPA | TRP3 | anthranilate synthase/ | 1.12 |
| | | KPA:TRP3 | replacement | | | indole-3-glycerol phosphate synthase/ | |
| | | | | | | phosphoribosylanthranilate isomerase | |
| 7001064494 | 7000869673 | p9_pENO1_ | promoter | p9_pENO1_KPA | ZWF1 | glucose-6-phosphate 1-dehydrogenase | 1.13 |
| | | KPA:ZWF1 | replacement | | | | |
| 7001065469 | 7000869673 | p5_pTDH3_ | promoter | p5_pTDH3_KPA | ZWF1 | glucose-6-phosphate 1-dehydrogenase | 1.14 |
| | | KPA:ZWF1 | replacement | | | | |

### REFERENCES

1. Masuo et al. (2016) Scientific Reports 6: 25764.

What is claimed is:

1. An engineered microbial cell that produces 4-aminophenylethylamine (4-APEA), wherein the engineered microbial cell has a high tolerance to toxicity associated with the production of 4-APEA, as defined by a concentration at which the growth of engineered microbial cell is slowed by half (Ki) of at least 30 grams/liter, wherein the engineered microbial cell optionally comprises a yeast cell, optionally a cell of the genus Komagataella, optionally wherein the yeast cell is a cell of the species pastoris or phaffi.

2. The engineered microbial cell of claim 1, wherein the engineered microbial cell heterologously expresses each of the following enzyme activities:
4-amino-4-deoxychorismate synthase;
4-amino-4-deoxychorismate mutase;
4-amino-4-deoxyprephenate dehydrogenase;
aminotransferase (AT); and
decarboxylase (DC);
wherein the enzyme activities are provided by heterologously expressing genes encoding the enzymes, and at least one heterologously expressed enzyme is non-native to the engineered microbial cell, optionally wherein at least two, three, four, or all of the heterologously expressed enzymes are non-native to the engineered microbial cell.

3. An engineered microbial cell of the genus Komagataella that produces 4-aminophenylpyruvate (4-APP), optionally wherein the engineered microbial cell is a cell of the species pastoris or phaffi.

4. The engineered microbial cell of claim 3, wherein the engineered microbial cell heterologously expresses each of the following enzyme activities:
4-amino-4-deoxychorismate synthase;
4-amino-4-deoxychorismate mutase; and
4-amino-4-deoxyprephenate dehydrogenase,
wherein each enzyme activity is provided by heterologously expressing genes encoding the enzymes, and at least one heterologously expressed enzyme is non-native to the engineered microbial cell.

5. The engineered microbial cell of any one of claims 3-4, wherein the engineered microbial cell additionally produces 4-aminophenylalanine (4-APhe).

6. The engineered microbial cell of claim 5, wherein the engineered microbial cell additionally heterologously expresses an aminotransferase (AT) activity.

7. The engineered microbial cell of any one of claims 3-4, wherein the engineered microbial cell additionally produces 4-aminophenylethanol.

8. The engineered microbial cell of claim 7, wherein the engineered microbial cell additionally heterologously expresses an alcohol dehydrogenase/acetaldehyde reductase enzyme.

9. The engineered microbial cell of any one of claims 3-8, wherein at least two, three, or all of the heterologously expressed enzymes are non-native to the engineered microbial cell.

10. The engineered microbial cell of any one of claims 1-9, wherein the engineered microbial cell comprises increased activity of one or more upstream chorismate pathway enzyme(s), said increased activity being increased relative to a control cell, optionally wherein said increased activity is selected from the group consisting of glucokinase, transketolase, transaldolase, phospho-2-dehydro-3-deoxyheptonate aldolase, 3-deoxy-D-arabino-heptulosonate-7-phosphate (DAHP) synthase, 3-dehydroquinate synthase, 3-dehydroquinate dehydratase, shikimate dehydrogenase, shikimate kinase, 3-phosphoshikimate 1-carboxyvinyltransferase, chorismate synthase activity, and any combination thereof.

11. The engineered microbial cell of any one of claims 1-10, wherein the engineered microbial cell comprises increased activity of one or more nitrogen assimilation and utilization pathway enzyme(s), optionally wherein said increased activity is selected from the group consisting of isocitrate dehydrogenase, glutamine synthetase, glutamate synthase, glutamate dehydrogenase, ammonium permease, and any combination thereof.

12. The engineered microbial cell of any one of claims 1-11, wherein the engineered microbial cell comprises reduced activity of one or more enzyme(s) that consume one or more chorismate pathway precursors, chorismate, and/or one or more intermediates in the pathway leading from chorismate to 4-APEA, and/or more enzymes that consume 4-APEA, said reduced activity being reduced relative to a control cell, optionally wherein the one or more enzyme(s) that consume one or more chorismate pathway precursors are selected from the group consisting of dihydroxyacetone phosphatase, 3-dehydroshikimate dehydratase, shikimate dehydrogenase, and phosphoenolpyruvate phosphotransferase, optionally wherein the one or more enzyme(s) that consume chorismate are selected from the group consisting of anthranilate synthase and chorismate mutase, optionally wherein the one or more enzyme(s) that consume one or more intermediates in the pathway leading from chorismate to 4-APEA are selected from the group consisting of decarboxylase, aromatic amino acid decarboxylase, phenylpyruvate decarboxylase, pyruvate decarboxylase, aromatic amino acid ammonia lyase, and alcohol dehydrogenase/acetaldehyde reductase, optionally wherein the one or more enzymes that consume 4-APEA are selected from the group consisting of phenylpyruvate dioxygenase, diamine oxidase, amine oxidase, and amino acid oxidase.

13. The engineered microbial cell of any one of claims 1-12, wherein the engineered microbial cell additionally expresses a feedback-deregulated DAHP synthase.

14. The engineered microbial cell of any one of claims 1-13, wherein the engineered microbial cell comprises increased activity of one or more enzyme(s) that increase the supply of the reduced form of nicotinamide adenine dinucleotide phosphate (NADPH), said increased activity being increased relative to a control cell, optionally wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

15. The engineered microbial cell of claim 1, wherein the non-native enzymes comprise:
a 4-amino-4-deoxychorismate synthase having at least 70% amino acid sequence identity with a 4-amino-4-deoxychorismate synthase from Pseudomonas fluorescens (strain SBW25);
a 4-amino-4-deoxychorismate mutase having at least 70% amino acid sequence identity with a 4-amino-4-deoxychorismate mutase from Photorhabdus laumondii subsp. laumondii (strain DSM 15139/CIP 105565/TT01);
a 4-amino-4-deoxyprephenate dehydrogenase having at least 70% amino acid sequence identity with a 4-amino-4-deoxyprephenate dehydrogenase from Pseudomonas fluorescens (strain SBW25);
optionally, an aminotransferase (AT) having at least 70% amino acid sequence identity with an aminotransferase (AT) from Escherichia coli (strain K12); and
optionally a decarboxylase (DC) having at least 70% amino acid sequence identity with a decarboxylase (DC) from Papaver somniferum.

16. The engineered microbial cell of claim 15, wherein the:
4-amino-4-deoxychorismate synthase from Pseudomonas fluorescens (strain SBW25) comprises SEQ ID NO:4;
4-amino-4-deoxychorismate mutase from Photorhabdus laumondii subsp. laumondii (strain DSM 15139/CIP 105565/TT01) comprises SEQ ID NO:6;
4-amino-4-deoxyprephenate dehydrogenase from Pseudomonas fluorescens (strain SBW25) comprises SEQ ID NO:8;
aminotransferase (AT) from Escherichia coli (strain K12), if present, comprises SEQ ID NO:(SEQ ID NO:13); and
decarboxylase (DC) from Papaver somniferum, if present, comprises SEQ ID NO:9.

17. The engineered microbial cell of claim 1, wherein the non-native enzymes comprise:
a 4-amino-4-deoxychorismate synthase having at least 70% amino acid sequence identity with a 4-amino-4-deoxychorismate synthase from Streptomyces sp. CB01635;
a 4-amino-4-deoxychorismate mutase having at least 70% amino acid sequence identity with a 4-amino-4-deoxychorismate mutase from Streptomyces pristinaespiralis;
a 4-amino-4-deoxyprephenate dehydrogenase having at least 70% amino acid sequence identity with a 4-amino-4-deoxyprephenate dehydrogenase from Pseudomonas sp. 2822;
optionally, an aminotransferase (AT) having at least 70% amino acid sequence identity with an aminotransferase (AT) from Petunia hybrida; and
optionally, a decarboxylase (DC) having at least 70% amino acid sequence identity with a decarboxylase (DC) from Papaver somniferum.

18. The engineered microbial cell of claim 17, wherein the:
4-amino-4-deoxychorismate synthase from Streptomyces sp. CB01635 comprises SEQ ID NO:3;
4-amino-4-deoxychorismate mutase from Streptomyces pristinaespiralis comprises SEQ ID NO:5;
4-amino-4-deoxyprephenate dehydrogenase from Pseudomonas sp. 2822 comprises SEQ ID NO:7;
aminotransferase (AT) from Petunia hybrida, if present, comprises SEQ ID NO:14; and
decarboxylase (DC) from Papaver somnferum, if present, comprises SEQ ID NO:9.

19. The engineered microbial cell of claim 1, wherein the non-native enzymes comprise:
a 4-amino-4-deoxychorismate synthase having at least 70% amino acid sequence identity with a 4-amino-4-deoxychorismate synthase from Streptomyces sp. CB01635;
a 4-amino-4-deoxychorismate mutase having at least 70% amino acid sequence identity with a 4-amino-4-deoxychorismate mutase from Photorhabdus asymbiotica subsp. asymbiotica;
a 4-amino-4-deoxyprephenate dehydrogenase having at least 70% amino acid sequence identity with a 4-amino-4-deoxyprephenate dehydrogenase from Xenorhabdus doucetiae;
optionally, an aminotransferase (AT) having at least 70% amino acid sequence identity with an aminotransferase (AT) from Corynebacterium glutamicum; and
optionally, a decarboxylase (DC) having at least 70% amino acid sequence identity with a decarboxylase (DC) from Papaver somniferum.

20. The engineered microbial cell of claim 17, wherein the:
4-amino-4-deoxychorismate synthase from Streptomyces sp. CB01635 comprises SEQ ID NO:3;
4-amino-4-deoxychorismate mutase from Photorhabdus asymbiotica subsp. asymbiotica comprises SEQ ID NO:25;
4-amino-4-deoxyprephenate dehydrogenase from Xenorhabdus doucetiae comprises SEQ ID NO:29;
aminotransferase (AT) from Corynebacterium glutamicum, if present, comprises SEQ ID NO:16; and
decarboxylase (DC) from Papaver somniferum, if present, comprises SEQ ID NO:9.

21. The engineered microbial cell of any one of claims 2-20, wherein the engineered microbial cell additionally comprises a genotype change selected from the group consisting of:
p9_pENO1_KPA:GLN1,
p35_pKEX2_KPA:NUFM,
p9_pENO1_KPA:NUFM,
p115_pTHI11_KPA:PDC2, and
p5_pTDH3_KPA:PDC2.

22. The engineered microbial cell of any one of claims 1-2 and 10-21, wherein, when cultured, the engineered microbial cell produces 4-APEA at a level of at least 11 gram/liter of culture medium, optionally wherein, when cultured, the engineered microbial cell produces 4-APP at a level of at least 20 milligram/liter of culture medium, optionally wherein, when cultured, the engineered microbial cell produces 4-APhe at a level of at least 5 milligram/liter of culture medium.

23. A method of culturing engineered microbial cells according to any one of claims 1-22, the method comprising culturing the cells under conditions suitable for producing 4-APP, 4-APhe, and/or 4-APEA, optionally wherein the culture comprises:
4-APP at a level of at least 20 milligram/liter of culture medium;
4-APhe at a level of at least 5 milligram/liter of culture medium; and/or
4-APEA at a level of at least 15 milligram/liter of culture medium, wherein the culture comprises 4-APEA at a level of at least 11 gram/liter of culture medium.

24. The method of claim 23, wherein the method additionally comprises recovering 4-APP, 4-APhe, and/or 4-APEA from the culture.


SEQUENCE LISTING (269 sequences)

SEQ ID NO: 1 - protein, 370 aa, S. cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 2 - protein, 1545 aa, K. pastoris
MSGLIEKVSILGNDSIHVGYNMSSHIVEEILTKKASSTYVLITDSNIVKMGHLQTFIDEFNRLIPSRRPGSRILTYVVPPGEANKNRATKAAIEDYLLEKGCTRDTFILAIGGGVIGDMIGYVAATFMRGVRFVQVPTSLLAMVDSSIGGKTAIDTPLGKNFIGAFWQPDYVFVDVAFLETLPEREFINGMAEVVKTAAIWNEEEFSRLETYSKRFLNVIRNRRADSSVDLSSLKEHIIKLVLESIKVKAEVVSLDEREGGLRNLLNFGHSIGHAIEAILTPQALHGECVAIGAVLEAELSRYLGILSPVAVSRLYKCFAAYGLPVSLADKTVQQRTNGKKCPVDVLLQKMAIDKKNDGSKKKVVLLSKIGKCYEPKASYVNDEDLRFILTDEVLVKDFNSAPSTAVVVPPGSKSISNRALILAALGKGECKIKNLLHSDDTEHMLNAVAALKGADISFDDNGETVVVTGNGGNFTATNAEIYLGNAGTASRFLTSVASIVQPDSTTTHVVLTGNARMQERPIGPLVDALRNNGSEIEYLNREGSLPLKIESGNGLKGGRIKLAATISSQYVSSVLMCAPYAAEPVTLSLVGGKPISLLYVDMTIAMMKSFGIEVSKSTTEPYTYHIPQGHYTNPTEYVIESDASSATYPLAFAAMNGTQVTIPNIGSSSLQGDARFAIDVLKPMGCKVEQTATSTTVQGPTKGTLRPLPLVDMEPMTDAFLTACVVAAIANDANQSTSIVGISNQRVKECNRIEAMITQLAKFGVRAKELEDGIEVFGIDYRHLKTPADGLYPYDDHRVAMSLSLLAGFTDSPVLIQERHCTGKTWPGWWDILHTTFNVELDGHEALVESATAKANGNKSIIIIGMRAAGKTTLSHVIAQTLKGFKVIDLDDVFVEKYGDIREFIKENSWELFREKEALIANDAFKNNSTDTVISTGGGIVETEASRKLLRHQMKNGHIVLHLHRDLEETVVFLSQDKTRPAYVDEITQVWERRKNLYKDCSNYFFFSPHCQTEREFFTLKKTFGKFIDRITGEAVSSIPDKRSAFVCLTYEDLAPVSSKLTKVTNGCDAVELRVDLLKQYDSHFISNQIGVLRNQTSVPILFTIRTKSQGGRFPNDNVEDIERLLNLAVKLGVEYVDLELSLPESLLDSMASNRKFTKIIGSHHDFSGTVKWNSVEWENKYLLASKLNVDIIKFVGTATSLNDNWELEHFRSLHTDKPFIGINMGPLGKVSRVFNTILTPVTHKDLPSSAAPGQLTLKEINEYFGQFGGSSPKKFYIVGKPISHSKSPELHNIFYNEFGLSHTFDKFETDDAAKVFNDLMKGNDELGGCAVTIPLKIDMLKYVDELSDSAKSIGALNTVIPFGHGRFIGDNTDWIGIRDSLYQAGCEITPGFSVGLVVGGGGTSRAAVYAFHQMGCSKIYMLNRTPSKLTEIKNHFPSDYNIQIVDSLDAIDEGDKLDAAVSTIPGDQPLDEQLKSLLQNLLEKKRGHAVLLEAAYKPRETPIMALAFSRGWKVVPGSKMLINQGIEQFYKWTGYQFSSHISL

SEQ ID NO: 3 - protein, 714 aa, Streptomyces sp. CB01635
MRTVRTLLIDNYDSFTYNLFQMLAEVNAEVPVVIKNDDHAAWAALDPADFDNVVVSPGPGHPGTDTDLGLSRRVIEEWDLPLLGVCLGHQALCLLGGGEVGHAPEPVHGRTSRIEHDGRELFAEIPSPLRVVRYHSLTVHELPAELESTASTRDGLLMAVRHRGAPRWGVQFHPESVSSEYGHRLLANFRDLSLRALGRAPLGVEVPGVAGDDTRQDRPGGPGDLRLHVRELAEAPDADAAFEELFGDAPARFWLDSSRVDPGLSRFTFLGAPQGPLGEILTYDVAERLVRVRDRSGDEVLRRPGTLFAHLERELAARSTPAPELPFEFNLGYVGYLGYETKADSGGDRTHTAELPDGAFVFADRMVAVDHADGRAWLLALSSDRHPASARDADDWLADAARALAGTVPRPPFRLLPDEELLPLDVEFRHDAAEYRELVEECRSLIRDGETYEVCLTNLLRVPGRVDPLTAYRALRAISPAPYAAYLQFPEGAVLSSSPERYLRIDAAGTAESKPIKGTRPRGATPAEDEALARGLAAAEKDRSENLMIVDLVRNDLGQVCEIGSVHVPKLFDVESYATVHQLVSTVRGRLAADVSRPEAVRAAFPGGSMTGAPKVRTMQFIDRLEGGPRGVYSGALGYFALSGAVDLSIVIRTIVATADAATIGVGGAVIALSDPDDEVQEMLLKARTTLRALRQAQAVAPAGKRELLAGSVR

SEQ ID NO: 4 - protein, 689 aa, Pseudomonas fluorescens
MKILLIDNFDSFTQNIAQYLYEVTGICADIVTNTVTYEHLQIEQYDAVVLSPGPGHPGEYLDFGVCGQVILHSPVPLLGICLGHQGIAQFLGGTVGHAPTPVHGYRSKITHSGSGLFRDLPEQFEVVRYHSLMCTHLPQELRCTAWTEEGVVMAIEHESRPIWGVQFHPESIDSEYGHALLSNFIGMAIEHNGNHRTSATQNPDASASANEHYRAVGGLLNMQLAYRTYPGPFDPLALFTQRYAQDHHAFWLDSEKSERPNARYSIMGSGQAQGSIRLTYDVNSESLTLAGPKGSRIVTGDFFTLFSQIVESVNVAVPQYLPFEFKGGFVGYMGYELKALTGGNKVYRSGQPDAGFMFAPHFFVFDHHDQTVYECMISATGQSPQWPQLLTSMTTLNNATDRRPFVPGAVDELELSLEDGPDDYIRKVKQSLQYITDGESYEICLTNRARMSYSGEPLAAYRRMREASPVPYGAYLCFDSFSVLSASPETFLRIDEGGLIESRPIKGTRARSKDPSEDQRLRSDLQASTKDRAENLMIVDLVRHDLNQVCRSGSVHVPHIFAVESFSSVHQLVSTVRGHLRNDISTMEAIRACFPGGSMTGAPKKRTMEIIDGLETCARGVYSGALGWISFSGSAELSIVIRTAVLHKQQAEFGIGGAIVAHSDPNEELEETLVKASVPYYSFYAGSEK

SEQ ID NO: 5 - protein, 129 aa, Streptomyces pristinaespiralis
MTPPAIPAAPPATGPAPATDPLDALRARLDAADAALLDAVRTRLDICLRIGEYKRLHQVPMMQPHRIAQVHANAARYAADHGIDPAFLRTLYDTIITETCRLEDEWIASGGAPVPTPVHASASARGAVS

SEQ ID NO: 6 - protein, 119 aa, Photorhabdus laumondii subsp. laumondii
MTKIKHMTQAIINKQEILQPYRESLDVINMQILALLSERMKVCMKIAEVKAGQDIPMMQPQRITSLLNMLRDKSTDFGLRPEYTESIFQLVIEETCRREEELIDQLLHEKVKNNENTAH

SEQ ID NO: 7 - protein, 290 aa, Pseudomonas sp. 2822
MNTQAVVVLGGAGLIGSLISRILKQYGYIVRVVDRRPPDSDCEFHEMDVTQPFNHSGAVFRDAIAVVFALPESVAVSAIAWVTRCLPSDAVMIPTCSVQGPFFKALKAAAPQQRFVGVNPMFSPKLSVQGRTVAVCVEDVQTPQSFIERHLMEAGMKVRRMTPAAHDELMALCQALPHAAILGFGMALAQSSVDLDIVAEVMPPPMRTMMALLSRILVNPPEVYWDIQLENDQATAQREALVQGLERLQENIVEQDYERFRSDLQSVSKVLGKRLNAGAVDCQHLFSLLN

SEQ ID NO: 8 - protein, 290 aa, Pseudomonas fluorescens
MNTNTVVVLGGAGLIGSMISRILKQYGYFVRVVDRRPAEFECEYHEMDVTKPFNDTGAVFRNATAVVFALPESVAVSAIPWVTTFLSSEVVLIPTCSVQGPFYKALKAAAPRQPFVGVNPMFSPKLSVQGRSVAVCVEDTQAAQTFIERHLMEAGMKIRRMTPSAHDELMALCQALPHAAILGFGMALAKSSVDMDIVAEVMPPPMRTMMALLSRILVNPPEVYWDIQLENDQATAQRDALVHGLERLQENIVEQDYERFKSDLQSVSTALGKRLNAGAVDCQHLFSLLN

SEQ ID NO: 9 - protein, 531 aa, Papaver somniferum
MGSLNTEDVLENSSAFGVTNPLDPEEFRRQGHMIIDFLADYYRDVEKYPVRSQVEPGYLRKRLPETAPYNPESIETILQDVTTEIIPGLTHWQSPNYYAYFPSSGSVAGFLGEMLSTGFNVVGFNWMSSPAATELESVVMDWFGKMLNLPESFLFSGSGGGVLQGTSCEAILCTLTAARDRKLNKIGREHIGRLVVYGSDQTHCALQKAAQVAGINPKNFRAIKTFKENSFGLSAATLREVILEDIEAGLIPLFVCPTVGTTSSTAVDPISPICEVAKEYEMWVHVDAAYAGSACICPEFRHFIDGVEEADSFSLNAHKWFFTTLDCCCLWVKDPSALVKALSTNPEYLRNKATESRQVVDYKDWQIALSRRFRSLKLWMVLRSYGVTNLRNFLRSHVKMAKTFEGLICMDGRFEITVPRTFAMVCFRLLPPKTIKVYDNGVHQNGNGVVPLRDENENLVLANKLNQVYLETVNATGSVYMTHAVVGGVYMIRFAVGSTLTEERHVIYAWKILQEHADLILGKFSEADFSS

SEQ ID NO: 10 - protein, 625 aa, Enterococcus faecium
MSESLSKDLNLNALFIGDKAENGQIYKALLNELVDEHLGWRQNYMPQDMPIITPEEKSSASFEHTVNKTKDVLSEISARMRTHSVPWHNAGRYWGHMNSETLMPSLLAYNFAMLWNGNNVAYESSPATSQMEEEVGMEFAKLMSYKDGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLNDLSFKVGDKEIEVHPLTYPDFNMVDYVFKEKGNDDLVAMNKLNHDVYDYSSYVKGSIYGNEFLTSHTDFAIPDYGNSPLQFVNQLGFSDEEWNRAGKVTVLRASVMTPYMNKEEHFEEYAEKIKAALQEKLEKIYADQLLASEAK

SEQ ID NO: 11 - protein, 471 aa, Solanum lycopersicum
MGSLSLEMDFEPSPMTPRSLAAMTPRSLARRRLFPNVDNKKQKMAQPGAGPRKNLELEVMEPALKNDGPSLDTILVNYLDTLTQRVNYHLGYPVNICYDHYATLAPLLQFHLNNCGDPFLQNTVDFHSKDFEVAVLNWFAKLWEIEKDQYWGYVTNGGTEGNLHGILLGRELLPEGILYASKDSHYSVFKAARMYRMDSETINTSVNGEMDYSDLRAKLLQNKDKPAIINVTIGTTFKGAIDDLDVILEILKECGYSQDRFYIHCDAALCGLMTPFINNMISFKKPIGSVTISGHKFLGCPMPCGVQITRKSYINNLSTNVEYIASVDATISGSRNGLTPIFLWYSLSAKGQVGLQKDVKRCLDNAKYLKDRLQQAGISVMLNELSIIVVLERPRDHEFVRRWQLSCVKDMAHVIVMPGITREMLDNFMSELVQQRKVWYQNGKTDPPCVGEDIGAQNCACSYHKIDYICP

SEQ ID NO: 12 - protein, 471 aa, Solanum lycopersicum
MGSLTLEMDFEPSPMTPRSLAAMTPKSLARRRLFPNVDNKKQKVEQSGAGPRKNLQLEVMEPSLNNNGPSLDTILVNYLDTLTQRVNYHLGYPVNICYDHYASLAPLLQFHLNNCGDPFLQNTVDFHSKDFEVAVLDWFAKLWEIEKDQYWGYVTNGGTEGNLHGILLGRELLPEGILYASKDSHYSVFKAARMYRMDSETINTSVTGEMDYSDLRAKLLQNKDKPAIINVTIGTTFKGAIDDLDVILETLKECGYSQDRFYIHCDAALCGLMTPFINNMISFKKPIGSVTISGHKFLGCPMPCGVQITRKSYINNLSTNVEYIASVDATISGSRNGLTPIFLWYSLSAKGQVGLQKDVKRCLDNAKYLKNRLQQAGISVMLNELSIIVVLERPRDHEFVRRWQLSCVKDMAHVIVMPGITREMLDNFVSELVQQRKQWYRDGKAEAPCVGEDIGAQNCACSYHKIDYIGP

SEQ ID NO: 13 - protein, 397 aa, Escherichia coli
MFQKVDAYAGDPILTLMERFKEDPRSDKVNLSIGLYYNEDGIIPQLQAVAEAEARLNAQPHGASLYLPMEGLNCYRHAIAPLLFGADHPVLKQQRVATIQTLGGSGALKVGADFLKRYFPESGVWVSDPTWENHVAIFAGAGFEVSTYPWYDEATNGVRFNDLLATLKTLPARSIVLLHPCCHNPTGADLTNDQWDAVIEILKARELIPFLDIAYQGFGAGMEEDAYAIRAIASAGLPALVSNSFSKIFSLYGERVGGLSVMCEDAEAAGRVLGQLKATVRRNYSSPPNFGAQVVAAVLNDEALKASWLAEVEEMRTRILAMRQELVKVLSTEMPERNFDYLLNQRGMFSYTGLSAAQVDRLREEFGVYLIASGRMCVAGLNTANVQRVAKAFAAVM

SEQ ID NO: 14 - protein, 447 aa, Petunia hybrida
METNVVKVKSNMVDGDTNQDIETPNNITIKGILGLLMANTETASDDEDEDIVNGGGNLLIKMKKKKKVISLGMGDPTAFSCFHSPAVAQDAVLETLASHKFNGYAPTVGLPQTRKAIADYLSRDLPDKLCADDVYITAGCTQAIETALSILARPGSNILLPRPGFPIYALCAAFRHIEVRYFDLIPDKGWEVDLNAVEALADRNTIAIVVINPGNPCGNVYSYQHLKEIAETAKKLRTIVIADEVYGHLVFGAKPFVPMGVFGAIAPVLTLGSLSKRWLVPGWRLGWLVTNDPNGTFKNPKFVERIKKYCDICGGPATFIQAAVPRIIEKTEEVFFRKTINLLRWTSDICCEKMKEIPCISCPHKPEGSMAVMVKLNLSILSDISDDIDFCFKLAKEESVIVLPGLAVGLKNWIRITFAADPSSLEEALGRLKSFCHRHSYQQNGHC

SEQ ID NO: 15 - protein, 420 aa, Arabidopsis thaliana
MGENGAKRWNFGANEVVERSNSLTIRDYLNTLINCLDGGDVRPVIPLGHGDPSPFPSFRTDQAAVEAICDAVRSTKFNNYSSSSGVPVARKAVAEYLSSDLSYQISPNDVHITAGCVQAIEILISALAIPGANILLPRPTYPMYDSRAAFCQLEVRYFDLLPENGWDVDLDGVEALADDKTVAILVINPCNPCGNVFSRQHLQKIAETACKLGILVIADEVYDHFAFGDKPFVSMAEFAELVPVIVLGAISKRWFVPGWRLGWMVTLDPHGIMKDSGFVQTLINVVNMSTDPATFIQGAMPDIIGNTKEEFFSSKLEMVKKCAEICYEELMKIPCITCPCKPEGSMFTMVKLNFSLLEDISDDLDFCSKLAKEESMIILPGQAVGLKNWLRITFAVELELLIEGFSRLKNFTERHSKNQP

SEQ ID NO: 16 - protein, 341 aa, C. glutamicum
MIRADLATIPTYVPGRRLVDATKLSSNEVSFSPLPAAVDAVTEATWGANRYPDMGAVELREALAEHLEVEFDQVTVGCGSSALCQQLVQATCAQGDEVIFPWRSFEAYPIFAQVAGATPVAIPLTADQNHDLDAMAAAITDKTRLIFICNPNNPSGTTITQAQFDNFMEKVPNDVVVGLDEAYFEFNRADDTPVATEEIHRHDNVIGLRTFSKAYGLAGLRVGYAFGNAEIIAAMNKVAIPFAVNSAAQAAALASLNSADELMERVEETVEKRDAVVSALGAAPTQANFVWLPGEGAAELAAKLAEHGIVIRAFPEGARISVTNAEETDKLLRAWEAINAG

SEQ ID NO: 17 - protein, 825 aa, Aspergillus flavus
MAPLLGSWTASSAGGPSKAQLHTSTRKNILYVDAYDSFSYNVVAMLEEVLGVKVTVLMIDSEWPEGNKAEFLQYFEAVVLGPGPGDPNVPQDIGIMRDIWDLSDADLLPVFGICLGFQSLCLHHSIAIERLSYPLHGRVYRISTAEKDIFKDLQDVEVTLYHSLYAKLDDSLEAALAEYSGKQIKANADLDLLAWLPIEQDNVSERTIIPMAVRHREKPFWGVQFHPESCKSDRGVCSELLRKWWEMSLDYNKKHGRGGYGFLPEDITRGPSDMTSLPEIAFTMLNWCASTSRSSAFRSLPTSRLNAEFICERLNAPGSPTVLFQSNGRYSIISVPGPTSWRLEYYAQAEALLVERLQGHCNGHTNSGIKKSLSVYDLWETLRYLMDMKKVEFGDDAVPFWGGFLGYFSYEMGLACLARPKTHSDDNHGYIPLQNAAATTDPADVSLLWTERSVVVDNETGRITIQSTRNADDKSTGWLDETLQYLTELSRVDVLENENANSDAGYLDTILRQCVIRFPNEQTYKQQLEACKAELEAGESYELCLTCETSITLPSPSTDSERVAFPWKLYKRLREYNPAAFSAYARLGTVKVVSSSPECFLNWDRSFTLEMKPMKGTVRKSASMNMKKAKEILGSTKEMAENLMIADLIRHDLYGICGSGRVRVEKLLEVEDHGRVYQMITHVKGDVDPGRLGFAVRNLPHLHSSNMSAYGLTALQRCLPPGSMTGAPKERSCMHLSLIEARKRGVYSGVMGFLDLGGGGSFSVLIRTAFSCSDDKDNEQTWRIGAGGAVTILSTADGEWNEMLTKLTTVCGVFAPSDLQGSGKV

SEQ ID NO: 18 - protein, 703 aa, Arthrobacter sp. ATCC 21022
MTPAPVIIAVDGRSGAGKTTLAVELAARLREHHKVSLFHLEDIYPGWNGLASGIERYVATVLAPLRDGKPAEWISWDWEKHYDGRTQVTLPAEIVIVEGVGAAAQAARGMLDAVVWVEASGEDRRRRALARDGGSYEPYWDAWAQQEEEWLAVDPVTEAADIRVQNPADGSAPAHVLQAMTYLAPLAAVLSPELSARRGLQLRAERIDAAPDAEALFQDVYGSSTNAVWLDSSNADTAITGGTQAAARSRFSIMADDGGTFGQSVRHSSGTTWVTTGSATVRSSGPFFRWLDSVWGRRAVRSPEGYDGQFTLGWLGYLGYELKRETGGNDVSSATPDAALVFAGRALVLDHREHAVWLLALEAPDADEWFSVARAAVGAADAHPAVHPATVRPVSGLPVSGRRAVGVPIDTTGQAVAAASPEFACRDSATDYRRKITEAQHEISEGNSYEVCLTTTLEAPAGELDPWQSYQALRRRNPAPFASYLRFGGLAIASTSPERFLRITSDGGMRAEPIKGTRRRSADDAEDIRLRHDLETSLKDRAENIMIVDLLRNDLSHFAVPGSVTVSRLCAIESYATVHQMVSTVDAHLRPGAPRAEAVAAAFPAGSMTGAPKISTMDILDRLEAGPRGVYSGAIGYFSMNAATDLAVVIRTLVINPDGGGGRTLSLGVGGAVTADSTPDDEYDEIRTKAFGVLSTLGATFPS

SEQ ID NO: 19 - protein, 805 aa, Fusarium oxysporum f. sp. lycopersici
MGTIKVLIIDHYDSYTNNILQLLQGTQKGHDGQSYPEWSVSIVRFDQFSWDHFKSEILPHLDAIILSPGPGTPQREADFGFNARLIREVNIPILGICLGHQGIGTSFGANIIHAPNIKHGQICQIHHRGTGILSGLPQKFDGVRYNSLVLDIEDVPPELQVTAWTYDPENPSKKVLMGLQHRERPVFGTQWHPESVCSAHGKQILSNFRDIVLDFWVTSNPRNQWTKRRVLENASLPDHILQQNAVEQTDLGKALVAEANLARVVSPYYVKSARVGKGPSAEIVFQTLFRGRSLDGEAWLDSAKVRDSHSRNSYLSWASFALSYSTKSRHVSFYQHGKNIKSERLSVSYWSWLEQFQQGTIVANTETLPSESLDQETDVGQPLLQVGLIGYFGYELKRESLPGYQFTPPDEKDVSLKHSDSELLFANTVLRLDNYTGEWTLFSLIRRGEEDPIGNAIGASSKIGIDEAEFDLYLTRVREVFDAPPSPPYVEAHPLPNFVALDNEASYSNTIWAAKEAIKEGETYELTLTTKFRAKSPEVDPYSLYLDLRKRNPAPYSAYIHFAAHDKTILSSSPERFISIDSNGVAEMKPIKGTLAVDSDPVEDERRKHQLATDVKELAENLMIVDLIRSDLHNISPPSSISVPKLLHVETYQTVHQLVTTIQSHVASSVGGTKVIERCFPPGSMTGAPKLRSVQILDSLEGQRERGIYSGSIGYICASGTVDQSVVIRTIVKSGNELELGAGGAITWLSEADKEWDEVMVKANAVARGKGKAHVPDVAISPLGPFVEEEGHLRQEHPLRAAISA

SEQ ID NO: 20 - protein, 724 aa, Streptomyces pluripotens
MRTLLIDNYDSFTYNLYQLLGEVNGIPPVVVKHGEDWNALDLDAYDAIVVSPGPGRPDRERDFGISARAILDSGLPVLGVCLGHQGITHLFGGEVGLAPEPMHGRVSAVRHTGVDVFAGLPSPYDVVRYHSLAATRLPGELEAIAWTDDGVVMGVRHRTLPIWGVQFHPESISSQYGRELLANFRDLALAHGRRAPHTAVGASGASATAPYAVHVAKVDALPDTEAAYQALFATGSHGFWLDSSRVIDGLSRFSFLGNGSGPLAEYVTYRIADGVVSVQRPGQPEAGGEIISQGFFDYLDEQLGQRAVPTPEGLPFDFNLGYVGYLGYELKAEAGAVATHKAETPDAAVLFADRMLAVDHAEGNCYLLALSHGGDDADALSWLAETEQSLRRLPTPAGRSAQGQARFNGTGMTDPAAAGIVHLGHDREAYLDRIVRSMEEIRQGETYEVCMTNTVTMNATIAPLTTYGYLRQISPVPYGALLDYPGVAVLSASPERFMTIGTDRVAESKPIKGTRPRGATPEEDEALRQDLLGQEKDRAENLMIVDLIRNDLNVVSEIGSVHVPRLFHVESYAPVHQLVSTIRGTLRSGETAVTCVRAAFPGGSMTGAPKLRTMEIIDRLEGSARGVYSGSLGWFSLSGAADLNIVIRTLVATEDTVSFGVGGAIVALSDPEEEFEETDVKSRAMVSAVLATMRRDPSGADADAHPEAAAELAAVTVQSAGEAP

SEQ ID NO: 21 - protein, 787 aa, Saccharomyces cerevisiae
MLSDTIDTKQQQQQLHVLFIDSYDSFTYNVVRLIEQQTDISPGVNAVHVTTVHSDTFQSMDQLLPLLPLFDAIVVGPGPGNPNNGAQDMGIISELFENANGKLDEVPILGICLGFQAMCLAQGADVSELNTIKHGQVYEMHLNDAARACGLFSGYPDTFKSTRYHSLHVNAEGIDTLLPLCTTEDENGILLMSAQTKNKPWFGVQYHPESCCSELGGLLVSNFLKLSFINNVKTGRWEKKKLNGEFSDILSRLDRTIDRDPIYKVKEKYPKGEDTTYVKQFEVSEDPKLTFEICNIIREEKFVMSSSVISENTGEWSIIALPNSASQVFTHYGAMKKTTVHYWQDSEISYTLLKKCLDGQDSDLPGSLEVIHEDKSQFWITLGKFMENKIIDNHREIPFIGGLVGILGYEIGQYIACGRCNDDENSLVPDAKLVFINNSIVINHKQGKLYCISLDNTFPVALEQSLRDSFVRKKNIKQSLSWPKYLPEEIDFIITMPDKLDYAKAFKKCQDYMHKGDSYEMCLTTQTKVVPSAVIEPWRIFQTLVQRNPAPFSSFFEFKDIIPRQDETPPVLCFLSTSPERFLKWDADTCELRPIKGTVKKGPQMNLAKATRILKTPKEFGENLMILDLIRNDLYELVPDVRVEEFMSVQEYATVYQLVSVVKAHGLTSASKKTRYSGIDVLKHSLPPGSMTGAPKKITVQLLQDKIESKLNKHVNGGARGVYSGVTGYWSVNSNGDWSVNIRCMYSYNGGTSWQLGAGGAITVLSTLDGELEEMYNKLESNLQIFM

SEQ ID NO: 22 - protein, 126 aa, Pseudomonas putida CSV86
MSLDTCDGHGHGHGHGHGNGNASAELQPYRKQLDRINELLVDLLAERMQVCRLIARVKSARGIAIMQPLRVNSTLDQVRALSGPRKLRPEYLDGLFRLIIEETCDEERRVMAVLEASPEGREPCAR

SEQ ID NO: 23 - protein, 111 aa, Pseudomonas sp. Leaf48
MKQQGQLSQTDMSNILQSQRERLDLINLQVVDLLSERMKVCMGIADLKAIHGIAMMQPGRISYVLDMIKIQSEAVGLRPAYTESIFKLIIEETCIQEDLLINQYLGPGQSS

SEQ ID NO: 24 - protein, 104 aa, Photobacterium halotolerans
MNDAVVVRELKPYRDKLDEINEQIVALLASRMDICRTIANVKSRENIPMMQKDRVEFTLNKMKSLSVDYQLNPSYIGDIFERIIHETCEEELVIIKALSEEKEK

SEQ ID NO: 25 - protein, 119 aa, Photorhabdus asymbiotica subsp. asymbiotica
MTKSEYMTQSIITKQDVLQPHRESLDVINMQILSLLSERMKICMKIAEIKAEQDIPMMQPQRITSLLAMLKDKSTDFGLRPEYTELIFQLVIEETCRQEETLIDKLLREKKNNNENIAH

SEQ ID NO: 26 - protein, 129 aa, Streptomyces pristinaespiralis
MTPPAIPAAPPATGPAAATDPLDALRARLDAADAALLDAVRTRLDICLRIGEYKRLHQVPMMQPHRIAQVHANAARYAADHGIDPAFLRTLYDTIITETCRLEDEWIASGGAPVPTPVHASASARGGVS

SEQ ID NO: 27 - protein, 288 aa, Streptomyces sp. CB01635
MFEHCVVAGGTGEVGRMFAGRLAGSGAQVLCLDPAASAPAALADDIRAPGPEAARAIAAADLVLLAVPEPVALDAIGPVTRLMRADALFADTLSVKGGIAERLHALAPGIQAVGLNPMFAPSLPLPGRPVAAATVTDGPAVRAFLALISGWGARVVEMSAQEHDALTAAQQAATHAAVLAFGLALGELGADIGALRATAPPPHRTLLQLLARISSGTPEVYWDVQSGNPHAQAARRAVGHGLERLGRAVASGDPSAFETLFQELRATLGERHRTELADACAELFAQLR

SEQ ID NO: 28 - protein, 297 aa, Paenibacillus wynnii
MSQMIRRCVIVGGTGGIGGMFATLLAEAGVEVCLIDQNYLIVNFELLQCDITSPTPAAISVIRMADMVILALPERVAMNALAIVSNLMKPESLLAHTLSVQLPIATKIDYLGISLEVVGLNPMFAPNLSIVGRPVAAIIHNEGPRVAELLQLLSDVGARVVRIDAEEHDQLVAAMQALTHASIISFGLALSTLNVDIAKLSLLAPPPHSTMLALLARISSGTPEVYWDVQTANQQASMARIALVNAIHRLNALTTNESALDDFIDLLSETREVLGNEITSYQDLCALIFNGSQSATQ

SEQ ID NO: 29 - protein, 293 aa, Xenorhabdus doucetiae
MNNLKIVILGSNGAIGSFLGRIFSTSGHTVHGIDKQLSGNYTSYSQIDLLDPISNIDEIFNEADVIIFTLPETVAIKALPWVLSAINDNVLIVSTCSVQEPFYSSLKTISPQQPFIGINPMFSPSLQEKNRPVILIIEDMHNEHGWIELVLTKFQMRITKMRPKEHDKVMALCQVLPHALILAFGLALKANSIDINVLYNITPPPMRTMLALLSRLLVNPKEIYWDIQYENYMAVEIRKNILDELSLLDSMVQSGDKVEFNHSLDEIKLKLGEWLYTSYTDCQHIFQILNEDK

SEQ ID NO: 30 - protein, 290 aa, Pseudomonas frederiksbergensis
MMSDTVVVLGGAGLVGSLISRILMQYGYDVRVVDRRPSEQESGFYEVDVTSPFSNTGHIFQGAVAVVFALPENVAIRAIPWVVGSVSNDVVFIPTCSVQEPFYRALKNASSKQPFVGVNPMFSPKLSVQGRTVAICLEDGDSPSTFIEQHLMGAGMKIKRMTPVAHDELMALCQALPHAAILGFGLALARSSLDLESALEVMPPPMRTMMALLSRVLVNPPEVYWDIQLENDQANRQRAALAEGMERLMSNVSNQNFDEFRRDLQGVSDALGSRLNPGAVDCQHVFSLLN

SEQ ID NO: 31 - protein, 289 aa, Photorhabdus temperata subsp. temperata M1021
MSNHKVVILGGQGAIGSLLGHLFTQSGCTVYRVDQRTQGEHSFCYQLDILNPAADSEAIFTEAKAVVFALPEAVAIQALPWVLSVIGDDVQIVSTCSVQTPFHSALRAAAPGQPFIGVNPMFSPSLSEQKRPVAVILDNCHVNEHWIELVLTQAGMRISRMTPEEHDRVMAFCQALPHAAILIFGLVLAKSPLDIGTLVTVAPPPMRTMLALLSRVLLNPMEVYWDVQYENPMAAEPRQELLHAIQQLDSIVQAGDQTEFSNNLRHIAQQLGEQLNISAADCLAYFFNA

SEQ ID NO: 32 - protein, 386 aa, Escherichia coli
MTNNPLIPQSKLPQLGTTIFTQMSALAQQHQAINLSQGFPDFDGPRYLQERLAHHVAQGANQYAPMTGVQALREAIAQKTERLYGYQPDADSDITVTAGATEALYAAITALVRNGDEVICFDPSYDSYAPAIALSGGIVKRMALQPPHFRVDWQEFAALLSERTRLVILNTPHNPSATVWQQADFAALWQAIAGHEIFVISDEVYEHINFSQQGHASVLAHPQLRERAVAVSSFGKTYHMTGWKVGYCVAPAPISAEIRKVHQYLTFSVNTPAQLALADMLRAEPEHYLALPDFYRQKRDILVNALNESRLEILPCEGTYFLLVDYSAVSTLDDVEFCQWLTQEHGVAAIPLSVFCADPFPHKLIRLCFAKKESTLLAAAERLRQL

SEQ ID NO: 33 - protein, 351 aa, Caldanaerobacter subterraneus subsp. Tengcongensis
MIENLLREEIKGFKNYEVENVPYKYKMDANETPFELPEEVMKNIGDIVKSIHVNIYPDPTAEKLREELARYCSVTPKNIFVGNGSDEIIHLIMLAFVDKGDTVLYPHPSFAMYSIYSKIAGANEIAVNLNEDYTYNVERFAEAVERYKPKLVFLCNPNNPTGSVIDEEDIIRIIEKARGIVIVDEAYFEFYGKTLVPYIDRFENLIVLRTLSKAFGIAGLRVGYALSNGEIVKYLNLVKSPYNLNSLSQRIALEVLKSGVLKERVNYIINEREKLVKELNKINGIKVYPSHANFVLCKFENANDVHKRLVERGILVRNFSNVKGLEGTLRITVSSSDANDYLINALREILS

SEQ ID NO: 34 - protein, 513 aa, Saccharomyces cerevisiae
MTAGSAPPVDYTSLKKNFQPFLSRRVENRSLKSFWDASDISDDVIELAGGMPNERFFPIESMDLKISKVPFNDNPKWHNSFTTAHLDLGSPSELPIARSFQYAETKGLPPLLHFVKDFVSRINRPAFSDETESNWDVILSGGSNDSMFKVFETICDESTTVMIEEFTFTPAMSNVEATGAKVIPIKMNLTFDRESQGIDVEYLTQLLDNWSTGPYKDLNKPRVLYTIATGQNPTGMSVPQWKREKIYQLAQRHDFLIVEDDPYGYLYFPSYNPQEPLENPYHSSDLTTERYLNDFLMKSFLTLDTDARVIRLETFSKIFAPGLRLSFIVANKFLLQKILDLADITTRAPSGTSQAIVYSTIKAMAESNLSSSLSMKEAMFEGWIRWIMQIASKYNHRKNLTLKALYETESYQAGQFTVMEPSAGMFIIIKINWGNFDRPDDLPQQMDILDKFLLKNGVKVVLGYKMAVCPNYSKQNSDFLRLTIAYARDDDQLIEASKRIGSGIKEFFDNYKS

SEQ ID NO: 35 - protein, 426 aa, Dictyostelium discoideum
MIRSARLISNIKFGQKNIRQFSTNTNWWANVQKGPEDPILGVSIAYNKDTSPSKINLGVGAYRDENGKPYVLDCVKKADKKIYEANVDHEYAPIVGVAAFNQLAAQLALGEECKHIKEKRIATVQSISGTGALRIAADFFARFLKGKTAYVPNPTWGNHNVIFNDAGIPVKSYGYYNPATCGLNFEAMTKDIAAAPEGSIILLHACAHNPTGVDPTAEQWKKISEICKERGHFVLFDFAYQGFASGSPEKDAAAVRMFVEDGHNIALCQSFAKNFGLYGERIGAFSILTETSDQALNVESQLKILIRPMYSNPPVYGARLVQAILKDKELTNEWRSEVKGMADRIINMREQLVKYLKKHGSTRDWSHITTQIGMFCFTGLTPEQVDRLANEYHIYLTRNGRISIAGINSTNVEYLAKAMAAVTKDN

SEQ ID NO: 36 - protein, 397 aa, Klebsiella pneumoniae
MFQKVDAYAGDPILSLMERFKEDPRSDKVNLSIGLYYNDDGIIPQLQAVAEAEARLNAEPHGASLYLPMEGFSGYRQAIAPLLFGAEHTALKQNRIASIQTVGGSGALKVGADFLKRYFPESHVWVSDPTWENHIAIFEGAGFEVSTYPWFDKATNGVRFENLLAMLQTLPARDIVLLHPCCHNPTGADLTPAQWDRVVEVLKARQLIPFLDIAYQGFGGGLEEDAYAIRAIASAGMPMLVSNSFSKIFSLYGERVGGLSVVCEDSETAGRVLGQLKATVRRNYSSPPSFGAQVVATVLNDAALKATWQAEVDAMRAHILTMRQALVDALQQVAPGSKVDYLLKQRGMFSYTGFSAAQVDRLRDEFGVYLIASGRMRVAGLNSRNVQQVAKAFVAVM

SEQ ID NO: 37 - protein, 394 aa, Chlamydia trachomatis
MKRNPHFVSLTKNYLFADLQKRVAQFRLENPQHTVINLSIGDTTQPLNASVAEAFASSIARLSSPTTCRGYGPDFGLPALRQKLSEDFYRGFVDAKEIFISDGAKVDLFRLLSFFGPNQTVAIQDPSYPAYLDIARLTGAKEIIALPCLQENAFFPEFPEDTHIDILCLCSPNNPTGTVLNKDQLRAIVHYAIEHEILILFDAAYSTFISDPSLPKSIFEIPDARFCAIEINSFSKPLGFAGIRLGWTVIPQELTYADGHFVIQDWERFLSTTFNGASIPAQEAGVAGLSILPQLEAIHYYRENSDLLRKALLATGFEVFGGEHAPYLWVKPTQANISDRDLFDFFLREYHIAITPGIGFGRSGSGFVRFSSLGKREDILAACERLQMAPALQS

SEQ ID NO: 38 - protein, 394 aa, Paracoccus denitrificans
MLGNLKPQAPDKILALMGEFRADPRQGKIDLGVGVYKDATGHTPIMRAVHAAEQRMLETETTKTYAGLSGEPEFQKAMGELILGDGLKSETTATLATVGGTGALRQALELARMANPDLRVFVSDPTWPNHVSIMNFMGLPVQTYRYFDAETRGVDFEGMKADLAAAKKGDMVLLHGCCHNPTGANLTLDQWAEIASILEKTGALPLIDLAYQGFGDGLEEDAAGTRLIASRIPEVLIAASCSKNFGIYRERTGCLLALCADAATRELAQGAMAFLNRQTYSFPPFHGAKIVSTVLTTPELRADWMAELEAVRSGMLRLREQLAGELRDLSGSDRFGFVAEHRGMFSRLGATPEQVKRIKEEFGIYMVGDSRINIAGLNDNTIPILARAIIEVGV

SEQ ID NO: 39 - protein, 370 aa, S. cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPKYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 40 - protein, 370 aa, S. cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPKYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 41 - protein, 516 aa, Aspergillus oryzae
MDRDQFRAAAHATVDDIINYFDSVPDRRVCPTVTPGYLRPLIPEQPPTEPEEWSQIQADVDTKIKPGLTHWQHPNFMAFYPATVTYPSILGEMYSAAFNAPAFNWLCSPACTELETIVLDWVAQALNLPKCFMSSSENRGGGVLQVSASDTIATVMVAARERRVRELALAEGLKEGTLEYEDRVMDLRPRLVAMGSNQAHSSTAKGALIAGTRYRSATAKLENNMELTGDDVRAVLEQCEKDNLTPYYITLSMGTTSTCAVDRISEVTAVLREKPSWQRIWVHIDAAYAGSALVADEFQYLAKDLAEGVDSFNFNMHKWLLVNFDASCLFIRNRFDLTDALDITPAYLRNPYSESGQVIDYRNWSISLGRRFRALKIWFVMRSYGLNGLKAHVRKTIRVGDEFTNLVRSRSDLFELITKPAFGLTVFRIKDPRAQANGSAVNRTTAVKPNEKNDALTKEVYELINERGEIFITSTVVSGIYAIRVICANEAAEEKYLRRAFEILVETTDEVLGRSN

SEQ ID NO: 42 - protein, 396 aa, Methanocaldococcus jannaschii
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 43 - protein, 617 aa, Enterococcus silesiacus
MKNFNTDDTNLKALFIGDKGENVDLFKELLNKMIDEHVGWRQNYMPQDLPVITPQDRSSKSFQETADNMRSVFNVLSSRLRTESLPWHSAGRFWGHMNAETLMPSIIAYTTAMLWNGNNVAYESSPATSQMEEEVGMEFATLMGYEDGWGHIAADGSIANLEGLWYARNMKSLPFAIKAVVPEMVAGKSEWELLNMSTTEVLDILDQVQDNFDEIKAQSARSGKNLDKLGKWIVPQTKHYSWLKAADIIGIGLDQVIAGEVNSEYRMDIDKLEAQIRDLAAQGIPTLGVVGVVGSTEEGQVDRIDQIIALRDKLAKEGIYFYVHVDAAYGGYGRSIFLDENDKFIEWDKIEEVYAKNGIFMEKNDWLTREVYESFKAIEQAESVTIDPHKMGYIPYSAGGVVIKDIRMRDVISYFATYVFEKGADIPALLGAYILEGSKAGATAAAVWTAHKVLPLNVTGYGKLMGASIEGAYHFYHFIDGLEFQVGDKTIEMHALTKPDFNMVDYVFNEKGNTDLVKMNKLNHDFYDYASYAKGGLYTNEFITSHTDFAIEEYGHSPFEFVNSLGFSRAEWERADKVTILRAAGMSPYMNDKEIFDEYAEKIKTAIQEKLEAIYAE

SEQ ID NO: 44 - protein, 360 aa, Komagataella phaffii
MTSTPVQEEYDDVRIAGYDPLVSPALLQQEIPATKVALQTVVKGRKDAMNVVSLKDDRLLVVVGPCSIHDSDAALEYVARLKALSEELKDELVIIMRAYLEKPRTTVGWKGLINDPDLDNSFSINKGLKISRKLFVDLTNSGMPIASEMLDTISPQYLADLLSFGAIGARTTESQLHRELASGLSFPIGFKNGTDGTLDVALDAVQAASHAHHFMGVTLHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDAMLRELAEAVQKRRALKNAA

SEQ ID NO: 45 - protein, 360 aa, Komagataella phaffii
MTSTPVQEEYDDVRIAGYDPLVSPALLQQEIPATKVALQTVVKGRKDAMNVVSLKDDRLLVVVGPCSIHDSDAALEYVARLKALSEELKDELVIIMRAYLEKPRTTVGWKGLINDPDLDNSFSINKGLKISRKLFVDLTNSGMPIASEMLDTISPKYLADLLSFGAIGARTTESQLHRELASGLSFPIGFKNGTDGTLDVALDAVQAASHAHHFMGVTKHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDAMLRELAEAVQKRRALKNAA

SEQ ID NO: 46 - protein, 360 aa, Komagataella phaffii
MTSTPVQEEYDDVRIAGYDPLVSPALLQQEIPATKVALQTVVKGRKDAMNVVSLKDDRLLVVVGPCSIHDSDAALEYVARLKALSEELKDELVIIMRAYLEKPRTTVGWKGLINDPDLDNSFSINKGLKISRKLFVDLTNSGMPIASEMLDTISPKYLADLLSFGAIGARTTESQLHRELASGLSFPIGFKNGTDGTLDVALDAVQAASHAHHFMGVTLHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDAMLRELAEAVQKRRALKNAA

SEQ ID NO: 47 - protein, 378 aa, Komagataella pastoris
MTVSFLPIEPNNRTSPLTLQSTPVQEEYDDVRIAGYDPLVSPALLQQEIPATKVALQTVVKGRKDAMNVVSLKDDRLLVVVGPCSIHDSDAALEYVARLKTLSEELKDELVIVMRAYLEKPRTTVGWKGLINDPDLDNSFSINKGLKISRKLFVDLTNSGMPIASEMLDTISPQYLADLLSFGAIGARTTESQLHRELASGLSFPIGFKNGTDGTLDVALDAVQAASHAHHFMGVTLHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDSMLRELAEAVQKRRALKNAA

SEQ ID NO: 48 - protein, 378 aa, Komagataella pastoris
MTVSFLPIEPNNRTSPLTLQSTPVQEEYDDVRIAGYDPLVSPALLQQEIPATKVALQTVVKGRKDAMNVVSLKDDRLLVVVGPCSIHDSDAALEYVARLKTLSEELKDELVIVMRAYLEKPRTTVGWKGLINDPDLDNSFSINKGLKISRKLFVDLTNSGMPIASEMLDTISPKYLADLLSFGAIGARTTESQLHRELASGLSFPIGFKNGTDGTLDVALDAVQAASHAHHFMGVTKHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDSMLRELAEAVQKRRALKNAA

SEQ ID NO: 49 - protein, 378 aa, Komagataella pastoris
MTVSFLPIEPNNRTSPLTLQSTPVQEEYDDVRIAGYDPLVSPALLQQEIPATKVALQTVVKGRKDAMNVVSLKDDRLLVVVGPCSIHDSDAALEYVARLKTLSEELKDELVIVMRAYLEKPRTTVGWKGLINDPDLDNSFSINKGLKISRKLFVDLTNSGMPIASEMLDTISPKYLADLLSFGAIGARTTESQLHRELASGLSFPIGFKNGTDGTLDVALDAVQAASHAHHFMGVTLHGIAAITTTKGNEYCFVILRGGKKGTNYDPESVAEAKAKLPADGVLMIDCSHGNSNKDYRNQPKVSKVVADQVAAGEDRIIGVMIESNINEGKQSIPAEGRKALKYGVSVTDGCVSWETTDSMLRELAEAVQKRRALKNAA

SEQ ID NO: 50 - protein, 367 aa, Komagataella pastoris
MFIQNDHVGDRSRLEDWRIRGYDPLTPPDLLQHEYPLTAKAEENILKGRNEAVDILKGKDDRLLVIVGPCSIHDPQAALDYCERLAKFNETIKGELHIIMRAYLEKPRTTVGWKGLINDPDIDGSFQINKGLRISRELFVKLTEQIPIAGEMLDTISPQFLSDLFSLGAIGARTTESQLHRELASGLSFPVGFKNGTDGGLTVAIDAMRAASHPHHFLSVTLPGVVAIVGTEGNEDTFVILRGGKNGTNYDEASVDAAHAQLEKTGILGSGPAIMVDCSHGNSNKDHRNQPKVAEVVAEQIRKGSRKICGLMIESNIVEGRQDVPKEGKSHLRYGCSITDACISWEDTEKVLRVLADSVIERRNLKN

SEQ ID NO: 51 - protein, 375 aa, Komagataella phaffii
MPVPSSHKMFIQNDHVGDRSRLEDWRIRGYDPLTPPDLLQHEYPLTPKAEENILKGRNEAVDILKGKDDRLLVIVGPCSIHDPKAALDYCERLAKFNETIKGELHIIMRAYLEKPRTTVGWKGLINDPDIDGSFQINKGLRISRELFVKLTEQIPIAGEMLDTISPQFLSDLFSLGAIGARTTESQLHRELASGLSFPVGFKNGTDGGLTVAIDAMRAASHPHHFLSVTLPGVVAIVGTEGNEDTFVILRGGKNGTNYDEASVDAAHAQLEKTGILGSGPAIMVDCSHGNSNKDHRNQPKVAQVVADQIRKGSRKICGLMIESNIVEGRQDVPKEGKSHLRYGCSITDACISWEDTEKVLRVLADSVIERRNLKN

SEQ ID NO: 52 - protein, 370 aa, Saccharomyces cerevisiae
MFIKNDHAGDRKRLEDWRIKGYDPLTPPDLLQHEFPISAKGEENIIKARDSVCDILNGKDDRLVIVIGPCSLHDPKAAYDYADRLAKISEKLSKDLLIIMRAYLEKPRTTVGWKGLINDPDMNNSFQINKGLRISREMFIKLVEKLPIAGEMLDTISPQFLSDCFSLGAIGARTTESQLHRELASGLSFPIGFKNGTDGGLQVAIDAMRAAAHEHYFLSVTLPGVTAIVGTEGNKDTFLILRGGKNGTNFDKESVQNTKKQLEKAGLTDDSQKRIMIDCSHGNSNKDFKNQPKVAKCIYDQLTEGENSLCGVMIESNINEGRQDIPKEGGREGLKYGCSVTDACIGWESTEQVLELLAEGVRNRRKALKK

SEQ ID NO: 53 - protein, 350 aa, Escherichia coli
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLNMITLQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 54 - protein, 356 aa, Escherichia coli
MQKDALNKVHITDEQVLMTPEQLKAAFPLSLQQEAQIADSRKSISDIIAGRDPRLLVVCGPCSIHDPETALEYARRFKALAAEVSDSLYLVMRVYFEKPRTTVGWKGLINDPHMDGSFDVEAGLQIARKLLLELVNMGLPLATEALDPNSPQYLGDLFSWSAIGARTTESQTHREMASGLSMPVGFKNGTDGSLATAINAMRAAAQPHRFVGINQAGQVALLQTQGNPDGHVILRGGKAPNYSPADVAQCEKEMEQAGLRPSLMVDCSHGNSNKDYRRQPAVAESVVAQIKDGNRSIIGLMIESNIHEGNQSSEQPRSEMKYGVSVTDACISWEMTDALLREIHQDLNGQLTARVA

SEQ ID NO: 55 - protein, 348 aa, Escherichia coli
MNRTDELRTARIESLVTPAELALRYPVTPGVATHVTDSRRRIEKILNGEDKRLLVIIGPCSIHDLTAAMEYATRLQSLRNQYQSRLEIVMRTYFEKPRTVVGWKGLISDPDLNGSYRVNHGLELARKLLLQVNELGVPTATEFLDMVTGQFIADLISWGAIGARTTESQIHREMASALSCPVGFKNGTDGNTRIAVDAIRAARASHMFLSPDKNGQMTIYQTSGNPYGHIIMRGGKKPNYHADDIAAACDTLHEFDLPEHLVVDFSHGNCQKQHRRQLEVCEDICQQIRNGSTAIAGIMAESFLREGTQKIVGSQPLTYGQSITDPCLGWEDTERLVEKLASAVDTRF

SEQ ID NO: 56 - DNA, 1113 bp, S. cerevisiae
atgtcagaatcccccatgtttgcagctaacggcatgcctaaagtcaaccaaggagctgaggaggacgtaagaatacttggttatgatcccctggcttcacctgccttgttacaagtgcaaatccctgccacaccaacctcattagaaacggccaaaaggggacgtagggaggcaatagatattattaccggtaaagacgatagggttttggttattgtgggtccatgttccattcatgatctggaggcagcccaagaatacgctttgcgtttaaagaagttgagtgatgaattaaaaggagatctgtctattataatgagggcatacttagagaaacccagaacaaccgtgggctggaaaggattgataaatgaccctgatgtgaacaacacgtttaatatcaacaagggcctgcagtccgctagacagctatttgtgaatttgaccaacatcggactgcccattggctctgagatgttggataccatatctccacaataccttgcagacctggtaagttttggagctatcggcgccagaactacggaatcacagttacatcgtgaattagcatctggactgtccttccctgtgggattcaaaaacggtacggacggcacacttaacgtggccgttgatgcttgtcaagctgctgcccactctcatcacttcatgggagtcactctgcacggcgtagctgcaatcacgacgaccaaaggcaatgagcattgcttcgtaatattacgtggtggcaagaagggcaccaactacgacgctaagtccgtggccgaggcaaaagctcaactgccagctggctccaatggtttgatgattgactattcacatggaaattctaataaagattttagaaatcaaccaaaggtgaacgatgtggtatgcgagcaaatcgccaatggcgaaaacgccattactggcgtgatgatagaatctaatatcaacgaaggaaaccaaggaatacctgcagaaggtaaagccggtctaaagtacggagtttcaattaccgatgcttgtattggctgggaaacaactgaagatgttctgaggaagcttgctgctgctgttagacaaagacgtgaggtgaataaaaaataa

SEQ ID NO: 57 - DNA, 4638 bp, K. pastoris
atgtccgggcttatcgaaaaggtcagcatccttgggaatgactccattcatgttggttacaatatgtcgtcgcacattgtggaagagattttgacgaaaaaggcttcttccacctatgtcttgattacggattccaacattgtcaagatggggcatttacagactttcattgatgagttcaacaggcttattccttccagaaggccgggctccagaattctcacttatgttgttccccctggtgaagccaacaagaacagagctacgaaggctgctattgaagattatctgcttgaaaagggctgtaccagagataccttcattcttgccattggaggtggggttattggtgatatgattggatatgttgcagctacattcatgcgtggggttagatttgtacaggttcctacttcccttttagctatggtcgactcttccattgggggtaaaactgcaatagacacacctttaggtaagaacttcattggtgctttctggcaacctgactatgtctttgttgatgtggcattcttagaaacgttgcctgagagagagtttattaacggtatggctgaggttgtcaagacagcggctatctggaacgaggaagagttctccaggttggaaacttactcaaagaggtttttgaatgttataaggaatagaagagccgatagctcagttgatttatcttctttgaaagagcacatcattaaattggtactggagtccatcaaagtcaaggctgaagtagtctccttagatgaacgtgaaggaggattaagaaatctcctaaattttggacactctatcggtcatgctatcgaagccatactgacccctcaggcccttcatggagagtgcgttgcaataggtgcagttttggaagccgaactgtctagatacttgggaatcctgtcaccagttgcagtttcaaggttatacaaatgtttcgcagcttacggtttgcccgtgagtcttgctgataagacagtgcagcaaagaacaaacggtaaaaaatgccctgttgatgttttactacaaaaaatggcaattgacaaaaagaatgatggttcgaagaagaaggttgtcctgttatctaaaattggaaagtgttatgaacctaaagcatcttatgttaacgatgaggatctaagattcattttaactgatgaggttcttgttaaagatttcaactctgctccatccactgctgtagttgtgccccccggatccaaatcgatttccaatagagccttaatccttgctgctttgggtaaaggtgaatgcaagataaagaatctacttcattctgatgacaccgaacacatgttaaatgccgttgccgctcttaagggagctgacatatcttttgatgacaacggagaaactgttgttgtcacaggtaacggaggaaattttacagcaactaacgctgaaatttacttgggaaatgctggaactgcttccaggtttttgacctccgttgcatccatagttcagcctgattcaaccaccacgcatgttgtcctcacaggcaatgccaggatgcaagagagaccaatcgggccattagttgatgcccttagaaataacggctctgaaattgaatatttaaacagagaaggttcgctaccactgaaaattgaatctggaaatggactcaaaggaggccgtatcaaacttgccgctacgatatcttcacagtacgtttcttctgtgctaatgtgcgcaccatatgctgcagagcctgtcacgttgtctctcgtagggggaaaacctatttctctgctttacgtcgatatgaccattgcgatgatgaagagttttggtattgaagtctcaaagtccaccactgagccttacacttaccacatccctcaaggtcattatactaaccctacagagtatgttattgaatctgatgcctcctctgctacttatccattagcatttgctgccatgaatggcactcaggtgacaattccaaatattggatcttcttctttgcagggtgatgcgagatttgcaattgacgtcttgaaacctatgggatgtaaagttgaacaaactgctacttccactactgtccagggccctaccaagggcacattgagaccattgccactagttgacatggagccaatgacggatgcgtttttgaccgcatgtgttgtagccgctattgccaatgatgccaaccaatcaacctcaattgtcgggataagtaatcaacgtgttaaggaatgcaacagaatcgaagctatgatcacacaacttgccaaatttggcgtaagagccaaagagctagaagacggaattgaagtctttggaatagactatcgtcacttaaagactccagcagacggcctttacccctacgatgaccatagagttgccatgagtctttcactattggcaggttttactgactctccggtacttattcaagagcgtcattgtaccggtaagacatggcccggatggtgggatatattgcacaccacattcaacgttgaacttgatggacacgaagctcttgtcgaatctgctacagccaaggctaatggaaacaaaagcattatcattattggtatgagagccgcaggtaaaactacactttcccatgttattgctcagaccttgaagggtttcaaagttatagatttagatgatgtgtttgttgagaagtatggtgatatcagagaatttatcaaagagaacagttgggagctgttcagggagaaggaggccttgattgccaacgatgcttttaaaaacaactctacggatactgtgatttccactgggggtggaattgtcgagactgaagcatctcgcaagttattgaggcatcaaatgaagaatggtcatatcgtccttcatttgcatcgtgaccttgaagaaactgttgtctttttatctcaagacaagactcgtccagcatacgttgatgagattactcaagtttgggaaagaagaaaaaatttatacaaagattgttccaactacttcttcttctcccctcattgccaaactgaaagagaattttttactttgaagaaaacctttggtaaattcattgacagaattacgggtgaggccgtttcaagtattcctgacaagcgttcagcttttgtgtgtttaacatatgaagatctggctccagttagctcaaagctgactaaggtcaccaatggttgtgatgcagtagaacttagagttgatttgctgaaacaatatgactctcatttcatcagcaatcagattggtgttttaagaaaccagacaagtgtgcctattcttttcaccatcaggacaaagagccagggaggaaggtttccaaatgataatgttgaggacattgaaaggcttctcaatttggccgtcaaactaggtgttgagtatgttgatttggagttgtctctaccagagtccttgcttgacagtatggcgtctaacagaaagtttactaaaattattggatcgcatcatgacttttccggtaccgtaaagtggaacagtgtggaatgggaaaacaagtatctcttagcttcgaagttgaatgttgatattattaaatttgttggaactgcgacgagcttgaacgacaactgggagttagagcattttagaagccttcatactgataagcctttcattggaatcaatatgggacctttgggaaaagtttccagagttttcaacacaattttgaccccagtaactcacaaagatcttccgtcaagtgcagctccaggtcaattaaccttgaaagagattaacgaatatttcggacaatttggaggaagcagccctaaaaagttttacattgtgggaaaaccaattagccattctaagtctcctgaactgcataatatcttctacaacgaatttggtttgagtcacactttcgacaaatttgaaacagatgacgccgctaaggttttcaatgatctcatgaaaggtaacgatgaactgggcggctgcgcagtgaccattcccctcaagatcgacatgctgaagtatgtggatgagcttagtgattcagcaaaatctattggtgcattgaatactgtaattccatttgggcatggtaggttcattggagataataccgactggattggaatcagagattctctttatcaggccggatgcgaaataacccctggatttagtgttggcttggtagtaggaggtggcggaacctcaagagcagcagtttatgctttccatcagatgggatgctctaagatctacatgttgaatagaactccatctaagctgactgagatcaagaatcatttcccatcagattataacatacaaatcgttgactctctggatgcaattgatgagggtgacaagctggatgctgctgtatcaacaattccaggagaccaacccctagatgaacagttgaagtctttgcttcaaaacttattagagaagaaacgtggtcacgctgtgctcttagaagcggcttacaaaccaagagagacgccaattatggcattggcattttccagaggttggaaagttgttcctgggagtaagatgctgatcaaccaaggaattgagcaattctacaagtggactgggtatcaattctcctcccacattagtctttag

SEQ ID NO: 58 - DNA, 2145 bp, Streptomyces sp. CB01635
atgagaactgtcagaacactactaattgataattatgactcttttacgtataacttgtttcaaatgctagctgaagtcaacgccgaagttcctgtagtaatcaagaatgacgatcacgcagcctgggctgcattggacccagctgacttcgataatgtggtggtttctcctggcccaggccatccaggcactgacacggacttaggcctttctagacgtgttatagaagagtgggatttaccacttctgggagtgtgcttgggtcatcaagccctatgtttgctgggtggaggtgaggttggtcacgctcctgaaccagtccacggcaggacctctaggatagagcacgatggtcgtgagctgttcgcagagataccttctcctttacgtgtagtgcgttatcattcattaaccgttcacgagcttccagccgagcttgagtccaccgctagtacccgtgatggacttctgatggccgttaggcacagaggagctcccaggtggggagttcaatttcaccccgaatctgtttcctctgaatacggccatagactgttagctaacttcagggacctatctttgcgtgcattaggccgtgcacccctaggcgtcgaggtacctggcgtggctggagatgatactcgtcaagacaggcctggcggacctggagacttacgtctacacgttcgtgagttagcagaagcccctgacgcagacgcagcatttgaggaattatttggcgacgcccctgctcgtttttggttagattcaagtagagttgaccccggattgtccagatttacgttcttaggtgcaccacagggcccactaggtgagatactaacgtacgacgtcgctgaaagactagttagggttagggacagaagtggcgatgaggtcttgaggcgtcctggtaccttattcgcacatcttgaacgtgaattagcagcaaggtcaactcccgctcccgaattaccatttgagttcaaccttggatacgtcggctatcttggctatgaaactaaagccgactcaggaggagatcgtacccacacggcagagttacccgatggtgctttcgttttcgctgaccgtatggttgctgtggatcatgctgatggaagagcctggctactagcactttcatctgatagacaccctgccagtgcaagggacgcagacgattggcttgctgacgctgcccgtgcattggctggcacggtccccaggcctccatttcgtttacttcccgacgaagagctgctgccactagatgtggaatttcgtcacgacgccgccgagtacagagagctagttgaagagtgcaggtctttaatacgtgatggagagacatatgaggtgtgcctaaccaacctgttacgtgtcccaggcagggtagaccctttgaccgcatatagggcactaagggcaatttcacctgctccatatgcagcttacctgcagtttccagagggcgctgtgttgtccagtagtccagaaagatatttaagaatagacgcagcaggcacagctgaatcaaagccaatcaagggaacacgtccaaggggtgcaacaccagccgaagacgaggcattggctagaggattagcagcagctgagaaagacaggtccgaaaatttgatgatcgtggaccttgttcgtaacgatctgggacaagtgtgtgagattggttcagtacatgtccccaaacttttcgatgttgaatcctatgcaacagttcaccagctggtttcaacagtacgtggcagactggccgccgatgtcagtcgtcccgaggccgtcagggctgcatttccaggtggttccatgaccggcgcacctaaagtgagaacgatgcaatttatcgacaggttggaaggtggtcccagaggcgtttactctggcgctttaggctactttgccctatctggcgccgtagacttgtcaatcgtaatccgtacaatcgtggctacagcagacgctgctaccattggtgttggaggagccgtgatcgctctatctgacccagacgacgaagtccaagagatgttgttaaaggcaaggacgactttgagggctttgagacaagcccaggccgtcgctccagccggcaagcgtgagttactggctggttccgtccgttaa

SEQ ID NO: 59 - DNA, 2070 bp, Pseudomonas fluorescens
atgaagatcttgctaattgataatttcgactcattcactcagaatatcgcacagtacctgtacgaagtgacaggtatatgtgccgacatcgtaacaaacacagtgacgtatgaacaccttcagattgaacagtacgatgccgttgtgctttcaccaggtcccggacatcccggtgaatacctggattttggtgtgtgcggacaggttatacttcatagtcctgtgcccttactgggtatctgtcttggccatcaaggaatcgcccaatttttaggcggtacagtaggccatgcaccaacacccgtgcacggctatagatccaaaataacacactctggctccggcttatttcgtgatttgccagagcagttcgaggtggtacgttatcactcattaatgtgtacacaccttccacaggagttaaggtgcaccgcctggacggaggagggcgtagtgatggctatcgaacatgaatctagaccaatatggggtgttcagttccaccccgaatcaatagattctgagtacggtcatgcacttctatctaatttcataggcatggccattgagcacaacggtaaccacagaacgtccgctactcaaaacccagatgctagtgcctcagctaacgagcactaccgtgccgttggaggtttattaaacatgcagttagcctataggacatatcccggaccctttgacccccttgctttgttcacgcaaaggtacgctcaggaccaccacgcattttggctggatagtgaaaagagtgagaggcctaacgctaggtactcaataatgggatcaggccaagctcaaggttctattagacttacatatgacgttaattctgagagtctgactctggcaggcccaaagggttcaagaatagtaacaggagactttttcactcttttctctcagatcgttgaatccgttaacgtagctgtcccccagtatttacctttcgagtttaaaggcggtttcgttggttacatgggctatgaattaaaggcattgacaggcggaaataaagtctacagatccggacagccagatgcaggatttatgttcgctccccacttctttgtgttcgatcatcatgatcagacggtatacgaatgcatgatttctgccacaggacagagtccacaatggccccaacttctgacgtcaatgacgaccctgaataatgcaaccgataggagacccttcgtaccaggcgcagttgatgagttagagttgtctctagaagatggcccagatgactacataagaaaagttaagcaaagtttgcaatatatcacggatggtgaatcctatgagatttgccttactaaccgtgcaagaatgtcctacagtggtgagccactagcagcctacaggaggatgagagaagcctccccagtcccctatggcgcttacttatgttttgactcattttctgtcctatccgcttcacctgagacgttccttagaatagatgagggtggattaatcgagagtcgtcctatcaaaggaacccgtgcacgtagtaaagatccttccgaggaccaaaggttaaggtcagatctgcaagcatctactaaggatcgtgcagaaaatttgatgatagtcgacctggtcaggcacgatctgaatcaggtttgcagatccggttccgttcacgttccacacatctttgccgttgaatcattctcttcagtgcatcagctagtctcaactgttagaggacatctacgtaatgacatctctacgatggaggctataagggcctgctttccaggtggttccatgaccggcgctccaaaaaagcgtacgatggaaattatcgacggcctagagacttgcgcaagaggcgtgtactctggcgccttaggctggatttctttcagtggaagtgctgaactatctattgtgattaggaccgctgtcttacataagcaacaagccgaatttggaataggtggcgctatcgtggcccattccgatccaaatgaagaacttgaagaaacacttgtgaaagcatctgtaccttactatagtttttacgcaggcagtgagaagtaa

SEQ ID NO: 60 - DNA, 390 bp, Streptomyces pristinaespiralis
atgacacctccagccattcctgccgcccccccagcaactggacctgctccagctactgatcctttggatgctctgagggcaagattagatgccgcagatgctgcattgttagatgcagtgagaacgcgtctggatatttgtctaagaatcggagagtacaagagactacatcaagtgcccatgatgcaacctcaccgtatagcacaggtccatgctaacgcagcacgttatgccgctgatcatggtatagaccccgcctttctaaggacgctgtatgatacgattataacggaaacttgtcgtcttgaagacgaatggattgcctctggcggtgcacctgttcctactccagtccacgcatctgcaagtgcccgtggagcagtctcctaa

SEQ ID NO: 61 - DNA, 360 bp, Photorhabdus laumondii subsp. laumondii
atgacaaagataaaacacatgacccaggcaattattaacaaacaggaaattttgcaaccctatcgtgagtcccttgatgtcatcaatatgcaaatattggccttgttaagtgaacgtatgaaagtctgcatgaagattgctgaagtaaaggctggacaggatatacctatgatgcaaccccagaggataacatcattattgaatatgttgcgtgacaaaagtaccgacttcggtctaagacccgagtacaccgagagtattttccagttagtcattgaggagacttgccgtagagaggaagaacttatcgaccagttactacacgagaaggtaaaaaataacgagaataccgctcactaa

SEQ ID NO: 62 - DNA, 873 bp, Pseudomonas sp. 2822
atgaatactcaggccgtagttgtacttggcggagccggactgattggctcattgatctccaggattctgaaacaatatggttatattgtaagagttgttgatagaagacctcccgactctgattgcgagtttcacgaaatggatgtgacccaaccattcaaccactctggcgctgtatttcgtgacgcaatagccgtcgtgtttgccttgcctgagtcagtcgcagtttccgccatcgcatgggttacgcgttgcttgccaagtgatgcagttatgattcctacctgcagtgtacagggtcccttcttcaaggctcttaaagccgctgcaccccaacagagattcgtcggagtcaacccaatgttctctcctaaactatcagttcaaggtagaaccgtagctgtatgtgtcgaagatgtacagacaccccaatctttcatcgaaaggcacttaatggaggctggcatgaaagttcgtagaatgacccctgcagcacatgatgaattgatggctttgtgccaagcactgccccacgctgcaatccttggatttggcatggcactggctcagtcctctgtagatttggatattgtagccgaggtgatgccaccacccatgaggaccatgatggccttgctatcaaggatcttggtcaaccctcctgaggtttactgggacattcagttagagaatgaccaagccaccgctcaaagagaagcccttgttcaaggccttgaaagacttcaagagaacatcgtggaacaagattatgagaggtttagatccgatctacagtccgtatccaaggtgctaggcaagaggctaaacgccggtgccgtggactgccaacacctgttttctttgttaaactaa

SEQ ID NO: 63 - DNA, 873 bp, Pseudomonas fluorescens
atgaacactaatactgtcgtagtgctaggtggcgcaggtttgatcggaagtatgattagtagaatactaaagcaatacggttactttgtaagggttgtcgacaggaggcccgccgagttcgaatgtgaataccatgagatggatgtcactaaacccttcaatgacaccggtgctgtcttcagaaatgctacggccgtagtctttgctctacccgaatcagttgcagtatccgccatcccctgggtaacgacctttctatctagtgaggtcgtgctgattcccacgtgtagtgtccagggaccattttacaaagccttaaaggccgccgctccaaggcagcctttcgtcggcgtaaatcctatgttcagtccaaagttgtccgtgcaaggaaggagtgtggctgtgtgcgtggaagacacgcaggccgctcaaactttcattgaacgtcacctgatggaggctggtatgaaaattagacgtatgacaccatctgctcatgatgagctaatggccctttgccaagcattgcctcatgcagccatactaggattcggcatggctttagctaagtcctcagttgacatggatattgttgctgaagtaatgcctcctcccatgagaacaatgatggctttgttaagtcgtattttggtgaaccctcccgaggtctattgggatatccaattagagaatgatcaagccacagctcaaagggacgcacttgtgcatggtttggaaagattacaagagaatatagtagagcaagattacgagagattcaaaagtgatcttcaaagtgtatcaactgctctgggaaaacgtctaaacgcaggagccgttgactgtcaacacctattttccttattaaattaa

SEQ ID NO: 64 - DNA, 1596 bp, Papaver somniferum
atgggttcacttaacaccgaggacgttctggaaaactcatcagcttttggtgttaccaatcccttggatcccgaagagtttaggagacaaggtcatatgataatcgactttcttgctgactactatcgtgacgtagaaaagtacccagtccgttctcaggtggaaccaggttatttaagaaagagactacccgagacggcaccctacaaccccgagtctatcgagactatccttcaggacgtaacaacggagataatacctggcctaacccactggcaaagtcctaactattatgcatatttcccttccagtggatcagtagctggtttcctgggcgagatgttatccaccggtttcaatgtcgtaggcttcaactggatgtctagtccagccgccacagagttagaatccgtagttatggactggttcggtaaaatgctaaatctgccagaatcattcttgttctcaggatcaggaggtggcgtattacagggaacttcttgcgaagcaattctttgtacactaactgctgccagggatcgtaaacttaataagataggccgtgaacacataggcagattggtcgtttatggctcagaccagacgcactgcgctcttcaaaaggctgctcaagtagccggaataaacccaaagaatttcagagcaatcaagacttttaaggaaaacagttttggtttgtccgctgccactctgagggaggtcatcttggaagatatagaagcaggtttgatacctcttttcgtctgtcctaccgtgggcactacctcttccactgctgtagatccaatttctcctatctgcgaagtagccaaggaatacgaaatgtgggttcatgtggacgctgcttacgctggctctgcatgtatatgtcctgaattcagacacttcattgacggtgtagaagaggccgactctttttcactgaatgcccacaagtggttcttcaccactttggattgctgctgcttatgggtaaaggatccatccgctctagtcaaagcattatcaacaaatccagagtacttaaggaataaagctacagaatcccgtcaagtagtggactacaaagattggcagatagcactttccagacgttttcgttcattaaagctgtggatggtgttgcgttcatatggcgtaacaaacctaaggaatttcctgagatcccatgtcaaaatggctaagacgttcgagggcttgatctgtatggatggcaggttcgagatcacggtcccaagaacatttgcaatggtgtgcttcaggcttttaccccccaaaacaataaaagtttatgataacggcgtccatcagaatggcaatggtgtagtccctttgagagacgaaaacgaaaacttagttttggccaataaattaaaccaggtttacttggaaaccgtcaacgccaccggcagtgtatatatgacacacgctgtagtgggcggtgtctatatgataagattcgcagttggatcaacacttacggaggaaaggcacgtaatatatgcttggaagatcttacaagaacacgcagacctaatacttggaaaatttagtgaggctgatttttcttcttaa

SEQ ID NO: 65 - DNA, 1878 bp, Enterococcus faecium
atgagtgagtcactttcaaaagacttaaatctaaacgcactgtttataggcgacaaggccgagaatggccagatatataaggccttgttaaatgaactggtcgacgagcatctaggatggagacagaactacatgcctcaagacatgcccattattactcctgaggaaaaaagtagtgcatcttttgagcatactgtcaacaagactaaagacgtattatctgaaatatctgccagaatgcgtacacattccgttccctggcataatgctggccgttactggggtcatatgaactccgaaactctaatgccttctttactagcttacaattttgccatgctttggaatggcaataatgttgcttacgagagttcaccagcaacaagtcaaatggaggaagaagtcggaatggagtttgcaaaattaatgagttataaggacggatggggccatatcgtcgctgatggctccctagcaaaccttgaaggtctgtggtacgccaggaacataaaaagtcttcccttagcaatgaaagaggttacaccagaattggttgctggaaaaagtgattgggaactaatgaacctgtctacagaggagattatgaaccttttggattctgtccccgaaaaaatcgacgagatcaaggctcattcagcaaggtccggcaaacaccttgaaaagcttggaaaatggctagtcccacagactaaacattattcttggctaaaggccgccgatattatcggtatcggtttggatcaggtgattccagtacccgtcgaccacaactatcgtatggatataaatgaacttgaaaaaatcgtacgtggattggctgctgagaaaacccccatccttggcgtagtcggagtagtgggatccacagaggagggagctattgacggaatagataagattgttgccctgagaagggttctagagaaggacggcatctacttctatctgcatgtcgatgcagcctatggcggttatggcagggcaatttttttggatgaggacaacaactttattcccttcgaggacctaaaagatgttcattacaagtacaacgtcttcaccgaaaataaagactacatattagaggaagttcactccgcatataaagccatcgaagaagcagaaagtgtgactatagaccctcataaaatgggatatgtgccatactcagcaggaggaatagtcataaaagacattaggatgcgtgacgtaataagttatttcgccacatatgtcttcgagaaaggcgccgatatccccgcacttcttggtgcatatatccttgaaggttccaaagctggcgctacagcagcatccgtgtgggcagcacaccacgtcctgcccttaaatgtcaccggttacggtaagttgatgggtgcatctatcgaaggtgctcatagattctataatttcctgaatgatttgagtttcaaagtcggagataaagaaatagaggtacacccactgacgtatccagacttcaacatggtcgactacgtgtttaaagagaagggcaatgacgatttggtcgccatgaacaaactgaaccatgacgtctacgattactctagttacgtgaagggtagtatctacggcaacgagtttttaacgagtcatactgatttcgcaattcctgattacggcaattctcctctgcaattcgttaaccagttgggtttttccgacgaagagtggaatagggccggaaaggtcaccgtgttgagagcatctgtaatgacgccttacatgaataaggaggagcacttcgaagagtatgccgaaaaaatcaaggccgctctgcaggagaaactggagaagatttacgcagatcagctgctggcctcagaagccaagtaa

SEQ ID NO: 66 - DNA, 1416 bp, Solanum lycopersicum
atgggttccctatctttggagatggattttgaacctagtccaatgaccccacgttccctggctgccatgactcccagatctctggcaagacgtcgtcttttccccaatgttgacaacaaaaaacagaagatggcacaacccggcgctggaccaagaaaaaatcttgaacttgaagttatggagcccgctttgaaaaatgatggaccatctttggataccatcttggtcaactatttagacaccttaacacaaagggttaattatcatctgggttatcccgttaatatatgctacgatcactacgccacattggcccccctattgcaatttcatcttaataattgcggagatccatttcttcaaaacaccgtggactttcactccaaagatttcgaggtggccgtgctaaattggttcgctaagctgtgggagatcgagaaggatcagtactggggatacgtgacaaatggtggtactgaaggtaatttgcatggtattctattaggtagggagctattacccgaaggaatactttatgccagtaaagactcacactactcagttttcaaagcagccagaatgtacaggatggattcagaaacaataaatacgtccgtgaacggagaaatggattattctgaccttagagcaaagttgctgcaaaataaggataaacctgccattataaatgttaccataggaacaaccttcaaaggagccattgatgacctagacgttattctggagatactaaaggaatgcggctattcccaggataggttctacatacactgcgacgcagccctatgtggactgatgacgccttttattaataatatgattagttttaaaaaacccattggcagtgtgacgattagtggacataaattcttgggctgccctatgccctgtggtgttcaaatcaccaggaaatcatatatcaataacctaagtactaacgtcgaatatattgcttcagtagatgctacaatctctggatctagaaatggtctaacacctatattcctttggtattccctttcagctaagggtcaggtaggcttacaaaaagatgttaaaagatgcttggacaacgcaaagtacttaaaagacagattacaacaagccggaatctcagtaatgttaaacgagctttcaattatagtagtactggagcgtcccagagatcacgagttcgtaagaagatggcaactgtcttgcgtgaaagacatggctcacgttatagttatgccaggtatcacgagagagatgttggacaactttatgtctgagcttgtgcagcagaggaaggtttggtaccaaaacggtaagacggaccccccttgtgtcggagaagacatcggcgcacaaaattgcgcatgctcttaccataaaatcgactacatctgcccctaa

SEQ ID NO: 67 - DNA, 1416 bp, Solanum lycopersicum
atgggatctttgacgttagaaatggattttgagccctcacctatgacgccaaggtccttggcagcaatgacccctaagtctctagccaggcgtaggctttttcctaatgtggacaataaaaaacaaaaggtggaacaatcaggagcaggacccaggaaaaatctacagttagaggtcatggaaccaagtttaaataataacggaccaagtttagatactatcctggtgaattatttggatacattaacgcagagagtcaactatcatcttggatatccagtaaatatttgttatgaccattacgcttcactagctcctctgttacagtttcatctaaataactgtggcgatccatttttacagaacactgtggactttcattctaaagatttcgaggtagcagttcttgactggttcgcaaaattgtgggaaattgagaaggatcagtattggggctatgtcacgaatggtggcaccgagggtaatcttcacggaattctactaggccgtgaattactgccagagggaatcctatacgcttccaaggattctcattattctgtgttcaaagccgcaaggatgtatagaatggattcagaaacaattaacacctcagtaactggagagatggattactcagatcttagggccaagttactgcaaaacaaagataagccagccataatcaatgtgactattggtacaacgtttaaaggagccatcgatgatttagatgtcattctggaaacgcttaaggaatgtggctactcccaggatagattttacatacactgcgatgcagccctttgcggacttatgacgcccttcattaataatatgatttcattcaaaaagccaataggttccgtcacaatctcaggtcataagttcttaggatgtcctatgccatgcggtgttcagatcactagaaagtcatatatcaacaatctttctacaaatgttgagtacattgcatctgtagatgcaacaattagtggttcaagaaacggattaaccccaatattcctatggtacagtctatctgctaagggtcaagtgggtttacaaaaagatgtgaagagatgtctggacaatgctaaatacttaaagaacagactgcagcaggccggtatcagtgtaatgcttaacgaactttctatcattgtcgtacttgaacgtccaagggaccacgaattcgtcagacgttggcaattaagttgtgtaaaggacatggctcatgtgatagtgatgcctggtataacacgtgagatgctggacaatttcgtatccgagctggttcaacagcgtaaacaatggtatcgtgacggcaaagctgaagctccctgtgttggagaggatataggcgcacagaactgtgcctgtagttaccacaagatagattatataggaccataa

SEQ ID NO: 68 - DNA, 1194 bp, Escherichia coli
atgtttcagaaagtagatgcctatgccggtgaccccattctgacgcttatggagaggtttaaggaagatcctaggtcagataaagtaaatctttccataggcctatattacaacgaagacggtatcataccacaattacaagccgttgcagaggccgaggctcgtctaaacgctcaaccacatggcgcaagtctatacttgccaatggaaggattgaattgctacaggcatgctattgccccccttctttttggcgctgaccacccagtacttaaacaacaacgtgttgccacgattcaaactctaggcggctctggcgcactaaaagtaggagcagatttccttaaacgttattttcccgaatccggcgtatgggtatcagatccaacttgggagaatcatgttgcaatattcgcaggtgctggcttcgaagtctcaacgtatccctggtatgacgaggccacgaatggtgtgagatttaacgatctgctagcaacgttgaaaacactgccagcaaggagtatcgtcctgcttcacccatgttgccacaaccctaccggtgccgatttaacaaacgaccagtgggacgctgtaatcgagatcctaaaggctagggagttgataccatttttggacattgcataccaaggatttggagccggcatggaggaagatgcatacgctatccgtgctatcgcaagtgcaggtctgcccgccctggtctctaacagttttagtaaaatattttctttatatggagaaagggtaggtggcctaagtgtcatgtgtgaggatgccgaggctgctggtcgtgtactgggccaactgaaggcaactgttagacgtaactattcctccccacccaacttcggcgcacaggtagtcgccgcagtgctgaatgatgaggccctaaaagcctcttggctagctgaggtcgaggagatgaggacacgtattctagccatgagacaagaacttgttaaagtcctgagtacagaaatgcccgaaaggaacttcgactacttactaaatcaacgtggtatgttttcctataccggactgtctgctgcccaagtggatagattgagagaagaattcggagtatatcttatagcctcaggaaggatgtgtgttgcaggcttaaacacggcaaatgtgcaacgtgtcgcaaaagcctttgcagcagtaatgtaa

SEQ ID NO: 69 - DNA, 1344 bp, Petunia hybrida
atggaaactaacgttgttaaagtcaagtcaaatatggtggatggagataccaaccaggatatcgaaaccccaaataacatcacaataaaaggtatcctaggtcttctaatggccaatacggaaacggctagtgacgacgaagatgaggatatcgtgaacggcggtggcaatctactaataaaaatgaaaaagaagaagaaagtgatctcactaggcatgggcgatcccacggccttctcatgttttcactcccctgccgtagcacaagacgctgtcctggagactttggctagtcataaatttaatggatacgcccccacggtgggcttgccccagaccagaaaagccatcgctgactatttatcacgtgaccttcctgacaaactgtgtgcagacgacgtttacattacagctggttgcacacaagctatagagacagctctttccatccttgctagacctggttcaaacatactactacccagacccggatttccaatatacgcattatgcgccgctttccgtcacatcgaagttagatactttgatttgatacccgacaagggctgggaggtggatttaaacgccgtcgaggccctggcagaccgtaatacaattgcaattgttgtcattaatcccggcaacccttgcggcaatgtgtactcctatcaacatttgaaggagatagcagagacggcaaagaagctaaggacaatcgtaatagcagatgaagtctatggccacctagtgttcggagcaaaaccttttgttccaatgggagtatttggtgctatagccccagtcctgacactgggttccttgtctaaaagatggctggtcccaggatggagattgggctggttggtaacaaatgatccaaatggcacctttaagaatccaaaatttgtagaaagaataaagaaatattgcgatatatgcggtggtcctgccacttttatacaagctgccgtacccagaattatcgaaaaaactgaggaggtgttcttccgtaagactatcaatttgttaagatggacgagtgacatatgttgtgaaaagatgaaagagattccctgcatatcatgccctcataaacctgaaggctcaatggcagtaatggtgaagttgaatttatcaatattatctgacatatccgacgacatagacttctgcttcaaattagcaaaggaggagtccgtaatcgttttaccaggattggccgtcggactgaagaattggatccgtatcacgttcgcagcagatccatctagtcttgaagaagccctgggtcgtctaaaaagtttttgtcaccgtcattcctaccagcaaaacggacactgttaa

SEQ ID NO: 70 - DNA, 1263 bp, Arabidopsis thaliana
atgggcgaaaatggcgccaagaggtggaacttcggtgctaacgaagtcgtcgagagaagtaactctctaactattcgtgattaccttaatactttgatcaattgtcttgacggcggcgacgttaggcccgtgattcctctgggacatggcgatcccagtcccttcccttcttttaggaccgatcaagccgccgtagaggccatttgcgacgcagtacgttccacgaagtttaacaactatagtagttcatcaggtgtcccagtggctaggaaggctgtagccgagtatctttccagtgatttgtcatatcagatatcaccaaatgatgtccatataaccgctggatgcgtccaggctatcgaaatactaatatctgccctagctatacccggagctaacatccttttaccaaggcctacataccccatgtatgactcaagagccgctttttgccagttggaagtaagatatttcgacttactacccgagaacggctgggatgtggacttagatggcgtcgaggctctggccgatgacaaaacagtcgctatcttagtaataaacccctgcaatccttgcggtaatgtcttctccagacagcatcttcagaaaatagcagaaaccgcttgtaaacttggcattctggtgatagctgacgaggtttacgaccatttcgcattcggtgataaaccctttgtttctatggctgaatttgcagagctggtgcccgtgatagttctaggcgccatttcaaaaaggtggttcgtacctggctggagattgggttggatggtcacacttgacccccacggtataatgaaggattccggatttgttcaaacgctaataaatgttgtaaacatgtccaccgaccccgctacattcatccaaggtgctatgcctgatataataggcaatacaaaagaggaattcttttcaagtaagttggaaatggtgaaaaagtgcgctgaaatttgttatgaggagctgatgaagattccctgcatcacttgtccctgtaagcccgaaggcagtatgtttactatggtcaagttgaatttctctttgttagaggacatctctgacgacctagacttttgtagtaagctagcaaaggaagagtctatgatcattcttcctggtcaagctgttggactgaaaaattggctgagaatcacttttgcagtcgagttggaacttttaatcgaaggatttagtaggttgaagaatttcactgaaagacattcaaaaaatcaaccctaa

SEQ ID NO: 71 - DNA, 1026 bp, C. glutamicum
atgataagagcagatctggctacaatacctacttacgtcccaggaagaaggttagttgatgctaccaagttatcatctaacgaggtgagtttttctcctctacccgccgccgtggacgctgtaactgaagccacatggggagccaaccgttatcccgacatgggcgccgtggagttgagagaagctctagctgaacacctggaggtcgaattcgatcaagtaaccgtcggatgtggaagttcagcactatgtcagcaattagtccaggctacatgcgcccagggtgacgaagtaatcttcccctggagaagtttcgaggcctatcccatatttgcccaagttgccggtgccacgccagtggccatccctttgaccgctgatcagaatcacgatctggacgcaatggcagcagcaattactgacaagacaagattgatttttatctgtaatcctaacaatccctctggaactactatcacgcaagcacagtttgataactttatggaaaaagtccctaatgatgtagttgtgggactagatgaggcttactttgaattcaacagagcagatgacacacctgttgcaacggaagagatacacaggcatgataatgtcataggattaagaacgttcagtaaggcttacggtctagcaggccttcgtgtcggatatgctttcggtaacgccgagatcattgcagctatgaataaagttgcaatcccattcgctgtaaatagtgccgctcaggccgctgccctagcttcccttaactccgcagacgagttgatggaaagagtagaggaaactgtggaaaaaagagatgctgtagtcagtgctttaggtgctgccccaacacaggctaatttcgtatggcttcctggtgagggtgcagcagaactggcagccaagttagcagagcacggtatagtcattcgtgctttcccagagggcgcacgtatctcagttactaacgccgaagaaacggacaagctacttcgtgcctgggaggctattaacgctggataa

SEQ ID NO: 72 - DNA, 2478 bp, Aspergillus flavus
atggcacctctacttggatcatggacggcttcctccgcaggaggaccctcaaaggctcagcttcatacttccaccagaaaaaatattttatatgtcgacgcctacgactccttctcctataacgtcgtagctatgcttgaggaggtattgggtgtgaaagtcacggtgctgatgatcgattcagagtggcctgagggtaataaggctgagtttttgcaatacttcgaagctgtagtcctaggtcctggtccaggtgatcctaacgtgcctcaagatattggcattatgagagatatctgggacctgtccgacgcagatttactgcccgtgttcggcatatgcttaggtttccagtcactttgccttcatcactcaattgctattgaacgtctaagttacccattgcatggtagagtgtataggatatccacggctgaaaaagatatctttaaagatctgcaagacgtcgaagtaaccctgtatcattcactttatgcaaagcttgatgactctttagaagccgcactggctgagtactccggaaaacagataaaagccaacgctgacctagatttgctagcatggcttccaatagaacaagataacgtttcagagcgtacgattatccccatggcagtcaggcatagagaaaaacccttttggggtgtacagttccaccctgagtcatgtaaatctgatagaggtgtctgctctgaattgctaagaaaatggtgggaaatgagtctggactacaacaagaagcacggcagaggaggctatggctttttaccagaagatatcaccaggggcccttccgacatgacctcattaccagagatcgcttttacgatgttgaactggtgtgcctccacttctagatcttctgcattccgttcattacccacatccaggttgaatgcagaattcatttgtgaacgtttgaatgccccaggttctccaaccgttctatttcagtccaacggacgttacagtataatctcagtacctggtccaacttcctggcgtcttgaatactatgcacaagccgaggccttacttgtcgaacgtcttcaaggtcactgcaacggccataccaatagtggcattaagaagtctttaagtgtttatgacctatgggagacactaagatatctaatggatatgaaaaaggttgaatttggagacgacgcagttcccttttggggcggctttttaggatacttctcttacgagatgggcttagcatgcttggctcgtcccaagacacatagtgatgataatcacggttacattcctttgcagaatgcagctgcaaccaccgatcctgcagatgtctccttactatggactgagcgtagtgtagttgtggacaatgaaactggaagaataactattcaaagtactcgtaacgccgatgacaagtcaacaggatggcttgacgagactttacagtaccttacagaactgtctagagttgacgttctggaaaatgagaatgctaattctgatgccggctatctagacaccatcctacgtcaatgtgtcatcagattccccaatgagcagacctacaaacaacagcttgaagcctgcaaagctgaattagaggcaggagaatcatatgagctgtgtttaacatgcgaaacgtccataactttaccatccccctcaactgactccgaacgtgtggcattcccatggaaattatataaacgtctaagggaatataatccagcagctttctccgcctatgccaggttaggtactgtcaaggtggtatcatcatctccagagtgctttttgaattgggatcgttcctttaccctggaaatgaaaccaatgaaaggcacggtgcgtaaaagtgccagtatgaatatgaaaaaggctaaggagatattaggcagtacgaaggaaatggcagagaaccttatgattgccgacttgataaggcatgacttatacggtatatgcggttcaggaagggtaagagtcgagaagttattagaggtagaggatcatggaagagtatatcagatgattacacacgtcaagggcgatgtggatcccggtaggttgggcttcgctgtgaggaacttaccacatttacattcttccaatatgagtgcatacggcttgacagccctacaaaggtgccttcctcctggctcaatgacgggagcaccaaaggagaggagttgcatgcacttatccttgattgaggcaaggaagagaggtgtatattctggcgttatgggattcctagacttaggcggaggtggctccttttcagtccttatacgtactgcattctcatgctccgacgacaaagacaacgaacagacgtggagaattggcgccggaggcgcagtcacaatactatccacagccgatggtgagtggaatgagatgttaacaaagctaacgactgtatgcggagtttttgctcccagtgatctgcaaggatccggtaaggtttaa

SEQ ID NO: 73 - DNA, 2112 bp, Arthrobacter sp. ATCC 21022
atgacacctgcccccgttataatcgctgttgacggccgtagtggagctggcaaaacaacattagcagtggaattagctgctcgtctaagagaacatcacaaggtatcccttttccatttggaagatatataccccggttggaatggtctagccagtggtatagagagatatgtagctacagtattagcccccctacgtgacggtaagccagccgaatggatctcctgggattgggagaagcactacgatggtaggacccaagttacgctgccagccgagatcgttatagtagagggagtcggtgccgcagctcaagctgctcgtggtatgctagacgctgttgtatgggttgaggcttcaggtgaggatagaaggaggcgtgctttagcaagggatggtggaagttacgagccctattgggatgcctgggctcagcaggaagaggagtggctggccgtagacccagtcactgaggccgccgacattagagtccagaatcctgcagatggatctgctcccgcccatgtgctacaagccatgacatatttagcaccacttgccgctgtcctgtctccagaactatcagcacgtcgtggtctacaattacgtgctgaacgtatcgatgccgcccctgatgctgaagctttgttccaagatgtctacggttcatccaccaacgcagtttggttagattccagtaatgcagatactgctattacaggtggtacacaggctgcagcaagaagtcgttttagtattatggccgacgacggtggcaccttcggacaatctgttaggcattcatcaggcactacatgggtgaccaccggaagtgctaccgttaggtcttcaggtcctttttttagatggcttgactctgtatggggcagacgtgcagtcaggtcccctgagggttatgacggtcaatttaccctaggttggttaggctacttaggctatgaactaaagagggaaaccggaggtaatgacgttagtagtgcaacacctgatgccgccctggtgtttgcaggccgtgccctggtccttgatcacagggagcatgcagtatggctgttagcactagaagcacctgatgccgatgaatggttctccgtagcaagagcagctgttggcgctgctgacgctcaccctgctgtgcatcctgcaacggtgaggcccgtgagtggtcttccagtatctggaagaagagcagtaggcgtacctattgacacgaccggacaagctgtagcagccgcctcaccagaattcgcctgcagggactccgccacggactataggaggaaaatcacggaggcccaacacgagatctctgagggaaactcatacgaagtctgtctgaccacgactcttgaggcacccgcaggagaacttgacccatggcaaagttatcaggcactgagaaggcgtaaccctgccccttttgctagttacttacgtttcggcggtcttgcaatagcatcaacctcccctgagagattcttgaggataacgtctgacggaggaatgagagcagaacctatcaaaggcaccaggagacgttctgcagatgatgcagaggacattaggcttagacatgatcttgaaacatcattaaaggacagagccgaaaatatcatgatcgtggatctgctacgtaatgacctgtcccattttgctgttcccggttcagttacggtgtcaaggctatgcgctattgagtcatacgcaaccgtacaccagatggtctctactgtggacgcacacctaagacctggtgctccaagggctgaggctgtggcagctgccttcccagccggcagtatgacaggcgcacctaaaatttcaaccatggatatcttggataggttggaggccggtccacgtggcgtatactccggtgctatcggttatttctcaatgaatgcagcaactgatctggccgtagtgatcagaactctggtgataaaccctgacggcggaggaggaagaacgctgtcacttggagtaggaggagctgtaacagctgatagtacgcccgacgacgaatatgatgagatacgtacaaaggctttcggtgttttatctacccttggtgccacctttccctcttaa

SEQ ID NO: 74 - DNA, 2418 bp, Fusarium oxysporum f. sp. lycopersici
atgggcacgattaaggtgttaataatcgaccattatgacagttacacgaataatatccttcaactgctgcaaggtacccaaaaaggccacgacggacagagttatcctgagtggagtgtttccatcgtgcgttttgatcaattctcctgggatcactttaaatccgaaattctaccccacttagatgcaataatcctgagtcccggccctggtacaccccaaagggaggctgatttcggtttcaatgccagattaataagagaggtcaacattcccatcttgggcatttgtctgggtcatcaaggaataggcaccagtttcggcgcaaatatcatacatgcacccaatataaaacacggacagatatgtcaaattcatcataggggaacgggaattttatccggtttaccccagaaatttgatggtgttaggtacaatagtctggtcttagatatagaagacgtgccaccagaattacaagtgacggcctggacttacgatcctgagaaccccagtaaaaaggtcctgatgggattacagcatcgtgaaagacctgtctttggcacacagtggcatcctgagagtgtttgctctgcacacggtaagcagatactgtccaactttagagacattgtattggacttctgggtgacttccaatccaaggaatcagtggaccaagagaagagttttggagaatgccagtttacctgatcacatcttacaacagaatgcagttgaacagacggaccttggaaaagcactagtcgctgaagccaacctagcaagggtagtatccccatattatgtcaaatctgccagagtcggcaagggtccatccgcagagattgtgttccaaactctgtttcgtggtcgtagtttagacggagaagcctggttagatagtgctaaagttcgtgattcacacagtagaaacagttacctttcctgggcttccttcgctctttcttactctactaagtctcgtcacgtgtcattttatcaacatggaaaaaatattaagtcagaacgtttatctgtctcatattggtcatggttagaacaattccagcagggaacgattgtagctaataccgagacattgccatccgagtccctggatcaagagaccgacgttggtcagcctttattacaagtgggtttgatcggatacttcggttatgaattaaagcgtgaatcattaccaggatatcaatttaccccacccgatgaaaaggacgtttctctgaaacactccgactccgaacttcttttcgctaacactgttctacgtctagataattataccggcgagtggactcttttttctttaattagaaggggtgaagaagaccctattggcaacgcaataggtgccagttctaagattggtattgatgaggctgaattcgacttgtatttgactagagtcagagaagtatttgatgcaccccctagtcccccttacgtagaggcacatccattgccaaactttgttgcattagacaatgaggcatcctatagtaatacgatctgggctgcaaaagaagccatcaaagaaggagaaacatacgaactaactctgactacgaagtttagagctaagtcccctgaagtagacccatattctctttacttggacttaagaaaaaggaatcctgcaccttactctgcttacatacatttcgcagcacacgataagacgatcttatcatcttcccccgaacgttttatttctattgatagtaacggtgttgccgagatgaaaccaattaagggcactcttgctgttgatagtgaccccgtagaggatgaacgtcgtaaacaccagctagctactgatgtgaaagagctggcagaaaatttgatgattgttgatttaattcgttctgatctacataacatttctcccccttcatccatttctgtgcccaagttgttgcacgtcgagacataccagacagttcaccagctagtaactaccattcaatcccatgtagcatcatctgtgggcggtacgaaggttatcgaaagatgctttccacccggctcaatgacgggagcaccaaagttaagatcagtgcaaatattggattcccttgaaggacaaagggagcgtggaatttactccggaagtattggctatatctgtgccagtggaacagttgatcagtctgtagtcattagaactatcgttaaatccggcaatgaactagagttgggagctggtggcgcaatcacgtggttgtccgaggccgataaagagtgggacgaagtgatggttaaggctaacgcagtcgctaggggcaaaggtaaggcacacgttcctgacgtggctataagtcctttgggcccttttgtggaggaagagggtcacttacgtcaagaacatcccctaagagccgcaatttccgcttaa

SEQ ID NO: 75 - DNA, 2175 bp, Streptomyces pluripotens
atgaggacattgctaatcgacaactacgatagtttcacgtacaatttgtatcaattgctaggcgaagtcaacggcattcctcctgttgttgtgaaacacggcgaagattggaacgccctagatctggacgcatatgacgcaatagtagtctcccccggacccggccgtcctgacagagagagggattttggcatctccgcaagggcaattttggattctggtcttccagtccttggagtttgtcttggccaccagggtattactcatttgtttggaggtgaggtaggtcttgcaccagaacccatgcatggacgtgtttcagctgttcgtcacacgggtgttgatgtctttgccggtctgccaagtccttatgatgtggtaagataccactcacttgctgcaaccagattaccaggagaattagaggcaatagcttggaccgatgatggagtggtaatgggagttagacaccgtaccctaccaatctggggcgtacaattccatcccgaatctatctctagtcaatacggcagggagttgttggcaaacttcagggacctagccctagctcatggccgtcgtgctccacacactgcagtaggtgcttcaggcgcttctgcaacggccccctacgcagttcatgtggccaaggttgacgctcttccagatacagaggccgcatatcaggctctgttcgctacaggcagtcatggtttctggttggattcttctagggtcatagatggcctttcacgtttttcatttttgggcaatggcagtggaccattagctgaatatgtaacctaccgtatcgctgacggtgttgtctctgtccagcgtcccggacagccagaagcaggtggagaaataatttcccaaggcttttttgactatctagatgagcaactaggtcaacgtgctgtccctactcccgagggtttaccatttgattttaatttgggatacgttggatacttgggttatgagttgaaagctgaagccggtgccgtcgccacgcacaaagcagaaacccccgacgctgcagtgttattcgccgataggatgttggctgtcgaccacgccgaaggcaactgttatttactagccctttcacacggtggcgatgacgctgacgccctatcttggttagccgaaacggaacaatccctaagaagactgccaacacccgcaggcaggtccgctcaaggacaagcaagatttaacggcactggcatgacagaccccgctgcagccggcatcgtccaccttggacatgatagagaagcttacctagataggatcgtgagaagtatggaggaaatcaggcagggagaaacatacgaggtctgtatgaccaataccgtaaccatgaatgcaaccatagcccccttaactacctatggatacctgagacagatatcccccgtgccatacggtgcactattggactatcccggagtggccgtgctttccgcatccccagaaagattcatgactataggtactgaccgtgttgcagagtccaaaccaattaaaggaacgcgtccccgtggtgccacgcccgaagaggacgaagctcttcgtcaagacctgctaggccaagagaaagatagagccgagaatttgatgatcgtcgacttaatcaggaatgacctaaatgtcgttagtgaaattggatctgtccatgtcccccgtttattccatgtcgagtcatatgcccctgttcaccaacttgtttccacgattaggggaactctaaggtcaggagagacagctgtgacatgcgtgcgtgctgcatttccaggtggttccatgacgggagccccaaagcttaggacgatggaaattattgaccgtcttgaaggcagtgctaggggagtatatagtggatctttaggatggtttagtctatcaggagctgccgatcttaacatcgtaattaggaccttagtcgcaaccgaagacaccgtcagttttggagttggaggagcaattgttgctctaagtgatccagaggaagaattcgaggaaacggacgtaaagtctagagccatggttagtgctgttctagccactatgaggagagacccatcaggcgccgacgctgatgctcatcccgaagctgccgcagagcttgcagccgtcactgtccagagtgctggtgaagcaccctaa

SEQ ID NO: 76 - DNA, 2364 bp, Saccharomyces cerevisiae
atgctatccgatacaatcgatactaaacagcaacagcagcaactacatgttctatttatagactcatacgacagttttacgtataatgtggtccgtttgatagagcaacaaacagatatttcacccggtgtaaatgctgtccatgtaacaacggtgcattccgacacgttccaatctatggaccagcttttacctctactacctctttttgatgcaattgttgttggccctggtcccggaaaccccaacaacggcgcacaagacatgggcatcatctccgaactttttgaaaatgccaacggaaaacttgatgaggttccaatattaggtatctgccttggatttcaggccatgtgccttgcccaaggagccgacgtctccgaactgaataccatcaaacacggtcaagtctatgagatgcaccttaatgatgccgcaagagcctgcggccttttttccggctatcctgatactttcaagtcaactagataccacagtctacatgtcaatgctgagggcatagatacgcttcttcccctatgtacaactgaggatgaaaacggcatcctgctaatgtctgcccagaccaagaacaagccctggttcggtgttcagtatcatcctgagtcatgctgctccgagctaggcggtcttctagtctcaaactttctgaaattgagttttataaataatgtcaagactggccgttgggaaaagaaaaagctaaacggcgagtttagtgatatactatccaggttagatagaacgatcgatagagatcccatttacaaggtaaaggagaagtatcctaagggcgaagatactacatacgtcaaacagtttgaagtgagtgaagacccaaagctaacgttcgaaatatgcaatattatcagggaggaaaagttcgtgatgtcatcctccgttatatcagagaacacaggagaatggtcaatcatagctttacctaactctgcctctcaagtgtttactcattacggagctatgaagaaaactaccgttcattattggcaagactcagagatctcctacacattgttaaaaaagtgcctggatggccaagattcagacctgcctggctcattagaagtgatccatgaagacaaatcccaattctggataacactgggtaaattcatggagaacaagatcatagataaccatagggagattccttttataggtggattagtgggcattctaggatatgagatcggccaatatatagcctgtggtagatgtaacgacgatgagaattctctagttcccgacgccaagctggtcttcattaacaatagtatcgttattaaccataagcaaggaaaattgtactgtatatctctggacaacaccttccccgtagctctagagcaatccttaagagacagtttcgtcaggaagaaaaacataaagcagtccctatcatggcccaaatatcttcccgaggaaatcgacttcataataactatgcccgataagctagactatgcaaaagcattcaagaagtgccaagactatatgcacaagggcgatagttatgagatgtgtcttaccacacagacgaaggtggtaccctccgccgtgattgaaccttggaggatatttcagaccttggtgcaaagaaatcccgctccattctcctcctttttcgaattcaaggatataattcctaggcaagacgaaacacccccagtgctatgtttcctgagtacgtcccccgaacgtttccttaaatgggatgcagatacttgtgagctaagacctattaagggcacagtaaaaaagggaccccagatgaacctagcaaaagccacgaggatactgaaaacgcctaaggaatttggagaaaatctgatgatactggatctgatcaggaacgatctgtacgagctagtacccgatgtcagagtcgaagaatttatgtcagtgcaagaatatgctacagtctatcagttagtgagtgtggtcaaggctcacggacttacttccgctagtaaaaagacgaggtacagtggtattgatgtattgaaacactctttgccacccggatccatgacaggtgctcctaaaaagattacagtacagctattacaggataaaatcgaaagtaaattgaataagcatgttaatggaggagcacgtggagtgtatagtggtgtcacaggttactggtccgtgaattccaatggcgactggtccgtgaatataagatgtatgtactcctataacggcggaacctcctggcaactaggtgctggaggcgctatcacggtactttcaactctagacggtgagcttgaagaaatgtataataagctggagtctaatttgcagatcttcatgtaa

SEQ ID NO: 77 - DNA, 381 bp, Pseudomonas putida CSV86
atgtcactagatacctgtgatggtcatggacacggtcacggacatggccacggtaacggcaatgcttctgccgagttacaaccttatcgtaaacagcttgacagaatcaatgaactactggtggacctactggctgaaaggatgcaagtatgcaggttaatagcaagggtgaaatccgcaagaggcattgcaataatgcagcccttgcgtgttaattctacattagaccaagtcagagctttatctggtccaagaaaattgcgtcccgaatacttggacggtcttttcaggctaataatagaggaaacatgcgacgaggagaggcgtgttatggctgtcttggaggcatcacccgagggcagagaaccctgcgccagataa

SEQ ID NO: 78 - DNA, 336 bp, Pseudomonas sp. Leaf48
atgaaacaacaaggtcagctttcacagaccgacatgtccaatatattacaaagtcaaagagagagacttgacttaataaacttacaggtagtggacttattgtccgaaagaatgaaggtgtgcatgggtatcgcagacctgaaagccatacatggtatcgctatgatgcaacctggcagaatctcctatgtactggacatgattaaaatccaatctgaggccgtcggcctaagacccgcttacaccgagagtatcttcaagttgatcatagaagagacttgcattcaggaggatctgttgataaaccaatacctgggtcctggtcagtcatcataa

SEQ ID NO: 79 - DNA, 315 bp, Photobacterium halotolerans
atgaacgacgctgtggtggtacgtgagctgaaaccctaccgtgacaaattagatgagattaacgaacagatcgtagccctgttggcaagtagaatggacatatgtaggacgattgcaaatgtaaagtccagggagaacatacccatgatgcaaaaggacagagtagagttcactttaaataaaatgaagagtctttcagtagattaccagttgaacccttcttacataggcgacatattcgagaggataatacacgagacatgcgaagaggagctggtaataataaaggcactttccgaagaaaaagagaagtaa

SEQ ID NO: 80 - DNA, 360 bp, Photorhabdus asymbiotica subsp. asymbiotica
atgacgaagtccgagtacatgacgcaatccattattacgaagcaggacgtgttacagcctcacagagagtctctggatgtgataaatatgcaaattctatccctattaagtgaaagaatgaaaatttgcatgaagattgccgagatcaaggccgaacaggacatccctatgatgcagcctcagagaatcacgagtttgttggccatgttaaaagataagagtacggactttggcctgcgtcctgagtacaccgagttaatttttcagttagtcatagaggagacgtgcaggcaagaggaaactttgatcgataagttactaagagagaagaagaacaataacgagaacatagcacattaa

SEQ ID NO: 81 - DNA, 390 bp, Streptomyces pristinaespiralis
atgacaccacctgctattcccgcagcccctcctgcaacaggacctgccgcagctactgacccacttgatgcacttagagcccgtttggatgccgctgacgctgccttgttggatgcagttcgtactcgtttagatatctgtctgagaatcggcgagtacaagaggctgcatcaagttccaatgatgcagccacaccgtattgctcaagtccatgccaacgctgctagatatgcagccgatcacggcattgatccagcatttcttagaacgctgtatgatacgatcattaccgaaacctgccgtctagaagacgaatggatcgcatctggcggcgcccccgtaccaacaccagtccatgcatccgccagtgcccgtggaggagtctcataa

SEQ ID NO: 82 - DNA, 867 bp, Streptomyces sp. CB01635
atgtttgagcactgtgtcgtggcaggtggtacaggtgaagttggaaggatgtttgctggtagattggctggttctggagcccaagtgctttgcctggaccctgctgcatcagcacccgcagccctggcagacgatattagggcacctggccccgaggccgctagagctattgcagccgcagaccttgttctattagcagtgcccgaacctgttgcccttgatgcaatcggtcctgtaacgaggctgatgagggccgacgctctgtttgcagacacgctttctgtcaaaggaggtattgccgagagactgcatgccttggcccctggtatacaagcagttggattgaacccaatgtttgcccccagtcttcctcttccaggtagacccgttgcagctgctaccgtcacagacggtccagccgtcagggcctttctggctctgattagtggttggggtgcaagggtggtggaaatgtcagcccaagaacacgatgctctaacagccgctcaacaagcagcaacgcatgctgccgtcctagcctttggtttagccttaggagaattaggcgccgatataggagcattaagagctaccgctcctcctccacaccgtactctattacaactgctggctagaatctccagtggcacgcccgaggtctattgggacgtgcaatccggaaaccctcatgcacaagctgctaggcgtgctgtaggacacggtttagagagattaggtagagctgtagcctctggtgaccccagtgcctttgaaaccctgtttcaagagcttagggctactttaggagagaggcatcgtaccgaacttgccgacgcttgtgctgaattgtttgcacagcttcgttaa

SEQ ID NO: 83 - DNA, 894 bp, Paenibacillus wynnii
atgtcccaaatgatcaggagatgcgtgattgtcggtggtactggcggaataggtggaatgtttgccactcttctagctgaggctggtgtcgaggtatgccttattgatcaaaattatctaattgtcaattttgaattattgcaatgtgacatcacgtctccaactcccgctgccatttccgtaataaggatggccgacatggtcattctggcattgccagagagagttgccatgaacgccttggcaatagtctctaatctgatgaaaccagagagtcttcttgcacatacactttcagtacagctgccaatagcaaccaaaatcgattatttgggtatctcattagaagtagtcggtctaaatcccatgttcgcacccaatctaagtatcgtcggtcgtcctgtggctgccattatccacaacgaaggtcctagagtggccgaattactgcaattattatcagacgttggcgctagggtagtgaggatcgacgccgaggagcacgaccagctggtagcagccatgcaggccttaacacatgcctcaatcatttcttttggcctagccctttccaccttaaatgttgatatagccaagttgagtctgttggctccacctccccactcaactatgctggccctgcttgctagaatttcctcaggtactccagaagtgtactgggatgtccagacggcaaaccagcaagcatccatggctcgtatcgctttggtcaatgcaatacacagattgaacgccttaacaactaatgaatcagccctggatgactttattgatcttttatctgaaacgcgtgaggtactgggtaatgagataaccagttaccaggacttatgcgctctgatatttaatggttcacagtcagccacacagtaa

SEQ ID NO: 84 - DNA, 882 bp, Xenorhabdus doucetiae
atgaacaatttaaagatagttatcttgggatctaacggcgctataggtagtttccttggaaggatcttttcaacgtccggacacaccgttcacggtatcgataaacagctatccggaaattatacctcctatagtcagattgacctgctggatcctataagtaacatagatgaaatcttcaacgaggcagatgtcattatctttactctaccagaaaccgtagctattaaggccctgccttgggtactatcagcaatcaacgacaacgtcctgatagtatcaacctgttcagtccaagagcccttctacagttcactaaagacgataagtcctcagcaacccttcatcggcattaacccaatgttctccccctctcttcaggagaagaatcgtcccgttattcttatcatcgaggatatgcataacgagcatggctggattgagctagttttaactaaatttcagatgcgtatcacaaagatgcgtcctaaggaacacgacaaggtgatggcactgtgtcaggtactgccacacgctttaattcttgcatttggacttgcattgaaggctaacagtattgatatcaatgtactgtacaacattacgcctcccccaatgagaaccatgttggcacttctttccaggctactggtaaaccccaaagaaatttactgggatatccaatacgagaattacatggcagtagaaattaggaagaatatccttgacgaattatcattactagattcaatggtacaatctggcgacaaagttgagttcaaccactcccttgatgagatcaaacttaaacttggcgagtggctttatacgagttataccgactgccaacacatattccaaatactaaacgaagataagtaa

SEQ ID NO: 85 - DNA, 873 bp, Pseudomonas frederiksbergensis
atgatgagtgacaccgtcgttgtcctaggcggagctggcttggtgggtagtctaatatcccgtatcctgatgcaatacggctacgatgtgcgtgtcgtcgatcgtcgtccttctgaacaggaaagtggattttacgaagtggacgttacgagtcctttctctaatacgggacacatcttccaaggcgctgtcgccgttgtattcgcattgcccgagaatgtagctatcagggccattccatgggttgttggatcagtatccaacgacgtggttttcatccctacctgttcagttcaggagccattttatagggcattgaaaaacgcttcttctaaacaaccatttgttggcgtaaatcctatgttcagtccaaagttgtctgtccaaggcaggacagtagctatttgcttagaagacggagattctcccagtacatttatcgaacaacatttaatgggagctggtatgaagataaaaaggatgacccccgttgctcatgacgaattgatggcactatgtcaggccctgccccatgccgcaatactgggctttggcttggcactggctaggtcatcattagaccttgagtccgcactggaagttatgccaccacctatgaggacaatgatggccttgttgagtagagtccttgtgaatccccctgaagtctactgggatatacaacttgagaacgatcaagccaataggcaaagggcagcccttgctgaaggcatggaacgtttaatgtctaacgtatctaatcagaacttcgacgaatttcgtcgtgatttgcaaggagtatcagatgccctaggaagtagacttaacccaggagctgtcgattgtcaacatgtgtttagtcttctgaattaa

SEQ ID NO: 86 - DNA, 870 bp, Photorhabdus temperata subsp. temperata M1021
atgtccaatcataaagttgtgattttgggtggtcagggagccataggttcacttctgggtcaccttttcacacagagtggatgtaccgtttacagagtagatcagaggacacaaggtgaacacagtttttgctaccagttagacatcctgaacccagcagctgattccgaggcaattttcacagaagccaaggcagttgtatttgccttgcctgaagccgtggccatccaagcacttccttgggtgctgtcagtaattggtgacgatgtgcagatcgtttccacctgttccgtccagactccctttcattcagctctaagggctgccgcaccaggtcagcccttcataggcgtaaaccccatgttttctccctctctgtccgaacaaaaaagacccgtggctgtgatactggataactgccatgtaaacgaacactggattgaattagtgctgacccaggctggaatgaggatttcccgtatgacgccagaggaacatgaccgtgttatggccttctgtcaagcactaccacacgccgccatattgatattcggtctagtacttgcaaagtcacctctggacatcggtacgttagtaactgttgcaccacccccaatgaggacaatgcttgccttactgagtagggttctacttaatcctatggaggtgtattgggacgtacagtatgaaaatcccatggctgctgagccaaggcaagaattgttgcatgctatccaacagctggattccatcgtccaggccggagatcaaaccgaattttctaacaatctacgtcacatcgctcagcagttaggtgaacaacttaacatatccgctgctgactgtttagcctactttttcaacgcataa

SEQ ID NO: 87 - DNA, 1161 bp, Escherichia coli
atgacaaacaaccctttaatcccccaatctaagttgccacaattaggcactacaatctttacacaaatgtcagccctagctcaacagcaccaggcaataaacttatctcaaggtttcccagattttgatggtcccagatatttacaggagcgtctagcccatcatgtagcacaaggtgccaaccaatacgcccctatgaccggcgttcaagccttgcgtgaagctattgcacaaaaaactgaaaggctgtatggataccaacccgatgccgatagtgacatcacggtgacggcaggcgctacggaggctttgtacgccgctattacggcactagtgaggaatggtgatgaggtgatatgttttgatccctcatacgactcttacgcccctgccattgccttatccggaggcatcgtaaagaggatggcattacagcctccacattttagggtagattggcaagaattcgcagcactactttccgaacgtacaagactggttatccttaataccccacacaatccttctgccaccgtatggcaacaggcagacttcgctgccttgtggcaggccatagccggtcacgagatctttgttatttccgacgaggtgtacgagcacataaatttctctcaacagggtcatgcctccgtattggctcatccacaactgagagagagggccgttgctgtgagttccttcggtaagacctaccacatgacaggatggaaggtgggttactgtgtagctccagcacctataagtgccgagattcgtaaagtacatcagtatttgactttctccgtaaacactccagctcaacttgcactagccgacatgcttcgtgccgagccagagcactatctagcacttccagacttctaccgtcagaagagagacattctggtgaatgctctaaacgaatccaggctggaaattcttccatgtgagggcacatacttcttacttgtagactattcagccgtatctactttagatgatgtggagttctgccagtggctgactcaagagcatggtgttgctgccatacccctgagtgttttctgtgccgacccatttcctcacaagttgataagattgtgcttcgccaaaaaggaatcaacacttctagccgcagccgagcgtctaagacaactttaa

SEQ ID NO: 88 - DNA, 1056 bp, Caldanaerobacter subterraneus subsp. Tengcongensis
atgattgaaaaccttctgagagaagaaattaaaggcttcaaaaattatgaggtagaaaatgtgccttataagtacaaaatggatgctaatgaaacaccattcgagttgcctgaggaagtgatgaagaatataggcgatatcgtcaaatccatacatgtgaatatttatcctgaccccacagccgagaagttaagagaggaattggcaaggtactgtagtgtgactccaaaaaacatctttgtcggcaatggatctgatgaaatcatacatcttataatgttagcttttgttgataaaggcgacaccgtcctataccctcatccctcattcgcaatgtactcaatttatagtaaaattgctggcgcaaacgagattgccgttaatcttaatgaggattacacatacaacgtcgagcgttttgcagaagcagtggagagatacaagccaaagttggtgtttctgtgcaatcccaataatcccaccggatccgtaattgatgaggaggatattatcagaattatagaaaaagctaggggcattgttatagtcgatgaggcttacttcgagttctacggaaagacactagtcccctatatcgatagattcgaaaatctaatagtcttgcgtactctttctaaagccttcggaattgcaggtctaagagttggttacgcactatctaatggcgagatagttaagtatttgaaccttgtcaagagtccctacaaccttaactctctttcacagagaattgcactagaggttttgaaatcaggtgtactgaaagaaagggtcaactacattatcaacgaacgtgagaagttggtaaaagagttaaataagatcaatggcatcaaagtctaccctagtcacgcaaacttcgttttgtgtaaatttgagaatgccaacgacgttcacaagcgtctggtcgaaagaggaatccttgttagaaacttttccaacgtaaagggattagaaggaacgttgagaataaccgtctcaagtagtgatgctaacgactatcttatcaacgcactacgtgagattctaagttaa

SEQ ID NO: 89 - DNA, 1542 bp, Saccharomyces cerevisiae
atgacagctggatcagcccctcccgttgactacacatcacttaaaaaaaactttcagcccttcctaagtcgtagggttgaaaatcgtagtttgaagtcattttgggacgcatcagacatctccgacgacgttatcgaattggcaggaggaatgccaaatgaaaggtttttccccatcgagtctatggatttaaaaatttctaaagtcccctttaatgacaaccctaagtggcacaatagttttacaactgcacacctggatttaggtagtccaagtgaattacctatcgctcgttcatttcaatatgccgaaaccaaaggcctgccccccttgttgcacttcgtgaaggacttcgtgagtcgtataaaccgtcccgctttttccgacgaaacggagtctaactgggatgtcatacttagtggtggctcaaacgacagtatgtttaaagtattcgagacgatatgcgatgaaagtacaactgtcatgatagaggaattcacttttacgccagctatgagtaatgtggaggctacgggtgcaaaggttatccccatcaagatgaaccttacctttgacagagaatcacagggcattgatgtagagtatcttacacagttgctggataactggtccaccggcccttacaaggacctaaacaaaccaagggtactttatacaatagccactggccagaaccctactggtatgtcagtaccacaatggaagagagagaagatatatcagttagcccagaggcacgatttcctaatagtagaggatgatccatacggttacttatatttcccttcatataatccccaggagcctctggaaaacccatatcattcctcagacctaaccaccgaaagatatctgaacgactttttgatgaaatccttcttaactttggacacggatgctagggtgattagacttgaaacattctctaaaatctttgctcccggtcttagattgagttttattgtagccaacaaatttctgctacagaaaatactggacctagccgacataactactagagccccaagtggtactagtcaagcaatagtatactcaactataaaagcaatggctgaatccaatctatcttcttctctttccatgaaggaggccatgttcgagggctggattagatggattatgcagatagcaagtaagtataatcaccgtaagaatctgacgcttaaagctctttacgagaccgagtcataccaagctggccagttcactgtaatggaaccatccgctggcatgtttattataatcaaaataaactggggtaattttgatcgtcctgacgacctgcctcaacagatggacatcttggataaattccttcttaaaaacggagtaaaagttgttcttggctacaaaatggccgtatgccctaattactccaagcagaattctgattttctacgtctgacgatcgcatacgcaagagacgacgaccaactgattgaggcaagtaagcgtatcggctctggaataaaggaattttttgacaactacaagtcctaa

SEQ ID NO: 90 - DNA, 1281 bp, Dictyostelium discoideum
atgattagatctgcccgtttaatttctaacatcaagttcggccaaaagaacataaggcagttctctaccaacaccaattggtgggccaatgttcaaaagggtccagaggacccaatcctgggcgtttccatcgcttataataaagacacaagtccttctaagatcaatctgggcgtaggagcatatcgtgatgagaacggtaaaccttatgtgctagactgtgttaaaaaagcagataaaaaaatctatgaggcaaatgttgaccacgaatacgcaccaatagtaggcgtcgcagcattcaatcaactagctgctcaattggcattgggcgaggaatgcaagcacattaaagagaagagaattgccacggttcaaagtatctcaggtaccggtgcattgagaattgccgccgacttcttcgccagattcctaaagggtaaaactgcctatgttcccaaccctacgtggggaaatcacaacgtcatcttcaatgacgcaggtataccagtaaaaagttatggatattataatccagcaacctgtggattaaattttgaagccatgaccaaggatatagctgcagccccagagggttccatcatactactacatgcatgtgctcacaatccaaccggagtagatccaacggctgaacaatggaagaagatctctgagatttgcaaagaacgtggtcacttcgtcctttttgacttcgcataccaaggattcgcttcaggctcaccagagaaggatgccgccgctgttcgtatgttcgtagaggatggtcacaacatagcactgtgccagagttttgccaagaacttcggcctttacggagagcgtatcggtgccttttccatattaaccgagacatcagatcaggccctaaatgtagaatcacaattgaagatattgataagaccaatgtactctaaccctcccgtctatggcgctaggcttgttcaagccatattaaaagataaggagttgactaatgaatggaggtccgaagtcaaaggcatggccgatagaataatcaacatgcgtgagcaactggttaaatatcttaaaaagcacggttccacgcgtgattggtcacatatcaccactcaaatcggaatgttttgctttaccggtctgacaccagagcaggttgaccgtttagctaacgagtatcacatctacttaacgaggaacggtcgtatcagtatcgccggaattaactcaactaacgtcgaataccttgctaaggcaatggctgctgtaaccaaggataattaa

SEQ ID NO: 91 - DNA, 1194 bp, Klebsiella pneumoniae
atgttccaaaaggtggatgcctatgcaggagatcccatactgagtcttatggaaagatttaaggaggaccctaggagtgataaggtaaacctttctattggcctgtactacaacgatgacggcataatcccacagctacaggctgttgccgaggccgaggctagattaaacgccgagccccacggtgcaagtttgtacctgcccatggagggattcagtggatataggcaggccatagcacccttattgtttggtgcagaacatacggccctaaaacagaataggatcgcatcaattcagactgttggtggttctggtgcattaaaggtcggtgcagacttcctgaaaagatacttcccagagagtcacgtctgggtatctgatcccacttgggaaaaccatatagcaattttcgaaggtgctggcttcgaagttagtacatacccctggttcgataaagcaacaaacggtgtacgttttgagaatcttttagccatgctgcagacgctacctgctcgtgacatagtattgttacatccctgttgccataatcctaccggtgctgatcttacaccagcccagtgggaccgtgtcgtagaagtcctgaaggctcgtcaattgattcccttcttagacatagcctaccaaggattcggaggtggccttgaagaagatgcctatgcaatccgtgccatagcctctgccggaatgcccatgttagtctctaactcatttagtaaaatatttagtttgtatggcgagagagtaggtggtttgtctgtggtctgcgaggattcagaaacagctggcagggtgcttggtcagctgaaggcaacggtcaggagaaactattcctcaccccccagtttcggtgctcaggttgtagcaaccgtattaaatgacgcagcactgaaagccacttggcaggccgaggtggatgcaatgcgtgctcacattcttacaatgaggcaagcactagtggatgccttgcaacaagtcgctcccggctccaaagtagactatcttttaaagcagcgtggcatgttctcctatacaggcttctctgctgctcaggtagataggctaagggatgaatttggtgtttatttaattgcctctggtagaatgagagtggctggtttgaactccagaaacgtgcagcaggtagcaaaagcattcgtggctgtgatgtaa

SEQ ID NO: 92 - DNA, 1185 bp, Chlamydia trachomatis
atgaaaagaaatccacactttgtcagtttgactaaaaattatttattcgctgatctgcaaaaaagggtagcccaatttagacttgagaatccccaacacactgttataaacctgtccataggcgacactacgcaaccactgaacgccagtgtcgcagaagccttcgcaagtagtatagcacgtttgagttctcctacgacgtgcaggggttatggaccagactttggactgccagctttgcgtcagaaactgtctgaggatttttacagaggttttgttgatgccaaagaaatatttatttccgatggcgctaaggtagacttgtttagattgctatcattctttggcccaaaccaaacagtagcaatccaagatccttcctatcctgcctacctagacatagcacgtttgacaggagccaaggagattattgctttaccttgtctacaggagaacgcattctttcctgagtttcccgaagatacgcacatagacattttgtgcctatgctcacctaataatcctacgggaaccgtcctaaacaaagaccagctgcgtgctatagtccattatgcaatagaacacgaaatcttaattctatttgacgccgcttactccacatttatctcagaccctagtttgcctaaatctatatttgagattcctgatgcaagattctgcgccatagaaatcaactcttttagtaagcctctgggctttgcaggcatcaggttaggttggacagtgattccccaggaactgacatatgccgacggccatttcgttatccaggattgggaacgttttttatctaccacgttcaatggtgcttccatcccagctcaggaggccggagtagctggactgtctatccttccccagctggaggccatccactattatagagaaaactctgacctgctgaggaaggccttacttgcaactggctttgaggtgtttggaggcgagcatgcaccctatttgtgggttaaacctacacaggctaatatctcagatagggatcttttcgatttctttttgagagagtatcatatcgcaatcactccaggaatcggttttggccgttcaggctcaggcttcgtgcgtttttccagtttaggaaaaagagaagacatcctggcagcctgcgaaaggttgcagatggcaccagctctacaatcttaa

SEQ ID NO: 93 - DNA, 1185 bp, Paracoccus denitrificans
atgttgggaaatttaaaaccccaagctcctgacaagatcctggctttaatgggtgaattcagagcagaccccaggcaaggtaaaattgatcttggtgttggagtctacaaagacgccacgggtcacacccctattatgagggcagtacatgcagccgaacaaagaatgcttgagacggaaacgactaagacttacgcaggtttgtccggtgaacccgaatttcaaaaggccatgggcgagttaatcctgggtgatggtctaaaatctgagacaactgccactttggctacagtcggaggtacaggagccctacgtcaagcactagagctagcaagaatggctaacccagatttgagggtattcgtcagtgatcctacttggccaaaccatgtgtctataatgaacttcatgggccttccagtccaaacgtacagatactttgacgctgagacaaggggagtcgattttgagggtatgaaggctgacctggccgctgcaaaaaagggcgatatggtccttctgcacggctgttgccataaccccaccggtgccaatctaacattagatcagtgggcagagattgcttcaattcttgagaagacaggtgcactgcctttgattgacctggcctatcagggctttggagacggacttgaggaagatgctgctggcacccgtctaattgcctctaggatacccgaagtcctgatcgctgcatcatgctcaaagaattttggtatttaccgtgaaagaacgggctgcttgcttgctctatgtgctgatgctgccacgagggagttggcacagggcgctatggccttcctgaataggcaaacgtactcttttcctccatttcacggtgctaaaattgtgtcaacggtacttacaactcccgaattgagagccgactggatggccgaactagaggccgtgaggtctggtatgctacgtttaagggagcagctagcaggagaacttcgtgacctttctggatctgataggtttggcttcgtagcagaacaccgtggaatgtttagtcgtctgggcgccactcccgagcaagtcaagaggattaaagaggagtttggtatatacatggtgggagactccagaatcaatatcgctggtcttaacgataacactataccaatactagccagagcaatcatagaggtgggcgtttaa

SEQ ID NO: 94 - DNA, 1113 bp, S. cerevisiae
atgtcagaatcccccatgtttgcagctaacggcatgcctaaagtcaaccaaggagctgaggaggacgtaagaatacttggttatgatcccctggcttcacctgccttgttacaagtgcaaatccctgccacaccaacctcattagaaacggccaaaaggggacgtagggaggcaatagatattattaccggtaaagacgatagggttttggttattgtgggtccatgttccattcatgatctggaggcagcccaagaatacgctttgcgtttaaagaagttgagtgatgaattaaaaggagatctgtctattataatgagggcatacttagagaaacccagaacaaccgtgggctggaaaggattgataaatgaccctgatgtgaacaacacgtttaatatcaacaagggcctgcagtccgctagacagctatttgtgaatttgaccaacatcggactgcccattggctctgagatgttggataccatatctccaaagtaccttgcagacctggtaagttttggagctatcggcgccagaactacggaatcacagttacatcgtgaattagcatctggactgtccttccctgtgggattcaaaaacggtacggacggcacacttaacgtggccgttgatgcttgtcaagctgctgcccactctcatcacttcatgggagtcactaaacacggcgtagctgcaatcacgacgaccaaaggcaatgagcattgcttcgtaatattacgtggtggcaagaagggcaccaactacgacgctaagtccgtggccgaggcaaaagctcaactgccagctggctccaatggtttgatgattgactattcacatggaaattctaataaagattttagaaatcaaccaaaggtgaacgatgtggtatgcgagcaaatcgccaatggcgaaaacgccattactggcgtgatgatagaatctaatatcaacgaaggaaaccaaggaatacctgcagaaggtaaagccggtctaaagtacggagtttcaattaccgatgcttgtattggctgggaaacaactgaagatgttctgaggaagcttgctgctgctgttagacaaagacgtgaggtgaataaaaaataa

SEQ ID NO: 95 - DNA, 1113 bp, S. cerevisiae
atgtcagaatcccccatgtttgcagctaacggcatgcctaaagtcaaccaaggagctgaggaggacgtaagaatacttggttatgatcccctggcttcacctgccttgttacaagtgcaaatccctgccacaccaacctcattagaaacggccaaaaggggacgtagggaggcaatagatattattaccggtaaagacgatagggttttggttattgtgggtccatgttccattcatgatctggaggcagcccaagaatacgctttgcgtttaaagaagttgagtgatgaattaaaaggagatctgtctattataatgagggcatacttagagaaacccagaacaaccgtgggctggaaaggattgataaatgaccctgatgtgaacaacacgtttaatatcaacaagggcctgcagtccgctagacagctatttgtgaatttgaccaacatcggactgcccattggctctgagatgttggataccatatctccaaagtaccttgcagacctggtaagttttggagctatcggcgccagaactacggaatcacagttacatcgtgaattagcatctggactgtccttccctgtgggattcaaaaacggtacggacggcacacttaacgtggccgttgatgcttgtcaagctgctgcccactctcatcacttcatgggagtcactctgcacggcgtagctgcaatcacgacgaccaaaggcaatgagcattgcttcgtaatattacgtggtggcaagaagggcaccaactacgacgctaagtccgtggccgaggcaaaagctcaactgccagctggctccaatggtttgatgattgactattcacatggaaattctaataaagattttagaaatcaaccaaaggtgaacgatgtggtatgcgagcaaatcgccaatggcgaaaacgccattactggcgtgatgatagaatctaatatcaacgaaggaaaccaaggaatacctgcagaaggtaaagccggtctaaagtacggagtttcaattaccgatgcttgtattggctgggaaacaactgaagatgttctgaggaagcttgctgctgctgttagacaaagacgtgaggtgaataaaaaataa

SEQ ID NO: 96 - DNA, 1551 bp, Aspergillus oryzae
atggaccgtgaccagttcagggctgccgctcacgcaactgtggacgatataattaattattttgatagtgtgccagatagaagagtgtgccccacggtaacgcccggctacctgaggccattaattccagagcaacctccaactgaacccgaagagtggtctcagattcaagcagatgtcgataccaaaatcaaacccggtttaacgcactggcaacatcccaacttcatggcattttatcccgcaactgtgacatatcccagtattcttggcgaaatgtactcagcagccttcaacgcccctgcatttaactggttgtgctcccccgcatgtactgagctagagaccatcgtattggactgggtcgctcaagctctaaatttgccaaagtgttttatgtcatcttctgagaataggggaggtggagttttgcaggtgtcagcttcagatacaatagccacagtgatggtggcagccagggaacgtagagtccgtgagctggctctagccgagggcttgaaagagggaactttggagtatgaagacagagttatggacctaaggccaaggttagtagctatgggatcaaaccaggcccacagttccaccgctaagggcgccctaattgctggtacaagatatagatccgccactgccaaactggagaacaacatggagcttacgggtgatgatgtgcgtgcagtgttggaacaatgcgagaaggacaatctaacgccttattacataacgttatctatgggaaccacgtccacctgtgccgtcgacaggattagtgaagtcaccgctgtcttgagagaaaaacccagttggcaaagaatctgggtacatatcgacgcagcatatgccggctcagctttggttgccgatgaattccaatacttagctaaggacctggcagagggtgtggatagttttaactttaacatgcacaagtggctactggtgaattttgacgcttcctgtttgttcatccgtaacaggtttgatctgaccgacgcattagatattactcccgcttaccttagaaacccctattccgaatcaggccaagtaatagactatcgtaactggagtatctccttgggtaggaggtttagggccctgaagatctggttcgtcatgaggtcttatggcctgaacggtttgaaggcacacgtgaggaagacgattagagttggagatgagtttactaacctggttagatcccgttccgatttgttcgagctaattaccaagccagcttttggcctgactgttttcaggattaaagatcctcgtgctcaagctaacggctcagctgtgaatcgtacaactgctgtgaaaccaaacgagaaaaatgatgctctaacgaaagaggtctacgaattgatcaacgagaggggtgaaatcttcatcacgtctaccgtcgtgtctggaatatacgcaatcagggttatttgcgccaatgaagccgccgaggaaaaatacctaagacgtgcctttgagatcctagtggaaactacagacgaggttctgggtagaagtaattaa

SEQ ID NO: 97 - DNA, 1191 bp, Methanocaldococcus jannaschii
atgaggaacatgcaagagaaaggagtgagtgaaaaagagatattagaggaattgaaaaaatataggtctctggacttaaagtacgaagacggcaacatctttggttctatgtgttcaaacgtcttacctataacgagaaagatcgtggatatcttcctagaaacgaatcttggagacccaggattatttaaaggtacaaagcttctagaagaaaaggccgtggcccttttaggaagtcttctaaataataaggatgcatacggtcatatcgtgtctggaggaacggaagctaatcttatggccctaaggtgtattaagaacatttggcgtgaaaagaggcgtaagggcctgtccaagaacgaacatccaaagataattgtgcccataaccgcccacttttcattcgagaaaggtagggaaatgatggacctggaatatatttatgctcccatcaaagaagattacaccatagatgagaagttcgtcaaggatgctgtggaggattatgatgtagacggaattataggcatagccggtactacggagctgggcactatcgataacatcgaagaactttccaaaatcgcaaaagaaaataatatctacattcacgtagacgctgcatttggtggcttagttatcccattcctggatgataaatacaaaaaaaagggtgtcaactataaatttgattttagtcttggagtggacagtataactatcgacccccataaaatgggacactgccccattcctagtggcggaatactgttcaaagacataggatacaagagatacttagacgtagacgctccctacttgactgagactagacaggctacgattttgggtacgagggtcggctttggcggcgcttgtacctacgcagtcctacgttacctgggcagggagggacaaaggaaaatagtaaatgagtgcatggagaataccttgtacttatataagaaattgaaagagaataacttcaagccagttatcgaacctattttaaacatcgtggcaatcgaagatgaagattacaaagaggtatgcaagaaattgcgtgatagaggcatctatgtgagtgtttgcaactgcgttaaagctcttcgtattgtggtcatgccacatattaaacgtgagcatatcgacaattttatcgagatattgaactccataaaaagagactaa

SEQ ID NO: 98 - DNA, 1854 bp, Enterococcus silesiacus
atgaagaacttcaacactgacgatacaaatttaaaagctttattcattggcgataagggtgagaatgttgatctgtttaaggaattgctaaacaaaatgatagatgaacacgtgggatggagacagaattacatgccccaagacctacctgtaattactcctcaggatagatccagtaaaagtttccaagagaccgcagacaacatgaggtccgttttcaacgttctgtcatcacgtctgcgtacagaatccttgccctggcacagtgctggtcgtttctggggccacatgaacgcagagaccttgatgccatctataattgcatacactaccgccatgttatggaacggtaataatgtggcctatgaaagttcacccgccacttcccaaatggaggaggaggtcggtatggaatttgctacacttatgggctatgaagatggatggggacacatagccgccgatggcagtatcgccaaccttgaaggattgtggtatgcacgtaatatgaagtcacttcctttcgctattaaggctgttgtgcccgagatggtagccggtaagtctgagtgggagttgttgaatatgagtaccacggaagtgctagacattcttgaccaagtacaggataatttcgacgaaataaaagctcaatctgccaggagtggaaagaatcttgacaagctgggaaagtggatcgtaccccagactaaacattacagttggctgaaagctgccgatataattggtatcggattagatcaagtgatcgccggtgaagttaactctgaatacaggatggacatcgataaattggaggctcagatcagagacttggctgcccagggtatccccacacttggtgtggttggtgtggtgggaagtacggaggagggacaagtcgatcgtattgaccaaatcattgccctacgtgacaagttggcaaaggagggcatttatttttacgttcatgtagacgccgcctacggcggatatggacgtagtatatttctagacgagaacgataaattcatcgagtgggacaaaatcgaggaagtctatgctaagaacggtatcttcatggagaagaatgattggttaacgagagaggtttatgagtccttcaaagccatcgagcaagcagagagtgtgactattgatccccataaaatgggttatattccctactccgctggtggagtcgtcatcaaagatatacgtatgagggacgtgatatcttactttgctacttacgtctttgagaagggagccgatatacctgcactgcttggagcctacattctggaaggttctaaggctggcgccacagcagctgccgtttggactgcacacaaagtgttgccactaaatgttacaggctacggaaagttaatgggtgcatctatcgagggcgcttaccatttttaccacttcattgacggcctagagttccaggtgggcgataagacaatcgagatgcatgcattaactaagcctgacttcaacatggtcgactatgtgttcaatgagaagggtaatacggaccttgttaagatgaataagctgaatcacgatttttatgactacgcttcttacgctaagggcggcttgtacactaatgagtttattacctcccatactgacttcgccattgaggagtatggacattctccctttgagtttgtgaacagtcttggcttctcaagagctgaatgggaaagagccgataaggtcaccattttgagagccgcaggcatgagtccatatatgaatgataaggaaatctttgacgaatatgccgaaaagatcaagactgccattcaggaaaagctagaggccatttatgctgaataa

SEQ ID NO: 99 - DNA, 1083 bp, Komagataella phaffii
atgacatctacgcccgtacaggaggaatatgacgacgtaagaatcgccggttatgaccccctggtgagtccagcattacttcagcaagagatacccgccactaaagttgctctacaaactgttgtgaaaggcaggaaagatgccatgaacgtcgtttctctaaaagacgacaggctacttgtcgtagtcggcccatgttctatccatgactccgatgccgcccttgaatacgtagccaggttaaaagctttatctgaagagctgaaagatgaattagtaataataatgagagcatatctggagaaaccaagaacaaccgtgggctggaagggactgattaacgatcctgacctagacaattccttcagtattaacaagggtctgaaaattagtagaaaactatttgtagacctaacaaatagtggcatgccaatagcctctgagatgttagacaccatttcccctcaatatttggctgacttgttgtcttttggcgctataggtgctcgtacaacagaaagtcagttacatcgtgaacttgcttccggtctttccttcccaatcggattcaagaatggcactgacggcacgttggacgtcgcactggacgcagtgcaagcagcttcacatgcacatcacttcatgggagtaacactacatggtattgcagctatcacgacgactaagggtaacgaatactgcttcgttatattaaggggtggaaaaaagggtactaactatgatcctgaaagtgttgctgaagccaaagcaaaacttcctgctgacggcgtgctgatgatagactgctcccacggaaattcaaataaggattatagaaaccaacccaaggtctctaaggtcgtggccgatcaagtcgcagctggtgaggatcgtatcatcggtgtaatgattgaatccaacattaatgaaggaaagcaatctatccctgccgagggacgtaaggctcttaaatatggagtaagtgtgacggatggatgcgtgtcctgggaaactactgacgctatgctaagagagttggcagaggccgtgcaaaaaagaagagctctaaagaacgccgcctaa

SEQ ID NO: 100 - DNA, 1083 bp, Komagataella phaffii
atgacctctacgccagtacaagaagagtatgatgatgtccgtatagctggctatgaccctctggtatctcccgctcttctgcaacaagaaatccccgcaactaaagttgcccttcagacagtcgtaaaaggcaggaaagatgccatgaatgtcgtgtccttaaaagatgatcgtctgcttgttgttgttggaccatgctccatccacgactcagatgccgctttggagtatgtcgcaaggttaaaggcattaagtgaagaactgaaagacgagctagtaattatcatgagggcctatttggaaaaaccccgtaccaccgttggttggaaaggattgatcaacgatcccgatctagacaattcattcagtataaataaaggtttaaaaatatctagaaaactgtttgtggatttaacgaacagtggtatgcccatcgcctcagagatgctagacacgatatcacctaaatatttggccgacttgctgtcctttggagctatcggcgcccgtaccactgagtctcagcttcatagagaattagcttcaggactaagttttcctataggtttcaaaaacggcactgatggtacactagatgtcgctctggatgccgtccaggcagcatcacatgctcaccactttatgggagtcacaaagcatggcatcgcagccataactaccacgaaaggcaatgaatattgctttgtcatcctacgtggtggtaaaaagggcacaaattatgaccctgagagtgtagccgaggccaaagctaagctgcctgccgatggtgtattgatgattgactgttctcatggaaactctaacaaggattataggaatcaacccaaggtgagtaaggtagtagctgaccaagtagccgctggcgaagatagaataataggcgtaatgatcgagtcaaatattaatgagggtaagcagtcaatacctgcagaaggtagaaaggctttaaaatatggtgttagtgtcacggacggttgtgtttcttgggaaacaacggatgctatgttgagggaattggccgaggctgtccaaaagagacgtgcactgaagaatgcagcctaa

SEQ ID NO: 101 - DNA, 1083 bp, Komagataella phaffii
atgacgtctacacctgtgcaagaggagtatgatgacgtgcgtatagctggatacgatcctctggtctcacctgcccttcttcagcaagaaattcctgctacgaaagttgcccttcaaaccgtcgtcaagggcagaaaggacgcaatgaacgttgtctctttaaaagatgatcgtttgctagttgtcgtaggtccctgctcaatacacgactcagatgctgcattggaatacgtggcccgtctgaaagcactatccgaggagctaaaagacgagttggtcattattatgcgtgcttaccttgagaagccccgtacaacggttggttggaaaggtcttataaatgatccagacctagacaattctttctctatcaacaagggcctaaaaatttcaaggaagttgttcgtggaccttaccaactcaggaatgccaatcgcttctgagatgttggatacgatctcccctaaatacttagctgatttgttatcctttggtgctattggcgcaaggaccactgaatcccaacttcatagggaactagcctctggcctgagtttcccaattggatttaagaatggtaccgatggtactcttgatgttgctttagatgctgtgcaagccgcttctcacgcccatcattttatgggtgtcacgttacatggtattgctgccatcacaactactaagggcaatgagtattgcttcgttattctaaggggcggaaagaaaggcacaaactacgaccctgaaagtgtcgccgaggctaaagctaaactgcccgctgatggcgtcttaatgatcgattgttcacatggaaattccaataaagattacaggaaccaaccaaaggtaagtaaggttgtagcagatcaggtggctgctggcgaagatcgtatcataggcgttatgatagaatccaatataaacgaaggaaaacagtcaatccccgcagagggaagaaaagccttaaagtatggagtctcagtaaccgacggctgcgtttcttgggagaccacggacgcaatgctgagggagttggcagaagccgtacagaagagacgtgctcttaaaaacgctgcataa

SEQ ID NO: 102 - DNA, 1137 bp, Komagataella pastoris
atgaccgtgtccttcttaccaattgaacccaacaaccgtacgagtccattaaccctacagtcaactcccgtacaggaggaatatgatgatgttagaattgctggctacgacccattagtcagtccagccctacttcaacaagagattcccgccactaaagtagcattgcagacggtggttaagggaagaaaggacgcaatgaacgttgtatccctaaaggatgaccgtctactagtcgtcgttggtccttgttcaattcacgattccgacgccgcactggaatacgtcgcacgtttgaaaacgttgagtgaagagttaaaagatgagttagtcatagtgatgagggcttacttagaaaaacctagaacgacagtcggatggaaaggtctgataaacgaccccgatttggataactccttttctattaacaagggtctgaagatttccagaaagctttttgtcgatctgaccaacagtggtatgccaattgcttcagaaatgctggacacgatatcaccccagtacttggcagacttactgtcttttggagcaatcggtgctagaacaacagagtctcaattacatagagaattggcctctggactgtccttccctatcggctttaagaatggcacggacggtacgctagacgttgcacttgacgcagttcaagctgcctctcatgcccatcacttcatgggtgttaccctgcacggaatagctgctattacgacgacgaagggtaacgaatactgttttgtaatcctgcgtggcggaaaaaagggaacaaattatgatcctgagtctgtggctgaggcaaaagctaaactgccagccgatggagtattgatgatagactgttctcacggaaattcaaacaaagattatcgtaatcagcccaaggtctctaaagttgtcgctgatcaggtggctgccggagaagatcgtatcattggcgttatgatcgagtcaaatatcaatgaaggaaagcaatctattcccgctgaaggcagaaaggcccttaaatatggtgtctccgtcactgatggttgcgttagttgggaaacaacagactctatgctgagagaattagccgaggctgtgcaaaagcgtagagctttgaagaatgcagcctaa

SEQ ID NO: 103 - DNA, 1137 bp, Komagataella pastoris
atgacggtcagtttcttacccattgagccaaacaaccgtacttcacccttaacgctgcagtccacccctgtgcaagaagaatatgatgatgttcgtatcgcaggttatgaccccctggtttcccccgctttacttcagcaagagataccagcaactaaagtagccttacaaacggtagtcaagggtagaaaagacgcaatgaatgtagtatctcttaaggacgaccgtttgttggttgttgtcggaccctgttcaatacatgactccgacgctgcattggaatacgttgcccgtcttaaaactcttagtgaggaattaaaggatgaactagttatcgtgatgagagcatacctagaaaaaccaaggactacggtgggatggaaaggacttataaatgaccctgacctagataacagttttagtattaataagggcctaaaaatctctcgtaaactgttcgtggatttaaccaactccggaatgccaatagccagtgagatgttagacaccatttctcccaaatatttagctgacttactgtcattcggcgctatcggtgctaggacgacagagtcccagttacatagggaattggcctctggccttagtttcccaataggcttcaaaaacggtacagacggaactttggatgtggctctggacgcagtccaggcagcatcccacgcacatcattttatgggtgtaacgaagcatggtatcgccgccattacaacaacgaaaggtaacgagtactgttttgtaatcttgagaggcggcaagaagggtacgaactacgatcctgagagtgttgctgaagccaaggctaagctgcccgctgacggagtcttaatgatagactgctctcatggcaattctaacaaggattaccgtaatcagcctaaggtatccaaggtagtcgcagaccaggtcgctgccggagaggacagaataattggagtcatgatcgaatctaacataaatgagggtaaacagagtattcctgccgagggcagaaaagccctaaagtacggtgtctccgtcacagatggttgtgtctcctgggagactacagatagtatgctaagagaattggccgaagctgttcaaaagagaagggctttgaaaaatgctgcttaa

SEQ ID NO: 104 - DNA, 1137 bp, Komagataella pastoris
atgacggtgtcatttctacctatagagcctaacaaccgtacgagtcccctgaccttacaatcaactcctgtgcaggaggaatacgatgacgtacgtattgctggttacgacccccttgtatcacctgccttgctacagcaagagattcctgctaccaaagttgctctacaaactgtagttaaaggtcgtaaggacgcaatgaacgtagtgtccctaaaagacgacagacttcttgtggtagtgggaccttgctccatccatgattctgatgctgccctagaatacgtcgctcgtcttaagacattgagtgaagaacttaaggatgagttggtcatagtcatgcgtgcatatcttgagaagcccagaactacagtgggctggaaaggtttgataaacgaccctgatcttgacaattcattctctataaataaaggcttaaaaatatcacgtaaactgttcgtggatcttactaacagtggtatgcccatagcctccgaaatgttggacactattagtcccaaatatctagccgatctgttatcttttggtgcaatcggtgctagaacgacggaatcccagttacatcgtgaattagcctcaggtctatcttttccaataggcttcaaaaatggtacggacggaactcttgacgtagctttagacgcagttcaggccgcaagtcacgcccatcactttatgggcgttactctacacggaatcgccgccatcaccacgaccaaaggtaacgagtattgttttgtcatacttaggggaggcaagaaaggtaccaactatgaccctgagtcagttgccgaagccaaggcaaaattacccgctgatggtgtgcttatgatagattgcagtcatggcaactcaaataaggactacagaaaccaacccaaagtatcaaaggtcgtggcagaccaggttgctgctggagaggatagaataatcggagtgatgatagagagtaatataaacgaaggaaaacagagtattccagctgaaggtagaaaagctcttaagtacggcgtgtctgttactgacggctgcgtgtcatgggagactactgactccatgttaagggaattggccgaagcagttcaaaaaaggcgtgcacttaaaaatgccgcataa

SEQ ID NO: 105 - DNA, 1104 bp, Komagataella pastoris
atgttcattcaaaacgaccatgtcggcgacaggagtagattggaagactggaggattagaggctatgacccacttactccacctgatttgttgcagcacgagtaccctttaacggctaaagccgaagaaaatatattaaaaggcagaaatgaagcagtggacattctgaagggtaaagacgatagactactagtcattgttggaccatgctccatacacgaccctcaggctgcattagattactgtgaaaggctggctaagtttaatgaaaccattaaaggagaacttcatataatcatgagagcctatttagaaaaaccaaggacgaccgtgggatggaaaggattgataaatgatccagacattgacggatcctttcagattaataagggcttacgtatctctagggaactattcgtaaagttaactgaacagatacctattgcaggcgagatgcttgatacgattagtccacagtttttgagtgatctattctccttaggtgccataggcgcaagaactactgagagtcaattgcaccgtgaacttgcatctggactatctttcccagtgggttttaagaatggtactgatggcggtcttactgtggcaattgatgcaatgagagctgctagtcatccacaccacttcctttcagttaccttgcccggagttgtcgccatagtcggcaccgaaggaaatgaagatacgtttgtcattctgcgtggtggaaaaaatggtacgaattacgacgaggcctcagtagacgccgctcacgcacagctagagaagacgggcatcttaggctctggcccagcaataatggtagactgttcccatggtaattctaacaaggaccacaggaatcagcccaaggtggccgaagtggtggcagagcaaatcaggaagggatcccgtaaaatatgtggcctgatgatagagtcaaatatagtagagggtagacaggatgtcccaaaggaaggaaagtcacatctacgttacggctgttcaatcacagacgcatgcatctcttgggaggacactgagaaagtactgagagtgttggcagactcagttattgaacgtaggaaccttaaaaattaa

SEQ ID NO: 106 - DNA, 1128 bp, Komagataella phaffii
atgccagttccctcctcccataagatgtttatacagaatgaccatgtgggcgatcgttccagacttgaggactggaggataaggggatacgaccctttaaccccacctgaccttcttcagcacgagtaccccctgacgccaaaggccgaggaaaacatcttgaaaggtcgtaacgaggccgtggacatattaaagggtaaggatgacaggctgttagtgatagtaggaccatgttctatccacgacccaaaagcagccttggattattgcgagaggctagctaagttcaatgagaccatcaaaggcgaactgcatattatcatgcgtgcctacttagagaagcctaggacaaccgtgggctggaagggcctgataaatgatcctgacatagacggtagtttccagattaacaagggtctgagaattagtagggagctgttcgtaaagttgaccgaacaaatccctatcgcaggcgagatgctagacactatcagtccccagtttttatcagacttgtttagtctgggcgctataggcgctagaacaactgagtcacaactacatagagaattagcctccggtctgtccttcccagttggttttaagaacggcaccgatggtggactgacagttgcaattgacgctatgcgtgccgcttcacacccacatcactttttatcagttactcttcccggtgtagtggctatagttggtaccgaaggtaacgaggacactttcgtcatattgcgtggaggaaagaacggtacgaactatgacgaagcctctgtcgatgccgctcacgcccagttagaaaagaccggaatccttggatcaggccctgcaattatggtggactgctctcacggcaactcaaacaaagatcacagaaaccaaccaaaggtagcacaggtggttgccgatcaaatacgtaaaggaagtagaaagatctgtggacttatgattgaatcaaatatagtggagggtaggcaggatgttcctaaagagggtaagtcccaccttcgttatggctgttcaataacagacgcttgcatttcatgggaagatacagaaaaggttcttagggttctggctgattccgttatcgaaagaagaaatttgaaaaattaa

SEQ ID NO: 107 - DNA, 1113 bp, Saccharomyces cerevisiae
atgtttataaagaatgaccacgccggagatagaaaacgtctggaagactggaggataaaaggctatgatcctctaacgccccccgacctattgcaacacgaatttcctatttcagccaaaggagaagagaacataattaaagcacgtgattccgtatgcgacatattaaacggtaaggatgaccgtttggtcattgtaataggtccttgctccctgcatgatcctaaagctgcatatgactatgcagaccgtcttgctaaaatttcagagaagttgtccaaagatctattaattattatgcgtgcatacctggagaaaccccgtactactgttggatggaaaggtctgattaatgaccccgatatgaacaattcatttcaaatcaataaaggactaaggatttctagagagatgttcataaagttggtcgagaaacttccaatagccggagagatgttagatacaatttccccacaatttctgtccgactgtttctctctaggtgccattggtgctaggaccacagagtctcagttacacagagaactagccagtggcctttcatttcccataggattcaagaacggcacagacggcggactgcaggtcgccatcgacgccatgcgtgcagcagcccatgaacattactttttgtccgttacgttgcccggagttacagccatcgttggcaccgaaggtaataaggatacgtttttgattttgcgtggtggaaaaaacggaaccaacttcgataaggaatcagtgcagaacacgaagaaacaattggagaaagctggcttgactgacgactcccagaaaagaataatgatagattgcagtcatggtaactccaacaaggacttcaagaatcaacccaaggtcgctaagtgtatctacgatcaattgaccgagggtgaaaactctctatgtggcgttatgatcgagagtaatatcaacgaaggccgtcaagatatacctaaagagggtggcagggaaggcttaaagtatggctgctctgtaacagacgcctgcataggctgggagtcaactgaacaggtcttggaactgttggctgagggcgtgaggaacagaagaaaggctttgaagaaataa

SEQ ID NO: 108 - DNA, 1053 bp, Escherichia coli (strain K12)
atgaattaccagaacgacgatctgcgtatcaaggagatcaaggaactgctgccaccagtagctctgcttgagaagtttcctgctaccgaaaacgcagcaaacaccgtggcacacgctaggaaggctatacacaagatcctgaagggcaatgatgacagacttctagtagtcataggaccatgttctattcatgatcctgtggctgcaaaagagtacgcaactaggctactagcattgagggaagaattaaaagacgagctagaaatcgtcatgagggtatatttcgaaaaacctcgtacgacggtgggatggaaaggcctaattaatgatccacacatggacaattctttccagattaacgacggtcttcgtatcgctagaaaattactattggatattaacgacagtggattaccagcagcaggcgagttccttaatatgattactcttcaatacttagctgatctgatgagttggggagcaataggcgcaaggacaaccgagtctcaagtgcatcgtgaactggcatccggtttgagttgtccagtaggcttcaaaaacggcacggatggcacaataaaagttgcaatagatgccataaacgctgctggcgctccccactgcttcttatccgttacgaaatggggacactctgctattgtcaatacgtctggtaatggcgactgtcacataatacttagaggcggcaaggaacctaattattctgctaaacacgtagcagaagtcaaagaaggcttgaacaaggccggattgcctgctcaagttatgatagatttttcacacgcaaactcctcaaagcaattcaagaagcaaatggacgtttgcgccgatgtctgtcagcaaatagctggaggtgagaaggccatcattggtgttatggttgagtcccacttggtcgagggcaatcaatccttggagtctggtgagccccttgcctacggaaagtctatcactgatgcttgtataggctgggaggatactgatgctttgcttagacaacttgctaatgccgtaaaggcccgtagaggctaa

SEQ ID NO: 109 - DNA, 1071 bp, Escherichia coli (strain K12)
atgcaaaaggatgccttgaataaagtccatattacagacgaacaggtccttatgactcccgagcaattgaaagcagctttcccattgtctcttcaacaagaagcccaaatagccgattcacgtaagtctatttcagacatcatcgccggaagggatccaagattactagtcgtgtgcggaccttgcagtattcacgaccctgaaacagcccttgagtacgccagaagatttaaagccttagccgcagaagtgtccgattctctatacctggtaatgagggtttactttgagaaacctcgtacgactgttggttggaaaggtcttattaacgaccctcatatggacggatcttttgacgtagaggcaggtctacagattgctagaaagttactgcttgagctggtcaacatgggactgcctttggcaacggaagctttagaccctaactcaccccaataccttggcgatctgttttcttggtcagcaattggcgctagaacgaccgaatcccagacgcacagagagatggcatctggcctttccatgccagtaggattcaagaatggtactgacggatctttggcaaccgcaattaatgctatgcgtgccgcagcacaacctcacaggttcgtcggaatcaatcaagctggccaagtcgcacttctacaaactcagggtaatcctgatggtcatgtaattctgaggggcggtaaggctccaaattactctcctgcagatgttgcccagtgcgagaaagagatggaacaggctggtttgcgtccttctctaatggttgattgttcacacggaaattccaacaaggactacaggaggcaacctgctgttgcagaatcagtagtcgctcaaattaaagatggtaataggagtatcatcggtcttatgatcgagtccaacattcacgaaggtaaccagtcctctgagcaaccaagatcagagatgaagtacggagtgagtgtgaccgacgcttgcatctcctgggagatgacagacgccctacttagagagatacaccaagatctaaatggtcagcttacggccagggtcgcataa

SEQ ID NO: 110 - DNA, 1047 bp, Escherichia coli (strain K12)
atgaatcgtacggacgagcttaggactgctagaatagagtccttggttaccccagctgagctagctttgagatatccagttacgcccggtgtagcaacccatgtcactgactcaaggaggcgtatagaaaagattcttaacggcgaagacaagaggctacttgtcataatcggaccatgttcaatccacgaccttactgccgcaatggagtatgccacaagattacaaagtctaaggaaccagtaccagtccaggttggaaatagtaatgcgtacttattttgaaaaaccccgtacggttgttggatggaagggtctgatttcagatccagacctgaatggctcatacagggtgaaccatggcctggagcttgctcgtaagttactacttcaagtcaacgagttaggtgtgcctaccgcaacggagttcctggacatggtgacaggacaattcatcgctgacttgatctcatggggtgccatcggcgcaaggacgaccgagagtcagatccatcgtgaaatggcctccgctttaagttgtcccgtcggcttcaaaaatggaaccgacggcaatacacgtatagccgttgacgcaataagagctgccagagcaagtcatatgttcttgtcaccagataagaatggccaaatgacgatttaccaaacctctggaaacccctacggacacattatcatgcgtggcggtaagaagcccaattaccacgccgatgacatcgctgctgcatgcgatactctacatgagtttgacttacctgagcacctagtagttgactttagtcacggaaactgccagaagcaacaccgtcgtcagttagaagtatgcgaggacatatgtcagcagattaggaacggcagtactgcaatcgcaggaatcatggccgagagtttcttaagagaaggcacccagaagatagttggatctcaacctttaacctatggtcaatcaatcacggacccatgtttgggatgggaagacaccgaacgtctggttgaaaagttggcatccgctgtcgacacacgtttctaa

SEQ ID NO: 111 - DNA, 700 bp, Komagataella pastoris
accacgactttattagcctgtccattctggttcctggcgagacttattcttgtttgtttattttcgaatgcaacaaagctccgcattacatccgaacatcactttagatgagggctttctgagtgtggggtcgaatagtttcatgttcccccaatggcccaaaactgacactttaaacgctgtcttcgaacttaatatggcaaaagcgtgatctcatccaagacgaactaagtttggttcgttgaaatgctaacggccagttggtcaaaaagaaacttccaaaagtcggcatatcgtttgtcttgtttggtattcatagacgaatgctcaagaatattctcattaatgcttagcgcagtctctgtatcgcttctggaccccggtgcagttgtgccgaaacgcaaatggggaaacacccgcttttcggatgattatgcattgtctccacattgtatgcttccaagattctggtgggaatactactgatagcctaacgttcatgatcaatatcaaactgttctaacccctacttgaactgcaatatataaacaggaggaaacttcccagtcgaaaaccttctttcatcatcattattagcttactttcataattgtgactggttccaattgacaagcttttgattctaacgacttttaacgacaatttgagaagatcaaaaaacaactaattattcgaaacg

SEQ ID NO: 112 - DNA, 1000 bp, Komagataella pastoris
gcttgcacgactcagttacctgaaaatttcagcctgtcctctttaataaaatttcacccgttcctctgcatgtccactcagctctattcatctatccttgagccttctcgagcgtctaatgaacagcctgcgaattcaggttacaacccctcattttttcgtctcggtcgatctctacaaagtcaacagccaactttgatgttaagcgagtcatcaccagccagcgatagtgaaaaactttaagcatagattgatggtgggtttatgtcacttggcggctgcattagagtttgaaactatggggtaatgcatcacatccggaactgatccgactcggagatcatatgcaaaccacgtgatgtaccccgtaaactgctcggattactgttccaattcatcgtcttaaacagtataagaaactttattcatgggtcattggactctgatgaggggcacatttccccattgatttttgggacagtaagccataaaaggactgttaagcgaagcagacaagacaacgaacagctagaataacaactatctaccgccttgtggaccgttgggagtttccaattggttggttttggatttctgagcccatgttgtgttgtccatgcttctccttgcacacaatgcaagttgataagatatcaccttccaagataggctatttttgtcgcataaattttggtctcagagtgaaacccccttttatgtgaacggattagagaagcctcctacccttcaccggctgagatggggagaaattaagcgatgaggagacgataattgctataaaagaagcaaccaaaaccccttattgtctctttctgatcagcatcaaagaatattgtcttaaaacgggcttttaactacattgttcttacacattgcaaacctcctccttcaatttcggatcagctgtattgactacattgatcttttttaacgaagttcacgacttactaaatccccataaacaaaccaactgagaaaa

SEQ ID NO: 113 - DNA, 560 bp, Komagataella pastoris
aagcagcggttttaactaagaattgagtgagtgggtctgctctgttttcaaagggccataaccatctgtttgcatccccttatcaccacaccatcctcctcatctgcccttcaattgtggggaacgacacccatcccaacaccagagtaactccacctagatgaaacagcccaattgtcgcttaccagtcattgaatgttgagtcaacgtttcttaaaactcgaatgatcccagccttgctacgtagcaaccttccgttattccgccgcttggtccaaccatgtttccgtttttttcgaacaagtcaaaatactcgtctttggcagggcttttcctcctgccttttttagcctcaggtatcggttagcctctaggcaaattatggtcttcatacctatatcaacctttcatcagatagcctttgggttcagaaatgaactaaagcaggatgcctgatatataaatcccagatgatctgctttgcaaactgtttttactatcttgattcgtttagtttcaaacatcttttgttgattttatctggagaataatcaaacaaa

SEQ ID NO: 114 - DNA, 700 bp, Komagataella pastoris
accccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcacttctatcaaactatcaaacatcaaaa

SEQ ID NO: 115 - DNA, 700 bp, Komagataella pastoris
aaactattccataagataaggcagatgtgcttaagtaattgagatcaattggtgtgattaggtaagcacgatgcttgcctggaactggtatcgcaactagtttgacaggatggaagtcagggtagatcaggatgcttagagaaaaggattagctaatatgaagaccatcattagcaacacttatttaattagatcaatggtggggtactgtgaattgactttcaccaggacttcgaacttcaattagttgaacgttctaaaagaccaacatatactttggtcggtaatgtgatcagaagatgattactagatcccaaactgaaatgctatcttttctggacagcaattttatcctacgatcaaagaatcttgctcttttgagaagaacattttcattggtgggaactctaaaagaaagtgaatattccccgatgaccctatgaatcagaaccttatcgtctctccttttgccctgtcaaactcacatttgaattttccagccactcacactgtcaatgatgactttccagtccccttctacccgtattcgtaccatgtaaccctacacggtttttcccatttcggttcggatgcgacttccagtctgtcgggtacgtagcctattctcttagccggtactaaaacatacaaatttcacccaatttcctctttaacaaggtaaccaattaacttcataaac

SEQ ID NO: 116 - DNA, 700 bp, Komagataella pastoris
gacgatacccgagattttgacgattttcccaatatgacttgaagataagtctctcattgaaaccttgttatcgttactaagcaacacgagctgacaagaggggaaagtggtcggtatttcctcgttgttcaagtatataattctcctggcaatatctgtaatggcctgttcaaaaagaggaatcatttctgcaggatcatccaccaactttttattgagctcttcgttaaatacgattaggtggtcattttgaatcgttagtaggtatttgtttacaagtaggttctgtctgagttgttctctgtagatgtactgattttccatacgaaactccaagatgaacgaccggaatgccttaatgacctcactgaactggtcatcgttttgttctccgggaaggacacttgtgttaaagactgatgctctatcgaaggacattgcaacaaagtataatcggttgtgggtggtaaaaagatgggtagataattgtagtagatcggactaattcagttggaaaacgcgtcttgcattctttttttcttcattacacttcctaatcgggacaaaataaatttatagacgtggttatgtagtacacgcagcgataggctatccccataccaaaacacaattttaccccattcttcacaggttataaatatgcgatagtatgaagatcttaaagttcagctagaatatt

SEQ ID NO: 117 - DNA, 483 bp, Komagataella pastoris
gttcggagtgtcaaaaaagtaatgataaaaccaagctataagggaggttttctagggacacgcggaggagagtggttcaaacagcccttccaaaaaaagaaaaaaaaaagggggaatctctgttctttccaattgaaatgacgtaactttaactgaaaaataccccaaccagaagggtttaaactcaagagggattgcgtaatttctagggtagtttagtcccagctgaaggcagcttaacgataacgcgagggggggggggggggggggggattggtgcacgactgaaaggtggtatcttagtcttgtaacctcttttctccagaggctattcaagattcgtaggcgatatcgatgtggagaagggtaaacaatataaaaggcaagagggatgtcaatggagcaactggatagatttcaaattctctagatttcagagtaatcacacaaaagaaggaatcccaccaagaaaaaaaaagaaag

SEQ ID NO: 118 - DNA, 700 bp, Komagataella pastoris
tgataagttcaagtcatcaaggtaaaggagctctaagtttacgccttccaacaacgttatttgaggactcaatacattgttgaattcaaaaccaatcgtctttgaactttctaccggagcttctctacagaaattgattttgatcccattggcatcaaagagacccgtatgataactccatagaaagggaagatgataagccttaaattcatcggtgatctgcaaatttatttctgacatgtcttgctaaagaattatacctcagaaaactggaaatgagctcagaaaactggaatcgagccaatatttgaagattcatgcgttgctcgtgaattgattgataaagagctccgtgattcactttgtcaatgattacccctctcttacccgatttgggagactttgttgaggcttgagaatctttttcaatatgacttgttcttgctttcccaatagggaaggactcatccatggatgattaggtttgaattactcttttaggaaatagtagccatgaatcaaattgaatcataccatcatgaaataaaagttaggctgtaaatgcctcaaaaatggttcttgacgctggatattgagcattggaatgttggcagcaattactataaaaagccattagtattgctcttttttatcctccatattttactctcctcaactttgaaaacttcaataaatcacc

SEQ ID NO: 119 - DNA, 700 bp, Komagataella pastoris
catatctagttttcataaacatataatgataaacacggttttgaaaggttgctcaaactgttagggtaagtcgtctcctacccacagttctatatgtatagcaaggaactaacaacggaactaacaacaggacttacaagttcagttgctgtccaagtggttcatagaagtaaccgtctcatctcgcattggacggcagtaataggatattcttctcgatgcaatcgatgaatttgggttgggcaagccgtttaaagttaaatcctacacaattcccgatccctgtaagttgaactatttatcatcaactcggatatcaatatcaataggttgaaagtgcaacagtgatgcctcctattcagatctttcagctgaagatgaggcacaacgtagcgattcccatcacctggggttgattagccaatggccaattcctcggatacaatgaagcccattccccctatgacttcaattgaacaatgaataaccatgaacattgcaattactattattattattgtcacagaggttgggatgttggaaagagtcagttcatggaagactaccggtcggtaggtagtcatttccccattgggaagtctgtttagtcgtgactcagcaaataccgttttcttatttaagttgtaagatcccgaattgcgagaggctcatctcggaggacgctaatattcatccttac

SEQ ID NO: 120 - DNA, 700 bp, Komagataella pastoris
ggagacgagctaaagtctgtagcattgatttctcctcgttgcgggtttctcttcctggcagagctctcctctccaagagaagaactgaaatgtacaggggaagacattcttgtagcacggcttcttgcagaagcgttggatcttggatgttctctaggtgtatgtggcctatcgttatctgaacttggtgggaagaacaagggtgaagagggaacgttggcaggacctgttggagacgacggaatgggatttgagtctctcatagtggaatcaaaattcgtattattggagtcatattgaggtgaggagtcaatcctgcctgtgtctctattgggagaagaggatcttgacattgctgacggtgaagtttgagactaactggaaacaaagaaatgctacgtttgatgaaggaaagtgcgatgagatattgtgccaaaacgcgcgtgcacataatttatcaccaaaacgttacgttaactgaaaaatattccctgatcgggaaatataattagcattggcgcataaacgatccagattcatatctacaaccacacgtgatatatcacatgaccattaggatgagtacaccccgtctcctccaccatggatttaggctacccacctaaagggtgccaatatctctccaggaagctctgtcggttataaatcgcatactcttccaaaaatccttcaatagttc

SEQ ID NO: 121 - DNA, 700 bp, Komagataella pastoris
gacagaggttactgggaagccacgggccttggcagccaaggtatgaatcatagccttcagacccatgtcaacctttccagatccgattagttcagtgacatcagatgggttgcctggttctaagatagcaatatcaagaccctcatctttgaagtaacccaatttttgtgcgaggtaaattggggcgtggtatggggcagcttgccagttaagacagaaagtgatcttgttagtagacatggtgatttattgaagttttcaaagttgaggagagtaaaatatggaggataaaaaagagcaatactaatggctttttatagtaattgctgccaacattccaatgctcaatatccagcgtcaagaaccatttttgaggcatttacagcctaacttttatttcatgatggtatgattcaatttgattcatggctactatttcctaaaagagtaattcaaacctaatcatccatggatgagtccttccctattgggaaagcaagaacaagtcatattgaaaaagattctcaagcctcaacaaagtctcccaaatcgggtaagagaggggtaatcattgacaaagtgaatcacggagctctttatcaatcaattcacgagcaacgcatgaatcttcaaatattggctcgattccagttttctgagctcatttccagttttctgaggtataattctttagcaagac

SEQ ID NO: 122 - DNA, 700 bp, Komagataella pastoris; identical to SEQ ID NO: 118

SEQ ID NO: 123 - DNA, 700 bp, Komagataella pastoris
tatcgtaccagtcggattcgctgcttggatgggtgactcctgtccagttgactcaacctagtcaaagtaggcctggagacccttaataggtcgatgagtagcctactatgaaaaacccctcaccacaattggactataaaaaggcccgtcaatccccaaagcaaccctctctttttatcctttacttcactactttatcctttgatctttattgaagaaaattaaaaacaattgtaagcagcaatccacacctccctagcaatgactcaatttactaacccggttgacagaaaatgtgagcatctttttagatgtcatgatgataggtggagtattcttaattattgctttcagcaaaccggtgcccataaagtgtctaccattaattcaatgggaggcattaaggctgagattaaacggttgaacttgaactagataattctagcggaaagaattgctctttttattacgtcgaatgacgctatttctttgttattgagaaatactaacaatttttagggctcctactgcaattgaaactcaaactcaaaccactccagctttcaacgaaccccaggttttgaagcttaagcagaatgttagaaacccagactctctggttgctaacgctgttaccccagcttttgactggaacacctttgaattcgctcccatccgtgaatcaaccgtttctcgtgct

SEQ ID NO: 124 - DNA, 700 bp, Komagataella pastoris
gattacgtgtatctaggttcgtatgcccgtgtccacgggttagctgccggagttgcacccgatatcgaaagcataaaggcagaagcccatataatcgactccatcatggaggaaatgcatagacatagagacgtattgaactctcgtggaattgtgaaacttgacgaactgagaccctccaaggcctgcaagcagtattccgactatctcctaaacattgccaaaagttcagactgggtgaccatcaagattgccttggcaccatgtatctttggctactattacgctgccaaatatgctaggtcgtttatcaaggatgaagctgacgtggacgaagaattcatgaagtggatcaatacgtataccggtgattggtacaaagatgctgttgacgaggcaaggcaatcgctagaaagccatatgcaggatgtttctcccgtccaattggccgagctagtcaagatttttgcagacgtctgtcaattggaggtaaacttctggacttcgccaatggaactaccagaggaggatctatagagacaaatagtattacgtaatgagtagtcattgaatggttatcccgcgactccgttggctcatcgcttaggctaaataggaaactaatgaagaccgacgactcacacctagccgaaaaatatcataagaatcctgtacgacttgatttatccaccactcaaac

SEQ ID NO: 125 - DNA, 700 bp, Komagataella pastoris
cacaaattgcaactcccaggggagccgtatcttcgatttcttgtacttgctgagcattttgctgtccatggatgtgcttgaagaggagccaggagccccaggagcccccgatgctgctacttgggatgtagaattgtccagcagatcatcacctccaatatctgccaaatctccactttgttgatctttggcatttttacgaacatctttccaactaagataggttctcaaacgattcaccttggccttatcgtgtctaattaaaaaaatgacatcctcggggacaatagactttgtacctctagagagggcagtttgcgaagagtggagtacgatctccaccacttgggacctgacgatgtcttcaatcaacgatgttgtctccacaggcggatcgtttgtttctccagacacaaacatcatctgttgtatctccatacgatacctgtacttgtgatccttgtccttggtgtcagtcatttcatgcactggacgttcaacatgaaattatcttgtcttgacgagttggtcttgggtagatggtagatgcgggagagcgcgaccaggttgggagagtgaaaatgctggtagcagttaatggaaatcaattccgcgcatcatataatggctccgttccccgaacgatatagcctcctaccggttggaaaaattggaagattgcgataatcctgtcaaaaga

SEQ ID NO: 126 - DNA, 700 bp, Komagataella pastoris
taggttcatgtagagtcacgagacttgacgaagcgtaggatctctgtacgaactttcttcaagaagacttggactaaaatgtgacctgccctgagggtatatgctggtggcaccacaggggaaacttcactatctttgaccacagtcttccagtatcactatgtggcagtcacattttatcagtcagatcaaagattattccagcagtctcgggtaagccaagtttatcgtatgtctcgattcagtaatttcactgcaatgactacatgccgaggtcactaaacctacatggaattgcatatcaaccgtagaaactattagacagcatactgctcaagttgccacaaatcaaaaattatttctgttatatactgtcctgtctataaatacagcgccctccccgaacctgccaggtatactagtgcgaagaaataggcaaacgtttccaacacttcaagtgactccaaccggtattcgcccaactccctactttttcgcggctgatatccaacaccctagattagacaggtcattgcactttctgctattaatctcctatccgaacaacggcagtccctgattttcgctgtcattttttttttattttgacagtaattttttgcttcccccagttatacctaaaagactcattttggtgtcctagtctttgctaacttaaaacaggaaacccttattcaag

SEQ ID NO: 127 - DNA, 700 bp, Komagataella pastoris
acagtggtgcatgaatcacaggtcacagcggtttgagcatgagagaagacggtggtgatctcaaaacaacctggacacttgacatccatgaagaaggatcttggagcttggaccaaagtctttagtttgtgttgcttggcctcggagactgggttggggttcaataaatcttggactaaaaccatggtgtaggatacttgatgcagaaaagatttttgcctctgaaaaagaattaagtgagagaacatcccaaaaaagttacccggaacaaaattaggcttcagccctatcacgtgataacgcttttttttatctggtttctctttcccctaccaagagtttttgactggcagagttgcctaaagggggttgagtcgtttgattcatcatgtacgaaggaacactgcaatcatcagtcttaatccatcgctagtgtggactgcaagggagctgcaagggttaaaagtactgtaggctccattttatgcattcttgatacactctgcccggctatttgaagcaaatgattgttttttccccttttttctttttcggctatatgtagctgactcaccttaaagtccaaatagggcaatacgagatgcctacggctcaggttaccccatgataggccctgataatttttttgatttaatataagagggtccgtcgtaggtaagttcgtttttcatcaagcaca

SEQ ID NO: 128 - DNA, 700 bp, Komagataella pastoris
cagaacaacatgcatacagtgccaagctgtaccaagaagattcagaagtgattgatccactgtttgaggagatcgttgctccacctagaaatgccgaaagattcaaaacacaatttatcgtcatgcccttagacaacaatggtgtttccacattgaagcttaccttattagtcaacgagagtgtattggatgccaagcaaagagctcagttattccttgatgaagtcaaggatcagttgacgcaaggcaatggtggcgtttcgtcttctcctcaacttgaagagttgttctgaattgcaataagcctaggtagtaagtaattaaaccgtcatgatcccatgatcatgatcattcattaaagcacagtatggcacatttttgacgggaaaacgtcagtgtggtggtagaattatctctacagaccgtctcaaagtccaccccacgaagttcggaccctctcatctgtaatgcatttttcctgtaacttcccctagccacacgtcaagggctctgaatctgcccaagagttgtggcgtctgccacctttaacgtctacatgagcgatgggcacgtgatattgtctgtccctttttcaaagcttcgaaaaaacgcatctcaatgcgagaagcagatcgatatatatagagaactagtccagtgaaagatctctcaatttcacccgaaaccgactcagaaagaa

SEQ ID NO: 129 - DNA, 638 bp, Komagataella pastoris
taccgttttgatctcagtacgagaggaacaggagatgagctgaaacttggaacaggaacgatcaattgccgaagagcagtctcggaagagagagtggcaaagaacacagggtcacttactatccaggcttacaacaagatctcctgaaatatgacatacaacagaacaacaaagagggggtgcaacaaaggaagttacacttttttcccaccctttccaacgaaaccccacaccagtgaagtgaacagatttaacgggtcttagataaacgaaagaaaaacactaacaatacatcctccgtgaggggggtggtcgccgactgccacatttcctaacagatcaccccctccgcttcccacattgtgattgttggtttcaaatggtactgcagtcaaacgccacagcttaaaaacttaatttgaattctttatcaattaccgtaatattagtcgggccttgccgcatcacgtgaccattttacactataaaactcccgttcccataaagtgttttaccacatcacgtgagttgtcaacattgaaacccctcgatgtaatgcttcacaggttggttatataaatcatccaatcgccgaccaaatgaaatgatttccaacgcttccttattccaatacaaag

SEQ ID NO: 130 - DNA, 700 bp, Komagataella phaffii
acaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 131 - DNA, 700 bp, Komagataella phaffii
ggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaa

SEQ ID NO: 132 - DNA, 552 bp, Komagataella phaffii
agcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 133 - DNA, 700 bp, Komagataella phaffii
gctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 134 - DNA, 700 bp, Komagataella phaffii
taatccataagataaggcaaatgtgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaat

SEQ ID NO: 135 - DNA, 700 bp, Komagataella phaffii
atacccgaaattttgacgattttaccaatatgacttgaagacaagtctctcattgaaaccttattatcgttactaagcaaaacgagctgacaagaagggaaggtggtcggtatttcctcgttgttcaaatatatgattctcctggcaatatctgtgatggcctgttcaaaaagtggaatcatttctgcaggatcatctaccaactttttattgagctcctcattgaatacgattaagtggtcattttgaatcgtcagtaagtacttgtttacaagtaaattctgtctgagttgttctctgtagatgtactgattttccatacgaaactccaaaatgaacgaacggaatgccttaatgacctcactgaactggtcatcgttctgttctccgggaaggacacttgtgttaaagactgatgctctatcaaaggacattgcaacaaagtataaacggttgtgagcgggaaaaagatgtgtaggtaattgtcgtagatgagactgattcagtagaaaacgcgtcctgcactattttttttctttcttcattacatttcctaatcgggacaaaatgaatctaaagacgtggttatgtagtacacgcatcgataggctatccccataccaaaacactattttaccccatccttgacaggttataaatatgcgatagtatgagtatcttcaaattcagctgaaatatc

SEQ ID NO: 136 - DNA, 700 bp, Komagataella phaffii
tctcaatcaccttatatccagtcgaatatatctggatagggtcgggtctcactgtggaagattcaaatgggttagatccctgtaatttcagcgatggagactcagtatgatggggcaaggaaaacggcaattggatattcaattggtcaagagatggtatcaaaagcgagtgtgccagggtagccacggtagccactgatgctaatctgataattttcatttctggagtgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaag

SEQ ID NO: 137 - DNA, 700 bp, Komagataella phaffii
tcagataaatttaagttatcaaggtaaaggagctctaagtttaccccttccaacagggttatttgagaactcaataaattgttgaattcaaaaccaattgtctttgaattctccactggagcttccttgctgaaattgattttgataccattggcatcaaagagacccgtatgataactccataaaaaggggagatgataggccttaaattcatcgttaatctgcaaatttattcctgacatgtctttgtaaatagttatagttcagaaactggaattgagctcaaaaaactggaatcgagcggatatttgaagattgatgccttactcatgaattgattgataagagctccgtgattcactctgtcaatgattacccctctcctacccgatttgggactttttcttcagtcttggggactttttttcatatgacttgaccttgctttcccaatagggaaggactcacccatggatgattaagtttggattactcgtttaggaaatagtagccatgaatcaatttgaatcataccatcatgaaatagggttaggctgtaaatgcctcaaaaatggctcttgaggctggatttttgggtattggaatgttggtagcaattggtataaaaggccatttgtatttcacttttttgtccttcatactttactcttctcaactttggaaacttcaataaatcatc

SEQ ID NO: 138 - DNA, 700 bp, Komagataella phaffii
attgcagaacaacatgcatacagtgccaagctgtaccaagaggattcagaagtgattgatccactgtttgaggagatcgttgctccacctagaaatgccgaacgattcaaaacacaatttatcgtcacgccctcagacaatagtgtttccacattgaagcttaccttattagtcaacgagagtatattggatgccaagcaaagagctcagttattccttgatgaagtcaaggatcaattgacgcaagataatggtggcgtttcgtcgtctccccaacttgaagagttattctgagttgcaacaagtctaagtagtaagtaattaaaccatcatgatcctatgatcgtgatcattcattaaagcacggtgtggcaattattgctagggagatcgtcactgtatggtggcagaattatctctacaagatgtctcaaagtccccacaaagcttggaccctctcatctgtaatgcattttcctgtaactccccttagccacacgtcaagggctctgaatccgttgaaaagctgtggcgtctgccacctttaacgtcttcatgagggatgtgcacgtgatattgtctttcccttctctaaagcttcgaaaaaaacgcatctcaatgcgagaagcagatcgatatatataaagaactagtccattgaaagatctctcaatttcactggaaaccaactcagaaagaa

SEQ ID NO: 139 - DNA, 613 bp, Komagataella phaffii
cccagtatgagaggaacaggagatgagctggaatttggaaacaggaacgttcaattgccaaggagaagtttgagaggagagagtggcaaagagaatggagtcacttcctatccatgcttacaacaagatctctggaatatgacatacaacatagcaacaaagagggggtgcatcaaaaaaaattacacgttttcccaccctttccaacgaacccccacaccagtgaggtgaacagatttaacgggtctcagataaacgaaaaaatgctaacaataccatctatcgtgagggggcggcccactgccacatttccaaaagatacccccctccgcttcagattgtaattgtctgttttatagtactgcagtgaagcgccacagctccaaaacttaatttgacttctttatcaattaccgtaatattagtcgggccttgccgcatcacgtgacccgatttcactataaaactctccgttcccataaagttttaccacatcacgtgagttgtcaacattgaaacccctcgatgtaatgcttcacaggttggttatttaaatcatccaatcgccgaccaaatgaaatgatttctaacgtttccttattcacatacaaag

SEQ ID NO: 140 - DNA, 700 bp, Komagataella pastoris
gcttgaatgctcctttctatattcaataggtgtttttagcacctttgcatgcactccgtggccaatagcgcgtttcatacgcactttaatcccctcctttgttgagtgcaatctctgtgagatttggtggggtgcgaaccgttagacgcagcacgttaacataaaactcctgaacaagctgcagatagtcttcaagatcctgatgattctcactctttggtataatgtttattgactgaatgtaaactttaacctaaaacatgtatctccgctattttttttttttttttttgatgaccctaattctcgtgacaaattaatttccagcaggggtttgtcccgataagagaattttttaaattatccgttcggataaatggacgtctactccatatttttccgtatattaccccacctggaagtgcccagaattttccggggatttcggataatacggtggtctggattaattggtgcgccaagtcttctattttgttgcagtctcgtgtgagtatgtgcaacaacaaacaagatgagccaatttattggattagttgcagcttgaccccgccatagctaggcatatccaagtgctatgggtgttagatgacgcacttagatgctgtgagttttggagtataaaagatccttcagattccacgcttatttcctcagtttttcaaaacaaccaagaagtatc

SEQ ID NO: 141 - DNA, 700 bp, Komagataella pastoris
tccaaagccagaaggtctagcggtttcaacgacaaactcttgttgccatagctctggaacaaaaggacaccgaacgactcgacagcgcccacaaagactgcgttgtagaagaacgaattgatggccccagagcttctaatagtcagaaggtaacccaagcctcctagcaaggttagcacatgacctaagaaccatgcgaagtgcagagtctggaataacgacacccagtctgtttttcctgaactcctggttggattggcagaagcgtttgatttgcttggggtggttttatttgaagatggagttgaagccatagttgctaaagggtggtcggagtttgcgttagagtttgttaagcaaaggaggaaaaactgcgccgtttggcgttccagtggttttcgcgtgtgtgaagccagcgaggaaagcttccttttggtaattttttttcttttgcagttccggacggaataagcttcaggttatgaggggggcggtatccaattccggaaacattattgcgtcgaagctagtcaccccgccataaaattgtgcagaattgaggtataaaattgatatcccttagtaattgatacgcttcagtggcaaatgtggtctagtgacgtaaggcccgactattgtctataaaaggagctgcaattttccgtagttttgagaactagtgggcaatttttgagacaaa

SEQ ID NO: 142 - DNA, 700 bp, Komagataella pastoris
gaaaataaaggattcatgtgggattctctcaagtttgttttgaagctgtaacagggtttcaaacattccctgcgcttgtccacaattgaatcatttattgttactagatacatgaataaagtgtgaagcgggccccgttactatcattcttgcgcaaccgttgtttcaaagcccatattctttgacaaattgttttgttacaccccccgtttgatgtacatcgcttgcaatgatttgcgtcccggagtatttaacatattcagtttaaaatggtatactcaaccaatatctgggggtatgtttttaagtacactatacaagtcagttttcctatttttacctttcgcccaatcatctaccatcttgttaagttttgcctcctatatccctgaccctgacatcacccatgattccgctcaacggttctcctctacatcgtccctcttttggagagggtgttcactttgactttcaaattaccccccgccatcacgcgcaaccgagaccgcacccccgaattttcacaaattaccccacaccctatactccaccactatgagggttattagttctgatcacgtataaatatcaccccaagttcccaatggatacccttcttctctctcattcgcaatcaaatttctgtcttttccagttcagattaattcctttacacttgctttttccctaacctttatcc

SEQ ID NO: 143 - DNA, 670 bp, Komagataella pastoris
agggggccacctggttggttggtcagagaagtggaatagttgtctcctcatcaaagaatcgcgatatagcaccctgtggggggcattgtagggcctccctcccaaaccttcgtccttctcgttcttgattacatatcaaacccaaactggttgcaagttgagctccaaatttttaatagtaaacccctgcaagataacctcggtatggcgtccgaccccgctcaattactttaactccttttcaacgaggacttcgtaatttttgattagggagttgagaaacggggggtcttgatacctcctcgatttcagatcccaccccctctcaatcccaagtgggacccccctcggccgtgaaatgcgcgcactttagtttttttttgcatgtaaacgccggtgtccgtcatttaaaagtcgccaacttagtgaactttaccctttttgtcgcactccgtctcctcggaataggggtgtagtaattttgcagtagtgcaatttttaccccgccaaggggggggcgaaaagagacgacctcatcacgcattctccagtcgctctttacgcctacagcaccgacgtagttaactttctcccatatataaagcaattgccattcccctgaaaactttaacctctgctttttcttgatttttccttgcccaaagaaaag

SEQ ID NO: 144 - DNA, 700 bp, Komagataella pastoris
atcctccaaaacgatacagtagctccatactatgctatccacgtgctgttgggtactacctgtgggtcctcataccccactccgtctttctccaacaatccacgcagtgccgagatttatcagcagactcaaattagtcaaacactgtgtttttccttgcccgcataatttatgggtttcaggcctcctcgtgtccagtttactcgaagtatattggtttgcggaaaaagtggtaaggactaccccctcgcaagtggatccagtttttccgaaatgttccgatccgtacgtcatccccgaagccgtacatttttcactcaaacgacacagctttggactcagggtagcggaaagtgccagaacagctttcttcagatgatgtttccttgtagactatgtacttctttttccaacttttgcggcttcgtttcaaacctggaatttcatcgctaaatcgctccagccctatttttctcggaaaaataagtcgtactatccccccttaaacctaaagcaaagcggaaaaattaccccaaaaccaggtgatgtttctcgcccctttctttttttccgagattttctttttttgtctgccaaattcctctcctgaccttagcgtccccggaaaaaaaattgactacttaagaaccgaattagccccagcttttccccttctttgtattattccccatactacatt

SEQ ID NO: 145 - DNA, 700 bp, Komagataella pastoris
attaagtgggccaagctaattggtagggcgactggctggagctgttgttctaccatctgagattgagtttgatgtgaagtcacagcgccaaagcagtacaacaaccttatctccgcattattgttcttgcgcaatagcttgggaagagacaggggtaattgcgcggattaagtgtaaggtgcgcatactatggtcattgacctctttctatttctcgaaaaagccacaccgttagctagaataggacgcatctaccccaaaatagaatgtggggaaataggacgcgcaactacctttcaatcactggacgtcagaaaaacaaatgcgcaatcgagtcaccctctgtgatatcctccgtaatcccccctctccgtctattctgaaagcgtccccccatgtttcttcaatctactcagaaaacaattcgtttttatttctttagttcacgatttggttttctgcaagggtctggaggatataaccaacctgcgactccataacttaatcctgagtttatatttatgcttcgtctgatgggtaggaaaaaactatcacgaaaattcccccgccaacttgcccttcggaataagcagccactctccttctgcccatagtaagcttgcgcgaggccccaacatggccagaaactttaaatatgccaaacaatctcccccaatctaagttctctctctcttaaaaa

SEQ ID NO: 146 - DNA, 700 bp, Komagataella pastoris
tatggtatcatgttaccacctgtaaggtccatcaagtggagggcaaaaaagttttcgacacgaaaaaagaatctcactgaggtttggctctaaccacctggaagatataatctatccgagaattactccctttggtctagtctatcttgatctgatatcattatagaaatgatggcagtactacttttgaaactgcctttattatctttaagaagatttgatgtctacattgtaactgttacgattcaaacagagtgaatagttactctcctaacataagttgaaaacccttctgaaccagtaattaaggcatgaatcaatgacgcttcaatacaagttgcttcaagttggattattaacagtcatcttcatgccggataaagacgttaaactgaatgcaagactatgttatggccggaggtttattattcgcagagtaaggagacacacttatgaatcaaaaaaagctattctaggcttcaaagattctgattccatctagttcgtagagtctatcagactaacagtgaacagtggatgagccgactcatggcttgggtctaccacctaagctggctcaattccacaaagattaagtgagcattgcattaccaaattaggtaatttttcaagaatgtgtcatactaccatataaagcgggtcgtttttctcgttccacgagaaaacaatctagcagaat

SEQ ID NO: 147 - DNA, 700 bp, Komagataella pastoris
gctacaatgtcgaacaagtccttgccgttctagatcctgataacgaaggacgaggattttgagaaagtctagaaccaaatagtgggaagagactagaaaagagagggacaaatgcgaaccaactatctactatgcttttattcttgaaaaaaaacgaacgaagacggattgtcatcttctacaactatgccataccaatggcagtgttcttaccaaaagcatgacctcaatcaaagcctccaagtattcacttcaaaggaatgtcccgttttatcaaaagtacagaacccattatctaggcaaagacttctgcaaaacgtatagccccgtgaaggagctaatcaatttttgggattcgtttaccaccactactttaggttagcatagctatggagataagttcccatgatcagagctccacaactgtgaaggaactaatcataggctcaaatctaaaaatcggagaggagaaaaaataagctgaaaagaatcaaaaatcaaaatttttcggtttttcagtttacatctttcccattccgatagaaaacgtcgttacgtaatccatcccgtcatcgaaagcaactacttaggcgcgctcagatcttccacattgggatgttgaatagtcatagaaataaatctgtccctgatttattctttaaaactttactgtgaaaaattttcgataaaaatcagaaga

SEQ ID NO: 148 - DNA, 700 bp, Komagataella phaffii
agtatcatgttaccacctgtaaggtccatcaagtagaatgcagaaaagttttcgacttataggaagaatctctctactgaggtttggctctaacaacccggaagatattatttatccaaaacttattcccttaatcttgttctagcttgatctgatatcattatagaaacggtgacaatactacttttgaaacggcgctttttatctccaaaaatatcagaagtctatacatatgtgttgtctacaatttaaacagagtttacagatactctcctaacatgagttgaaaacgcttctggccaataatgaggacatgaatcaaatcatgcttgtaagtactttggtgcaaattgggcttttagtagccaccttcattccggatgaaggcattaaattggatgcaagacaaatgttatgacctgaaatttactgttcgtggagtaaaggacacatgcatgaatcaaaaaaaaaaatacactgggcttcaaaaaagttggtttcatctactttgatagagtctttcagagtagcattggatgagccgactcattgtttggatctactacttacgttggctgaattcagcaaagaggattaagtagggattgcattaccaaattaggtaattctcaagaatgtgtcatactagccatataaagagtcgtttttctcactacaagagaaatcaatctcgtagaat

SEQ ID NO: 149 - DNA, 700 bp, Komagataella phaffii
gaatatcgaacatgtccttgaagttataggtcctaataacgaagggggaggattctgagaaagtctagaaccaaatggtaggaagataccagaaaaaataggaacaaatggaaccaactgtctaccatgcttttataaaaggaaacaaaacaagatcgtcatctcctacaaatatcccatactaatggcagtgttcttaccaaaagcatgacctcacaatcaaagcctccaagtattcactttaaaggaatgtcccgttttatcaaaagtacagaacccattatctaggcaaagacttctgcaaaacgtatagccccatgaaggaactaatcaatctttgggattcgtttaccaccactactttaggttagcatagctatggagataagttcccatgatcagagctccacatctgtgaaggaactaatcataggctcaaatttaaaaatcgaaggaagaaaaaagaaataagctgaaaagaaacgaagaatcaatatttttcggtttttcaattttcattctttcccatcccattcccgatggaaaacgtcgttacgtaatccatcccgtcatcgagagcaactacttaggcgcgcccagatcttccacattgggacgttgaatagtcatagaaataaatctgtccctgatttattctttgaaactttactgtgaaaaattttcgatcaaaatcagaaga

SEQ ID NO: 150 - DNA, 700 bp, Komagataella pastoris
agaaatcgttggcgaatatcctagatctctgaaaattgttggactgggattcaaaaaggcaaggtttgtccaccagcattttccagcccttagttgggaaggagagtatgaatatatatcggtcgggcctacgttactggattcatttttcgacgactgcgtcactgaagcgcaacgcaaagagaaagaggaaaactactattctgatcttgaatcttccgagatgaggtatgctgtatctgagttttccagagatccatttggcaagggtccagtgttatccgcaaaaagacgactaagagatcctttcaagagaggatgcccttacagagacgaaaaatactctgacatgttagactccaattaacgaacactactcgtcttccagaaactcccaggcaattataaacgaggctgcaacccataaccatgagtgctcaaaatgccaattccttccatctcattgtcgtaccagattatgactctttaggcggggagaattccgtccagcatagcgaacatttctttttttttttcttcgtttcgcatctctctatcgcattcagaaaaaaatacatataattcttccagtttcctccattcattgcatttaaaactacgaaagttttagctctcttttgtttttgtttccttaattagagacatttttctttattgtgcttaatttgcgtggcagaca

SEQ ID NO: 151 - DNA, 700 bp, Komagataella pastoris
tggctttaccagcaataacttttccaaacactacatgcttgttatccagatgaggagtagggacagtggtaataaagaattgagatccattagttcctggtccagcgtttgccatggagagaagaaacggcttgtcatgagtcaattggaaattttcatcttcaaatttttctccatatatggactcacctccagtaccgttaccattggtaaagtcaccaccttgaaccatgaaatctttaatgattctatggaaggtagagcccttgtagtgcaatggttttccagagctacctacacctttctcaccagtagagagagctctgaaattctctgctgtcttaggaacaatgtcattaaagagctcaaagattattctaccagctggctgcttgttaatggatatgtcaaagaaaatgtgagaacgaggagtcattattaagaaaggattggatggggagtaggcgatggatgagcctttgatttatacggattcgatccctttgttgaagagtaaggcgcgattgtctagaattttctgagacagcgcccacggcagatatcgtcaggggagcgacttctgcgtggttatcctcctagatcaactgaatcgcgccaacctgacatctgactctaagcaattgatagtttgacccttcgtcttatcttacttcgttatctcaattaatcatacaataca

SEQ ID NO: 152 - DNA, 700 bp, Komagataella pastoris
tgaaaggtcttaatgtagtggtattgttcatgagaagcttacgtaacacaaaattagctggcttggggcaaagatacatcacactaacaagcgattggacaatagaaggaagccttggtgtcaatatttctctggtaagtgcgactttatgtctgattttgtccataataaaatccagagtggatatagctgattccaaacaggttatctgtcttacatcaatggttgatgtgataaaacactttgacccgataatcttggtcaaagcactaataaaattaccagcatcagatgcgataatccttgggtgaaggcaaagaacatgaacaagttttgtgccaaaccaacggacttcaggttgatttgatctgagataatttgcggcacgagaggtaacatgggtcaagtctgcctttgaagcctcatttacagcgttctcatcagcaatgatcggcagtatttccatcagatctgattcaacggaattgtccttcagatgcggaatgaccacatcgagtccaacaaatccttggtaacccatcttggaagtccacttgtatgatctggagttacctcatattattttttttttttggagtttttcagtccactatcagtgagtgtaccgaccaatacgtatcaggcacagataaatttgagtctcacgcaacatggaaatttacttgtctctaggtaggtc

SEQ ID NO: 153 - DNA, 700 bp, Komagataella pastoris
aaaacgcggatttgtatatagggctagctttgtttcaactgaattgatcagattgggaaaaacaacaacaaaaacagaatggtgcttggatgttatttgaaatctgattttaacgtgttgcaacatcgctggtgttgtgatgttggatcgagggtcagagaatttaaccaaaaaaaaagaaagaaaagggaagagggaaaagcagtagtggtaagggcgatggtcttccatcaactcgaaattaagcacatggatgttcggtccgaaataaggaaagttaacctttgtggtgcttgcaatctcggatctctattgttactcttgcttaacattccaatatttttcaacggctattgattaacctcctcctctgcccatgtgctacatccaaatctggtaatctgctttctgttactaccaaaatagttaatcacctctgagacatgtggccaattgtgcatcagatcgaatgaacatcttctttgcgtagcactccaagtatacccctattagctcttattgacctgttacggcttgacgcagtggccattattgtttccacttgaaaagcttatttttttttttgatagcacagaaacgtgggctccgataagctgaacttcaacgaaaatataaaagcaggaaagatttttgtcaaggacttgtacaacgaccactaagtctctcaagaacattagac

SEQ ID NO: 154 - DNA, 700 bp, Komagataella pastoris
aagaaatttcaagggcatgcactaacatgacatgtgacataagcagataggaaagtaaggcacataatttaagtgacagtagaacaacaatgttatttttttttttacaacaatgcccacgttgttgccgatgctcgcacgtagattcccttaccggccaagcaacaggcgcgagagaaatgtaaaattgactgaatctgataaaagaaaaaaaaaaaaaaatttatccggtgtatgggtgggactatacgcctcgtgtgacaacgtgggtggtatctttttcttcccccacgtgcgcgtgcaacctattgtatggaattagagggccatgtgtaaagacccttgaacccttgacaacagcaaagaccggggaagctgaccctgagagacatgagcagacaagtctcttcaatttccgccagctgtgagtgggccaatggtgtagttatctcgttgaacagatttcaacagctcaagacccagatacccactttcctttttgatctcttttgatgcaggacctactcttcaaagtcttggcacggccacatgtctcaatttttaattcaaaaccgttagaaggagaagccgtaattaggtataaatagtaggaaccccagcagttagtgctgatcacttgtaaacctttctctgatacacacaagcatctatttgagtactcatcttataatcattcaca

SEQ ID NO: 155 - DNA, 700 bp, Saccharomyces cerevisiae
ctgataacaataatgttacagattatagtcagtcggcgaagaacaaaaatgttctcttgaaattccccgttagtgaactgaacagaatctatttaaaatttaaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 156 - DNA, 700 bp, Pycnoporus coccineus
cagtaagttcagtagcatcacttcctcgcgcgaaaacgcctcctccctcccttgctctctcagggtccacgttcatgttcacgaccatctgtccggtgttccgctatggcattgactacatgtatagatgacatggaggaaagccgtgtaatctgtcatcacgcctttctcggaccacgtcacatctcactggcactagtctatcgccgcagattatcaacgctttcaaagcgaaatcccgggtctcaacactcggcaggaacgtcttcaaagcgccaatgatttgatcctgtagggcttttaggaacagcctgaatagctacgaaaacgtggggagactggtgggctcatcgcagtccgtagtcccgagatttgccaacgcagagtcccgagcagcagggtctgctcgtacaacgtaagtaatgatcacccggtagcgatggcttcgccgctcagtgcctctccgttccggacacagcaaaacgcttgctacatagcgcgcgcctcgctcactccgacatggacgcatctcgccgactcggccttctctcttcgaaaccgtcctgagttttgcatacatttctcaaacgggctctggcgcgaccaattccgcggccttgttaccgctcttaccttggcacggacaccgccctctataccaaaccgggcctcgtcccacccagaactcagcagtcgccgt

SEQ ID NO: 157 - DNA, 700 bp, Saccharomyces cerevisiae
gaaaccaattattaaaggcttacttactgatagtagatcaacgatcagtataattaagtctacaaatgaagagaaatttagaaacagattttttggcacaaaggcaatgagacttagagatgaagtatcaggtaataatttatacgtatactacatcgagaccaagaagaacattgctgatgtgatgacaaaacctcttccgataaaaacatttaaactattaacaaacaaatggattcattagatctattacattatgggtggtatgttggaataaaaatcaactatcatctactaactagtatttacgttactagtatattatcatatacggtgttagaagatgacgcaaatgatgagaaatagtcatctaaattagtggaagctgaaacgcaaggattgataatgtaataggatcaatgaatattaacatataaaatgatgataataatatttatagaattgtgtagaattgcagattcccttttatggattcctaaatcctcgaggagaacttctagtatatctacatacctaatattattgccttattaaaaatggaatcccaacaattacatcaaaatccacattctcttcacttctccgatagacttgtaatttatcttatttcatttcctaacactttgatcgaagaagagggataacaacagacgaaaacacatttaagggctatacaaag

SEQ ID NO: 158 - DNA, 700 bp, Komagataella pastoris
ttaaggatcagcggagaaatgtcagaaaaatcatacaactgcttggcgggaatgcttagtagtgcctggtgcctaggcaagattgagtttacgtttcatggatctagtcataaataacgttccccatatttctgcgggtgggaaatgccgtttgtcagaaaaatgccatttgatttaaataaattacgaagcgcatttgaccttgcatttgaagaaatagtgccaactcttttcctctcggcatgctccaacacgagaaacgcccgttgaaatttttgatctgtccgagccttttcttttttattgcagcctctaatcaaaaatgataggcagaacaaccaataaagccatttcaggagcctcagatgtacggctgcattgatagggaaaaagtaaaaaaagttgattctcagaatgtgggattttccgtctgcaaggcttttttcagcttacaatgcttggcacatgcatgatgaccgataaagacagaaatgccattccccttatgtagtgatctgtcaaaccggtgggatagccagaaaatgcccaacccggtgcgctgataatcatgaccctaatcttgattgctggaaggagataagattgtagatagtggtttcataccagaacttatataaaagttgataattctcctcttcatagtctacctttcaattagcgtaaacacaaatatcttata

SEQ ID NO: 159 - DNA, 700 bp, Komagataella pastoris
gttgacatccggcgaattcggggcaaatgtgctgttccggtagcttgtaggaagcggcatccgtggggcgatatatgactatagcttctgaagcgtagtgcgatgaaatgttcgtaggaacaacaaacggatttttttttttttttgtaggctcaaccccgttgagttataactcactagcgaaagggaaagattgattgttcgcaggcttcaaggggaaagagagggaaaaataaaaatacaccattgattacataatcacttatttttctcacgatgattttgaatcgaattccttaaagggttgaggtagtgcaccaatcgaaataggtagatagagttcgcataatacccaaatagggggtgtgcgggcgccccctaaaacgtctctcatcatgatcgttctgcgacccccatcagagttctttaagtcacatgacttgtgcgagcctcggtcaagctgttccgtgcacccattccaccccgggctgaccaacgcaaggttctccgagagtccgctaccccagatttatatcagcaaccagtcacctttttccgggcacgactggttatgccctggaaaaccggagacgatgagcctgactataaaaggtgacagaacccccgattctgctcaaactctacaacaaatactttcttttctttcaattaaaagaacacagtatcaagtatatcaaga

SEQ ID NO: 160 - DNA, 379 bp, Ashbya gossypii
agcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 161 - DNA, 939 bp, Komagataella pastoris
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgatacg

SEQ ID NO: 162 - DNA, 554 bp, Komagataella pastoris
cttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttt

SEQ ID NO: 163 - DNA, 822 bp, Komagataella pastoris
caggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 164 - DNA, 579 bp, Saccharomyces cerevisiae
acaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 165 - DNA, 535 bp, Komagataella pastoris
ccatcaagattgaaatcttaaaattgcccctttcacttgacaggatccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 166 - DNA, 1000 bp, Komagataella pastoris
cgattgcccctctacaggcataagggtgtgactttgtgggcttgaaatttacaccccctccaagttttctggcttcaatcgatcctgttaacaatattgcatgcccggaggagacttgccccctaaattcgcggcatcgtcccggatcgcagggtgagactgtagagaccccacatagtgatatgattaggtaagaagaggggggtgattcggccggctatcgaactctaacaactaggggggtgaacaatgccagcagtcctccccactctttgacaaattagtatcaccgatcaacaccccaaatcttattctcaacggtccctcatccttgcacccctctttggacaaatggcagttagtagtggtgcactgactaactgcccaaccttaaacccaaattttctagtaggggcccatctagttagcgaggggtgaaaaattcctccatcggagatgtatagaccgtaagttgctgcttaaaaaaaaatcagttcagatagcgagactttttcgatttcgcaacgggagtgcctgttccatttgattgcagttctcaccccttctgcccagtcttgccaattggcctaaaacctcctaatatctttgtttcccacccccttttccagctccacaaattggccaatctcgttttccatttgggagtatctgcatgtcgaatacataaagcgaccggtgtccgaaaagatctggataattttcaacattttgtgctccccccgctgtttaaaaacgggggtgagcgctctccggggtgcaaattcgtgcccaattcctttcaccctgcctattgtagacgtcaacccgcatctagtgcgaatataccgcacccccaatgatcacaccaacaattggtccacccctccccattctctgatattcacaattcacctcactataaatacccctggcctgctcccatattcttttttttttattccatcagcttctagcttttatcttattttacttaaagaaa

SEQ ID NO: 167 - DNA, 700 bp, Komagataella pastoris
ttctcccatggaccatgttgctcctattttctcaaagaataccagcagtccgtaattgatttagttcagctttggcagtggaccgaatctttgtaaggttttttattttcctttcataaacacctttcagcctcacatgcaactattgtcgatccatgaaatccaccaacatggaaaaccgaaaaattgcccctttcagttgacaggctcctccttcgtagaattgtcttagtattcttgtccaatcaggtaaccatctctgaaatgtttagttccgctagaactccgaacgaccagctggcaactgaaaattctccggggtaaatcttaaatgtggagtaatggaaccaaaaacgttctctttcgttctatctccttccaccgtctgttcttgcccttcgaaaattttatcctgctggggcagctgcccaggattcccccttgcagcaaccctcttcccagcaccaccttgcgggtgaaacggaggtcgtgtacccgacctagcagcctagggatggaaaagtcccggccgtcgctggcaataatagcgggcggacgcatgtcatgagattattagaaaccaccagaatcgaatataaaaggcgaacacctttcccaattttagtttctcctgacccaaagactttaaatttaatttatctgtccctatttcaatcaattgaacaactatctaaacaca

SEQ ID NO: 168 - DNA, 700 bp, Komagataella pastoris
actaacaatattgtgccccggaaaaacatccttgagagggctgttcaagtacagttttcctctcacccgtttcaatttacctccgggtggtatccccaataacctctgctgtccccgggtgtcgatggggacagtgagggagattgaacagaagagaggatcattgtgaatatccctatcaattgggttctaaccacacatgctcaactttaagatcatcacgtcacgaaagcaattgagtcggcgcagagctatcctccccttcttgaccccatgcataggaccaaccttaaccgatggaacatgttcctccgctccgtcccctggtagtgtctttgcgcaagaaatcgttaaggtatgaagactgatctctcgcacccccctctcgggtgtgttataatgaattggcaaagccattggctagattgaaacatataattcgtatatagtcttgctcaattaacgatctacctatagcctcaatctagacgtctaataatggcgtgtgtgtctctagtcgatgagccatctcatgtgacgtctatacgcttcgatggcttccgtcgcgaatatagaaccacttgaaatatgctgcaaaccacgatccaccctggttctgaaaagatataaatacagcacatctagcaggcttttgtcttcttggttgaaacacacaattctaacaatctatatctaaaaa

SEQ ID NO: 169 - DNA, 439 bp, Komagataella pastoris
tatgagaataaaagtagcgtcgaagtgactcagactgttttctcttgagtgaatgtcttatcagaagaaatatatttgattttttttttctagcaagtgactaagattaataatcagtcgctcacaattgacagatgagaaaattacgtaatgtgattataaaatatgatgagttgctagtggagccaatccggggaaaacgatttcctccctcaatagatagttgcaatgtcacggtggttttttgccattcatttggacataaactccagttaaagtttgtcttccactggcctctcaaatccaattacacgtctaactctccctgaactgaaattctaaagattacctaatcaactatcgctcgaatggctcatccctaggtgatttgatttacgttacctaaattcagtacacctgaaacttaccagtctcacgg

SEQ ID NO: 170 - DNA, 472 bp, Komagataella pastoris
tatcaagctgaaaaaaaaaaaatttcaaatctttaacccaattaggtaataagtatcgcctcttttatctgccgcactgcatgaagtgtccccttagtgggaaagagtactggaccaaccctggaggacagcaagggaaaaatgccgccaacttgcctcaaaatggccatagaaacaaaccttgtcggagataagtaccttagactgccacttatcctctgcgatgctctgcttctcaaagtttgcgatttctctctattataattgccatcaagagactcaggactaatttcgcagtcccacacgcactcgtacatgattggctgaaatttccctagagaatttctttttcacgaaaattttttttttacacaagattttcagcagatataaaatggagagcaggacctccgctgtgactcttcttttttttcttttattctcactacatacattttagttattcgccaac

SEQ ID NO: 171 - DNA, 700 bp, Komagataella pastoris
cccaaatcttattctcaacggtccctcatccttgcacccctctttggacaaatggcagttagtagtggtgcactgactaactgcccaaccttaaacccaaattttctagtaggggcccatctagttagcgaggggtgaaaaattcctccatcggagatgtatagaccgtaagttgctgcttaaaaaaaaatcagttcagatagcgagactttttcgatttcgcaacgggagtgcctgttccatttgattgcagttctcaccccttctgcccagtcttgccaattggcctaaaacctcctaatatctttgtttcccacccccttttccagctccacaaattggccaatctcgttttccatttgggagtatctgcatgtcgaatacataaagcgaccggtgtccgaaaagatctggataattttcaacattttgtgctccccccgctgtttaaaaacgggggtgagcgctctccggggtgcaaattcgtgcccaattcctttcaccctgcctattgtagacgtcaacccgcatctagtgcgaatataccgcacccccaatgatcacaccaacaattggtccacccctccccattctctgatattcacaattcacctcactataaatacccctggcctgctcccatattcttttttttttattccatcagcttctagcttttatcttattttacttaaagaaa

SEQ ID NO: 172 - DNA, 700 bp, Komagataella pastoris
atgacgtaggcaccctttcggttcgacatgcgtggccggtcgacgccagaatgtttttgcgctaaggcggccatatcgagctaattctacaggcaccctgcgaggcaagcaaatgctactatctattttcgttattacctgactgtggtttcaaagcgctatcaggtggggggcaatcggaatacgagtggatagcgagggaaaatgctggctggggtcaggttttcgtgtatctgaagcgcgcgctgcgattggtcgcgaggactgagcggaatatattaaccgtcgatcgcatttttgagcgagaataaatcttgatcgttgggaggtgaaatctatgttctttcctggaagccctttccgtcgacggttaggtgcatcctcgtggggatctcttccaggctttcgaacaataggccgaaattgctaagaaaacggacccttccagcgattagttctcactcatggttccgtcttctccctcctttcctcgcgcctggcaataaagctccttcggaggtgctccgtctagaggaaaattgctgcctttctgactttcggactagcgccaaccgcgaaccacaccaccacaccatcactgtcacccgtcacagttcatccctctctccttataaagcttctaataggttctcaattgtttgccaaaaaatctcttagcatagcccaattgattacgaaa

SEQ ID NO: 173 - DNA, 616 bp, Komagataella pastoris
agaatgtccaaagtacattggcagacagttcttagacagcaatccgagactttactcacagtgcaagcaagacttgtgtggaaagcctggggtcagacaattcatctcatcccccctttttacttctgctgatcaccgttagtccgacagttactccaatcaatatttattagtgttttagttgcgcaaaactcgagttctttttccttatctcttgatccttctagaatcgtactttttcagcgcaaattcactctacaacgtctaccgatagtagaccgcctatcctccccttaaatagcctattggaagggtgcaagaaggtatataatctggcgcgattcccccggacttttatgatccacatcacctcatcctactgccctcactttctttcctgatcctcccaggtccaccaaattcctcactatcgtcggatttctccttccagcgccctagagaattccgtaaccaccgcaaaaatagcagcccccccctcacccatttttttatttaaaagaacaccttactggcccgttttcgtttctcctttactacaattgatttttaaatttcagttttttttcattgatataccagatctatcacaaacaca

SEQ ID NO: 174 - DNA, 700 bp, Komagataella pastoris; identical to SEQ ID NO: 167

SEQ ID NO: 175 - DNA, 700 bp, Komagataella pastoris
ggttttccaaatgtgatctgaaatgtaatcttcgcaagtttggttcataaaacgtcaaaagaccacctgaagataaaaaataaagtggaaggagaagaaaaagaggtttttttccagatgagtctatgactaagatttctcttcaaagaatttttttcaaggtttgagagggatgaaagcgaggctgctgcacgattatcgatgtcattcaatagggaggtggaagagaactgagggagaaaaggctacaacttaacctaaaacgaacttaatttaaaatgagacctattacacgataggtgtggcgtcttaacacagtcattaggttcaactaattgctttttgaagcaataaacgatgccaatcacagaccgggtctccacccttacaacaattacttatctaccggctaggtgttgcatcgcggaccagcccaatcgggtaattctggttgaaaaggttttcagcgcgagaccagctattacccggatcatgtaatgctgaaaattgctcgcttatttctgatctgtacgtatattacagtacggggagcatcgacaaaaaatctttcaacaccaccatagaaaagactcctcatgcatttttcagcatcgcgaattcgcagtagagtgaaaaaaaaaacttcactttcacccattcttttctcctctacctttttaccctttactaacactgtata

SEQ ID NO: 176 - DNA, 700 bp, Komagataella pastoris
ccgctggtagtcacgatagatacaacccgttacgttttcacctcccctacaacgtagtaatttccccaattagtcttctggacaagctcactctggcagattgtcaggaagttctctaccatgtcttcaaacgagcacttcatactgatactaagactctaagctctttccaaaacttaacaagatacacagaatccaagaaggaactccccttacaaacattaaaccaggttgatgtgttagtaacgcacaatttgtggctcacaaaaaggcagacttcttatctgattggctctaaaggttcccgaattgaactcataagaacaagctccaaggcgaaaatcaagatcttcagtgcattagaagaggaattaatgcccaagcaacatatttctataactggaaccaagtctcaagttcagaaggctgtctttctgatccagttgtccctagacaaactacttcagggaatcagtgactggtgagtccatttattggcaagaaaaactataaagggctataaacgccatgccacagccgatggaccgggtaacctttcaatgttacccacaatttattgtttcgctaacctaaaaaaaaacctctagtggttggtgaaaaattttttttaagagagccttttttttcttgtttcctcttctttctcaaagaaactttcaagttttcgcatatatatata

SEQ ID NO: 177 - DNA, 666 bp, Komagataella pastoris
acttattaatgatgttggaaaattttgaaagagccgctggaaaagatcatgatcaagtacggctatgtccaactcaacggtaatatctttcacttcctggggtcatagacccttcaatatctctatcttcattaggggcccccaacaaaaaccagattagcacaagttttccttatcgggaaattcagggctgtatcgataactcatcgagagacagattccagctggatgttgtgtctgccatgctggaaacaaagatcggcccatcagcttcatcaattgagcctcccaatggccgaacgacgctttagctataacgacagcaccctaccatacttcatctgatagttgtaccatacattgcctctccccataacccacatgtttgagccttcgtttcctattcgattgagataacatccaccatccagataaatgttacccggacgacttgcgtatatgttagtctggcactcttctacagcgtagctgtcagactgaacgtgcggctcatgatctttcctttttttattatttttttttttcacgtttactgcagaaaaagaaaatttttttacctactttttttcttcgagggttaattggtataaaaaaaaactcacaaattttctctctcttttttttttttccctttaatcagtaaag

SEQ ID NO: 178 - DNA, 700 bp, Komagataella pastoris; identical to SEQ ID NO: 168

SEQ ID NO: 179 - DNA, 418 bp, Komagataella pastoris
ttttttttacttgggtataaataaaaaaagggaaacacaatctctgtttcaagaaatcagggattttagtctgcttatatacttcgcgctaccccgcgacccgagcaactactagccttacaaacgctttgcactcagaaaacaagtgcgatattttgctttttttcaaactgtgacgttagcgacaacgctggtttgaactcgttctcgaccaataagatcatcgcaaccgttcagcccggtctcaattctacgtgtacaactcagcatggtcgcaaataaggaacggtaccttttgtgactaaatgagtggcgttgctgctaacaaggtgagccatccactggtatatataaacaagttcgccccaactgctttcctcctcttttttttttattactcaactactatcaataaaac

SEQ ID NO: 180 - DNA, 318 bp, Komagataella pastoris
atgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacaaaaaacggggcagcttatctaaaacggagacaaactagcgcgtgctgtcccattccataaggtatcgtaggaaacgagagttctccccccatcacgtatatataaacagacagatatcccacatccgcttgtcaccaaactaatacatccagttcaagttacctaaacaaatcaaa

SEQ ID NO: 181 - DNA, 691 bp, Komagataella pastoris
tgatacttacttgagtctgtccatcttgattgatttgtatggaatcttattctcaagagttggtctcggatgctggaaggtttcacatagcgaaactcatggataccctcggctttatagtaatccgaccaaattcacgtgaacacttcatgaaatgctcatcgcttaggtaatccgctcacctttttctgggagatacttttctcagggttacaacccttcagtctctaatattcgcggagctaggtgaatgttgaaatttttttttcagtctctccttgtcattcaacacaatcatacacgttcacaatgggtaaagttcacacctccaagcagcacgttaaaacctcccaccgtaaggctccaaagtctgagaacacctacctgaagtccctggttgagttgtacagctacctcgctcgtaagtatcactgaagttgggaatttattattttttgtttctgccaaaaaagtttgcaaataatcacaaaagtggttctaatatcgatgtaggtgattatgtggttcataatcaccagttctgcgagtacttttgtttaccaaaagatgcgactttacagcagaacaaaagtgttatcttctctttttccctagtggaagaaatgagtaacatactaacagtaatctaggtcgtaccgacgctgagttcaacaagaagattagaagatct

SEQ ID NO: 182 - DNA, 297 bp, Komagataella pastoris
cccatgattccgctcaacggttctcctctacatcgtccctcttttggagagggtgttcactttgactttcaaattaccccccgccatcacgcgcaaccgagaccgcacccccgaattttcacaaattaccccacaccctatactccaccactatgagggttattagttctgatcacgtataaatatcaccccaagttcccaatggatacccttcttctctctcattcgcaatcaaatttctgtcttttccagttcagattaattcctttacacttgctttttccctaacctttatcc

SEQ ID NO: 183 - DNA, 670 bp, Komagataella pastoris; identical to SEQ ID NO: 143

SEQ ID NO: 184 - DNA, 700 bp, Komagataella pastoris
ctcgcttgaaattctttctacaggtacttacaaccggggtatgcagcatagatttagccaccatctcggtaaaccatgtgaccagtttgtgttggttatgctgccaacaccggtatcggcgtaatgtaggatgtactataaaccaaagcaagtcataaagggatttgattccgaaattattcatcccggtcacccccgagagggaccatgaaatcgttttgttggtgcaccggggtgcgaaagggatgcatattcagcttctgaccatcattccctattctccggtaggttgtattgtactgagcgtgttgtcaccatgatcttttctattacaatgacaaattgccactatcttcattgagtaacaattccaatgatgggagatgactttttgtgctgacaaatgtactgtgcattctggtgtgtgttgggctatgtagggtgggccagacttcaatacaatcttatttccacaataggcacccctcaccccaaaaaaaatgtctccggcaccccgttaatagggatgaagtgggggtttttcaacctctatggagagggtgagaaacttcctttccccagattctcgcagaacttcaagcattcggtcggaatataaaagcaatagcaaatccgttgttgattttccttttttctctttgtccctttcatcttatatatatagcctaacaatctgaaa

SEQ ID NO: 185 - DNA, 700 bp, Komagataella pastoris
agatgggtgcaccagtgcatcatatctcccatcacaggtgaagtgatacatttgtacaatagaccaggcaagatactttatagggtcttagcctgaaaggtgttttaaaagccagaatgaaagttaggataggtttggattctgagttgagaactgttttgtttgatctagactcttctgattcctgactcttctttctgattgccacacttatatatctactactgcaatctaatacataattgcatgattcttcaagttacaccttttctctcttctgtgcctcaactcacggattatgacacttgtacttttatccgttttcatgactcttaaaaactgtgacttatgtcagaattgccactatctttggttcttttcggttccagtatcataagttgaagttagcaggatattcccattcaagtgcagagaccctgtcccgttacttccctgtacaatgattatataatccgtacaataaaattcttctccgggtaacaactattatcctgtttgtgtaaaaaaaaaaaattttcagcgaacactactaagtcgtctactacaactacttgttccctcttccaaggttataccaataattaatcgtttagtgtctatctgttcatagaaacaactcggttatacccctccacgtttaaaccttaaaaaaaaaaaactataaaaaccaaacaaaaaa

SEQ ID NO: 186 - DNA, 700 bp, Komagataella pastoris
tggtctaaatcactatcgttatttttgcatagtcatgtacattaatgttcaagtgaagtgtaatattaaagatgaattgtaggaaagttggacgtagccagacttttgtctgttctagttgtaagaagttgctattgtagtttactcaggtcgtgtgattattaatagttgttcctctcgtctgcggtctagttgtcaaaccatagacagtgcgttcgagtccgagatactgcactaattaccaagtaccgtttaattctccctcgtttgtcgtaaagcttatccacccccctggatctagcgtggaatttcgcacgagctcaattcgttaaagggaccgttcgttccacatagagacgtcccgactagcagttcgttatataagatcggacccctggcataaatgtcacatgaattcgtcatcacactccgatattataacagtttcagcgtccggaagtaaattaaccacaaagatcgtctagtagggggtgaggagtcactatctactgagtctctagctctaaccgtgacgcgtctctcaccacttccgaacagtcgtgtcctggtcattttagatattacacgcgtcagtttgataacgcgcgtcttttaaataaactctctagcttaagtttttcctttataataaaacacacatccaagtaaacgttcctaatatcctctcaataaactaaca

SEQ ID NO: 187 - DNA, 700 bp, Komagataella pastoris
caaagtgaaaccactactaattactttttcgacacagagagtgaatccacagatagaatacatacactaaactatggacttcatagttctgccagcaatgacgacgacgataacgatgacgacaatgacgactcggattcggaatcaaacagagtgctagagataataccagggctgaccaaagctcagacaaaacaggtaattgacaatttctccggcaagcggaaaccactatctcagaaggtgagtaaattgggcaatttattgagtatctttcccaaccataacagttttcttcaagacatgaacataaacgaactagaagagtttaaagagatagcacaagaccttcaatgggagttatcacaagccagaaagaaccaccgtacgaaaacagatacttggcccttggacgaagcccagttctatcaaaacaaagaacaggtcatgtcattgctccgaaatagcgtcaagcaggacctagaatccgaaacatagcgtggctttgcggattctacgtcttgcctctactaatatcactgaactgcgccctgttcctacgaatgatccatctatgcacattgtatagcacgcctctataaagtggctgatttttatttaagtcgcgaatgaccttcttaaccccgaatacattctaatacaactcgcctacacaaaccattctttgccatccaaagct

SEQ ID NO: 188 - DNA, 397 bp, Komagataella pastoris
acgtatagttaatctaaccaaatccctgttcccaacccctggattgagttatcggattatcgtgaaccgtgataagctaaaagtgttctcgatgtacgacataagaaacgactcattctttatcgatttcatctcaagcagtgtattaacgcctcatgtggtaattactgttcattcaaaaccctcggaatctactgtctcccgattttttagtataggagacaccctcgtaaacacgccggataaccttgaattgcttcatagtgcgtcaaagaggccctggtaacgcgtcggccaaattacgtgttttttcgcgttccttttttacgtgctactataaaacggaaaccgatggccctctttactactttgtacaatcaacccacaactatcaatc

SEQ ID NO: 189 - DNA, 700 bp, Komagataella pastoris
tttccaaggctcagagctttattcattccaagaagttggagaagactcgcttgggaaggccttcccgaagagttattccgaacaatacctttttgagcgagaaggcaactgatgatacatatgtgagcatgaatagtaggatgctggaattcgacagctcgtctaacaaccagaatggcgagggagaattgttaagtggcttggatgacgagaaagcgttgaataagcaaaacctggaatatcttgacgaatcaactagggcaaaacagttggacacctggggtcttgaaaagtggagaaaactggcaggactcttaaaactatacggggttacaaaaaatgaggagctacttgatcccaagttcattcaatccgagcttgggtgtgaacctaatgagttggtagctcgatcaattattttagtcaagtggaaggagctgaagtcattgaggaaaatgcaaaggtcaagtgataaggagcgaagtaaatgatggaaccaagtaggtgatctctatatcaaactagagttgatgctaggcgtgtgacctttgggttggcgaaatgattcctactaagctttttttttgaaagcagcgaactttatcattcaaaaaaaaattgcgtggtatacatcgactttcacaacctcttctccttaattaaatcttcctgttctcctttggtttcaaaggacttagaa

SEQ ID NO: 190 - DNA, 700 bp, Komagataella pastoris
ccaatttcacatagtccggatcagcgattttagaggtatttccagctaaaagacattccttgatgcccaattgtatcatggcagactccacgttagaaaataaatcgttatcaacaaactccacaaccccaatgatcttgttaacagaatcgatatacgacaatccaatgttcttaccagtcaatcgaagggaggcaattaaagcctgggtgttcagatctgaagaattgatcaaatgatctacctcagctaaatttcctggactcgcagtcttggaaagcgtccatgtcttatcgtagatttcaatcttatggccaatgtctagaacacacatcttcagaaaattgacaaacacctgaggcgagagcgtgacatatttaatctccttatgctggcctgacgccagagtcgcgttgcttgttttcaccaccgaatttgtcttgtagatcaactctgccactagtatcgcatcctgatctagtaccgtgtagtagtcaccacggtcgaccgcccacaccgttctatcgggcttctcgcccaacttgaggtacttacggtaaaaccctgtctcatcagacttgtcttggaaccgtagttctggtcttatagacgacattgatggtaaagtagaaaggaaaagagagtataatggaatgagacactagatttcaagaacacgcagcgaaaccatttttaaagt

SEQ ID NO: 191 - DNA, 305 bp, Komagataella pastoris
tgagcccatagtgtgtactgaccaaatatgttcactgtttctcatgatgaccctcccgctcgggatggtaaggttttataagattgccctagccaagaaggcaaattaattatccttttcattccgtgtttcaagggcacaagttataactcaaacctgttaattattggacctggattgtgtaaggaatagaattgaatttaattgaataagcaagaatcgaatatgggtatgttattagttgcgaattgggcggaaatcagtaaatagcagaatgtactaacacgacaaaagctcccaaaaaa

SEQ ID NO: 192 - DNA, 700 bp, Komagataella pastoris
ttgtctactgccgagattgagagtgctttaatcgagcacaacttggtaggagagtctgctgttgtcggattccctgacgagttgactggttctgctgttgctgcgttcgtgtctttgaaaaaggacgttgacaatccagcagaagtcaaaaaagaattaatccttactgtcagaaaagagattggaccattcgccgcacctaaactcattattttggtaagtgatcttccgaagaccagatcaggtaagataatgagacgtattctcagaaaggttttggcaggagaagaagactccctgggcgatatctccactctctcaaacccttcgattgtggaagagataatctctactgttaagagggatgcccgcaaatgagcatccgattaggaattacacttatatttcacatatctattttatctattttactatcgtgaagtagtaatcaaatactgtatgtactattagttttaccttgtaattacttgtactgtgttgggaagtctcgaatacaccctaccatacatgccgcctgacagatctttttccttttattttaaacgtaggttaggttacttggtaaatgtggactgaatctgccttcgttttaagaaaacaaaaattttcagccttacggtgggactagcagcactttgcatgagcggtgatcgtttagcacgctattttacagcctaga

SEQ ID NO: 193 - DNA, 700 bp, Komagataella pastoris; identical to SEQ ID NO: 144

SEQ ID NO: 194 - DNA, 397 bp, Komagataella pastoris
tatcaataccttcgataaatagtggtgttcgttatttttctgtttaggttacgattttgccagattttttgggaggagggaaacaaaagaaccagtgctacacaaccagtaagtgccatcggacatttggccctcgacctcatctcatcacatcagcttcccttttaatctcgattgacccttgccctgctgcgcgcacaccatactctggctccctctcatgcttctggcgttaccccgggaatcgtacatccattgccgcgaatcccggacagcactcagacggatttcactattcctagtgggcaccacctccatatataaggatacactcccgccccctagcctgtgtgccctacttgtcggtaataacaactttcaattacataaataaaca

SEQ ID NO: 195 - DNA, 700 bp, Komagataella pastoris
aagctcccatattaaaatggaccttggtaggccgataaaccatgcaatcgatacatatcaaataacaggcgcatatatgcagggagtcggactttgcacaatggagcaaagtctctggctgcgcaacaatgggcgcctctttactaccgggccaggtgcttacaaagtgcctggattcagagatttacctcaaaaattccacgtttcgatcctcaaagaccgcgagttcaagcatctagacacaatatggagatctaagggcataggtgaaccgcctttatttctgggattcagcgttcatttcgcactacgtgatgctatagcaacagctagaaggagccaaggcatcgaggaaggatgtaatggcttaccctttagaagtccattgacaactgaacgtattagaacaatgatggctgatcctattcttctggcggctcaagtggctcccgaaggcaatgaatggttcattgaggcctagaagtgttgtgattatttaatatatttaatttatttttgtttcagttttttttttttttttcggctgccaccctggcgcgcgctcgcactaaaggctacttctccgtctccaacccagcgagacggaccccaaagttccttctagtaaatgtacttaaccagcacagtgagagcggtaccagttcattttctcagatcctcaagacattgtatcagaact

SEQ ID NO: 196 - DNA, 700 bp, Komagataella pastoris
gtttaatacaattcgtgcgggcgacgattaaggcccttacataacccggaaacatgttcccccaccaaattgtgaacttttcagccttcaccggtggtaacattttagggggagataataaggttttgactgggaatttcgaccagagaacactgaagagggaccactatgttgctagcctgacaccccgtaatgccttcgtaattttaaactgttaactttagtattactgttcattcttccccgtcaaaaaaccgctcgttgcctcgactaattgagtcttcaactcatcccgtccgtgccgaattacttgaattcatttcacctcaggctgcttacgtattctcaccggtctccggtgtacatggatccgctattgccagatgtttctcgtacaacaatcaccagatcaaggtcgtgaacggaccaatggcatccagagcaatcctgaagtgtttggggtccgggctgtataaaataaaacaacgtgacttgaactagcaaatatgtcccagttgtgctacacccaacacgcgattaccccggagctcaccaggcctcttccccctctcaatggcaccctcctagcgcttcgaaataatggctgcgtagtatttaacgagtgccagttccctctgacaatatcctttttcttttctcttagttccccacatatcaattgaaacatattttttacaca

SEQ ID NO: 197 - DNA, 366 bp, Komagataella pastoris
cattaatcactcagactcgttttactttgctattggccatgtctggagtcgccagctagcgtctcacaagttacataggccttccaatgtcatgtcctcaatgccttcttcaatctcgtgccccttccgtcaaaactccgttagtttcaccctacactgcccctggttgtgcagctctaaccacttcgcgccgctactatccgtagtggtcgagccgcatcaatatcacgttgaaatggaataactccctacaaaagccgcacgcaaccatcaaatctataaaaggaacctcaaatatctagcaacatcttttcaatttactataatatattcgttaatcatcaatcaattagctaatacacaaca

SEQ ID NO: 198 - DNA, 345 bp, Komagataella pastoris
ttaagtgtgcgggatactgtattccgtttcgggattctaaagaagtcatccagaccaaatcaaatcggatcagagattccgtgagtttctcacatccaaggatgagggttttccacgagttgactagtactccggtgtcctaccatcatttgcggaatgaacacttgtgcggagattacatagggagtggggacggacgcttcttaaccgttctcctggaaagtcgtcccctgatcaaaacttgatagcatccaactttgctgtaataggtatatataacctagcaggcgaccattcatgtacagtaaattcttctagactttttatcaattgaaatcaaatcca

SEQ ID NO: 199 - DNA, 382 bp, Komagataella pastoris
cataataggtagaattactagatgtcggatgtttgtgatcgcagtagcatagctttcccggtcagctgtctaccttaaatgctttcacgatatgctccccaccaagctcggacgcctttctcgcccacccaccacgcaaggttccgcccactttatgctttgtggaagtcttttcgccgcgcaccacacttgcccgttcgcagaaaaagaaattccgggtagcccttattccgtcaggcaataaatatataagcgattgcagacaatctgacgtcctatccccttgtctatataaacttcttgggttgcttaaatttaaaacttattcgagtcccaacctttccattcttccggaataattcagctccaaccaattgataaa

SEQ ID NO: 200 - DNA, 700 bp, Komagataella pastoris
attcacagccaatattcaccgataatccatatcattaatctgaaggaagataattcttggtcaattcacgggatgagtaagtcaggaaacggagcttcgtcagtagtgggcaagtttctaagttactacgggactctttccggtatggactgtgcttctaagtttcgaggctcaagatggagtacatcaaagtgaaaaaggcatacgatcctcgctctctgtcagaatatttcaaggctctaatggagctgggcagtttgggtgacgtggcagaaaactagacctgttgaactagcgagagagtttggattagcgttttttcacgggttgcttcaagaacaacttgttgtactggcaattctacacccaaaacaactcaaaaatccaaaaacacaacttatctgacgcaggcatatacacccctaaatccagcatgcattttggcacaccagccgcccttttcatttgacacgtcttctccaaactacgtgtgttttatcggaattaactggaaagatcacaggttacgtaatacctgtccgcgatcgcccgatctcatggctcccacaagagccaatcaaatcccgcgaatttgaaatcggaacatgtaggaactcaattgtcatatataaataattcccttcccgtggaaactgaaaaaaaagctcaccccgacttatttattcaatccaacacaaaa

SEQ ID NO: 201 - DNA, 470 bp, Komagataella pastoris
gcatgataccgtagcccgctggcagaattaagtgacactagatcgtaataacgaggaatcgcttgtggactcacttgtggaatgtggaattattgttggcagagtgtgataaagtatcgttcgcgatatgatttttccagaaacttcttgtggatcattgtctttaactatgctaaacgatgctgacaactcagacaatgtcgaaacctgctcatcaaagatgaacaatttcctactagcagaaaaatagcccatttgtcattaccgcagcactattactggatgattcttttgagttagtccttgtcagaaactctacaaaccacgatctatggccgggttacccggtcatagaagctggtggaatattccatgctgtctcgacgatgagcttgcgaaatatatttaagtgtaaaaactccgcgtcaataagactctggttaactttttaattttggaaaaatatcaat

SEQ ID NO: 202 - DNA, 688 bp, Komagataella pastoris
cttgattccgctcaatcgcccactccacttcatcttcgacattatcttcggtcatttctccttctacagaaacataggctgaaccgtctaaagaagacctttcagtaattttttgccttctttgttgcattgatgagccatttacactttgtgaaagtttctctaaaatagttgtttccagaggccaaacattccacccgtagtaaagcgcaaacgtaggaagaccaagactagcataaatcaggtataagtgtcgagcactaacaggagatcttctgaaagtttctaccaacagataagaaccagtagtcatgcatatggcaacaatgtatcgagtggatctaaggacgcgtgcaactaaccttcgaattcgttgatcaagtttgttgttatcgatcaacgtgacaagattatcaattccgcgtgaggatgcatacccaagcacccctgtggcaattccaagtgagccagtcccaataatttttgtaataagagagcacttcattgtgttgcgcttggaggtagaatgcgaacaaattaagggtaattctcgaaaccgcgacatcaattcaaacactaacgggtcgtgctctacacggtaagcgctccctcctatcacttctttctcgctttagaaaattctccgaaaaattttcttcccttctcttgcaaatattgtcttcgaaga

SEQ ID NO: 203 - DNA, 700 bp, Komagataella pastoris
ttgaacgagcgtgtcagaatgaaccagctagaaacctcaatcgcccttaacaatttaaatcaatctaccttatcaatccaggagattctcaagtatcgctctttcctggatataaacctaatttcaaacctggtcaaactaggagtgtggaaccaccgctggtatgctgaattttcccctaagcttatagaaagccttgcggttgttgtggagaacggggggcttatcaaagtagagaacgaggttaaggctaactattttgattcgcaagatggagtctacgacttgatgaacgaggtattcaagttcatgaagcattacaattatccagggacggacaactaagaggtgccagtgcagaacttaggatgtacatgataaacaattatagtgaaaatagaaaccataatacaatattctaatagagagaggaacccccctgttacggacccgttcacctgcacttcccaccctggcctattgttaacggcttgagaaaaatacacccttgcctcagctttgcaagttttcagagttgacaactcatccaaatgatctctggacccgcgaaacttcaaggtccatcgaactttctcgaattttcagtgttttcatacagcgtgtcatcttctttcgcgtacttatttaaatcgtacccagatcccttcttcttccttaatttcaatcccaacactcaaga

SEQ ID NO: 204 - DNA, 439 bp, Komagataella pastoris; identical to SEQ ID NO: 169

SEQ ID NO: 205 - DNA, 700 bp, Komagataella pastoris
tatttgttttttctgagactaaagagcctaggaaattcttcctcaggattttgcgttccatccaacatctgtgcgcaaactaaaagttccgagtctgacatctaccatcgctagaccctatgtaataccaatctcgcgtccgaagttttaactttcagtccttaaaacttcagagtattggttgtagtttccggatctgaggggtcgtattgaagacaaaaaaaagacggagctgcctccacagcgcgaaacgtcaaccctaacaccaaccataatttgcaatcaccatggggacaagtttcagcagtcaatgggtaattcagacgatgatacgctatcaatttgctaagctcaatgatgatttagctggcttcaccgaaaggtctttcattgatgtactctgctgttcattcgtattttatctctctcgttggacaatgcccgggtactcccggaacaagggagtcttccagccaagctgtacacagtgaaaaatagaaatacacctttgaaatcaagacgcgcgttggccaatcacaagacttaatcggtgcaaagaaggattaccaaaattttttttcccaaaatcgctatatagaaataatggaggaaaaagggttaatataaaggagaattcccccgtttttctccccttttcttttcttcttcaggctttcttacaaatctataatattccaaa

SEQ ID NO: 206 - DNA, 529 bp, Komagataella pastoris
tgagatcaaatttgaattctcggatgacccacttgaggaggaattagatacaaatttttgaaggtgcctgcatgtttacacccatgaaactttatctgttacagcaagcacagacgatgcttttcatgaatgcttgttcgtacaacttttcagatgaagccaaaattctcatgcgcctcactttgtgcggcgctaacgatcgtcctttctttgtacctcccggatggatcagcacaataacttgattgagaggtttttattccgcgatttttgttcaccccacacttttctcgaaggttctagcaatcaagataaacaccgcaagagagccgcaggaaccatatgtggtaccacaagtggtcttaaacaactctggtagaattcgatggaattcgatggaagccgatcgagtccgatcgaattgaagcaatccgtatatataaggagaacctagttccaccccttactcgagcattagtttacaagactaacttcacagaagcatagaaattaaacaaagttaaacatt

SEQ ID NO: 207 - DNA, 579 bp, Saccharomyces cerevisiae; identical to SEQ ID NO: 164

SEQ ID NO: 208 - DNA, 700 bp, Saccharomyces cerevisiae
gggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 209 - DNA, 700 bp, Saccharomyces cerevisiae
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 210 - DNA, 583 bp, Saccharomyces cerevisiae
tgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 211 - DNA, 698 bp, Saccharomyces cerevisiae
ataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 212 - DNA, 700 bp, Saccharomyces cerevisiae
ttgataggtcaagatcaatgtaaacaattactttgttatgtagagtttttttagctacctatattccaccataacatcaatcatgcggttgctggtgtatttaccaataatgtttaatgtatatatatatatatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaac

SEQ ID NO: 213 - DNA, 700 bp, Saccharomyces cerevisiae
gatgccgtgtctgctgccattacgtacaggtaggcaaaggcaaacaagaatagcactttttcacttgctgcttttttttgtgaatgaagtttatcgtatattctgttagtgccgctcgtatcagccacttacgttatcattctaaagtaagtatacgaaatgtgcaaaccttagtagacgttcaagaattgctcgataattgcgaacaaaccagctgctaagccgggaagttaagtcctcaacacagaaatgatgcaaacaatttattaaattcagtggtaatgcaacagcaagaggaaaggtggagggattaacgcatttcagacagctttatagggggaaagaaagcactcgcaaacttgctgcctgttcgcagtcattggttgcaaaaactaaactctactcacgcacactggaatgaatggcaatattcttttttaggttaaccggccggacagtaatatagtaatcgttttgtacgtttttcaagaagcgacgcacaactgttttccattttttttttttttttttcagtgatcatcgtccatgaaaaaaatttttcatttgtctctttcgtgcttcctggatatataaaatacgatttatttagttgtctttgtcaatcctcatctttctttactcattatttcatttcggttttgtcatctctagaacaacacagttactacaacaatcaatc

SEQ ID NO: 214 - DNA, 357 bp, Saccharomyces cerevisiae
acagctatataaaagttcctaattgtgcattttttcaataacaatacttattcatccttataattatattctagcttcgttgtcatgggaacatagcccatacaccgcagttatttatgatcatttcgaacgggaagtatggatgaatctttttttttttttttttatagcacgcaactgaaaaaaaaaaaaagaaaaatttttcatcttcgctcgacgtttcttttgtagtactcatctctttttatataaagattaattagttattgtcgctttgcttttccttctttaaaaaatgtttcttgcttttggattttcagatgtcccaagatcattacagtattttaattgaacaaa

SEQ ID NO: 215 - DNA, 695 bp, Saccharomyces cerevisiae
caactttcccttctgtttcaatctttttacctattcctttttaaaagatatatatacattaaactccttctacaagtatatattttatacatatctacagggcgtatatatacataacattttaagataagcaagtgaatgttgattcccgtttcttagtcaacacttctttctattttacccggtcgttaccctattaaaaaaacaacttacaatcattgttcgccccttccatacttactgccactcgcaaaagggcccaaccagggcaattacgtatcaaaaaatcatgacaggctgggtaataaatattcgtgaagaaagaagaaattaaaaaaagaaacgaagaagcaaaaaaaagaaaagactccgtttaatcactttcaaccgcggtttatccggccccacccatgcataaccctaaattattagatcacttagcacgtgaaaaagaaacgtttttaatgttttttttttttttttctttttctttttttgcgttggtgaaaattttttcgcttcctcgagtataattatctcatctcatctttcatataagataagaagttttataaaaaccttttgcatcaaaattttgtagaatatctctttttcttacgctctctttctttccttaattgttttctaaagaaccgtgtatttttctagttcgaatccatcgataacattaaaag

SEQ ID NO: 216 - DNA, 700 bp, Saccharomyces cerevisiae
attgaatacattagcaacgcgtccagcatttttcggaagtgtctcataaactttactcaagagttaagtactgaaaaattcgacttttatgatagttcaagtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 217 - DNA, 292 bp, Saccharomyces cerevisiae
ttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 218 - DNA, 676 bp, Saccharomyces cerevisiae
tgtggagtgtttgcttggatcctttagtaaaaggggaagaacagttggaagggccaaagtggaagtcacaaaacagtggtcctatataaaagaacaagaaaaagattatttatatacaactgcggtcacaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctcaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaata

SEQ ID NO: 219 - DNA, 646 bp, Saccharomyces cerevisiae
tgtttgcgtttatatatttatgttagatgtttttcttattaactagaaagaaagaatataaaaggttgaggaaagagatgtatcccgaagaatacacagtcttttatatatgtatttcaacaaggagccgtggagggtaccaaaaagaaaaatcgcccgggcatttcgttatcttccacgctaaaagtcaaggagagatattacggccaggatcgcaaaggtgcagagcaaggaaatgtgagaaattgtgagaacgataatgtatgggacaatgcgaaaatgtgagaacgagagcaaaaatcttttttgtatctccccgccgaatttggaaaccgcgttctgaaaacttcgcatcttcacatagtaaaactgttccgagcgcttctccccataatggttagtggtaaaaaccgaagttgtttactttagcaaatgcccgcgaatacggtggtaaattgccacccccccttccccattcattgggtaaagaccaatttgatggataaattggttgtggaaaaggtctaattctttttcctataaataccgagatattttttctatatgatggtttccgtcgcattattgtactctatagtactaaagcaacaaacaaaaacaagcaacaaatataatatagtaaaat

SEQ ID NO: 220 - DNA, 699 bp, Saccharomyces cerevisiae
tatatattaaatttgctcttgttctgtactttcctaattcttatgtaaaaagacaagaatttatgatactatttaataacaaaaaactacctaagaaaagcatcatgcagtcgaaattgaaatcgaaaagtaaaactttaacggaacatgtttgaaattctaagaaagcatacatcttcatcccttatatatagagttatgtttgatattagtagtcatgttgtaatctctggcctaagtatacgtaacgaaaatggtagcacgtcgcgtttatggcccccaggttaatgtgttctctgaaattcgcatcactttgagaaataatgggaacaccttacgcgtgagctgtgcccaccgcttcgcctaataaagcggtgttctcaaaatttctccccgttttcaggatcacgagcgccatctagttctggtaaaatcgcgcttacaagaacaaagaaaagaaacatcgcgtaatgcaacagtgagacacttgccgtcatatataaggttttggatcagtaaccgttatttgagcataacacaggtttttaaatatattattatatatcatggtatatgtgtaaaatttttttgctgactggttttgtttatttatttagctttttaaaaattttactttcttcttgttaattttttctgattgctctatactcaaaccaacaacaacttactctacaacta

SEQ ID NO: 221 - DNA, 700 bp, Saccharomyces cerevisiae
aagaggatgtccaatattttttttaaggaataaggatacttcaagactagattcccccctgcattcccatcagaaccgtaaaccttggcgctttccttgggaagtattcaagaagtgccttgtccggtttctgtggctcacaaaccagcgcgcccgatatggctttcttttcacttatgaatgtaccagtacgggacaattagaacgctcctgtaacaatctctttgcaaatgtggggttacattctaaccatgtcacactgctgacgaaattcaaagtaaaaaaaaatgggaccacgtcttgagaacgatagattttctttattttacattgaacagtcgttgtctcagcgcgctttatgttttcattcatacttcatattataaaataacaaaagaagaatttcatattcacgcccaagaaatcaggctgctttccaaatgcaattgacacttcattagccatcacacaaaactctttcttgctggagcttcttttaaaaaagacctcagtacaccaaacacgttacccgacctcgttattttacgacaactatgataaaattctgaagaaaaaataaaaaaattttcatacttcttgcttttatttaaaccattgaatgatttcttttgaacaaaactacctgtttcaccaaaggaaatagaaagaaaaaatcaattagaagaaaacaaaaaacaaa

SEQ ID NO: 222 - DNA, 288 bp, Saccharomyces cerevisiae
caccgaccatgtgggcaaattcgtaataaattcggggtgagggggattcaagacaagcaaccttgttagtcagctcaaacagcgatttaacggttgagtaacacatcaaaacaccgttcgaggtcaagcctggcgtgtttaacaagttcttgatatcatatataaatgtaataagaagtttggtaatattcaattcgaagtgttcagtcttttacttctcttgttttatagaagaaaaaacatcaagaaacatctttaacatacacaaacacatactatcagaataca

SEQ ID NO: 223 - DNA, 574 bp, Saccharomyces cerevisiae
aacaatttttcttcaccacattttccattgttccttccccccataactataaacgtatttatgtatatatatttgcgtgtaagtgtgtgtactatagggcaccgtaaagtaataatgcttaattagttactactatgaccatataagaggtcatactgtatgaagccacaaagcagatagatcaatcatgtttaacgaaaactgttaatcgaagattatttctttttttttttctctttcctttttacaaagaaaattttttttgcgctttttgccatcaccatcgcaagttctgggacaattgttctctttcgctccagttccaaggaaagaggtttctgttttacttaatagaaagtgtcatcttgtattttatatctcttctttcttgtgtaaaattctttagttttgattttgtatttttaggacagtgagctacgaagtaacatttttacttaataaccgtttgaagcatagagcaggccctggtaccaccacctaatatctggctttttattcaataaaaactcaaaaaaaaaaatccaaaaaaaactaaaaaaccaataaaaataaa

SEQ ID NO: 224 - DNA, 700 bp, Saccharomyces cerevisiae
acgatggcttcttatctcacttcaatagtactttccaccggttatacttccggcttttccctattaatacaagctacaatttcaatgggtggcaaataatgtgtagaatagaaaataagccgacagggtaataaagaaaatttttagaaaaaaaaggttagatggcttatttaagttacaggctagcgaaaaaaggaacttcagggcaagtaaagtgtttgattgggcactagcatggcttataaaggcgagcaattgtcgaaactaattaatgttgtacggactattgctgtcttctcgtggtaaatgcgtgttccaggtcgaatactacttgcacacaggcgagcggggccccataaaagtgttgccgatttgttaagttgtcttttcggtttttctactctgttattccttacttccctttttaagaactctttttatccttcatttaggatcttgcacgtttccgcctcatcacttgaattaaaacatgtctctgtcagtaaaccttggcgtttctattgttcttcatagttcaacttttattattacccgccctgcgcgtttacatttttccagcaacagccagcgaaaaattagaaaatctggttgttgacacctcaagaacaagggcaattagcctcagcgtcgaatatagatcatattagaatacctatagctccatcaaaagaaatacaca

SEQ ID NO: 225 - DNA, 700 bp, Saccharomyces cerevisiae
ggatatcgtaaacaaaggcgttaccatagaaatgtactgattggcagaattactcttcaggagaatctttcatacaaaggaattccattggggaaaatctcgttaccaagtcaatgctgaactttctatggcctttgtttactatcgttaattattttacgaccacttctgggtagaaatatttcgtagccctggaacgagcttgtttacgcgttttatcccattatatggcacccaaatcaaatttaaaaagaaaaaacgcgtaaacagtgtcgggtaagttcatcctctgttactttaattgcttctttttttgaaattctaagtaaacgcgtcattttgatcctcaggacacagaaatccttgcagaatcttattgggtgttgaatagaggacgcgtaaaaacgatatggaaatttttttcatatagtgtagaaagaataggttggcgtaggtagtttcgtgtttgatagaaacctccaacaaagtctgcagctcacgttttagaataacaagtttagagtttatcttgttgcctttgttaagtcagtaccattgaataaaaattatataaaggagctaatatttcattgttggaaaattactctaccataattgaagcatatctcatccttttcatccttttcaacgcaagagagacaccaacgaacaacactttatttgttgatatattaacatc

SEQ ID NO: 226 - DNA, 367 bp, Saccharomyces cerevisiae
tcatatcccttctcacattttttacacaggaagtaagcaagttatgttatatttccgacactataattaattcttagcagttaaaggtgctttgtctatattacatttacatacagcttgagtgatcctgaccggatatagggtcctattttcttacgtgaacggcttttcttcttgttcccgatggccttcatgtgaaaaagcactcctcgggaggcggaaaaatatcaaaagtacggggcgaagtttataatgaagatttatcgatataaattttggttatttcaggagaacaagaaagctctttacactaaaattatcagagaagaagctgatatattagccctaaggagtacaccaaaacaca

SEQ ID NO: 227 - DNA, 700 bp, Saccharomyces cerevisiae
agtcgaacaagaagcaggcaaagtttagagcactgcccctccgcactcaaaaaagaaaaaactaggaggaaaataaaattctcaaccacacaaacacataaacacatacaaatacaaatacaagcttatttacttgacatcgcgcgatcttccactattcagcgccgtccgccctctctcgtgttttttgtttacgcgacaactatgcgaaatccggagcaacgggcaaccgtttggggaaagaccacacccacgcgcgatcgccatggcaacgaggtcgcacacgccccacacccagacctccctgcgagcgggcatgggtacaatgtccccgttgccacagagaccacttcgtagcacagcgcagagcgtagcgtgttgttgctgctgacaaaagaaaatttttcttagcaaagcaaaggaggggaagcacgggcagatagcaccgtaccatacccttggaaactcgaaatgaacgaagcaggaaatgagagaatgagagttttgtaggtatatatagcggtagtgtttgcgcgttaccatcatcttctggatctatctattgttcttttcctcatcactttcccctttttcgctcttcttcttgtcttttatttctttcttttttttaattgttccctcgattggctatctaccaaagaatccaaacttaatacacgtatttatttgtccaattacc

SEQ ID NO: 228 - DNA, 283 bp, Saccharomyces cerevisiae
tgtaccgttaccaattctcctcgtatatcacggttagtttttaaacctcggggtgacgtttactattggcgtactaatatattcttattttcttttcttttttgttggcagtttcaagcaacacatgtactggataaccaacccccgcacgctcttggaaaaaattgagaaggcatcggacacttgctgatgagtatttcgaaaaattccatgaagatgaggccaagattgtttggaagagattgaaaagaagaagaagaaaaaaagataaaagcaaatcaaa

SEQ ID NO: 229 - DNA, 694 bp, Saccharomyces cerevisiae
ccttgtgaaattgcaaatatggtgatttgaaacgtttcctagtgcagcaggatcacagataacgtgtaaagggcttagcagttgataatcctctctagttgagatctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagactagtacccggctgaatcacacctcgttaacagtgactttcggtgacagatacccgattgggcacccggctggtaagttatgatagaaagccaacgctgtactattggcttagctatggcaatattttgattatcaactagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaat

SEQ ID NO: 230 - DNA, 635 bp, Saccharomyces cerevisiae
ggctctgttgaacttttatctgagagaggaaaaaaaggaaggaaaaaaaagaagaaacttcctttatttatttgtcttaaccacaacacacaatgcaataagatgcaatataatatcaaagccaatatcttatgttgctgatcctgagaaggaatatatacaatttatgtagtaaaataccttttcttctgcgagttgcaagaaatagaaaagactccgattgcgcatcgccagaataaaatttcacaaccacactttttggctgaactttttattacctgattaaacagagagagaaaaggtagaggtcaaaattttttaagcaaaactaaaaaagatgcaaaatcacgtgctgaaaatctaacataagggttaagattagagttttataggacttgttttgtaatatttcaaatacgagctaaccctactgatttcaattaggtctaatttagggttgagctgcactgaaatttcggaaattttgggttattttaaatgagacagaagaactacagagatacgttcttcagactttaaagcttatctccacaaagaattggtcaagaaatcatcctagaaaaacacgtttgctcactcgatcttaatcacatagagtgctggaacgggaagaa

SEQ ID NO: 231 - DNA, 700 bp, Saccharomyces cerevisiae
gtgttgttatccgatacaaccggatatttttcttttaatgagtctaaaccgtgatagcttcaggttaatacaatcaaaaaaagctcaaatattcttttaatgccgcgttcacagattccaattgaatacaactaggtagttcattatatgaagcctttgctactatttttcactatagtctgccttcaccttaatgcagacatccacatattttaatcactttaaaataaaaaggaagatatattagaagctatgatccaatctgtaagccagattaaaattcacgaactcttctttcatttgaattgaatgctttgagttggggtagattatcgcaaattactcatcacatttattgactacgaacttgctgatgtcctttttttatttatatttttcttcagtgaagcgattttttttttacacagaccaagacggaaaaaagtagctaaggaagaaaacaaaatcatgaaaaaaatgtgaagtgatcatgcacatcgcatcaacttaaacattggcttagagatatatagagttagagtttacggcaacctttaagcaccaataccttttggcatagtctaaagacctggttcttaattttaaacaaatttaactaaagatttccctatcaaagaagtaacgagttgacagattttctcaaaataaatcgatactgcatttctaggcatatccagcg

SEQ ID NO: 232 - DNA, 700 bp, Saccharomyces cerevisiae
tctagtttctgccttaaacaaagccgcagccagagccgtttttccgccatatttatccaggattgttccatacggctccgtcagaggctgctacgggatgttttttttttaccccgtggaaatgaggggtatgcaggaatttgtgcggggtaggaaatcttttttttttttaggaggaacaactggtggaagaatgcccacacttctcagaaatgcatgcagtggcagcacgctaattcgaaaaaattctccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 233 - DNA, 531 bp, Saccharomyces cerevisiae
cacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaata

SEQ ID NO: 234 - DNA, 700 bp, Saccharomyces cerevisiae
tcctccacaaaggcctctcctggggtttgagcaagtctaagtttacgtagcataaaaattctcggattgcgtcaaataataaaaaaagtaaccccacttctacttctacatcggaaaaacattccattcacatatcgtctttggcctatcttgttttgtcctcggtagatcaggtcagtacaaacgcaacacgaaagaacaaaaaaagaagaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctccccttccttttctttttccagttttccctattttgtccctttttccgcacaacaagtatcagaatgggttcatcaaatctatccaacctaattcgcacgtagactggcttggtattggcagtttcgtagttatatatatactaccatgagtgaaactgttacgttaccttaaattctttctccctttaattttcttttatcttactctcctacataagacatcaagaaacaattgtatattgtacaccccccccctccacaaacacaaatattgataatataaag

SEQ ID NO: 235 - DNA, 700 bp, Saccharomyces cerevisiae
tacaaactccggttattttatcggaacatccgagcacccgcgccttcctcaacccaggcaccgcccccaggtaaccgtgcgcgatgagctaatcctgagccatcacccaccccacccgttgatgacagcaattcgggagggcgaaaaataaaaactggagcaaggaattaccatcaccgtcaccatcaccatcatatcgccttagcctctagccatagccatcatgcaagcgtgtatcttctaagattcagtcatcatcattaccgagtttgttttccttcacatgatgaagaaggtttgagtatgctcgaaacaataagacgacgatggctctgccattgttatattacgcttttgcggcgaggtgccgatgggttgctgaggggaagagtgtttagcttacggacctattgccattgttattccgattaatctattgttcagcagctcttctctaccctgtcattctagtattttttttttttttttttggttttacttttttttcttcttgcctttttttcttgttactttttttctagttttttttccttccactaagctttttccttgatttatccttgggttcttctttctactcctttagattttttttttatatattaatttttaagtttatgtattttggtagattcaattctctttccctttccttttccttcgctccccttccttatca

SEQ ID NO: 236 - DNA, 630 bp, Saccharomyces cerevisiae
ataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 237 - DNA, 700 bp, Saccharomyces cerevisiae
ttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 238 - DNA, 700 bp, Saccharomyces cerevisiae
aaaaggaaagattattgaaagagaaagaaagaaaaaaaaaaaatgtacacccagacatcgggcttccacaatttcggctctattgttttccatctctcgcaacggcgggattcctctatggcgtgtgatgtctgtatctgttacttaatccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaaatgcatattttttctcttggtaaatgaatgcttgtgatgtcttccaagtgatttcctttccttcccatatgatgctaggtacctttagtgtcttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcaca

SEQ ID NO: 239 - DNA, 700 bp, Saccharomyces cerevisiae
attggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaa

SEQ ID NO: 240 - DNA, 700 bp, Saccharomyces cerevisiae
ttactgaagaaggcatactacgccaagactattttattctcagcacttgtcctacgtggggttagaggcgagtaagactttggccccgctgcggaagcaaagagaattttgccatcggacatgctaccttacgcttatatctctcattggaatatcgttttctgattaaaacacggaagtaagaacttaattcgtttttcgttgaactatgttgtgccagcgtaacattaaaaaagagtgtacaaggccacgttctgtcaccgtcagaaaaatatgtcaatgaggcaagaaccgggatggtaacaaaaatcacgatctgggtgggtgtgggtgtattggattataggaagccacgcgctcaacctggaattacaggaagctggtaattttttgggtttgcaatcatcaccatctgcacgttgttataatgtcccgtgtctatatatatccattgacggtattctatttttttgctattgaaatgagcgttttttgttactacaattggttttacagacggaattttccctatttgtttcgtcccatttttccttttctcattgttctcatatcttaaaaaggtcctttcttcataatcaatgctttcttttacttaatattttacttgcattcagtgaattttaatacatattcctctagtcttgcaaaatcgatttagaatcaagataccagcctaaaa

SEQ ID NO: 241 - DNA, 700 bp, Saccharomyces cerevisiae
ggaaaaaatttttaggctcgagaacaatagggcgaaaaaacaggcaacgaacgaacaatggaaaaacgaaaaaaaaaaaaaaaaacacagaaaagaatgcagaaagatgtcaactgaaaaaaaaaaaggtgaacacaggaaaaaaaataaaaaaaaaaaaaaaaaaaggaggacgaaacaaaaaagtgaaaaaaaatgaaaatttttttggaaaaccaagaaatgaattatatttccgtgtgagacgacatcgtcgaatatgattcagggtaacagtattgatgtaatcaatttcctacctgaatctaaaattcccgggagcaagatcaagatgttttcaccgatctttccggtctctttggccggggtttacggacgatggcagaagaccaaagcgccagttcatttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaata

SEQ ID NO: 242 - DNA, 700 bp, Saccharomyces cerevisiae
tacttttccttcttattctatgaaaatttcatatcatcgttcatggtgatattaccacggtgacatcaaatggaattgtcctatttttcttttcctgtaattagtggcgcgggactttttattcctcgtggcaaagaatggctactaagaggagaataagagatggttagaagtatttggaagcttgatgacagaatattttaaaatataacttagcttgcgtgtgcgtatttgtattcttcgtggttacgatatgaaccaacgtgcaagcgtgtgatatatcacattccggagggtgtcaccccccctctctcaacacagtaatccataaaccagttttacatacacgtaaaaaagaacaggaataaagcttaatcggattattaactcatacgcttgtcacatattgttcgaacaattctggttctttcgagtttcgcagaactttttgaatttttcttttttttctagaacgccgtggaagaaaaacacgcgcatggttttatgagcggttaattctcatcttaataccaaccaggtccttccgccaccccctaaaacatataaatatgcagcttatcccttcaattcttaacatctgtgacctcctcatttcttcccgctgtattagagttcaagaaatcatacctgatagaaaatagagtcctataaacaaaagcacaaacaaacacgcaaagat

SEQ ID NO: 243 - DNA, 700 bp, Saccharomyces cerevisiae
gagaagaggtatacataacaagaaaatcgcgtgaacaccttatataacttagcccgttattgagctaaaaaaccttgcaaaatttcctatgaataagaatacttcagacgtgataaaaatttactttctaactcttctcacgctgcccctatctgttcttccgctctaccgtgagaaataaagcatcgagtacggcagttcgctgtcactgaactaaaacaataaggctagttcgaatgatgaacttgcttgctgtcaaacttctgagttgccgctgatgtgacactgtgacaataaattcaaaccggttatagcggtctcctccggtaccggttctgccacctccaatagagctcagtaggagtcagaacctctgcggtggctgtcagtgactcatccgcgtttcgtaagttgtgcgcgtgcacatttcgcccgttcccgctcatcttgcagcaggcgaaattttcatcacgctgtaggacgcaaaaaaaaaataattaatcgtacaagaatcttggaaaaaaaattgaaaaattttgtataaaagggatgacctaacttgactcaatggcttttacacccagtattttccctttccttgtttgttacaattatagaagcaagacaaaaacatatagacaacctattcctaggagttatatttttttaccctaccagcaatataagtaaaaaataaaac

SEQ ID NO: 244 - DNA, 325 bp, Saccharomyces cerevisiae
agtgtgagtcctctagtttttaccgcagccaccagccgcttctcgagcaaagtgtagatcccattaggactcatcattcatctaattttgctatgttagctgcaactttctattttaatagaaccttctggaaatttcacccggcgcggcacccgaggaactggacagcgtgtcgaaaaagttgcttttttatataaaggacacgaaaagggttctctggaagatataaatatggctatgtaattctaaagattaacgtgttactgttttacttttttaaagtccccaagagtagtctcaagggaaaaagcgtatcaaacatacc

SEQ ID NO: 245 - DNA, 494 bp, Saccharomyces cerevisiae
atagtttcagcagctctgatgtagatacacgtatctcgacatgttttatttttactatacatacataaaagaaataaaaaatgataacgtgtatattattattcatataatcaatgagggtcattttctgaaacgcaaaaaacggtaaatggaaaaaaaataaagatagaaaaagaaaacaaacaaaggaaaggttagcatattaaataactgagctgatacttcaacagcatcgctgaagagaacagtattgaaaccgaaacattttctaaaggcaaacaaggtactccatatttgctggacgtgttctttctctcgtttcatatgcataattctgtcataagcctgttctttttcctggcttaaacatcccgttttgtaaaagagaaatctattccacatatttcattcattcggctaccatactaaggataaactaatcccgttgttttttggcctcgtcacataattataaactactaacccattatcag

SEQ ID NO: 246 - DNA, 490 bp, Saccharomyces cerevisiae
taacgaaataatttccttttcttcctcttatatatgtatttatatatatgtgttcactataaccatacatatattatacagaagtatagacacgatttttccatacgtaacaaataacatcacgatgcacgagcactgtttccaatggagaacaagcgaaaggcggtaaatgcgctatgctgattggtgtagagcgcgggccaatgagaaggccccactctcagagggtgaggtgcgattatcggggacatgtctccgtcgatcgggcccacgtcgtcgcacaatataaataccatttgagtttttaatattaatttttcacgacaacccactcaaaaaagtatatattcactgttagttatacggaacattgcaatacatctacaagtcaaaggagcccctctatattatttatatttgtcgttgttcttttttgtgtgctcttttatacttcagaatcatacattaacatacatataagcaaatagcc

SEQ ID NO: 247 - DNA, 700 bp, Saccharomyces cerevisiae
tgttgatagcattctaccgtcacacttatctagcagatctttgttaggtagatctgctaatatattttacgtaagattcgttgtatgtgcgggaaaatttgtatctgttgtggtagtcactcaccctttctatgtattaaaaactgataccttccatactagaaaagacattcagttgaatccttctccaaatcaaggatactattgtcacttctccattgagattcgaaaaacccctcgggtcttgttagaactaaattacgttcataggggtgggatttatattgtaattccgcgaggtttacacgaaagatatctcaactctagccgcacatccattccggtatgtactctcccaccattgggtattatagaatgtaataggtttcaaagcggatatcttttgcccggtgagttgttactttttcattcgagcaatgaagtacattctagaagttcctagaaccttatggaagcaccaagaaaaaaggaagttaaacaaaacactgattcaataagcaaggggggaagctccttagtttgacgacagtaacaaaatgttcgtataaattgaacgaaactcaagccaataaaggacttttcagaggcctatctcttctttctccacaactttcgaataaaaaccactaataaaaagtaaataacaaaaacaagaaaaaaaataaacaaaacaataatc

SEQ ID NO: 248 - DNA, 822 bp, Komagataella pastoris; identical to SEQ ID NO: 163

SEQ ID NO: 249 - DNA, 424 bp, Komagataella pastoris
ataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttttttttcacgaaaattttttttttacacaagattttcagcagatataaaatggagagcaggacctccgctgtgactcttcttttttttcttttattctcactacatacattttagttattcgccaac

SEQ ID NO: 250 - DNA, 750 bp, Komagataella pastoris
ctggggctctaactcgagcaagtgtcctatgctgtaggccgcagcccttttggttcgacgacgtgcgtggttatgagacgctcggctgttttgcgctaagctggccgtatcgagtaaattctacaggcacctgcgaggcaagcatctactaatgtttatttttcgtccaacctaattgtggtttcaaagcgctatcaggtggggggtaagaggaatgtgagtggaaagcgaaaataactggcagctggggtcagatcccgtgatgccacctcttgtggtattttgaaacgcgtgttgcgattggccgcgagaacggaaaggaatatatttactgccgatcgcattttggcctcaaataaatcttgagcttttggacatagattatatgttctttcttggaagctctttcagctaatagtgaagtgtttcctactaaggatcgcctccaaacgttccaactacgggcggaggttgcaaagaaaacgggtctctcagcgaattgttctcatccatgagtgagtcctctccgtcctttcctcgcgcctggcaataaagcctccttcggaggagctccgtctagagaataattgctgcctttctgactttcggactagcgccaaccgcgaaccacaccaccacaccatcactgtcacccgtcatagttcatccctctctccttataaagcatctaataggttccacaattgtttgccacaaaaatctcttagcatagcccaattgattacgaaa

SEQ ID NO: 251 - DNA, 535 bp, Komagataella pastoris; identical to SEQ ID NO: 165

SEQ ID NO: 252 - DNA, 250 bp, Komagataella pastoris
aacgtatcttagcatggttgtgcgacagattcactgtgaaagactgttcattatacccacgtttcactgggagatgtaagccttaggtgttttaccctgattagataatacaataaccaacagaaatacgagaatctaaactaatttcgatgattcatttttctttttaccgcgctgcctcttttggcaattctttcacctatattctaccttctctttccttttgttctaaacttattaccagctatct

SEQ ID NO: 253 - DNA, 558 bp, Komagataella pastoris
gatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttt

SEQ ID NO: 254 - DNA, 700 bp, Komagataella pastoris
ttgctaatttttttcccgcgcagcagaagaggcgttttcatcgtatcgtggtaacagaatggtcagttgatgtgccgtacaggcacatggcagcagtcactatttatttttttaaccttaaagtcgtccattaatcattacctgaccaatcagattttctgtatttgccacttatctaaaaatacttttgtatctcgcagatacggttcaatggtttccaggacaacgccagaaaaaagaagtatcaatgccaccaggcaagtcggttttatgtttggtagtcccacgcaaaggagcacccacctctttaaagttgtggttaaccacctgaagctccggaataaactactggctgtgaccacaacagtccaatgatgcagaatgctaatttaaacgagtgccaaatcaggattttcacagataaatcaatcgatccagagttacccattccagccttttctgggtcgagcttgcttcattcttttctcaggtgcatacgtctgcatgaacaatccagattagggcagattttgagtttaaattaggaaatataaacaaatataccgcgaaactggtttgtttatagcttttcgcccactgccgtatggtataaatacatactcttcttcccccctggttctcttcttcttttgttacttacattttaccgttccgtcactcgcttcactcaacaacaaaa

SEQ ID NO: 255 - DNA, 700 bp, Komagataella pastoris
ttccttgagcagaacggatcttcgagataagcccggagaaatattcgcgaatatgaaacagttagtgtgttatagtatatttattataatattgacaaggaagacggaggtttctgggttggaaatgggattgaatggattatttggagttgaccatgataaataaatattaattgtcaaaatgtcataagcagcccatcaacgcagaacacagccacttgagccccaaacataactaatactctccacccaacatcgtcagatctattacataatcccttttggatcgcctaaaccagcctgaatttgcccgaatttgccctggcttgcctaatatttatgtctcgcgacataaccctaaacatgaatttaagactaccagacaaaaccgatctccaccatctctcgcccgttcctctcagaccgttggcggaaaggctctccttctttctgaagcacatcggctaacaaatcgacaagaaagttccattgagttccacgcccgtttcagcgtcatagggccgttctctgtagtattgttgtacatgcaatcatgcccctccaatagccggatttctacgcgacctttcatttaaggagcgtcatgtcatttcctcctaagtatataaatagcttcatccccctgtggctttgtttattctagccctctcatttactacaccaattcaattgataatca

SEQ ID NO: 256 - DNA, 637 bp, Komagataella pastoris
attgaatataaagaacagaggcccagttcagcagttcagggttgatatatgaattaattatgtaatctgaactagtttggatttgggagcctgaattggcaaaagaaaccaaaaggtaccttccgagggtactttcgaccccacttacgagggcggatatgtgtcctcctgatgtctaattacgtcatagaatgttgatgcctagaccttttcacttccgtagtaatttattcgttggaaagaggcaaggtaaattattcaatccggcagtcatgagccaatactatctgaatttgaacgaactcttggtcaaattatccccgttgtagggcacactttttctcgtcttcattcagcccccattgcagtaaaccccaactttacattccggaaaaaaaatgctcctccacctggacgcagagtgtaccgctcaaagaaccctcgatgagtttcgaccatcttgacaaagtgaaataatatttatagtgagggaaagaacactttttcctccactagtttctttaccagttaactactacagaatcttccaaaatgaccaaaacattcttcgacgtttcttccaacggtatgtattgagattgaattatccgcactgcgcagcagagaccctaacacc

SEQ ID NO: 257 - DNA, 700 bp, Komagataella pastoris
tctgctaaaattggtcatattagtcaagctgattactaaataactattgaagacccggtattctcgtagacatagtgatttatatattaatgatgcaagtctttctcctacatcatcctgggcagctttgagtgacaagtaactaaaactacaatcttatcagctttatacgattgagactattcccagagttgttccagtgaaactgattgtgtctttacattaccgtaattttccaagcttcgttcctcatggcccaatggtcaaggcgtctggctacgattagccaacttcgagcaaactcggggtaataggaaaccagaagattccaggttcgactcctggtggggaagcttctttttttcttggctggatcgggttaggactgaggctcattgatctgtcatcatcaaattccactcccagatacgctacccatctgaaaccctctttacagcttcagtaccattaccactaccttccataaaccatctagcttccagtaattagtaaacatctcatttatgaccaattttttcaactccactcaccccgattacctaatctcatttagtgtgattagtaactaaagaatctcgaacaaccgatggccaacatgcttataagcggcacaccatcccactcctgaattctcaattcaatttcctgttttataccacagtgctgaaacaaacaaaca

SEQ ID NO: 258 - DNA, 283 bp, Komagataella pastoris
agaaaaatatgtatgcattaaattagatttcagtttaattcattaagagctttctgaaagtaccaacttttctgtctctcgacatccaacaacctgaatggtgttggctagaaaaatataatgtgaagtctgtcacatattcgctgacattcggaacgacgctttgatctcctttaaccaatcaaattactgttacgtaatccccgatcttctccgttatcccgtgaacaacttcctccccatattacgctcagttttttcgatatctctataaacaccaatg

SEQ ID NO: 259 - DNA, 595 bp, Komagataella pastoris
gctaaaggtgatgttagtaaaggaggcttcgggaaaaaaaaaatgggaaaaaaaggaaggaagaaatactggaacttaaaaaatcgaagcaatcatcggaaaactatcacttcgttgcttaacggtcatttcacttgaatcgtagcaatttttaaatcgtgtctttgtagccactagacgaaatctcccgtttgctatgtttgtttatactacttcgccacccagcagcacaatggatcagggtaagctcacctgtttttcgaaagcccaatcatccttctcaccttacacttgaactactcacttttaaactcaaattaatccgtagttactcaattaaaaaataattctttagaacctttgataataagtacgtgtgaacgtattcgcaaacgaatctgaggttcaacaaaggcccatattgttttgattggtacaccactatggaaatcatcagaaagaaataagaaatttcgacagccaccgttacccggagggagataaataaatcatctatttccacttcactaaatactactatttatggtggtgtttaggtgcaatgtatattctctattattacatggtgttttaa

SEQ ID NO: 260 - DNA, 370 bp, Komagataella pastoris
ttttactacgatagacagaaggaaagtaacagtgggagggagaggatctggatatttataagagtttcgcatagataacgatttggcactttttgtcagcaatgccaacagtatttcgcactgcgacactcccgactgaaacgggatgcaactttactatgacttttggtggcatagaaatgggacctgtgtttacagtttcaaatttacacacggcctaccacaaagagaacggctccagttcatcgcgtaccgtgtcgtaccaacattattgtttggcactgcatagtcgaccttaactcgtttcctctataaatacaagacgagtacgtcttttttagactcatccgtaaacaaataatcaataaat

SEQ ID NO: 261 - DNA, 265 bp, Komagataella pastoris
gcactgggtaacatttgaggatccttatcaacataagggcttgacatcgaaaatgaggtctacttctcaaagtcaaagaaggaaaaaaaaagaagcatcacatgatcaaggcaagggctacaaagcgtggtgtagagattgactactggatagcaggttttgctagcttaccagactagtattattcgcgctcctcgattccaagctacaagttgattaaacttttgttctgaaaagttttcctaaacaaaaaaggttacacaag

SEQ ID NO: 262 - DNA, 647 bp, Komagataella pastoris
tgattagaagtttaatgagggagtgaccactctaagcaagcaaaatgagatcgaagcaaaaaataaagtaaagcaggaattctggtacgcttagatcaaagcgatttagtttgagatggctggttttttccttagtcttgggaaatgaggtttgtgtgggcttcagcgaatattccccgatggggcaaattagttgcattgcaactaagagaacacgacatggtgatctgtggtaacaatctgggtcgtgccctctctgataatgatgatggtgttcgggcaacctcgaagatcggtgttcagtgagtgaaggaaggccaccagcgatcccagctatgatgtaatggaaattgctaagcaattgtgaaatgtgatgtatttggcagagcaattgtgcggtgcaacggggtgttattgtctccaggaggcattattgcgaattttcgattgaaaatggatattttagcacagggtgcttgacccctattgttgctcgctaaaccatgattgctaaatgatgacatagcaatcactctcctaaggttgctacaaggggatcattcttagtataaatggacactcttttcccctgatagctccttttatttttcacacttaaacagttacaaacacaaacacaaatagaa

SEQ ID NO: 263 - DNA, 700 bp, Komagataella pastoris
tgaatgctcctttctatattcaataggtgtttttagcacctttgcatgcactccgtggccaatagcgcgtttcatacgcactttaatcccctcctttgttgagtgcaatctctgtgagatttggtggggtgcgaaccgttagacgcagcacgttaacataaaactcctgaacaagctgcagatagtcttcaagatcctgatgattctcactctttggtataatgtttattgactgaatgtaaactttaacctaaaacatgtatctccgctattttttttttttttttttgatgaccctaattctcgtgacaaattaatttccagcaggggtttgtcccgataagagaattttttaaattatccgttcggataaatggacgtctactccatatttttccgtatattaccccacctggaagtgcccagaattttccggggatttcggataatacggtggtctggattaattggtgcgccaagtcttctattttgttgcagtctcgtgtgagtatgtgcaacaacaaacaagatgagccaatttattggattagttgcagcttgaccccgccatagctaggcatatccaagtgctatgggtgttagatgacgcacttagatgctgtgagttttggagtataaaagatccttcagattccacgcttatttcctcagtttttcaaaacaaccaagaagtatcatg

SEQ ID NO: 264 - DNA, 393 bp, Komagataella pastoris
tggtccctgaggtattccaggaagtttggagtcatcaactgtccctcgtaactggcatagcattcaccaactagataatggtgtatggatgtaacacaacaatttaatgttgtaaatcaacctaaggattgagtccggagatgctattgtcatccaaatctattggtaaatgctgcagttatatcatcgatagaggtctaaagattaacctattgttggtaaataaccccattacccgtttacgtagcaaggttcaaagttgtgctcagatcggagccaaaagttcgactgacttctttcgaaaatgtggattatgcaagcaacgttgctatctgtatagtatataaggtcgatctgccccatgccaaattgtaaagcaacaccctatacaaa

SEQ ID NO: 265 - DNA, 700 bp, Komagataella pastoris
agagtattgactatctgcccatattgaaggtatttatccagccgtactcttctttgacccatagtggaatggatctctccggtagcagaagcgataaacagatattgcgtgttttgcgttatcagtcaaggacgctcgggattgaagacgggaacgcgcgattttctagtggctagatagagagggccaaagagctatggaggtgggtgatagggtgacaaaaagtacgagaaggcatggataagcataagtctagatggtttagcgcaaatctcagcataaaaaaaaatcgatcttataatctaccagtgcataggacgctgatcatctatgttctcacaactaaacagtgcgcttccggaacaaattcccgattcatattgctggcatattgtggaaaaatgattactcgattcggatgctcgctagctaatactagctaattccgtatgcaagttcctggtacccgcgtactaaaatggaatttgtattgtttgcagatgaaactccctggcaataagcaaacaaccagagtaggtacgtgcaaagctattgttttaagctttggacatgtgtagggataaggttgatgcggtgatgggctgaccagctcgtttctcctcaaattattccctagcaacacaataacatactataaataggacggaaagtactgttcggctgccggtttattgttggc

SEQ ID NO: 266 - DNA, 302 bp, Komagataella pastoris
cccttacaaccaagcttcagtgttgtttcagcacttctctctacgtcgtataaatttcacagacttcgcagtaacagtagaatctagatgagggggggacaattccaaattgggtaagcacaacctgcacagtgttgtggtgtgaggaagatcttagaaaataatcacctgtattgttggttgtgtggttgctatgatgtacgagcaatgccagtgagtacttttccgacaagtataaatagagcagagggttggcccctgaaatcgatttgtggaataatcacagctcaatatacaaataa

SEQ ID NO: 267 - DNA, 1001 bp, Komagataella pastoris
cgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgttccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagcttctagcttttatcttattttacttaaagaaa

SEQ ID NO: 268 - DNA, 822 bp, Komagataella pastoris
caggtgaacccacctaactatttttaactgggatccagtgagctcgctgggtgaaagccaaccatcttttgtttcagggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatatatcgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 269 - DNA, 481 bp, Komagataella pastoris
cccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgttcattagaaagaaagcatagcaatctaatctaagggcggtgttgacaattaatcatcggcatagtatatcggcatagtataatacgacaaggtgaggaactaaacc